(19) United States
(12) Patent Application Publication
(10) Pub. No.: US 2024/0058455 A1
(43) Pub. Date: Feb. 22, 2024

(54) BIOORTHOGONAL LINKERS AND REACTIONS

(71) Applicant: The General Hospital Corporation, Boston, MA (US)

(72) Inventors: Jonathan Carlson, Boston, MA (US); Hannes Mikula, Pixendorf (AT); Ralph Weissleder, Peabody, MA (US)

(21) Appl. No.: 18/247,571

(22) PCT Filed: Oct. 4, 2021
(86) PCT No.: PCT/US2021/053439

§ 371 Date: Mar. 31, 2023

Related U.S. Application Data
(60) Provisional application No. 63/104,947, filed on Oct. 23, 2020.
(60) Provisional application No. 63/087,087, filed on Oct. 2, 2020.

(51) Int. Cl. A61K 47/54; A61K 47/68; A61K 49/00

(52) U.S. Cl. CPC A61K 47/545; A61K 47/54; A61K 47/6849; A61K 47/6889; A61K 47/542; A61K 49/0041; A61K 49/0052; A61K 49/0058

(57) ABSTRACT

The present disclosure provides bioorthogonal linkers and reagents, including trans-cyclooctene (“TCO”)- and tetrazine (“Tz”)-containing compounds. In a general aspect, the present disclosure provides reagents, conjugates, and bioactive molecules containing a trans-cyclooctene (“TCO”) fragment. Examples of TCO fragments include: Formulae (I) and (II).

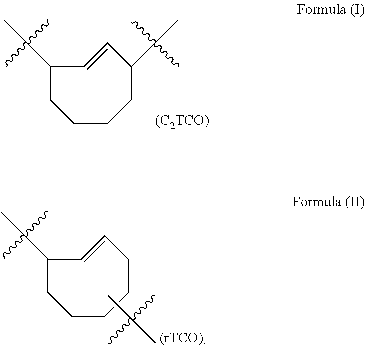

### CLAIM OF PRIORITY

This application claims priority to U.S. Provisional Patent Application Ser. No. 63/104,947, filed on Oct. 23, 2020, and U.S. Provisional Patent Application Ser. No. 63/087,087, filed Oct. 2, 2020, the entire contents of which are hereby incorporated by reference.

### TECHNICAL FIELD

This invention relates to bioorthogonal linkers and reagents, including trans-cyclooctene (“TCO”)- and tetrazine (“Tz”)-containing compounds.

### BACKGROUND

Biologically compatible click chemistries have become powerful tools for the design, synthesis, and tracking of labeled (bio)molecules. Among these chemistries is the inverse electron demand Diels-Alder (“IEDDA”)-initiated reaction of tetrazines (“Tz”) and trans-cyclooctenes (“TCO”). FIG. 1A provides an example of fluorogenic “turn-on” Tz-TCO reaction useful in no-wash cellular imaging. FIG. 1B provides another example, where the amino-group containing compounds functionalized with trans-cyclooctene modified in allylic position (release-TCO, “rTCO”) are released in their active form upon reaction with tetrazines. This latter example allows for prodrug activation and targeted drug delivery via Tz-triggered release of the active compound from cleavable TCOs, as well as methods to activate rTCO-caged biomolecules in live cells. However, methods and reagents that are currently available are unable to achieve both fast (i.e., seconds to minutes) and complete (>99%) cleavage and release of probes (bio)molecules from their TCO conjugates.

### SUMMARY

The present disclosure is based, at least in part, on a realization that bioorthogonal cleavage/release reactions are useful for controlled inactivation of probes or (bio)molecules in biological environments. In contrast to the “turn-on” (or activation) applications of the prior art (See FIGS. 1A and 1B), the compounds and methods within the present claims may be advantageously used to “turn-off” (or inactivate) molecular probes and (bio)molecules, providing useful deactivation strategies for chemical biology and biomedical research.

FIG. 3 (top section) provides a schematic example showing that bioorthogonal cleavage of fluorescent probes enables superior methods for applications that require the removal or deactivation of the dye after analysis, such as multiplexed imaging. Compared to other methods using either (i) harsh destaining/quenching conditions, (ii) complex DNA-based cycling and imaging techniques, or (iii) the combination of clickable fluorophores and quenchers, bioorthogonal cleavage methods and compounds within the present claims allow rapid, gentle, and biocompatible removal of the dye by using small molecules (e.g., TCO and Tz) as “chemical scissors.” FIG. 3 (lower section) also provides a schematic example showing that bioorthogonal cleavage of biologically active (bio)molecules containing a cleavable fragment in their structures using “chemical scissors” leads to disassembly and of the (bio)molecule and its “inactivation” by loss of its biological function. An example of the “chemical scissor” reaction schematically illustrated in FIG. 3 is shown in Scheme 1.

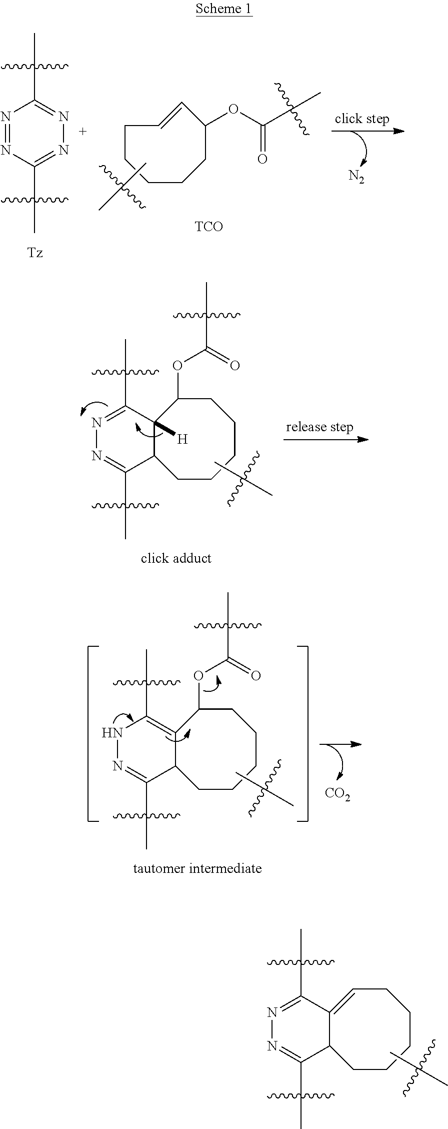

Referring to Scheme 1, a Tz reagent reacts with a probe or a (bio)molecule containing a TCO fragment in its structure, thereby forming a “click” adduct during the initial “click” step of the reaction. During the second (release) step, the click adduct first tautomerizes to form a tautomer intermediate. The tautomer intermediate then undergoes spontaneous decomposition reaction leading to molecular disassembly and deactivation of the molecular probe or the (bio)molecule.

More broadly, the incorporation of cleavable fragments (such as cleavable TCO linkers) into probes and (bio)molecules enables molecular inactivation of those probes and (bio)molecules for spatiotemporal control of their biological function. Importantly, the methods and compounds within the present claims achieve both fast (i.e., instantaneous, or seconds to minutes) and complete (>99%) cleavage of probes and (bio)molecules from their cleavable conjugates.

Unlike the target activation (or “turn-on”) approaches of the prior art (See FIGS. 1A and 1B), where a relatively broad range of yields (e.g., from slow and incomplete TCO cleavage reactions) may nevertheless generate discernible signal and/or relevant downstream activity, the combination of fast and complete cleavage/release is essential for “turn-off” (or inactivation) strategies to reach minimal residual signals and/or function within a useful timeframe. The compounds, reagents, and methods within the present claims allow for fast and complete bioorthogonal reactions which successfully enable the “turn-off” (or inactivation) applications. What is more, the reagents within the present claims provide independent control of click rates (first, click step) and cleavage rates (second, release step), which increases their utility in multiple applications, and provide rapid reaction kinetics to facilitate the cleavage reactions even at very low concentrations.

In some embodiments, the present disclosure provides a compound of Formula (I):

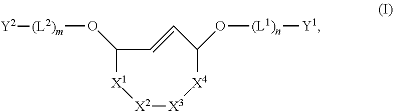

or a pharmaceutically acceptable salt thereof, wherein:
X¹ is selected from O and CHR¹;
X² is selected from O and CHR²;
X³ is selected from O and CHR³;
X⁴ is selected from O and CHR⁴;
R¹, R², R³, and R⁴ are each independently selected from H, halo, C₁₋₆ alkyl, C₁₋₆ haloalkyl, C₁₋₃ alkoxy, and C₁₋₃ haloalkoxy;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000;
each R^{N} is independently selected from H, C₁₋₃ alkyl, and C₁₋₃ haloalkyl; and
each Y¹ and Y² are independently selected from H, NH₂, OH, C(O)OH, a protected amino group, a protected carboxyl group, a protected hydroxyl group, a reactive chemical group, a fluorophore, and a fluorescence quencher.

In some embodiments, the compound has a formula:

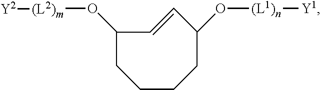

or a pharmaceutically acceptable salt thereof.

In some embodiments, the compound has a formula:

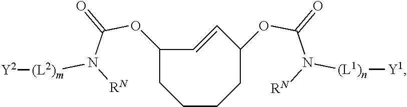

or a pharmaceutically acceptable salt thereof.

In some embodiments, the compound has any one of the following formulae:

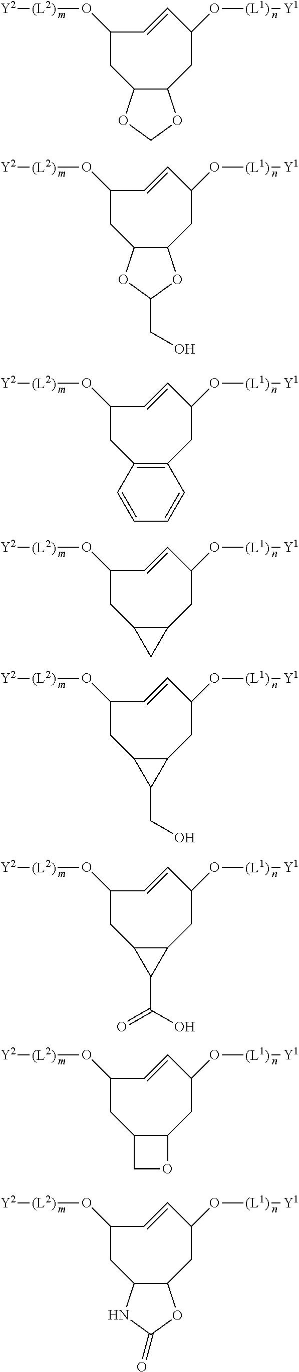

or a pharmaceutically acceptable salt thereof.

In some embodiments:
n is an integer from 1 to 10;
m is an integer from 1 to 10;
Y¹ is selected from NH₂, OH, C(O)OH, a protected amino group, a protected carboxyl group, and a protected hydroxyl group; and
Y² is selected from NH₂, OH, C(O)OH, a protected amino group, a protected carboxyl group, and a protected hydroxyl group.

In some embodiments:
n is an integer from 1 to 10;
m is an integer from 1 to 10;
Y¹ is selected from NH₂, OH, C(O)OH, a protected amino group, a protected carboxyl group, and a protected hydroxyl group; and
Y² is a reactive chemical group.

In some embodiments:
n is an integer from 1 to 10;
m is an integer from 1 to 10;
Y¹ is a fluorophore; and
Y² is a reactive chemical group.

In some embodiments, the compound is selected from any one of the following compounds:

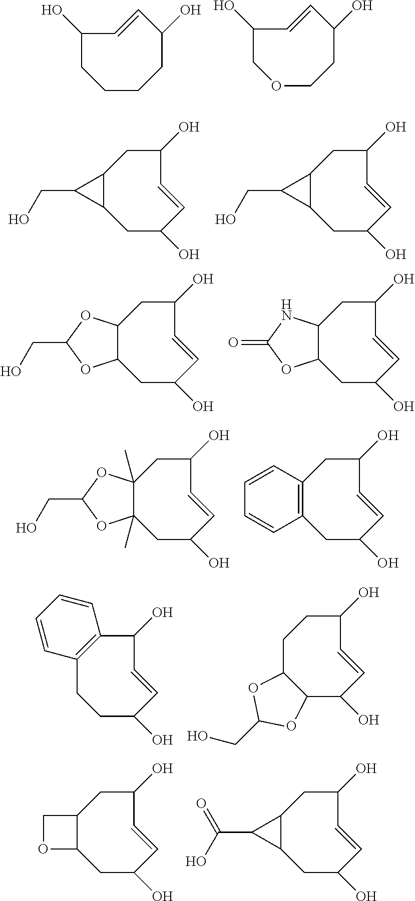

or a pharmaceutically acceptable salt thereof.

In some embodiments, the compound is selected from any one of the following compounds:

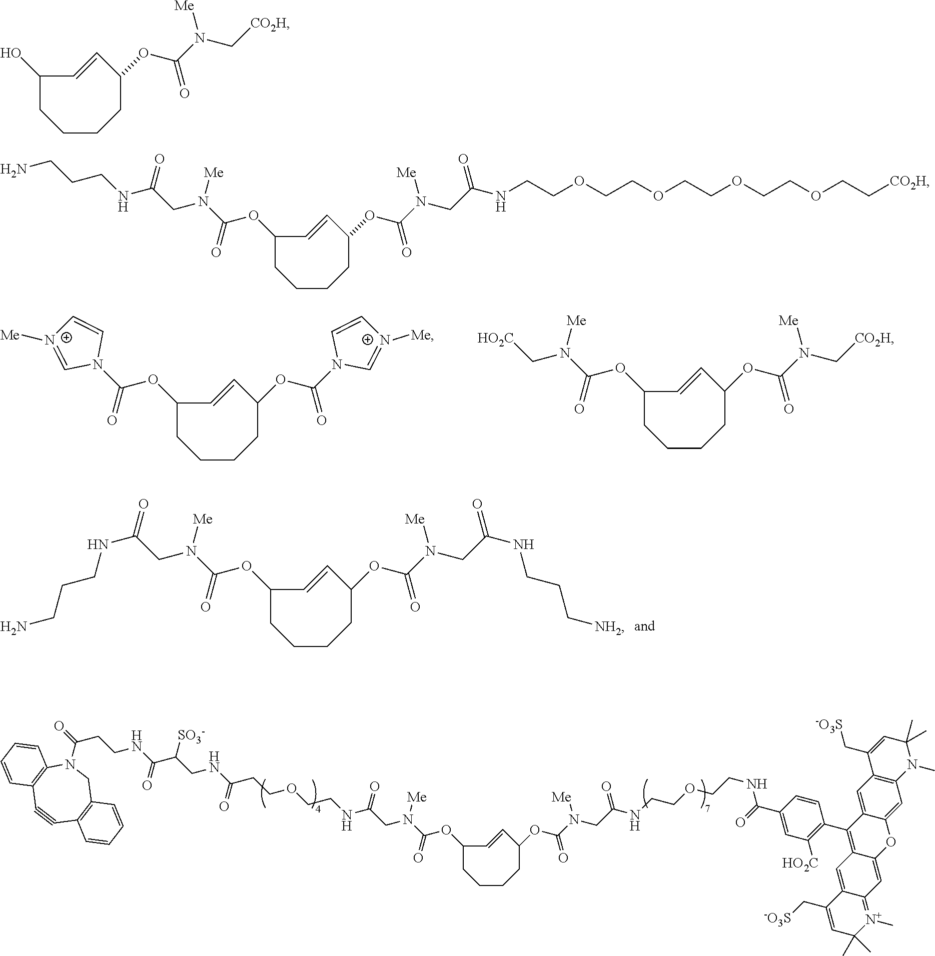

or a pharmaceutically acceptable salt thereof.

In some embodiments, the compound is selected from any one of the compounds depicted in FIGS. 37A-37D, or a pharmaceutically acceptable salt thereof.

In some embodiments, the present disclosure provides a compound of Formula (II):

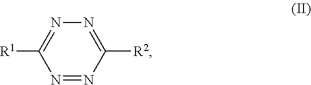

or a pharmaceutically acceptable salt thereof, wherein:
R¹ and R² are each independently selected from H, C₁₋₆ alkyl, C₆₋₁₀ aryl, 5-6-membered heteroaryl, L₁-W, and L₂-OH, wherein said C₁₋₆ alkyl, C₆₋₁₀ aryl, and 5-6-membered heteroaryl are each optionally substituted with 1 or 2 substituents independently selected from halo, C₁₋₆ alkoxy, C₁₋₆ haloalkoxy, a reactive chemical group, and L₃-X;
L₁ is C₁₋₃ alkylene, optionally substituted with 1 or 2 substituents independently selected from halo, C₁₋₆ alkoxy, C₁₋₆ haloalkoxy, a reactive chemical group, and L₃-X;
W is selected from OH, NH₂, and C(O)OH;
L₂ is C₆₋₁₀ arylene, C₃₋₁₀ cycloalkylene, 4-7-membered heterocycloalkylene, and 5-6-membered heteroarylene, each of which is optionally substituted with 1 or 2 substituents independently selected from halo, C₁₋₆ alkoxy, C₁₋₆ haloalkoxy, a reactive chemical group, and L₃-X;
L₃ is a linker;
X is selected from a fluorophore, a fluorescence quencher, a targeting group, a reactive chemical group, and a biologically active molecule, or a fragment thereof;
provided that
at least one of R¹ and R² is selected from L₁-W and L₂-OH, and
the compound of Formula (II) is not any one of the following compounds:

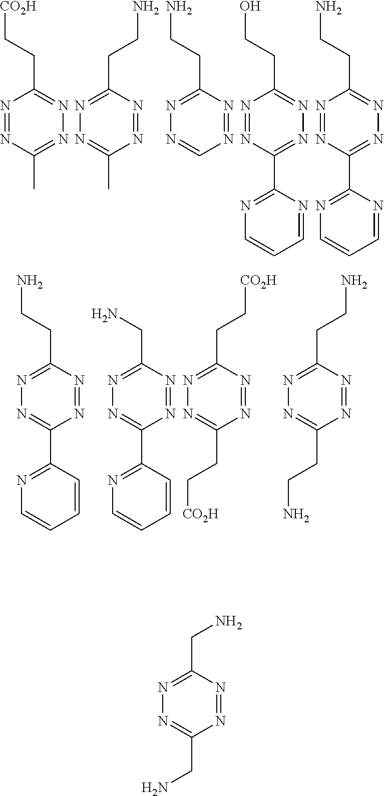

In some embodiments, the compound has a formula:

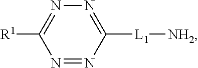

or a pharmaceutically acceptable salt thereof, wherein:
R¹ is selected from C₆₋₁₀ aryl, L₁-OH, L₁-C(O)OH, and L₂-OH.

In some embodiments, the compound has a formula:

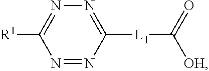

or a pharmaceutically acceptable salt thereof, wherein:
R¹ is selected from H, C₆₋₁₀ aryl, 5-6-membered heteroaryl, L₁-NH₂, L₁-OH, and L₂-OH.

In some embodiments, the compound has a formula:

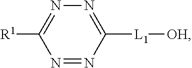

or a pharmaceutically acceptable salt thereof, wherein:
R¹ is selected from H, C₁₋₆ alkyl, C₆₋₁₀ aryl, L₁-OH, L₁-C(O)OH, L₁-NH₂, and L₂-OH.

In some embodiments, the compound has a formula:

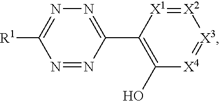

or a pharmaceutically acceptable salt thereof, wherein:
X¹, X², X³, and X⁴ are each independently selected from CH, N, and CR³; and
each R³ is selected from halo, C₁₋₆ alkoxy, C₁₋₆ haloalkoxy, a reactive chemical group, and L₃-X.

In some embodiments, the compound has a formula:

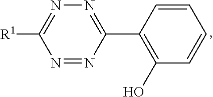

or a pharmaceutically acceptable salt thereof.

In some embodiments, the compound has a formula:

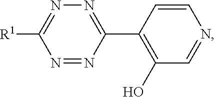

or a pharmaceutically acceptable salt thereof.

In some embodiments, the compound has a formula:

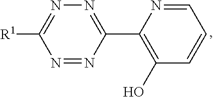

or a pharmaceutically acceptable salt thereof.

In some embodiments, 10 is selected from C₁₋₆ alkyl, C₆₋₁₀ aryl, and 5-6-membered heteroaryl.

In some embodiments, the compound has a formula:

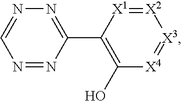

or a pharmaceutically acceptable salt thereof.

In some embodiments, the compound has a formula:

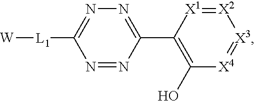

or a pharmaceutically acceptable salt thereof.

In some embodiments, the compound has a formula:

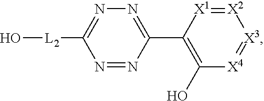

or a pharmaceutically acceptable salt thereof.

In some embodiments, the compound is selected from any one of the following compounds:

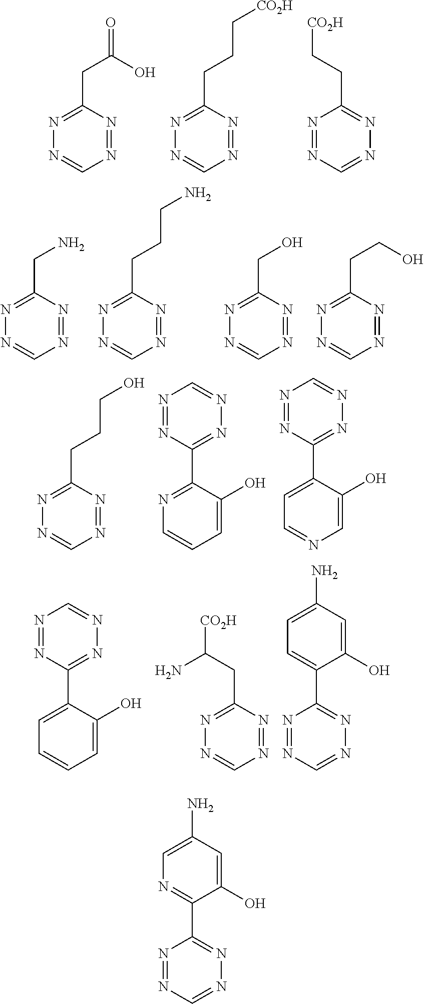

and any one of the compounds depicted in FIG. 36B, or a pharmaceutically acceptable salt thereof.

In some embodiments, the compound is selected from any one of the following compounds:

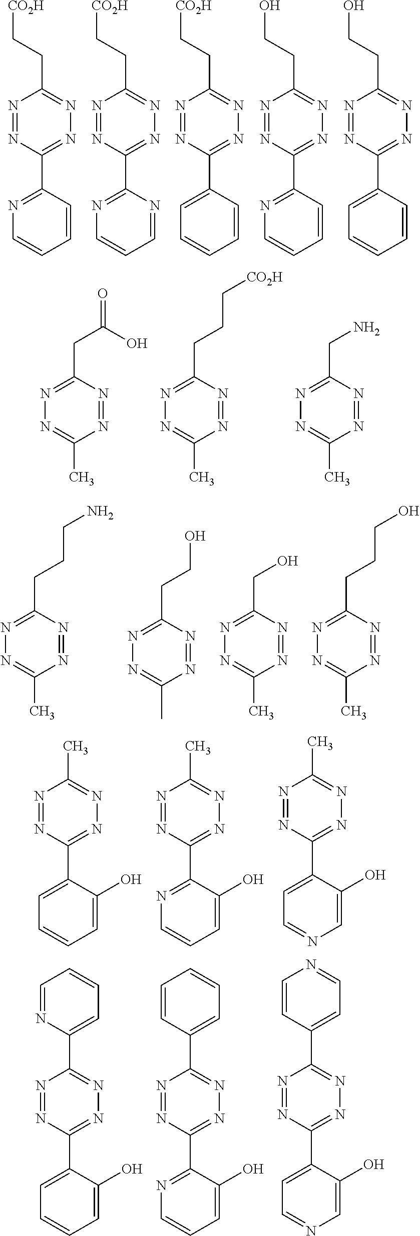

or a pharmaceutically acceptable salt thereof.

In some embodiments, the compound is selected from any one of the following compounds:

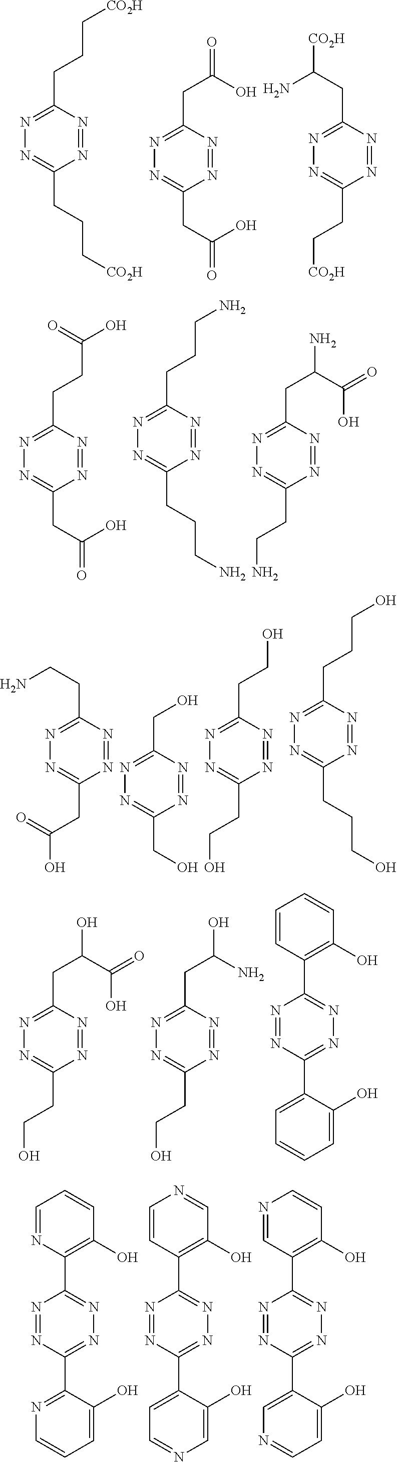

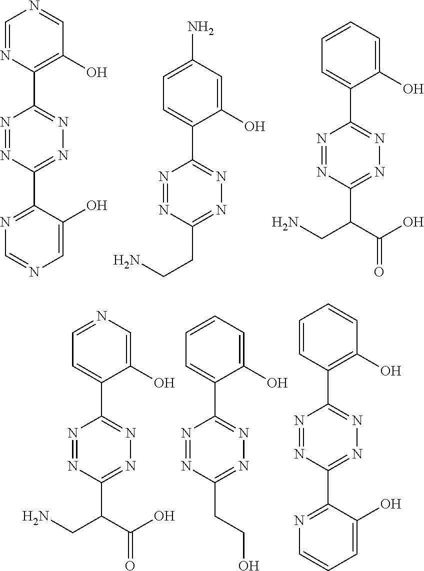

or a pharmaceutically acceptable salt thereof.

In some embodiments, the present disclosure provides a conjugate of Formula (III):

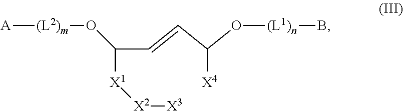

or a pharmaceutically acceptable salt thereof, wherein:
X¹ is selected from O and CHR¹;
X² is selected from O and CHR²;
X³ is selected from O and CHR³;
X⁴ is selected from O and CHR⁴;
R¹, R², R³, and R⁴ are each independently selected from H, halo, C₁₋₆ alkyl, C₁₋₆ haloalkyl, C₁₋₃ alkoxy, and C₁₋₃ haloalkoxy;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000;
each R^{N} is independently selected from H, C₁₋₃ alkyl, and C₁₋₃ haloalkyl; and
A and B are each independently selected from an antibody, an antibody fragment, an engineered antibody, a peptide, a biologically active protein, a small-molecule drug, a fluorophore, and a fluorescence quencher.

In some embodiments, the conjugate has a formula:

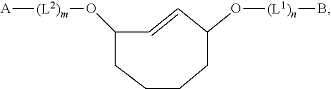

or a pharmaceutically acceptable salt thereof.

In some embodiments, the conjugate has a formula:

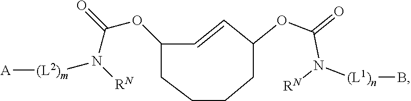

or a pharmaceutically acceptable salt thereof.

In some embodiments, the conjugate has any one of the following formulae:

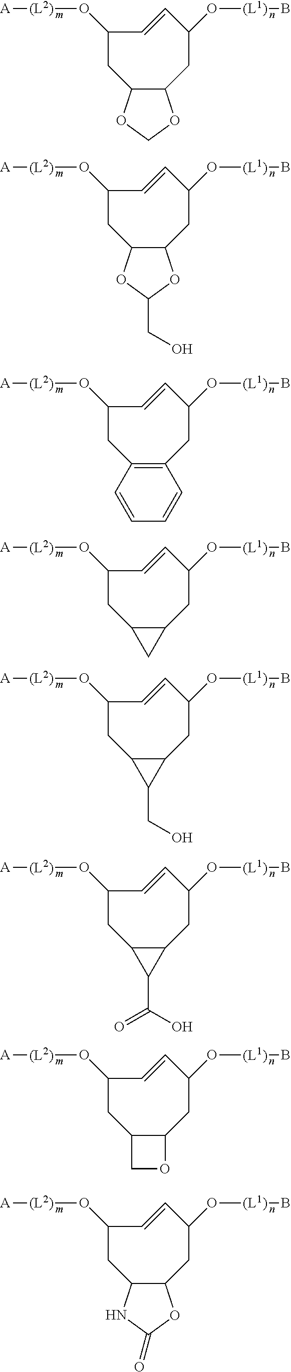

or a pharmaceutically acceptable salt thereof.

In some embodiments:
n is an integer from 1 to 10;
m is an integer from 1 to 10;
A is selected from an antibody, an antibody fragment, an engineered antibody, a peptide, a targeting moiety, and a small-molecule drug; and
B is selected from a fluorophore and a fluorescence quencher.

In some embodiments:
n is an integer from 1 to 10;
m is an integer from 1 to 10;
A is selected from an antibody, an antibody fragment, an engineered antibody, a peptide, and a targeting moiety; and
B is a small-molecule drug.

In some embodiments:
A is an antibody that is specific to an antigen which is a biomarker of a disease or condition; and
B is a fluorophore.

In some embodiments:
A is an antibody that is specific to an antigen which is a biomarker of a disease or condition; and
B is a small-molecule drug useful in treating the disease or condition.

In some embodiments, the conjugate is selected from any one of the conjugates depicted in FIGS. 16, 17, 19, and 36A, or a pharmaceutically acceptable salt thereof.

In some embodiments, the present disclosure provides a composition comprising a conjugate as described herein, or a pharmaceutically acceptable salt thereof, and an inert carrier.

In some embodiments, the present disclosure provides pharmaceutical composition comprising a conjugate as described herein, or a pharmaceutically acceptable salt thereof, and a

In some embodiments, the present disclosure provides a method of making a compound selected from:

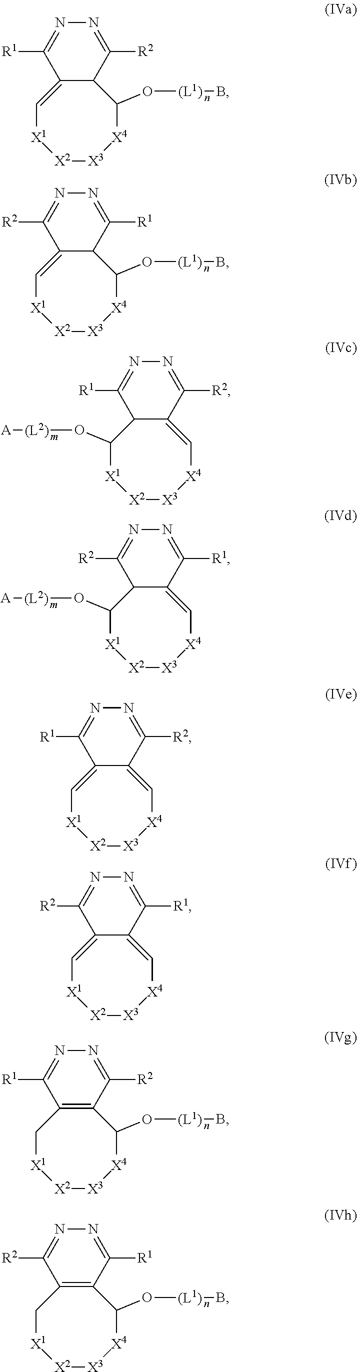

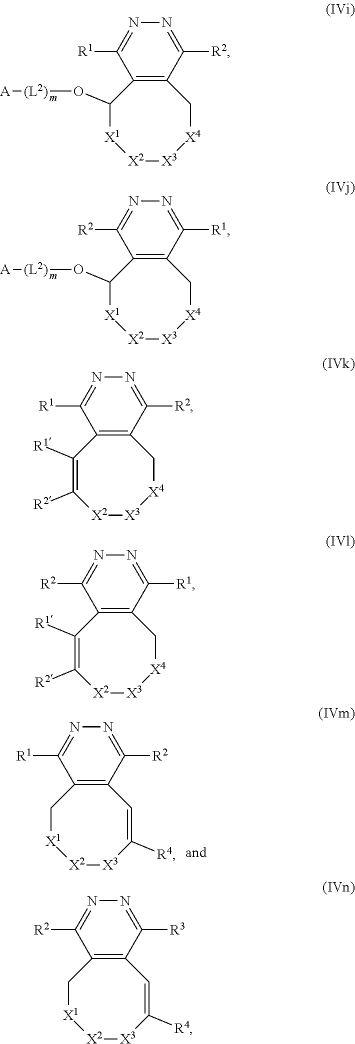

wherein R^{1′} and R^{2′} are as described for the conjugates disclosed herein, or a pharmaceutically acceptable salt thereof, or any combination thereof, the method comprising reacting a conjugate as described herein, or a pharmaceutically acceptable salt thereof, with a compound of Formula (II):

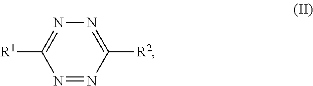

or a pharmaceutically acceptable salt thereof, wherein:
R¹ and R² are each independently selected from H, C₁₋₆ alkyl, C₆₋₁₀ aryl, 5-6-membered heteroaryl, L₁-W, and L₂-OH, wherein said C₁₋₆ alkyl, C₆₋₁₀ aryl, and 5-6-membered heteroaryl are each optionally substituted with 1 or 2 substituents independently selected from halo, C₁₋₆ alkoxy, C₁₋₆ haloalkoxy, a reactive chemical group, and L₃-X;
L₁ is C₁₋₃ alkylene, optionally substituted with 1 or 2 substituents independently selected from halo, C₁₋₆ alkoxy, C₁₋₆ haloalkoxy, a reactive chemical group, and L₃-X;
W is selected from OH, NH₂, and C(O)OH;
L₂ is C₆₋₁₀ arylene, C₃₋₁₀ cycloalkylene, 4-7-membered heterocycloalkylene, and 5-6-membered heteroarylene, each of which is optionally substituted with 1 or 2 substituents independently selected from halo, C₁₋₆ alkoxy, C₁₋₆ haloalkoxy, a reactive chemical group, and L₃-X;
L₃ is a linker;
X is selected from a fluorophore, a fluorescence quencher, a targeting group, a reactive chemical group, and a biologically active molecule, or a fragment thereof.

In some embodiments, reacting the conjugate as described herein, or a pharmaceutically acceptable salt thereof, with the compound of Formula (II), or a pharmaceutically acceptable salt thereof, comprises forming a compound selected from:

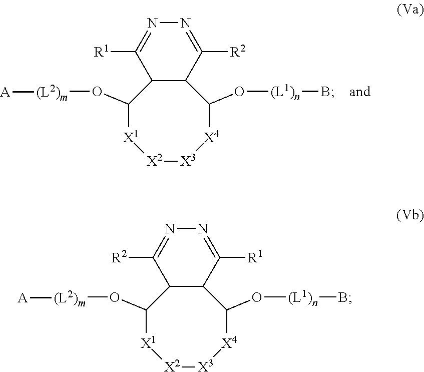

or a combination thereof.

In some embodiments, the method comprises forming the compound of Formula (Va) or Formula (Vb), or a combination thereof, in vitro, in vivo, or ex vivo.

In some embodiments, R¹ and R² are each independently selected from H, C₁₋₆ alkyl, C₆₋₁₀ aryl, 5-6-membered heteroaryl, wherein said C₁₋₆ alkyl, C₆₋₁₀ aryl, and 5-6-membered heteroaryl are each optionally substituted with 1 or 2 substituents independently selected from halo, C₁₋₆ alkoxy, C₁₋₆ haloalkoxy, a reactive chemical group, and L₃-X.

In some embodiments, R¹ and R² are each C₁₋₆ alkyl.

In some embodiments, R¹ and R² are each 5-6-membered heteroaryl.

In some embodiments, at least one of R¹ and R² is selected from L₁-W and L₂-OH.

In some embodiments, at least one of R¹ and R² is selected from L₁-W and L₂-OH.

In some embodiments, both R¹ and R² are independently selected from L₁-W and L₂-OH.

In some embodiments, the disclosure provides a method of making a compound selected from:

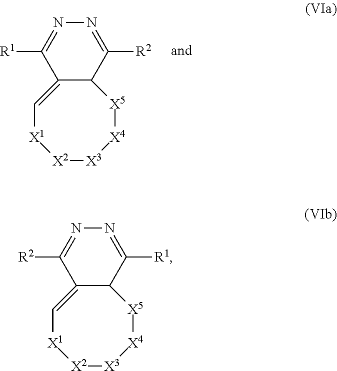

or a pharmaceutically acceptable salt thereof, or a combination thereof, the method comprising reacting a conjugate of Formula (VI):

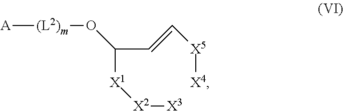

or a pharmaceutically acceptable salt thereof, wherein:
X¹ is selected from O and CHR¹;
X² is selected from O and CHR²;
X³ is selected from O and CHR³;
X⁴ is selected from O and CHR⁴;
X⁵ is selected from O and CHR⁵;
R¹, R², R³, R⁴, and R⁵ are each independently selected from H, halo, C₁₋₆ alkyl, C₁₋₆ haloalkyl, C₁₋₃ alkoxy, C₁₋₃ haloalkoxy, and (L¹)ₙ-B;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy; n is an integer from 0 to 20;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000;
each R^{N} is independently selected from H, C₁₋₃ alkyl, and C₁₋₃ haloalkyl; and
A and B are each independently selected from an antibody, an antibody fragment, an engineered antibody, a peptide, a biologically active protein, a small-molecule drug, a fluorophore, and a fluorescence quencher;
with a compound of Formula (II):

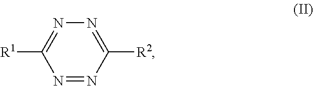

or a pharmaceutically acceptable salt thereof, wherein:
R¹ and R² are each independently selected from L₁-W and L₂-OH;
L₁ is C₁₋₃ alkylene, optionally substituted with 1 or 2 substituents independently selected from halo, C₁₋₆ alkoxy, C₁₋₆ haloalkoxy, a reactive chemical group, and L₃-X;
W is selected from OH, NH₂, and C(O)OH;
L₂ is C₆₋₁₀ arylene, C₃₋₁₀ cycloalkylene, 4-7-membered heterocycloalkylene, and 5-6-membered heteroarylene, each of which is optionally substituted with 1 or 2 substituents independently selected from halo, C₁₋₆ alkoxy, C₁₋₆ haloalkoxy, a reactive chemical group, and L₃-X;
L₃ is a linker;
X is selected from a fluorophore, a fluorescence quencher, a targeting group, a reactive chemical group, and a biologically active molecule, or a fragment thereof;
provided that the compound of Formula (II) is not any one of the following compounds:

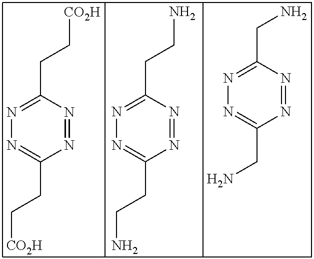 [SMILES: O=C(O)CCc1nnc(CCC(=O)O)nn1 ; NCCc1nnc(CCN)nn1 ; NCc1nnc(CN)nn1]

In some embodiments, reacting the conjugate of Formula (VI), or a pharmaceutically acceptable salt thereof, with the compound of Formula (II), or a pharmaceutically acceptable salt thereof, comprises forming a compound selected from:

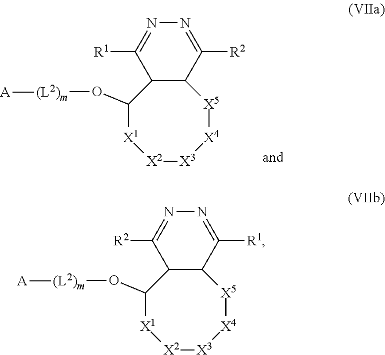

or a combination thereof.

In some embodiments, the method comprises forming the compound of Formula (VIIa) or Formula (VIIb), or a combination thereof, in vitro, in vivo, or ex vivo.

In some embodiments, the conjugate of Formula (VI) has formula:

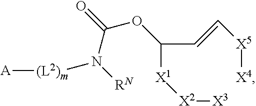

or a pharmaceutically acceptable salt thereof.

In some embodiments:
A is selected from an antibody, an antibody fragment, an engineered antibody, a peptide, a targeting moiety, and a small-molecule drug; and
B is selected from a fluorophore and a fluorescence quencher.

In some embodiments:
A is a small-molecule drug; and
B is selected from an antibody, an antibody fragment, an engineered antibody, a peptide, and a targeting moiety.

In some embodiments:
A is an antibody, an antibody fragment, an engineered antibody, a peptide, and a small-molecule drug; and B is absent.

In some embodiments, the compound of Formula (II) has formula:

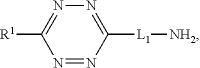

or a pharmaceutically acceptable salt thereof, wherein:
R¹ is selected from L₁-OH, L₁-C(O)OH, and L₂-OH.

In some embodiments, the compound of Formula (II) has formula:

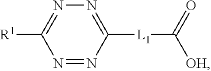

or a pharmaceutically acceptable salt thereof, wherein:
R¹ is selected from L₁-NH₂, L₁-OH, and L₂-OH.

In some embodiments, the compound of Formula (II) has formula:

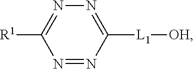

or a pharmaceutically acceptable salt thereof, wherein:
R¹ is selected from L₁-OH, L₁-C(O)OH, L₁-NH₂, and L₂-OH.

In some embodiments, the compound of Formula (II) has formula:

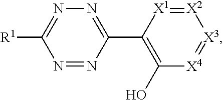

or a pharmaceutically acceptable salt thereof, wherein:
X¹, X², X³, and X⁴ are each independently selected from CH, N, and CR³; and
each R³ is selected from halo, C₁₋₆ alkoxy, C₁₋₆ haloalkoxy, a reactive chemical group, and L₃-X.

In some embodiments, the present disclosure provides a biologically active molecule comprising moiety of formula (i):

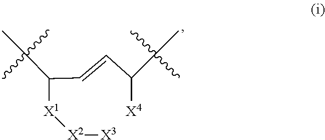

or a pharmaceutically acceptable salt thereof, wherein:
X¹ is selected from O and CHR¹;
X² is selected from O and CHR²;
X³ is selected from O and CHR³;
X⁴ is selected from O and CHR⁴;
R¹, R², R³, and R⁴ are each independently selected from H, halo, C₁₋₆ alkyl, C₁₋₆ haloalkyl, C₁₋₃ alkoxy, and C₁₋₃ haloalkoxy;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene.

In some embodiments, the present disclosure provides a biologically active molecule comprising a moiety of formula:

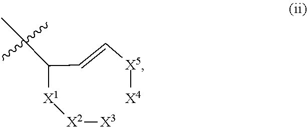

or a pharmaceutically acceptable salt thereof, wherein:
X¹ is selected from O and CHR¹;
X² is selected from O and CHR²;
X³ is selected from O and CHR³;
X⁴ is selected from O and CHR⁴;
X⁵ is selected from O and CHR⁵;
R¹, R², R³, R⁴, and R⁵ are each independently selected from H, halo, C₁₋₆ alkyl, C₁₋₆ haloalkyl, C₁₋₃ alkoxy, C₁₋₃ haloalkoxy, and a moiety of formula (iii):

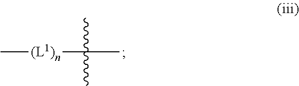

or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (iii);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (iii);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (iii);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (iii);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (iii);
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each x is independently an integer from 1 to 2,000; and
each R^{N} is independently selected from H, C₁₋₃ alkyl, and C₁₋₃ haloalkyl,
provided that the moiety of formula (ii) comprises at least one moiety of formula (iii).

In some embodiments, the biologically active molecule is a peptide or a protein.

In some embodiments, the biologically active molecule is the peptide or the protein which, prior to incorporation of the moiety of formula (i) or formula (ii) into its chemical structure, is selected from bivalirudin, a madanin, a haemathrin, a variegin, an integrilin, and a cytokine.

In some embodiments, the biologically active molecule is selected from any one of the biologically active molecules depicted in FIGS. 29E, 29F, and 34, or a pharmaceutically acceptable salt thereof.

In some embodiments, the present disclosure provides a method of modulating activity of a biologically active molecule of formula (i) or formula (ii) as described herein, the method comprising reacting the biologically active molecule with a compound of Formula (II):

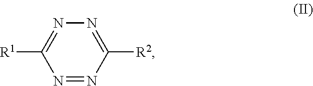

or a pharmaceutically acceptable salt thereof, wherein:
R¹ and R² are each independently selected from H, C₁₋₆ alkyl, C₆₋₁₀ aryl, 5-6-membered heteroaryl, L₁-W, and L₂-OH, wherein said C₁₋₆ alkyl, C₆₋₁₀ aryl, and 5-6-membered heteroaryl are each optionally substituted with 1 or 2 substituents independently selected from halo, C₁₋₆ alkoxy, and C₁₋₆ haloalkoxy;
L₁ is C₁₋₃ alkylene, optionally substituted with 1 or 2 substituents independently selected from halo, C₁₋₆ alkoxy, and C₁₋₆ haloalkoxy;
W is selected from OH, NH₂, and C(O)OH; and
L₂ is C₆₋₁₀ arylene, C₃₋₁₀ cycloalkylene, 4-7-membered heterocycloalkylene, and 5-6-membered heteroarylene, each of which is optionally substituted with 1 or 2 substituents independently selected from halo, C₁₋₆ alkoxy, and C₁₋₆ haloalkoxy.

In some embodiments, reacting the biologically active compound, or a pharmaceutically acceptable salt thereof, with the compound of Formula (II), or a pharmaceutically acceptable salt thereof, comprises forming a compound selected from:

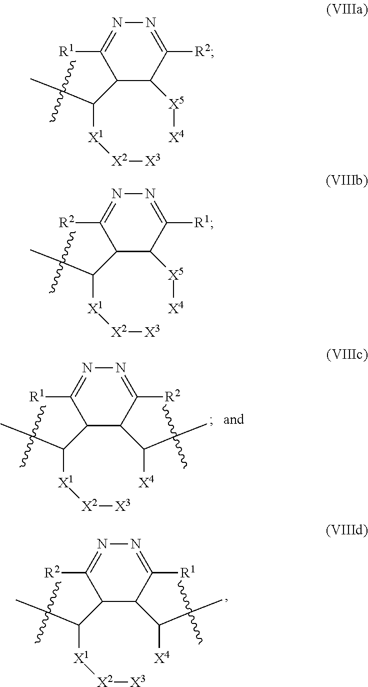

or a combination thereof.

In some embodiments, the method comprises forming the compound of any one of the Formulae (VIIIa)-(VIIId), or a combination thereof, in vitro, in vivo, or ex vivo.

In some embodiments, the method comprises reacting the biologically active molecule with a compound of Formula (II) results in decrease or loss of biological function of the biologically active molecule.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the present application will be apparent from the following detailed description and figures, and from the claims.

### DESCRIPTION OF DRAWINGS

FIG. 1A shows bioorthogonal ligation of tetrazines (Tz) and trans-cyclooctenes (TCO) for the fluorogenic labeling of biomolecules, targeting compounds, or ligands.

FIG. 1B shows Tz-triggered cleavage from trans-cyclooctene modified in allylic position (release-TCO, rTCO) for the bioorthogonal activation of prodrugs or rTCO-caged biomolecules.

FIG. 2 illustrates limitations of the prior art click-to-release compounds and methods with emphasis on Tz/TCO click rates, release yield and release kinetics.

FIG. 3 shows schematically a concept of bioorthogonal cleavage to disassemble molecular probes or inactivate fused biomolecules.

FIG. 4 shows the critical role of post-click tautomerization (left; DHP=dihydropyridazine); directed and accelerated formation of B1 as well as enhanced 1,4-elimination with directing groups (CO₂H, NH₃⁺) in head-to-head position (right).

FIG. 5 shows that stopped-flow measurements verified the significantly higher click reactivity of pyridyl-Tz 4 and 5 with the water-soluble rTCO-derivative 6.

FIG. 6 shows that investigation of the reactions of PyrMe (4) and PyrPA (5) with rTCO-Alexa Fluor 350 (7) by LCMS revealed a dead-end product that was isolated (after reacting 4 with rTCO-glycine) and confirmed by NMR as the irreversibly formed B2 tautomer with Pyr in head-to-head position (top right).

FIG. 7 shows design of a C₂-symmetric trans-cyclooctene enabling directed tautomerization and enhanced elimination for fast and complete cleavage irrespective of the click orientation.

FIG. 8 shows synthesis of bis-axial and bis-equatorial C₂TCO-diol (11) and overlay of schematic ¹³C NMR spectra of the separated and purified compounds, which indicate C₂-symmetry of both isomers resulting from the rigid ‘crown’ (or twist) conformation of the TCO core structure.

FIG. 9 shows modification of C₂TCO (bis-axial-11) via bis-imidazolium-carbamate 12 to obtain bis(sarcosinyl)-C₂TCO (13) and fluorescent AF594-C₂TCO-AF594 (15) for further investigation of the click-to-release of C₂TCO.

FIG. 10 shows results of reacting 15 with selected tetrazines at pH 7.4 and endpoint analysis by HPLC at 24 h, the analysis revealed high cleavage efficiency for DMT and quantitative cleavage with tetrazines modified with a directing group (MPA, PyrPA, PymPA, PymK).

FIG. 11 shows that second order rate constants as determined by stopped-flow spectrophotometry showed decreased reactivity of C₂TCO compared to rTCO (cf. FIG. 5), and significantly higher click kinetics for ammonium-functionalized PymK (3) compared to the respective Tz-acid PymPA (18).

FIG. 12 schematically shows that steric hindrance of the aryl substituent as well as interactions of directing groups affect the click kinetics with C₂TCO.

FIG. 13 shows that H-tetrazines modified with a directing group (HPA, HK) enable complete cleavage of C₂TCO with significantly improved click rates due to decreased steric hindrance.

FIG. 14 shows that click-to-cut experiments at low concentrations (10 μM C₂TCO+20 μM Tz) revealed instantaneous release upon click (no intermediates detected) and complete overall cleavage (99%) with HK (20) and PymK (3) after 30 min.

FIG. 15 contains a bar graph showing that unlike rTCO derivatives, C₂TCO and HPA/HK cleavage is readily accelerated by increasing the Tz concentration.

FIG. 16 shows DBCO—C₂TCO-AF594 (21) as a click- and cleavable fluorescent tag for conjugation with azide-labeled antibodies.

FIG. 17 shows staining of EGFR-positive A431 cells with Cetux-C₂TCO-AF594 (on-state) and subsequent click-to-cut with HPA (off-state).

FIG. 18 shows that cell imaging confirmed near-quantitative bioorthogonal disassembly of Cetux-C₂TCO-AF594 within 6 min.

FIG. 19 shows chemical structure of Ib-C₂TCO—SiR (22) as a cleavable fluorescent ibrutinib conjugate.

FIG. 20 shows cellular uptake and binding to BTK of 22 upon treating HT1080-BTK-mCherry cells, and subsequent intracellular click-to-cut with HK (19).

FIG. 21 shows that fluorescence microscopy imaging showed excellent colocalization of mCherry and SiR, confirming selective binding of Ib-C₂TCO—SiR (22) to BTK (on-state, top row); complete intracellular cleavage (>99%) was observed upon treatment with 20 μM HK for 30 min (off-state, bottom row).

FIG. 22 schematically shows chemical inactivation of a biologically active (bio)molecule using a bioorthogonal chemical reaction.

FIG. 23 schematically shows using tetrazine reagents to bioorthogonally cleave therapeutic molecules. R¹ and R² within the Tz reagent refer to various functional groups (e.g., alkyl, aryl, heteroaryl), including directing groups (e.g., CO₂H). Challenges of this approach, including formation of tricyclic dead end product, are also shown.

FIG. 24 schematically shows reaction of C₂TCO containing molecules with various Tz reagents, including click reaction rates.

FIG. 25 shows tetrazine (Tz) reagents containing various phenolic directing functional groups and their effect on C₂TCO release rates.

FIG. 26 shows independent tuning of click and release. Cleavage rage can be finely tuned by modulating Tz acid geometry (special proximity) and/or acidity. pKa can be adjusted systematically with substituent electronegativity and ring position. At lower right, sC₂TCO chemical structure is shown.

FIG. 27 contains spatiotemporal kinetics. Analysis of mean transit time through the systemic and regional circulation predicts a target half-life of 5-25 seconds for self-destructing anticoagulants.

FIG. 28 contains a table showing that bivalirudin is an exceptionally tolerant of the linker between its two binding elements and is inert once cut. Madanins and s-Variegins are extremely potent bivalent thrombin inhibitors.

FIG. 29A contains a 3D model of ClickBival bound to human thrombin. Subunits P4 and P12 are shown, as well as the C₂TCO fragment. A and B indicate sites flanking the C₂TCO linker where chemical modifications will be varied.

FIG. 29B contains line plots showing measurement of ClickBival Kᵢ by fluorogenic substrate cleavage.

FIG. 29C contains images showing bivalirudin alternatives: madanin-1 and s-variegin bind thrombin bivalently and offer prospective ClickLinker modification sites (dashed ovals).

FIG. 29D contains a schematic representation of programmed self-destruction of ClickBival by a Tz reagent.

FIG. 29E provides chemical structure of ClickBival compound with C₂TCO cleavable linker (P4-C₂TCO—P12).

FIG. 29F provides chemical structure of ClickBival compound with cTCO cleavable linker (P4-cTCO—P12).

FIG. 30A shows precision chemical control of plasmin and tPA. Protease recognition of (Lys, Arg) enables a TCO-substrate ON/OFF switch. Plasmin active site geometry: no space for Lys-rTCO.

FIG. 30B contains line plot showing a fluorogenic tPA substrate is cleaved only after Tz/rTCO release.

FIG. 30C contains an image showing release of free Lys of the protease-binding domain of the protease substrate tPA.

FIG. 30D shows results of testing Click-tPA. Introduced protease cleavage sites bearing rTCO-Lys residues are inert until Tz click, controlling plasmin cleavage via catalytically-amplified feedback inactivation.

FIG. 31 contains an image showing key tPA surface residues/loops.

FIG. 32 shows ClickLinker development for click-tPA applications. The molecular Tz/TCO infrastructure required for anticoagulant development translates to Plasmin/protease tools.

FIG. 33 shows click-eptifibatide. Reversible GPIIbIIIa inhibition by macrocycle cleavage.

FIG. 34 shows chemical structures of exemplified cleavable integrilins.

FIG. 35 shows 3D structure of IL2 pointing to sites of modification with a cleavable moiety.

FIG. 36A contains a reaction scheme showing fluorescent cycling techniques within the present claims.

FIG. 36B contains chemical structures of Tz reagents conjugated to a quencher.

FIG. 37A contains a chemical structure of a AF488 fluorescent C₂TCO probe within the present claims.

FIG. 37B contains a chemical structure of a AF555 fluorescent C₂TCO probe within the present claims.

FIG. 37C contains a chemical structure of a AF594 fluorescent C₂TCO probe within the present claims.

FIG. 37D contains a chemical structure of a AF647 fluorescent C₂TCO probe within the present claims.

FIG. 38 shows multiplexed temporospatial profiling in living cells. Serial bioorthogonal cycling can reveal complex cellular dynamics in living cells, enabling phenotypic characterization and longitudinal tracking of temporal evolution and/or neighbor-neighbor interactions. Inset: the design of scission-accelerated fluorophore exchange (SAFE) combines a hyperaccelerated click-quenching reaction with rapid fluorophore scission to gently, efficiently, and irreversibly erase fluorophores from the surface of immunostained living cells in seconds.

FIG. 39 To quantify cell viability across multiple cycles of staining and quenching, living mouse PBMCs were isolated, aliquoted, loaded into microwells for imaging. The cells were profiled with a SAFE panel of 12 immune markers, imaged in sets of three, followed by live/dead staining dyes to assess cell survival. Efficient scission performance produced images with high signal to background across all five cycles and no evident residual signal; both cell viability after bioorthogonal profiling (96.5% vs. 96.2%) and marker abundance matched well with flow cytometry controls (R2=0.99).

FIG. 40 SAFE antibodies. For the anti-c-Kit antibody, its secondary antibody (anti-rat IgG) was modified with SAFE for staining.

FIG. 41 Flow cytometry antibodies. For the anti-MHCI antibody, SAFE-conjugated AF647 was used. For the anti-c-Kit (biotin) antibody (BioLegend), streptavidin BV570 was used.

FIG. 42A Antibodies labeled with C2TCO-linked dyes are rendered non-fluorescent in a concerted two step sequence: noncovalent interactions between BHQ3 and the fluorophore can accelerate the Tz/TCO click reaction[Ko], leading to immediate quenching upon ligation; in turn, the functionalized ‘Tz-scissors’ drives instantaneous tautomerization and scission of the dye. The design of C2TCO itself ensures that the cleavage reaction proceeds irrespective of the Tz/TCO click orientation.

FIG. 42B Starting from the Boc-protected Tz (1), a hydrophilic PEG linker and BHQ3 are conjugated and then deprotected to yield BHQ3-N-Tz (2).

FIG. 43A Quantification of reaction kinetics for the click reaction of 2 with an AF647-C2TCO-labeled antibody in solution revealed an effective rate constant of >106, a marked 2,900-fold acceleration relative to the parent aminoethyl-functionalized Tz (3). This acceleration shifts the concentration/time relationship into the needed range for efficient cycling at 1 μM reagent concentrations.

FIG. 43B To validate the accelerated kinetics in the cellular context, we collected time lapse images of A431 cells stained with a SAFE647-anti-EGFR antibody and then treated with BHQ3-N-Tz (2). Background-subtracted images rendered at two window levels capture the rapid (t½=1.9 s) and quantitative clearance of the fluorophore signal, reaching 99.8% removal within 40 seconds, concordant with the in vitro assay measurements.

FIG. 44A Staining and scission with SAFE647-anti-N-Cadherin confirmed efficient scission and short-term nontoxicity in living A549 cells, a subset of which progressed through mitosis during the experiment.

FIG. 44B Longer term imaging of A549 cells was used to confirm the lack of toxicity and assess the impact of scission. Serial imaging over 24 hours demonstrated durable signal elimination and intact live cell calceinAM staining for SAFE. Control cells stained and quenched in parallel with a noncleavable anti-EGFR fluorophore-TCO conjugate and BHQ3-Tz were initially dark, but exhibited marked rebound of the fluorescent signal 24 hrs later, likely due to extra/intracellular degradation of the BHQ3 quencher, indicating a critical role for scission.

FIG. 45A Primary murine bone marrow cells were isolated, plated in adherent culture and imaged after 24 hours. Confocal microscopy of 12 markers profiled in 4 cycles enabled discrimination of intracellular and extracellular fluorescent staining at high spatial resolution. A minority of cells displayed intracellular staining that was not removed by scission, suggestive of antibody internalization prior to fluorophore scission with BHQ3-N-Tz.

FIG. 45B A focus on the myeloid markers in cycle 4 reveals an ensemble of myeloid precursors (CD11b+, Ly6C+), Ly6G-Hi neutrophils, and F4/80+ phagocytes (F4/80+) within the developing bone marrow cell population, highlighted in the selectively (de)colorized individual panels at right.

FIG. 46A Deep PDMS microwells efficiently retain suspension cells in culture for serial imaging. Bone marrow derived ER-Hoxb8 cells are conditionally immortalized when cultured with ß-estradiol; removal of ß-estradiol triggers synchronous expansion and neutrophilic differentiation. This developmental sequence induces broad changes in extracellular marker expression and cellular morphology (gray panel, inset). To track this process, CD45, cKit, CD11b, Ly6C, Ly6G, and F4/80 were serially profiled at days 0, 2, 4, and 6, before and during differentiation. CD45 (positive control) and F4/80 (negative control) expression were stable, while cKit (a progenitor cell marker) expression rapidly decreased, disappearing after DO. As expected, the fraction of cells expressing CD11b, Ly6C, and Ly6G rose in sequence over the first 4-6 days.

FIG. 46B Quantitative expression of differentiation markers across six days and four profiles: (i) averaged across a set of 12 microwells; (ii) quantified on day 4 to track proliferation and variation across individual microwells. Ring plots track marker expression (fraction of positive cells, encoded by color) and the total number of CD45+ cells per well (in white).

FIG. 47 Live bone marrow cells. In the last staining cycle, calcein AM (white), CD11b (blue), Ly6C (green), and Ly6G (red) were profiled. The calcein AM image (left) and a composite image of three protein markers (right) are included. Calcein AM staining performed at the end of live bone marrow cell profiling shows a complete overlap with protein marker staining, confirming 100% cellular viability after multiple cyclings.

FIG. 48 contains a scheme showing chemical synthesis of BHQ3-N-Tz (2).

FIG. 49 contains a scheme showing chemical synthesis of fluorophore-C₂TCO-PEG₄-CO₂H reagents.

FIG. 50 contains chemical structure of MB488-C₂TCO-PEG₄-CO₂H (S5).

FIG. 51 contains chemical structure of MB594-C₂TCO-PEG₄-CO₂H (S8).

FIG. 52 contains a scheme showing tetrazine-triggered double-release of C₂TCO.

FIG. 53 shows yields of the double-release reactions shown in FIG. 52.

FIG. 54 shows Click kinetics (second order rate constants) for the reactions of FIG. 52.

FIG. 55 shows structural elucidation by NMR of the double-release product.

FIG. 56 shows synthesis of AF594-C₂TCO-AF594.

FIG. 57 shows chemical synthesis of DBCO—C₂TCO-AF594.

FIG. 58 shows chemical synthesis of Ib-C₂TCO—SiR (22).

FIG. 59 shows general scheme for the synthesis of tetrazine acids.

FIG. 60 shows chemical synthesis of MMAE-C₂TCO-MMAE along with the double-click kinetic experiment.

FIG. 61 shows activation of MMAE-C₂TCO-MMAE in cell culture (HT1080 cells; cell viability experiments; n=4).

FIG. 62 contains chemical structure of C2TCO Bival.

FIG. 63 contains a synthetic scheme showing chemical synthesis of C2TCO Bival.

FIG. 64 contains chemical structure of cTCO Bival.

FIG. 65 contains a synthetic scheme showing chemical synthesis of cTCO Bival.

FIG. 66 contains chemical structure of cTCO-PEG-Bival.

FIG. 67 contains a synthetic scheme showing cTCO-PEG-Bival.

FIG. 68 contains chemical structure of iTCO Bival-P4-release.

FIG. 69 contains chemical structure of iTCO Bival-P12-release.

FIG. 70 contains chemical structure of siTCO Bival-P4-release.

FIG. 71 contains chemical structure of siTCO Bival-P12-release.

FIG. 72 contains chemical structures of additional scaffolds for TCO Bival release.

FIG. 73 contains chemical structure of cTCO Bival-P4 release (containing n Gly units).

FIG. 74 contains chemical structure of cTCO-PEG Bival-P4 release (containing n Gly units)

FIG. 75 contains chemical structure of sC₂TCO-Bival (containing n Gly units)

FIG. 76 contains chemical structure of dC₂TCO-Bival (containing n Gly units).

FIG. 77 contains chemical structure of aza-C2TCO-Bival (containing n Gly units).

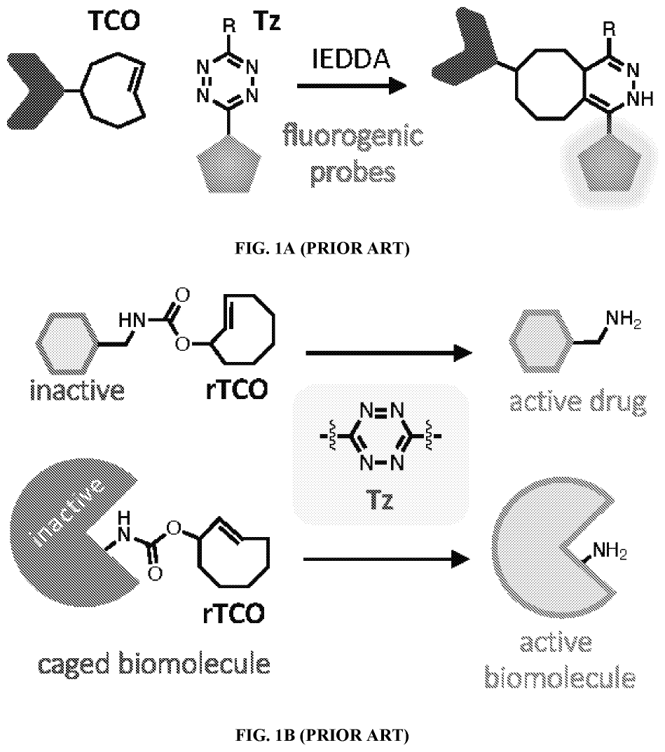

### DETAILED DESCRIPTION

In a general aspect, the present disclosure provides reagents containing a trans-cyclooctene (“TCO”) fragment. Examples of TCO fragments include:

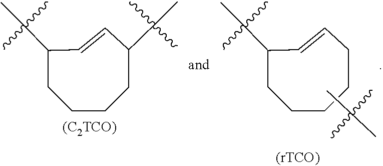

These reagents can be used to prepare conjugates, including antibody-drug conjugates, and (bio)probes, including fluorescently labeled probes, that contain TCO fragments in their chemical structures. In one example, the conjugates can be used to examine cells or components of cells, or to diagnose a patient or to monitor progression of a disease or condition in the patient. In this example, once the conjugate has performed its function, the conjugate can be cleaved (or disassembled) by reaction of the TCO fragment in the conjugate with a tetrazine (“Tz”) reagent. The cleavage (or disassembly) results in loss of function of the conjugate. In another example, the conjugates can be used to deliver biologically active molecules to target tissues. Such a conjugate may contain, in addition to the TCO linker, a targeting moiety and a biologically active drug. In this second example, the conjugate is effectively a “pro-drug,” where the biologically active molecule is released from the conjugate to perform its therapeutic function after the conjugate is cleaved (or disassembled) by reaction of the TCO fragment within the conjugate with a Tz reagent.

In another general aspect, the present disclosure also provides biologically active molecules containing TCO fragments (such as those shown above) in their chemical structures. Due to the presence of TCO fragments, the biologically active molecules can be disassembled “on demand” by reaction with a Tz reagent, leading to loss of the biological function. This can be advantageous if the biologically active molecule is circulating in the patient's bloodstream, but its biological function is no longer needed or its toxicity or other undesired side effects should be avoided.

Certain embodiments of the TCO-containing reagents and linkers, conjugates containing the TCO linkers, biologically active molecules containing TCO moieties, Tz reagents reactive with TCO moieties, compositions containing same and methods of use thereof, are described in the present disclosure.

Linkers and Reagents Containing TCO

In some embodiments, the present disclosure provides a compound containing a “C₂TCO” moiety within its chemical structure. In some embodiments, the compound has Formula (I):

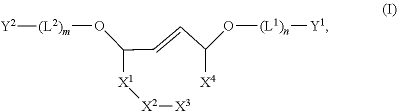

or a pharmaceutically acceptable salt thereof, wherein Y², L², m, X¹, X², X³, X⁴, L¹, n, and Y¹ are as described herein.

In some embodiments:
X¹ is selected from O and CHR¹;
X² is selected from O and CHR²;
X³ is selected from O and CHR³;
X⁴ is selected from O and CHR⁴;
R¹, R², R³, and R⁴ are each independently selected from H, halo, C₁₋₆ alkyl, C₁₋₆ haloalkyl, C₁₋₃ alkoxy, and C₁₋₃ haloalkoxy;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000;
each R^{N} is independently selected from H, C₁₋₃ alkyl, and C₁₋₃ haloalkyl; and
each Y¹ and Y² are independently selected from H, NH₂, OH, C(O)OH, a protected amino group, a protected carboxyl group, a protected hydroxyl group, a reactive chemical group, a fluorophore, and a fluorescence quencher.

In some embodiments, X¹ is O. In some embodiments, X¹ is CHR¹.

In some embodiments, X² is O. In some embodiments, X² is CHR².

In some embodiments, X³ is O. In some embodiments, X³ is CHR³.

In some embodiments, X⁴ is O. In some embodiments, X⁴ is CHR⁴.

In some embodiments, R², R³, and R⁴ are each independently selected from H, halo, C₁₋₆ alkyl, C₁₋₆ haloalkyl, C₁₋₃ alkoxy, and C₁₋₃ haloalkoxy.

In some embodiments, R², R³, and R⁴ are each independently selected from H, halo, and C₁₋₆ alkyl.

In some embodiments, R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, HO—C₁₋₃ alkylene, and NH₂—C₁₋₃ alkylene.

In some embodiments, R¹ and R², together with the carbon atoms to which they are attached, form an C₆₋₁₀ aryl ring (e.g., phenyl).

In some embodiments, R¹ and R², together with the carbon atoms to which they are attached, form a C₃₋₁₀ cycloalkyl ring (e.g., cyclopropyl, cyclobutyl, cyclopentyl, or cyclohexyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, R¹ and R², together with the carbon atoms to which they are attached, form a 5-14 membered heteroaryl ring (e.g., pyridinyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, R¹ and R², together with the carbon atoms to which they are attached, form a 4-10 membered heterocycloalkyl ring (e.g., azetidinyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, HO—C₁₋₃ alkylene, and NH₂—C₁₋₃ alkylene.

In some embodiments, R² and R³, together with the carbon atoms to which they are attached, form an C₆₋₁₀ aryl ring (e.g., phenyl).

In some embodiments, R² and R³, together with the carbon atoms to which they are attached, form a C₃₋₁₀ cycloalkyl ring (e.g., cyclopropyl, cyclobutyl, cyclopentyl, or cyclohexyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, R² and R³, together with the carbon atoms to which they are attached, form a 5-14 membered heteroaryl ring (e.g., pyridinyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, R² and R³, together with the carbon atoms to which they are attached, form a 4-10 membered heterocycloalkyl ring (e.g., azetidinyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, HO—C₁₋₃ alkylene, and NH₂—C₁₋₃ alkylene.

In some embodiments, R³ and R⁴, together with the carbon atoms to which they are attached, form an C₆₋₁₀ aryl ring (e.g., phenyl).

In some embodiments, R³ and R⁴, together with the carbon atoms to which they are attached, form a C₃₋₁₀ cycloalkyl ring (e.g., cyclopropyl, cyclobutyl, cyclopentyl, or cyclohexyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, R³ and R⁴, together with the carbon atoms to which they are attached, form a 5-14 membered heteroaryl ring (e.g., pyridinyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, R³ and R⁴, together with the carbon atoms to which they are attached, form a 4-10 membered heterocycloalkyl ring (e.g., azetidinyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, each L¹ is independently selected from N(R^{N}), O, C(═O), C₁₋₆ alkylene, —(OCH₂CH₂)ₓ—, —(CH₂CH₂O)ₓ—, an amino acid, a self-immolative group, and a moiety formed by a click reaction, each of which is optionally substituted with 1 or 2 substituents independently selected from C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy.

In some embodiments, each L¹ is independently selected from N(R^{N}), O, C(═O), C₁₋₆ alkylene, —(OCH₂CH₂)ₓ—, and —(CH₂CH₂O)ₓ—.

In some embodiments, at least one L¹ is N(R^{N}). In some embodiments, at least one L¹ is O. In some embodiments, at least one L¹ is C(═O). In some embodiments, at least one L¹ is C₁₋₆ alkylene, optionally substituted with C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy.

In some embodiments, n is an integer from 0 to 20. In some embodiments, n is an integer from 0 to 10. In some embodiments, n is 0, 1, 2, 3, 4, or 5.

In some embodiments, In some embodiments, each L² is independently selected from N(R^{N}), O, C(═O), C₁₋₆ alkylene, —(OCH₂CH₂)ₓ—, —(CH₂CH₂O)ₓ—, an amino acid, a self-immolative group, and a moiety formed by a click reaction, each of which is optionally substituted with 1 or 2 substituents independently selected from C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy.

In some embodiments, each L² is independently selected from N(R^{N}), O, C(═O), C₁₋₆ alkylene, —(OCH₂CH₂)ₓ—, and —(CH₂CH₂O)ₓ—.

In some embodiments, at least one L² is N(R^{N}). In some embodiments, at least one L² is O. In some embodiments, at least one L² is C(═O). In some embodiments, at least one L² is C₁₋₆ alkylene, optionally substituted with C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy.

In some embodiments, m is an integer from 0 to 20. In some embodiments, m is an integer from 0 to 10. In some embodiments, m is 0, 1, 2, 3, 4, or 5.

In some embodiments, each x is independently an integer from 1 to 2,000 (e.g., 10, 20, 100, 200, or 500).

In some embodiments, each R^{N} is independently selected from H, C₁₋₃ alkyl, and C₁₋₃ haloalkyl. In some embodiments, R^{N} is H. In some embodiments, R^{N} is C₁₋₃ alkyl.

Amino Acids and Amino Acid Linker Moieties

Suitable examples of amino acids that could be used in L¹ or L² include lysine, arginine, histidine, aspartic acid, glutamic acid, serine, threonine, asparagine, glutamine, cysteine, glycine, proline, alanine, valine, isoleucine, leucine, methionine, phenylalanine, tyrosine, and tryptophan. Compounds and linker moieties with protected side chain functional groups are also encompassed by the term “amino acid” as used herein. A linker may include any n number of amino acids, for example, the linker can include (Gly)ₙ, where Gly can be N-linked or O-linked to any part of the remainder of the linker, and n can be an integer from 1 to 100 (e.g., 2, 3, 4, 5, 6, 7, 8, 9, or 10).

As used herein, the term “amino acid” generally refers to organic compounds containing amine (—NH₂) and carboxyl (—COOH) functional groups, along with a side chain (R group) specific to each amino acid. The side chain may be hydrophobic or hydrophilic, charged or neutral, as well as aliphatic or aromatic. In natural amino acids, the amine and carboxyl functional groups attached to the same carbon atom, i.e., an amino group is attached to the carbon in α-position relative to carboxyl group. Any of the amino acids described herein may be in L configuration or in D configuration. In some embodiments, the amino acid is in L configuration. In some embodiments, the amino acid is in D configuration. The 20 natural amino acids are abbreviated herein as shown in Table A:

TABLE A

| Three-letter | One-letter | |
|---|---|---|
| abbreviation | abbreviation | Amino acid name |
| Ala | A | Alanine |
| Arg | R | Arginine |
| Asn | N | Asparagine |
| Asp | D | Aspartic acid |
| Cys | C | Cysteine |
| Gln | Q | Glutamine |
| Glu | E | Glutamic acid |
| Gly | G | Glycine |
| His | H | Histidine |
| Ile | I | Isoleucine |
| Leu | L | Leucine |
| Lys | K | Lysine |
| Met | M | Methionine |
| Phe | F | Phenylalanine |
| Pro | P | Proline |
| Ser | S | Serine |
| Thr | T | Threonine |
| Trp | W | Tryptophan |
| Tyr | Y | Tyrosine |
| Val | V | Valine |

Self-Immolative Groups and Linker Moieties

Suitable examples of self-immolative groups include those described, for example, in Alouane, A. et al., “Self-immolative spacers: kinetic aspects, structure-property relationships, and applications”, Angew. Chem. Int. Ed., 2015, 54, 7492-7509 and Kolakowski, R. V. et al., “The methylene alkoxy carbamate self-immolative unit: Utilization of the targeted delivery of alcohol-containing payloads with antibody-drug conjugates”, Angew. Chem. Int. Ed., 2016, 55, 7948-7951.

Examples of self-immolative groups include the following groups of formulae (a)-(i):

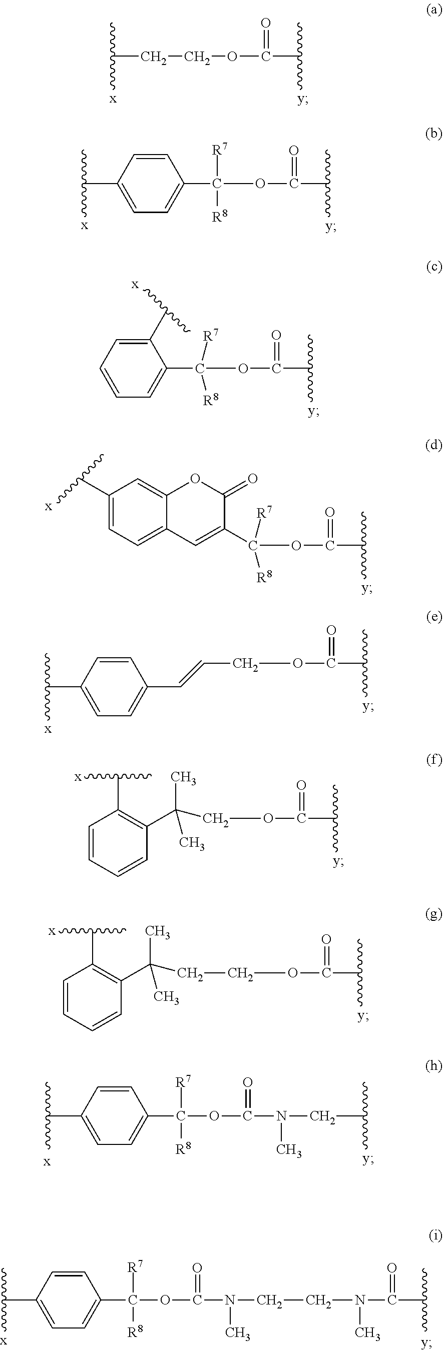

wherein x and y denote points of attachment to different L¹ or L², and R⁷ and R⁸ are each independently selected from H and C₁₋₆ alkyl.

Groups and Moieties that are Formed by a “Click” Reaction

In some embodiments, the moiety formed by a click reaction is a reaction product of any one of the well-known click reactions, such as Huisgen cycloaddition (also known as [3+2] cycloaddition of alkynes and azides to form triazoles), Staudinger ligation (i.e., a reaction between an azide and a phosphine), a reaction of oxanorbornadienes and azides to from triazoles, an inverse-demand Diels-Alder reaction of tetrazines (e.g., dipyridyl tetrazines) and trans-cyclooctynes, inverse-demand Diels-Alder reaction of tetrazines (e.g., monoaryl tetrazines) and norbornenes, a reaction of tetrazines and cyclopropenes, a reaction of cyclopropenes and nitrile imines, a photoinduced 1,3-dipolar cycloaddition of tetrazoles and alkenes, a 1,3-dipolar cycloaddition of nitrile oxides and norbornenes, a [4+1] cycloaddition isocyanides and tetrazines, or a 1,3-cycloaddition of nitrones and alkynes. In some embodiments, the moiety formed by a click reaction comprises a triazole. In some embodiments, the moiety formed by a click reaction is selected from:

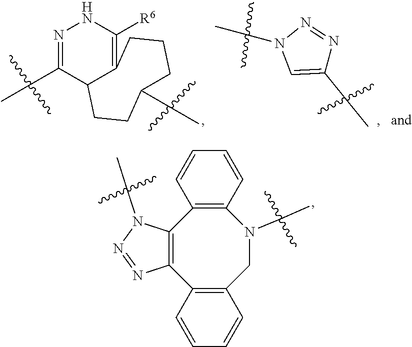

wherein R⁶ is selected from H and C₁₋₆ alkyl.

In some embodiments, each Y¹ and Y² are independently selected from H, NH₂, OH, C(O)OH, a protected amino group, a protected carboxyl group, a protected hydroxyl group, a reactive chemical group, a targeting group, and a fluorophore.

In some embodiments, Y¹ is NH₂ and Y² is C(O)OH.

In some embodiments, Y¹ is a protected amino group.

In some embodiments, Y¹ is a protected carboxyl group.

In some embodiments, Y¹ is protected hydroxyl group.

In some embodiments, Y² is a protected amino group.

In some embodiments, Y² is a protected carboxyl group.

In some embodiments, Y² is protected hydroxyl group.

Protected Chemical Groups

In some embodiments, a skilled chemist would be able to select and implement any of the amine protecting groups, alcohol protecting groups, or carboxylic acid protecting groups of the present disclosure. The chemistry of protecting groups can be found, for example, in P. G. M. Wuts and T. W. Greene, Protective Groups in Organic Synthesis, 4^{th} Ed., Wiley & Sons, Inc., New York (2006) (which is incorporated herein by reference), including suitable examples of the protecting groups, and methods for protection and deprotection, and the selection of appropriate protecting groups.

Suitable examples of amine-protecting groups include Carbobenzyloxy (Cbz) group, p-Methoxybenzyl carbonyl (Moz or MeOZ), tert-Butyloxycarbonyl (BOC) group, 9-Fluorenylmethyloxycarbonyl (Fmoc), Acetyl (Ac), Benzoyl (Bz), Benzyl (Bn) group, Carbamate group, p-Methoxybenzyl (PMB), 3,4-Dimethoxybenzyl (DMPM), p-Methoxyphenyl (PMP) group, Tosyl (Ts) group, Troc (trichloroethyl chloroformate), and nosyl group.

Suitable examples of alcohol-protecting groups include Acetyl (Ac), Benzoyl (Bz), Benzyl (Bn), β-Methoxyethoxymethyl ether (MEM), Dimethoxytrityl, [bis-(4-methoxyphenyl)phenylmethyl] (DMT), Methoxymethyl ether (MOM), Methoxytrityl [(4-methoxyphenyl)diphenylmethyl] (MMT), p-Methoxybenzyl ether (PMB), Methylthiomethyl ether, Pivaloyl (Piv), Tetrahydropyranyl (THP), Tetrahydrofuran (THF), Trityl (triphenylmethyl, Tr), Silyl ether (most popular ones include trimethylsilyl (TMS), tert-butyldimethylsilyl (TBDMS), tri-iso-propylsilyloxymethyl (TOM), and triisopropylsilyl (TIPS) ethers), Methyl ethers, and Ethoxyethyl ethers (EE).

Suitable examples of carboxylic acid protecting groups include methyl esters, benzyl esters, tert-butyl esters, esters of 2,6-disubstituted phenols (e.g., 2,6-dimethylphenol, 2,6-diisopropylphenol, 2,6-di-tert-butylphenol), silyl esters, orthoesters, and oxazoline.

In some embodiments, Y¹ is a reactive chemical group.

In some embodiments, Y² is a reactive chemical group.

Reactive Chemical Groups

Suitable examples of reactive chemical groups include those reactive with OH, NH₂, C(O)OH, and SH functional groups, as well as bioorthogonal reactive groups.

Suitable examples of groups reactive with OH include the following groups:

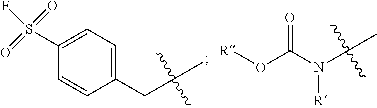

(R′ is H or C₁₋₃ alkyl, R″ is C₁₋₃ alkyl).

Suitable examples of groups reactive with SH include the following groups:

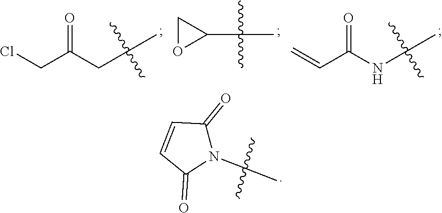

Suitable examples of groups reactive with NH₂ includes an activated ester of formula:

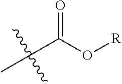

(R is, e.g., N-succinimidyl, N-benzotriazolyl, 4-nitrophenyl, or pentafluorophenyl).

Suitable examples of reactive chemical groups that are reactive in bioorthogonal chemical reactions include azide, alkene, alkyne, and tetrazine reactive groups, such as:

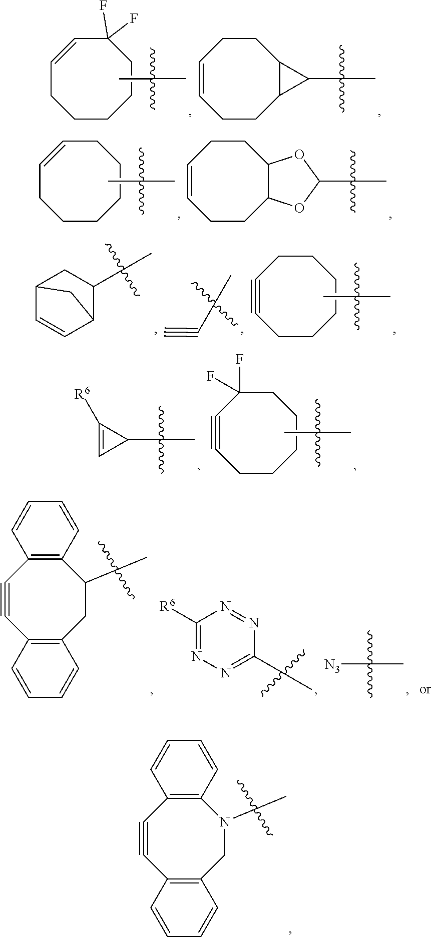

wherein R⁶ is selected from H and C₁₋₆ alkyl.

Other examples of reactive groups include a nitrone, an isocyanide, a diphenylphosphine, nitrile imine, a tetrazole, and a nitrile oxide.

In some embodiments, Y¹ is a fluorophore.

In some embodiments, Y² is a fluorophore.

Fluorophores and Fluorescent Groups

Suitable examples of fluorophores include any fluorescent chemical compound that can re-emit light upon light excitation. The fluorophores can by excited by a light of a wavelength form about 300 nm to about 800 nm, and then emit light of a wavelength from about 350 nm to about 770 nm (e.g., violet, blue, cyan, green, yellow, orange or red light), which can be detected by fluorescent imaging devices, including the ability to measure the intensity of the fluorescence. Suitable examples of fluorophores include AF488, Hydroxycoumarin blue, methoxycoumarin blue, Alexa fluor blue, aminocoumarin blue, Cy2 green (dark), FAM green (dark), Alexa fluor 488 green (light), Fluorescein FITC green (light), Alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red. Absorbance and emission wavelengths of these fluorophores are well known in the art.

In some embodiments, Y¹ is a fluorescence quencher.

In some embodiments, Y² is a fluorescence quencher.

Fluorescence Quenchers

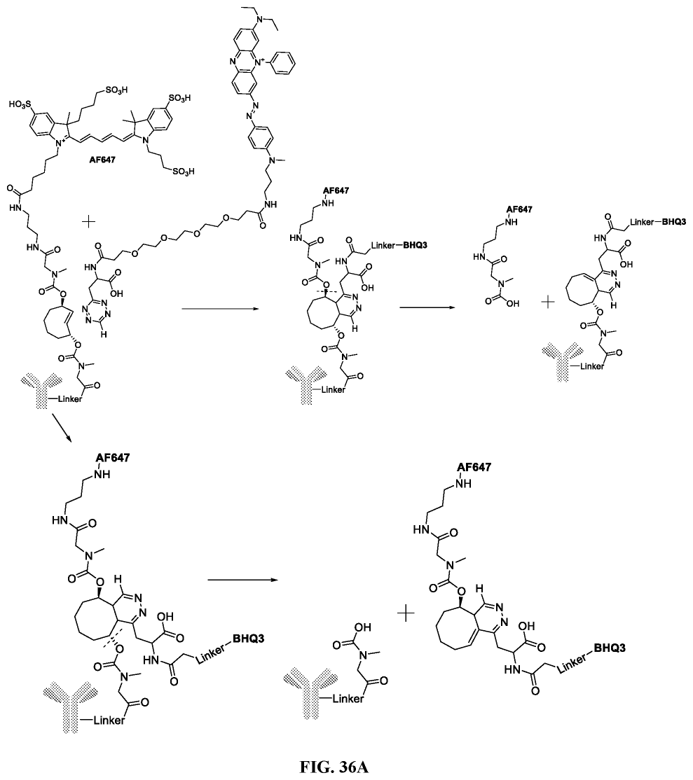

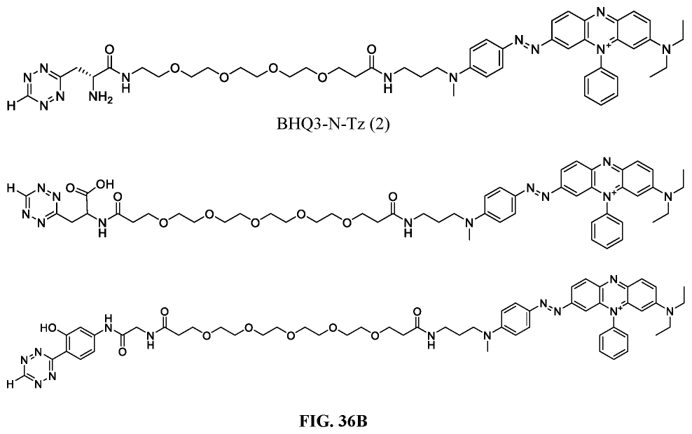

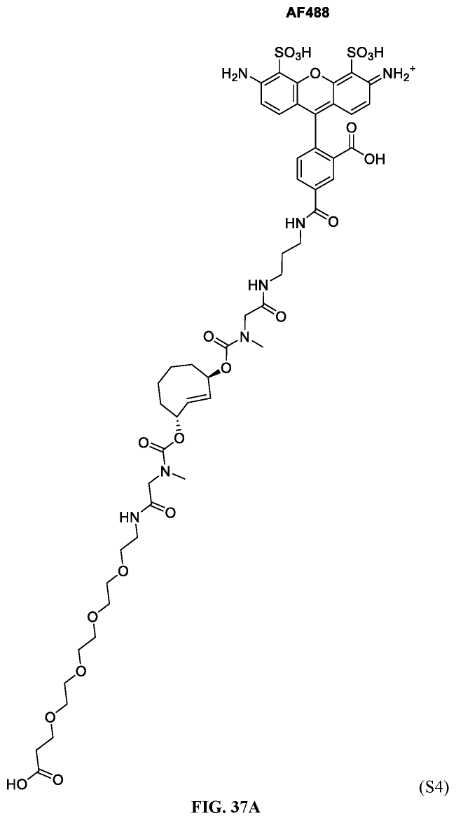

Suitable examples of fluorescence quenchers include aromatic azo compounds and phenazine derivatives. In some examples, the fluorescence quencher is BHQ0, BHQ1, BHQ2, BHQ3, BHQ10, or IRDye QC-1. Without being bound by a theory, it is believed that a quencher may quench fluorescence of a fluorophore through FRET quenching, that is, the excited fluorophore instead of emitting light transfers energy to the quencher through space (See, e.g., FIG. 36A). In the absence of the quencher, the fluorophore would have emitted the light, which could have been detected. In some embodiments, the quencher and the fluorophore are selected such that the emission spectrum of the fluorophore substantially overlaps with the absorption spectrum of the quencher.

In some embodiments, the compound of Formula (I) has formula:

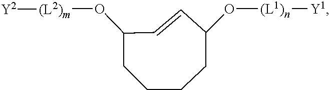

or a pharmaceutically acceptable salt thereof.

In some embodiments, the compound of Formula (I) has formula:

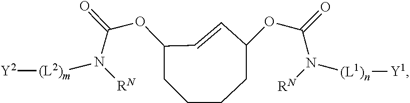

or a pharmaceutically acceptable salt thereof.

In some embodiments, the compound of Formula (I) is selected from any one of the following formulae:

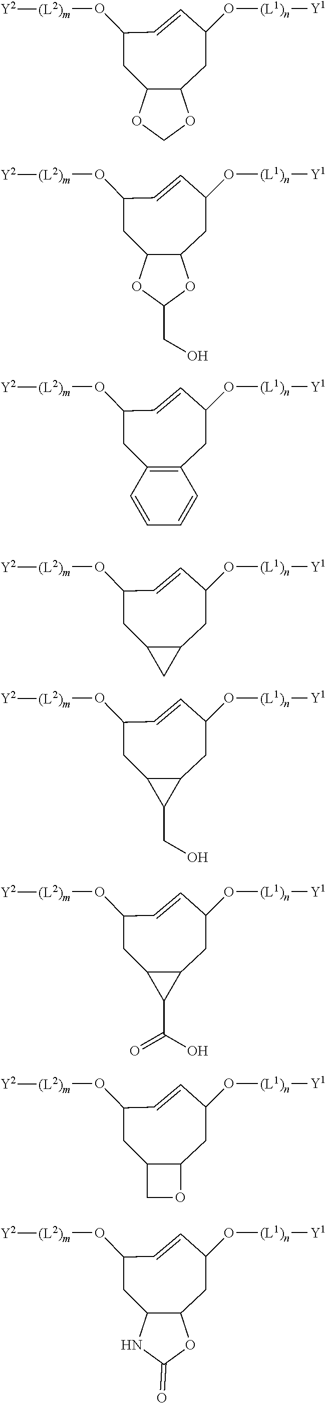

or a pharmaceutically acceptable salt thereof.

In some embodiments:
n is an integer from 1 to 10;
m is an integer from 1 to 10;
Y¹ is selected from NH₂, OH, C(O)OH, a protected amino group, a protected carboxyl group, and a protected hydroxyl group; and
Y² is selected from NH₂, OH, C(O)OH, a protected amino group, a protected carboxyl group, and a protected hydroxyl group.

In some embodiments:
n is an integer from 1 to 10;
m is an integer from 1 to 10;
Y¹ is selected from NH₂, OH, C(O)OH, a protected amino group, a protected carboxyl group, and a protected hydroxyl group; and
Y² is a reactive chemical group.

In some embodiments:
n is an integer from 1 to 10;
m is an integer from 1 to 10;
Y¹ is a fluorophore; and
Y² is a reactive chemical group.

In some embodiments, the compound of Formula (I) is selected from any one of the following compounds:

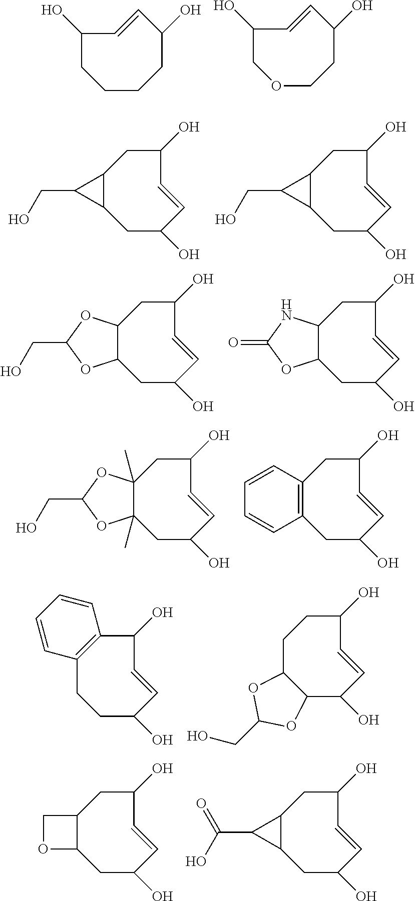

or a salt thereof (e.g., a pharmaceutically acceptable salt thereof).

In some embodiments, the compound of Formula (I) is selected from any one of the following formulae:

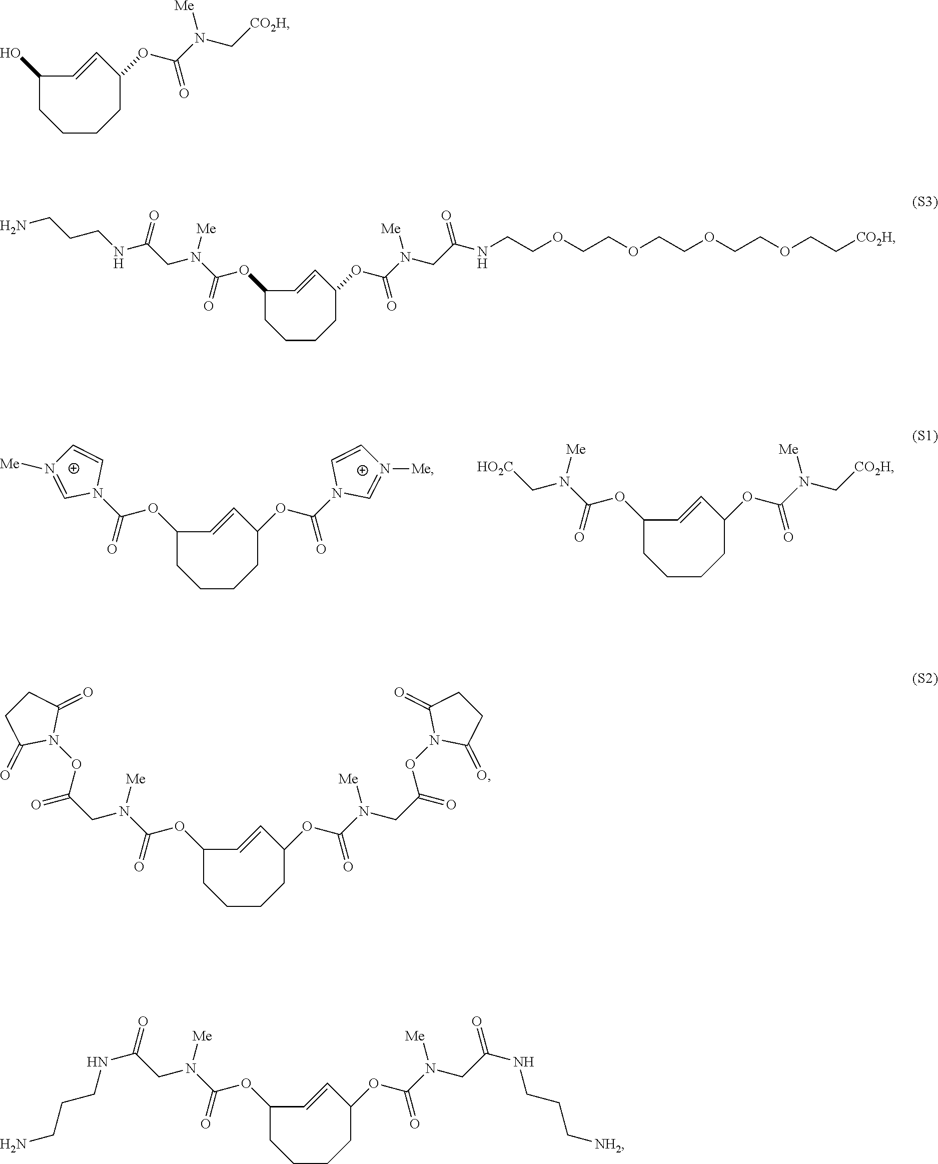

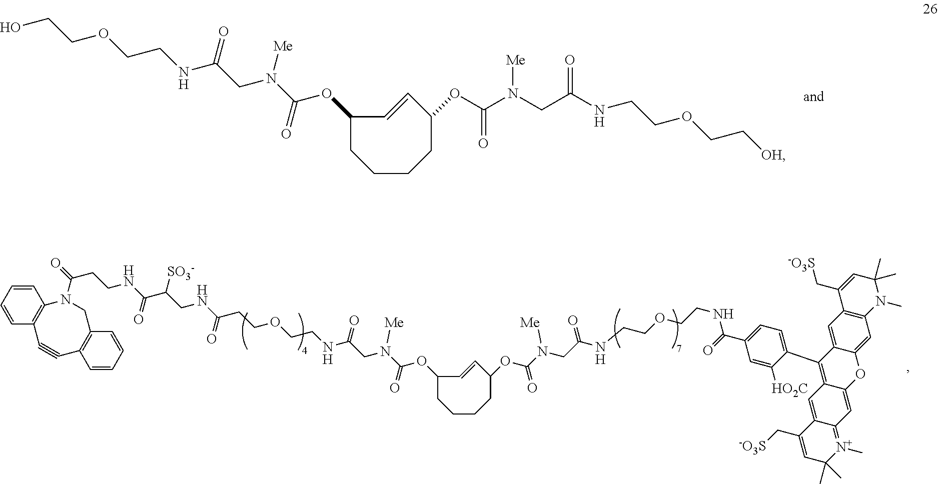

or a pharmaceutically acceptable salt thereof.

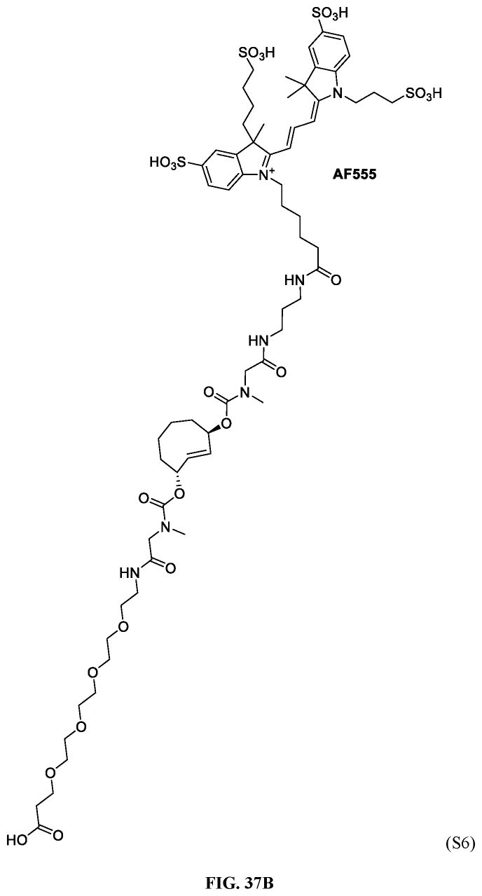

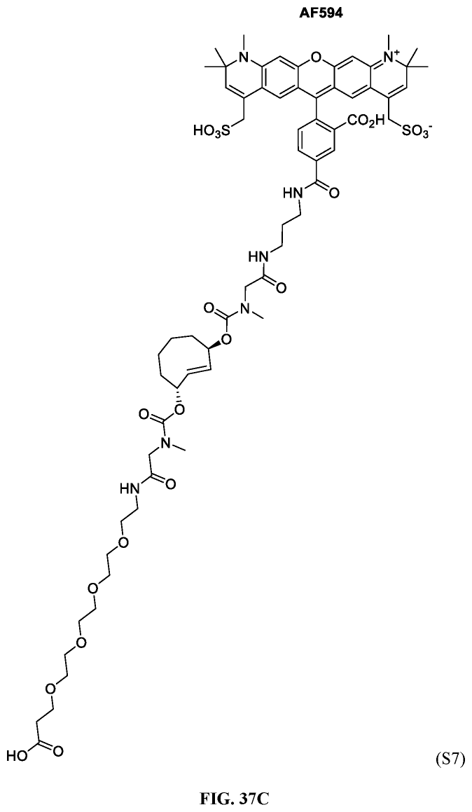

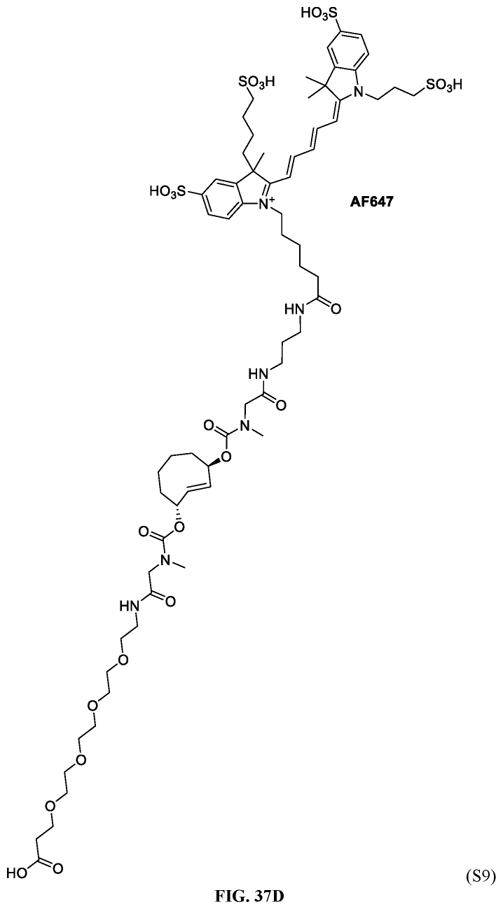

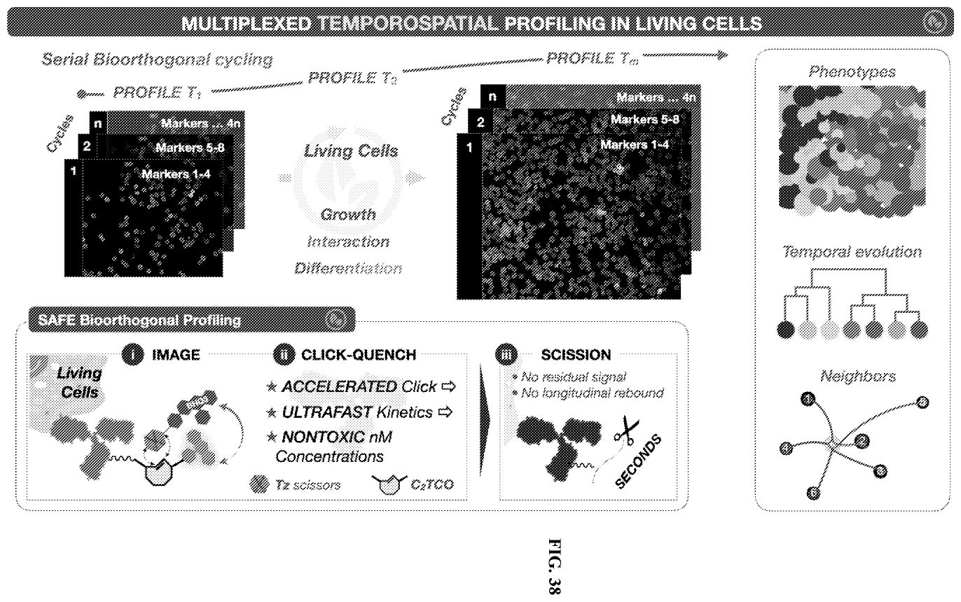

In some embodiments, the compound of Formula (I) is selected from any one of the compounds depicted in FIG. 37A-37D, 50, or 51, or a pharmaceutically acceptable salt thereof. In some embodiments, the compound of Formula (I) is selected from any one of the compounds depicted in FIGS. 37A-37D, or a pharmaceutically acceptable salt thereof.

Salts and Pharmaceutically Acceptable Salts

In some embodiments, a salt (such as a pharmaceutically acceptable salt) of a compound of Formula (I) (or any other compounds disclosed in the present disclosure) is formed between an acid and a basic group of the compound, such as an amino functional group, or a base and an acidic group of the compound, such as a carboxyl functional group. According to another embodiment, the compound is an acid addition salt (e.g., a pharmaceutically acceptable addition salt)

In some embodiments, acids commonly employed to form pharmaceutically acceptable salts of the compounds of the present disclosure include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfate, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the present disclosure provides a compound containing a “rTCO” moiety within its chemical structure. In some embodiments, the compound has Formula (A):

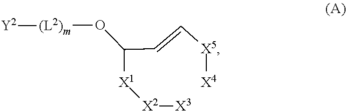

or a pharmaceutically acceptable salt thereof, wherein:
X¹ is selected from O and CHR¹;
X² is selected from O and CHR²;
X³ is selected from O and CHR³;
X⁴ is selected from O and CHR⁴;
X⁵ is selected from O and CHR⁵;
R¹, R², R³, R⁴, and R⁵ are each independently selected from H, halo, C₁₋₆ alkyl, C₁₋₆ haloalkyl, C₁₋₃ alkoxy, C₁₋₃ haloalkoxy, and (L¹)ₙ-Y¹;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-Y¹;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-Y¹;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-Y¹;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-Y¹;
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-Y¹;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C1-3-alkyl)amino, C₁₋₃ haloalkyl, C₁₋₃ alkoxy, and C₁₋₃ haloalkoxy;
n is an integer from 0 to 20;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C1-3-alkyl)amino, C₁₋₃ haloalkyl, C₁₋₃ alkoxy, and C₁₋₃ haloalkoxy;
m is an integer from 0 to 20;
each x is independently an integer from 1 to 2,000;
each R^{N} is independently selected from H, C₁₋₃ alkyl, and C₁₋₃ haloalkyl; and
each Y¹ and Y² are independently selected from H, NH₂, OH, C(O)OH, a protected amino group, a protected carboxyl group, a protected hydroxyl group, a reactive chemical group, a fluorophore, and a fluorescence quencher.

In some embodiments, X⁵ is O. In some embodiments, X⁵ is CHR⁵.

In some embodiments, R⁵ is selected from H, halo, C₁₋₆ alkyl, C₁₋₆ haloalkyl, C₁₋₃ alkoxy, C₁₋₃ haloalkoxy, and (L¹)ₙ-Y¹. In some embodiments, R⁵ is H. In some embodiments, R⁵ is selected from H, halo, and C₁₋₆ alkyl. In some embodiments, R⁵ is (L¹)ₙ-Y¹.

In some embodiments, R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, HO—C₁₋₃ alkylene, and NH₂—C₁₋₃ alkylene.

In some embodiments, R⁴ and R⁵, together with the carbon atoms to which they are attached, form an C₆₋₁₀ aryl ring (e.g., phenyl).

In some embodiments, R⁴ and R⁵, together with the carbon atoms to which they are attached, form a C₃₋₁₀ cycloalkyl ring (e.g., cyclopropyl, cyclobutyl, cyclopentyl, or cyclohexyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, R⁴ and R⁵, together with the carbon atoms to which they are attached, form a 5-14 membered heteroaryl ring (e.g., pyridinyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, R⁴ and R⁵, together with the carbon atoms to which they are attached, form a 4-10 membered heterocycloalkyl ring (e.g., azetidinyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, Y², L², m X¹, X², X³, X⁴, L¹, n, and Y¹ are as described herein for the compound of Formula (I), or a pharmaceutically acceptable salt thereof. In some embodiments, R¹ is (L¹)ₙ-Y¹. In some embodiments, R² is (L¹)ₙ-Y¹. In some embodiments, R³ is (L¹)ₙ-Y¹. In some embodiments, R⁴ is (L¹)ₙ-Y¹. In some embodiments, R⁵ is (L¹)ₙ-Y¹.

In some embodiments, the compound of Formula (A) has formula:

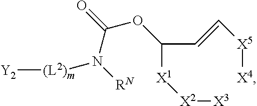

or a pharmaceutically acceptable salt thereof.

Conjugates and (Bio)Probes Containing TCO

In some embodiments, the reagents of Formula (I) can be used to prepare conjugates and (bio)probes containing C₂TCO moieties. In some embodiments, the conjugate has Formula (III):

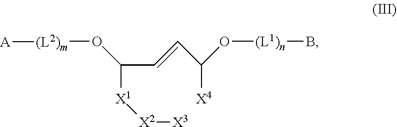

or a pharmaceutically acceptable salt thereof, wherein L², m, X¹, X², X³, X⁴, L¹, and n are as described herein for the compounds of Formula (I).

In some embodiments, the conjugate of Formula (III) has formula:

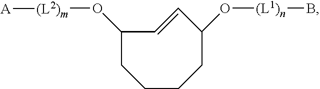

or a pharmaceutically acceptable salt thereof.

In some embodiments, the conjugate of Formula (III) has formula:

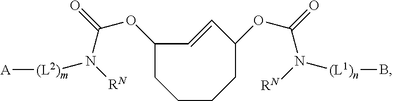

or a pharmaceutically acceptable salt thereof.

In some embodiments, the conjugate of Formula (III) has any one of the following formulae:

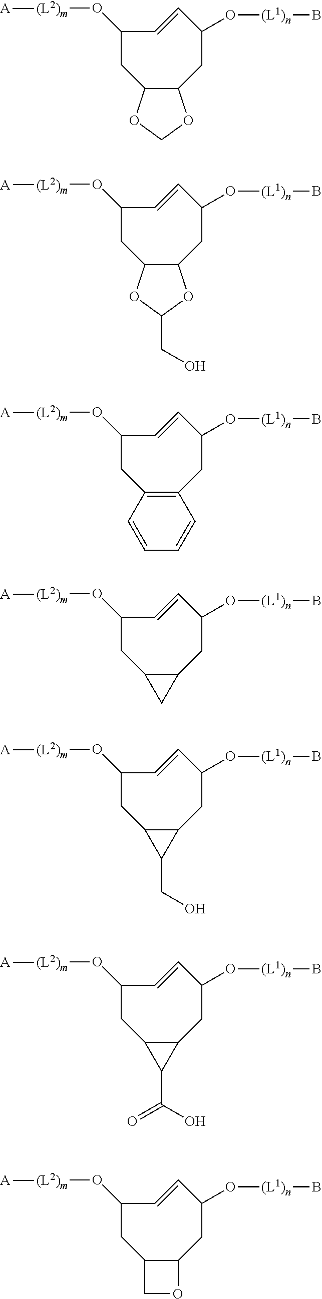

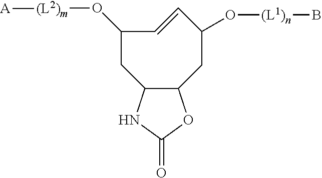

or a pharmaceutically acceptable salt thereof.

In some embodiments, A and B are each independently selected from an antibody, an antibody fragment, an engineered antibody, a peptide, a biologically active protein, a targeting group, a small-molecule drug, a fluorophore, and a fluorescence quencher.

In some embodiments, A is a fluorophore (e.g., any one of the fluorophores described herein for the compound of Formula (I)). In some embodiments, B is a fluorophore (e.g., any one of the fluorophore described herein for the compound of Formula (I)).

In some embodiments, A is a fluorescence quencher (e.g., any one of the fluorescence quenchers described herein for the compound of Formula (I)). In some embodiments, B is a fluorescence quencher (e.g., any one of the fluorescence quenchers described herein for the compound of Formula (I)).

In some embodiments, A is a targeting group. In some embodiments, B is a targeting group.

Targeting Groups

Typically, targeting groups are ligands that bind or react selectively with a receptor on the outside surface of a cell. Suitable examples of targeting groups include RGD, cRGD, folic acid, hyaluronic acid, lectin, biotin, clathrin, caveolin, and transferrin. In some embodiments, the targeting ligand promotes transcytosis or endocytosis of the conjugate. In some embodiments, the targeting ligand is a peptide. In some embodiments, the peptide is EGF, CANF, or Angiopep-2.

In some embodiments, A is a biologically active protein. In some embodiments, B is biologically active protein. In some embodiments, A is a biologically active peptide. In some embodiments, B is biologically active peptide.

Biologically Active Peptides and Proteins

Suitable examples of bioactive peptides and proteins include a hormone, a transmembrane protein, a growth factor, an enzyme, and a structural protein.

In some embodiments, A or B is a therapeutic peptide (e.g., containing 50 or fewer amino acids, 40 or fewer amino acids, 30 or fewer amino acids, 20 or fewer amino acids, or any number of amino acids that does not exceed 50). Suitable examples of peptides include Cpd86, ZPGG-72, ZP3022, MOD-6030, ZP2929, HM12525A, VSR859, NN9926, TTP273/TTP054, ZYOG1, MAR709, TT401, HM11260C, PB1023, ZP1848, ZP4207, ZP2929, dulaglutide, semaglutide, and ITCA. Other examples of therapeutic peptides are described in Fosgerau et al. Drug Discovery Today, 20 (1), 2015, 122-128; and Kaspar et al., Drug Discovery Today, 18 (17-18), 2013, 807-817.

Suitable examples of biologically active proteins include any one of the protein therapeutics described in Leader et al., Nature Reviews, 2008, 7, 21-39.

In some embodiments, the therapeutic protein is a cytokine, such as transforming growth factor-beta (TGF-beta), an interferon (e.g., interferon-alpha, interferon-beta, interferon-gamma), a colony stimulating factor (e.g., granulocyte colony stimulating factor (GM-CSF)), and thymic stromal lymphopoietin (TSLP).

In some embodiments, the cytokine is an interleukin, such as interleukin-1, interleukin-2, interleukin-3, interleukin-4, interleukin-5, interleukin-6, interleukin-7, interleukin-8, interleukin-10, interleukin-12, interleukin-13, interleukin-15, interleukin-17, interleukin-18, interleukin-22, interleukin-23, and interleukin-35.

In some embodiments, the therapeutic protein is a polypeptide hormone, such as amylin, anti-Müllerian hormone, calcitonin, cholecystokinin, corticotropin, endothelin, enkephalin, erythropoietin (EPO), darbepoetin, follicle-stimulating hormone, galanin, gastrin, ghrelin, glucagon, gonadotropin-releasing hormone, growth hormone-releasing hormone, hepcidin, human chorionic gonadotropin, growth hormone (GH), human growth hormone (hGH), inhibin, insulin, isophane insulin, insulin detemir, insulin glargine, pramlintide, pramlintide acetate, insulin-like growth factor, leptin, luteinizing hormone, luteinizing hormone releasing hormone, melanocyte stimulating hormone, motilin, orexin, oxytocin, pancreatic polypeptide, parathyroid hormone, prolactin, secretin, somatostatin, thrombopoietin, thyroid-stimulating hormone, vasoactive intestinal peptide, somatotropin, mecasermin, mecasermin rinfabate, human follicle-stimulating hormone, lutropin, teriparatide, exenatide, octreotide, dibotermin-α, bone morphogenetic protein 7, keratinocyte growth factor, platelet-derived growth factor, trypsin, nesiritide, and vasopressin.

In some embodiments, the therapeutic protein is tissue plasminogen activator (tPA), factor VIIa, factor VIII, factor IX, antithrombin III, protein C, drotrecogin-α, filgrastim, pegfilgrastim, sargramostim, lepirudin, bivalirudin, a madanin, a haemathrin, a variegin, an integrilin, or oprelvekin.

In some embodiments, the therapeutic protein is an enzyme. In some embodiments, the enzyme is agalsidase beta, imiglucerase, velaglucerase alfa, taliglucerase, alglucosidase alfa, laronidase, idursulfase, β-gluco-cerebrosidase, alglucosidase-α, laronidase, α-L-iduronidase, idursulphase, iduronate-2-sulphatase, galsulphase, agalsidase-β, human α-galactosidase A, α-1-proteinase, α-1-proteinase inhibitor, pancreatic enzyme, lactase, lipase, amylase, protease, adenosine deaminase, alteplase, reteplase, tenecteplase, urokinase, collagenase, human deoxyribonuclease I, dornase-α, hyaluronidase, papain, asparaginase (e.g. L-Asparaginase), rasburicase, streptokinase, anistreplase, or galsulfase.

In some embodiments, the therapeutic protein in useful in treating infectious disease. In some embodiments, the therapeutic protein useful in treating infectious disease is enfuvirtide.

In some embodiments, the therapeutic protein is useful in treating endocrine disorders (hormone deficiencies). In some aspects of these embodiments, the therapeutic protein is useful in treating diabetes, diabetes mellitus, diabetic ketoacidosis, hyperkalaemia, hyperglycemia, growth failure due to GH deficiency or chronic renal insufficiency, Prader-Willi syndrome, Turner syndrome, AIDS wasting or cachexia with antiviral therapy, growth failure in children with GH gene deletion or severe primary IGF1 deficiency, postmenopausal osteoporosis, severe osteoporosis, type 2 diabetes resistant to treatment with metformin and a sulphonylurea, or acromegaly.

In some embodiments, the therapeutic protein is useful in treating haemostasis and thrombosis. In some aspects of these embodiments, the therapeutic protein is useful in treating haemophilia A, haemophilia B, hereditary AT-III deficiency in connection with surgical or obstetrical procedures or for thromboembolism, venous thrombosis and purpura fulminans in patients with severe hereditary protein C deficiency, pulmonary embolism, myocardial infarction, acute ischaemic stroke, occlusion of central venous access devices, acute myocardial infarction, haemorrhage in patients with haemophilia A or B and inhibitors to factor VIII or factor IX, severe sepsis with a high risk of death, heparin-induced thrombocytopenia, blood-clotting risk in coronary angioplasty, acute evolving transmural myocardial infarction, deep vein thrombosis, arterial thrombosis, occlusion of arteriovenous cannula, and thrombolysis in patients with unstable angina.

In some embodiments, A is a biologically active small-molecule drug. In some embodiments, B is biologically small-molecule drug.

Small-Molecule Drugs

Small molecule drugs are low molecular weight organic compounds (typically about 2000 daltons or less). In some embodiments, the molecular weight of the drug molecule is in the range from about 200 to about 2000, from about 200 to about 1800, from about 200 to about 1600, from about 200 to about 1400, from about 200 to about 1200, from about 200 to about 1000, from about 200 to about 800, from about 200 to about 600 daltons, from about 300 to about 2000, from about 300 to about 1800, from about 300 to about 1600, from about 300 to about 1400, from about 300 to about 1200, from about 300 to about 1000, from about 300 to about 800, and/or from about 300 to about 600 daltons.

In some embodiments, the small-molecule drug is a cancer therapeutic. Suitable examples of small molecule drugs include antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), antifungal agents (e.g., butenafine, terbinafine, and naftifine), immunomodulating drugs (e.g., glatiramer acetate, fingolimod, teriflunomide, and dimethyl fumarate), and anti-mitotic agents (e.g., vincristine, vinblastine, paclitaxel, and maytansinoids).

Other suitable examples of small-molecule drugs include cytochalasin B, gramicidin D, doxorubicin, ethidium bromide, emetine, mitomycin, etoposide, tenoposide, colchicin, daunorubicin, dihydroxy anthracin dione, mithramycin, actinomycin D, 1-dehydrotestosterone, glucocorticoids, procaine, tetracaine, lidocaine, amphotericin B, propranolol, puromycin, and maytansinoids, e.g., maytansinol.

In some embodiments, A is selected from an antibody, an antibody fragment, and an engineered antibody. In some embodiments, B is selected from an antibody, an antibody fragment, and an engineered antibody.

Antibodies and Antibody Fragments

In some embodiments, the antibody is useful in treating cancer. In some embodiments, the antibody useful in treating cancer is abagovomab, adecatumumab, afutuzumab, alacizumab pegol, altumomab pentetate, amatuximab, anatumomab mafenatox, apolizumab, arcitumomab, bavituximab, bectumomab, belimumab, bevacizumab, bivatuzumab mertansine, blinatumomab, brentuximab vedotin, cantuzumab mertansine, cantuzumab ravtansine, capromab pendetide, cetuximab, citatuzumab bogatox, cixutumumab, clivatuzumab tetraxetan, dacetuzumab, demcizumab, detumomab, drozitumab, ecromeximab, eculizumab, elotuzumab, ensituximab, epratuzumab, etaracizumab, farletuzumab, figitumumab, flanvotumab, galiximab, gemtuzumab ozogamicin, girentuximab, ibritumomab tiuxetan, imgatuzumab, ipilimumab, labetuzumab, lexatumumab, lorvotuzumab mertansine, nimotuzumab, ofatumumab, oregovomab, panitumumab, pemtumomab, pertuzumab, tacatuzumab tetraxetan, tositumomab, trastuzumab, totumumab, or zalutumumab.

In some embodiments, the antibody is useful in treating an inflammatory disease or condition. In some embodiments, the antibody useful in treating an inflammatory disease or condition is adalimumab, alemtuzumab, atlizumab, basiliximab, canakinumab, certolizumab, certolizumab pegol, daclizumab, muromonab, efalizumab, fontolizumab, golimumab, infliximab, mepolizumab, natalizumab, omalizumab, ruplizumab, ustekinumab, visilizumab, zanolimumab, vedolizumab, belimumab, otelixizumab, teplizumab, rituximab, ofatumumab, ocrelizumab, epratuzumab, eculizumab, or briakinumab.

In some embodiments, the antibody is specific to an antigen which is a biomarker of a disease or condition. In some embodiments, the disease or condition is cancer. Suitable examples of biomarkers include CD45, CD3, CD4, CD8, PD-1, PD-L1, CD11b, F4/80, CD163, CD206, Ly6G, CD11c, and MHCII. Any other biomarker the presence of which in the cell (e.g., on the cell surface) is known in the art to be indicative of severity of the disease, or to be indicative of the presence of some disease state, can be used as an antigen for the antibody of any of the Formulae disclosed herein (e.g., Formula (I)). Some examples of cancer biomarkers include alpha fetoprotein (AFP), CA15-3, CA27-29, CA19-9, CA-125, calcitonin, calretinin, carcinoembryonic antigen, CD34, CD99MIC 2, CD117, chromogranin, chromosomes 3, 7, 17, and 9p21, cytokeratin (various types: TPA, TPS, Cyfra21-1), desmin, epithelial membrane antigen (EMA), factor VIII, CD31 FL1, glial fibrillary acidic protein (GFAP), gross cystic disease fluid protein (GCDFP-15), HMB-45, human chorionic gonadotropin (hCG), immunoglobulin, inhibin, keratin (various types), lymphocyte marker (various types, MART-1 (Melan-A), myo D1, muscle-specific actin (MSA), neurofilament, neuron-specific enolase (NSE), placental alkaline phosphatase (PLAP), prostate-specific antigen (PSA), PTPRC (CD45), 5100 protein, smooth muscle actin (SMA), synaptophysin, thymidine kinase, thyroglobulin (Tg), thyroid transcription factor-1 (TTF-1), tumor M2-PK, and vimentin.

In some embodiments of Formula (III):
n is an integer from 1 to 10;
m is an integer from 1 to 10;
A is selected from an antibody, an antibody fragment, an engineered antibody, a peptide, a targeting moiety, and a small-molecule drug; and
B is selected from a fluorophore and a fluorescence quencher.

In some embodiments of Formula (III):
n is an integer from 1 to 10;
m is an integer from 1 to 10;
A is selected from an antibody, an antibody fragment, an engineered antibody, a peptide, and a targeting moiety; and
B is a small-molecule drug.

In some embodiments of Formula (III):
A is an antibody that is specific to an antigen which is a biomarker of a disease or condition; and
B is a fluorophore.

In some embodiments of Formula (III):
A is an antibody that is specific to an antigen which is a biomarker of a disease or condition; and
B is a small-molecule drug useful in treating the disease or condition.

In some embodiments of Formula
A is a small-molecule drug; and
B is a fluorophore.

In some embodiments of Formula (III):
A is a targeting moiety; and
B is a fluorophore.

In some embodiments of Formula (III):
A is a biologically active protein or peptide; and
B is a small-molecule drug.

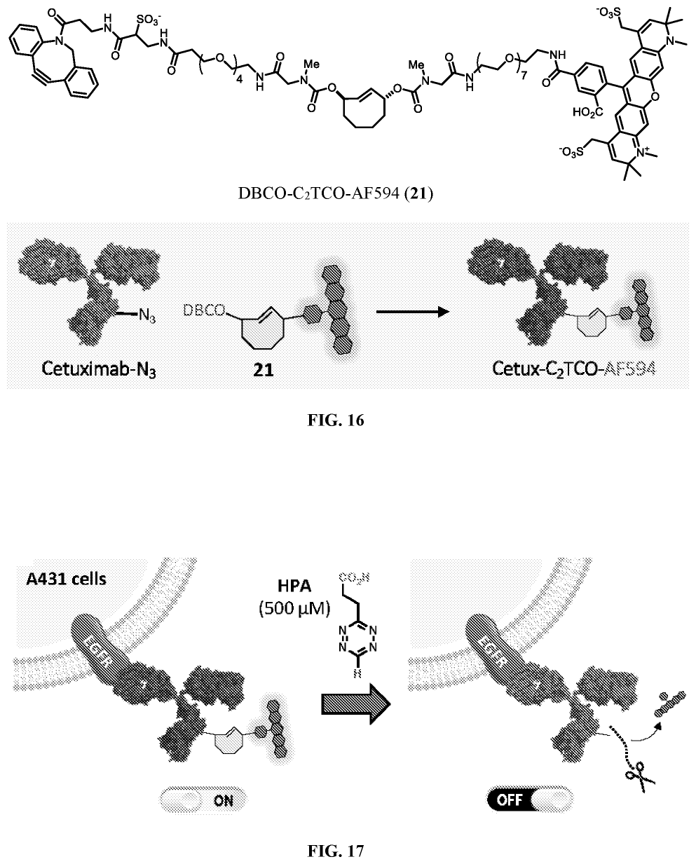

In some embodiments, the conjugate of Formula (III) is selected from any one of the conjugates depicted in FIGS. 16, 17, 19, and 36, or a pharmaceutically acceptable salt thereof.

In some embodiments, the reagents of Formula (A) can be used to prepare conjugates and (bio)probes containing rTCO moieties. In some embodiments, the conjugate has Formula (VI):

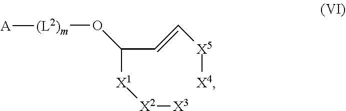

or a pharmaceutically acceptable salt thereof, wherein:
X¹ is selected from O and CHR¹;
X² is selected from O and CHR²;
X³ is selected from O and CHR³;
X⁴ is selected from O and CHR⁴;
X⁵ is selected from O and CHR⁵;
R¹, R², R³, R⁴, and R⁵ are each independently selected from H, halo, C₁₋₆ alkyl, C₁₋₆ haloalkyl, C₁₋₃ alkoxy, C₁₋₃ haloalkoxy, and (L¹)ₙ-B;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000;
each R^{N} is independently selected from H, C₁₋₃ alkyl, and C₁₋₃ haloalkyl; and
A and B are each independently selected from an antibody, an antibody fragment, an engineered antibody, a peptide, a biologically active protein, a small-molecule drug, a targeting moiety, a fluorophore, and a fluorescence quencher.

In some embodiments, L², L¹, m, n, X¹, X², X³, X⁴, and X⁵ are as described herein for Formula (A). In some embodiments, R¹ is (L¹)ₙ-B. In some embodiments, R² is (L¹)ₙ-B. In some embodiments, R³ is (L¹)ₙ-B. In some embodiments, R⁴ is (L¹)ₙ-B. In some embodiments, R⁵ is (L¹)ₙ-B.

In some embodiments, the conjugate of Formula (VI) has formula:

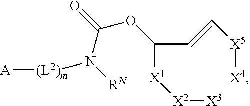

or a pharmaceutically acceptable salt thereof.

In some embodiments of the conjugate of Formula (VI), A and B are as described herein for the conjugate of Formula (III).

In some embodiments of the conjugate of Formula (VI):
A is selected from an antibody, an antibody fragment, an engineered antibody, a peptide, a targeting moiety, and a small-molecule drug; and
B is selected from a fluorophore and a fluorescence quencher.

In some embodiments of the conjugate of Formula (VI):
A is a small-molecule drug; and
B is selected from an antibody, an antibody fragment, an engineered antibody, a peptide, and a targeting moiety.

In some embodiments of the conjugate of Formula (VI), A is an antibody, an antibody fragment, an engineered antibody, a peptide, and a small-molecule drug; and B is absent.

In some embodiments of the conjugate of Formula (VI), A is a small-molecule drug; and B is absent. In some embodiments of the conjugate of Formula (VI), A is a biologically active protein or peptide (e.g., tPA), and B is absent. In some embodiments of the conjugate of Formula (VI), A is a biologically active protein or peptide, and B is a small-molecule drug. In some embodiments of the conjugate of Formula (VI), A is a biologically active protein or peptide, and B is a targeting moiety.

Biologically Active Molecules Containing TCO

In some embodiments, the present disclosure provides biologically active compounds comprising a bioorthogonally reactive functional group. In one example, the bioorthogonally reactive functionality (e.g., tetrazine or trans-cyclooctene) is embedded within the chemical structure of the therapeutic molecule. For example, a trans-cyclooctene moiety can be embedded within an oligomeric (e.g., peptide) or polymeric (e.g., protein) therapeutic molecule.

In some embodiments, the reagents of Formula (I) can be used to prepare a biologically active molecule comprising a moiety of formula (i):

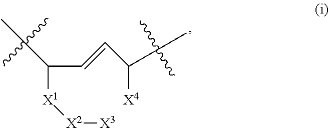

or a pharmaceutically acceptable salt thereof, wherein:
X¹ is selected from O and CHR¹;
X² is selected from O and CHR²;
X³ is selected from O and CHR³;
X⁴ is selected from O and CHR⁴;
R¹, R², R³, and R⁴ are each independently selected from H, halo, C₁₋₆ alkyl, C₁₋₆ haloalkyl, C₁₋₃ alkoxy, and C₁₋₃ haloalkoxy;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene.

In some embodiments, X¹, X², X³, X⁴, R², R³, and R⁴ are as described herein for Formula (I).

In some embodiments, the biologically active molecule is a peptide. In some embodiments, the biologically active molecule is a protein. In some embodiments, the biologically active molecule is an antibody. In some embodiments, the biologically active molecule is a small-molecule drug. In some embodiments, the biologically active molecule, prior to incorporation of the moiety of formula (i) in its chemical structure, is any one of the proteins, peptides, and small-molecules described for A or B in the conjugate of Formula (III).

In some embodiments, the biologically active molecule, prior to incorporation of the moiety of formula (i) in its chemical structure, is selected from bivalirudin, a madanin, a haemathrin, a variegin, an integrilin, and a cytokine (e.g., IL-2).

In some embodiments, the reagents of Formula (A) can be used to prepare a biologically active molecule comprising a moiety of formula (ii):

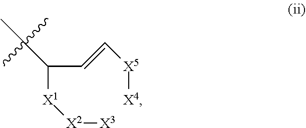

or a pharmaceutically acceptable salt thereof, wherein:
X¹ is selected from O and CHR¹;
X² is selected from O and CHR²;
X³ is selected from O and CHR³;
X⁴ is selected from O and CHR⁴;
X⁵ is selected from O and CHR⁵;
R¹, R², R³, R⁴, and R⁵ are each independently selected from H, halo, C₁₋₆ alkyl, C₁₋₆ haloalkyl, C₁₋₃ alkoxy, C₁₋₃ haloalkoxy, and a moiety of formula (iii):

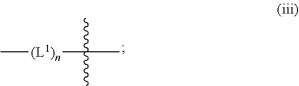

or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (iii);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (iii);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (iii);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (iii);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (iii);
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each x is independently an integer from 1 to 2,000; and
each R^{N} is independently selected from H, C₁₋₃ alkyl, and C₁₋₃ haloalkyl, provided that the moiety of formula (ii) comprises at least one moiety of formula (iii).

In some embodiments, X¹, X², X³, X⁴, X⁵, R¹, R², R³, R⁴, R⁵, L¹, n, x, and R^{N} are as described herein for the compound of Formula (A) and the conjugate of Formula (VI). In some embodiments, R¹ comprises a moiety of formula (iii). In some embodiments, R² comprises a moiety of formula (iii). In some embodiments, R³ comprises a moiety of formula (iii). In some embodiments, R³ comprises a moiety of formula (iii). In some embodiments, R⁴ comprises a moiety of formula (iii). In some embodiments, R⁵ comprises a moiety of formula (iii). In some embodiments, any of the C₆₋₁₀ aryl, C₃₋₁₀ cycloalkyl, 5-14 membered heteroaryl, and 4-10 membered heterocycloalkyl rings formed by R¹, R², R³, R⁴, and R⁵ is substituted with a moiety of formula (iii).

In some embodiments, the biologically active molecule is a peptide.

In some embodiments, one [symbol] in formula (i) or formula (ii) is attached to a peptide sequence comprising from 3-6 amino acids, from 4-9 amino acids, or from 2 to 15 amino acids, and the other [symbol] in formula (i) or formula (ii) is attached to a peptide sequence comprising from 3-6 amino acids, from 4-9 amino acids, or from 2 to 15 amino acids.

In some embodiments, the compound (or biologically active molecule) has formula:

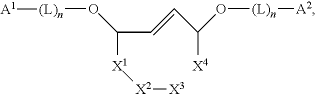

or a pharmaceutically acceptable salt thereof, where in each n is independently an integer from 1 to 100, each L is independently any of the L, L¹, L², and L³ groups described herein, and A¹ and A² are each an amino acid sequence comprising from 3-6 amino acids, from 4-9 amino acids, or from 2 to 15 amino acids.

In some embodiments, A¹ comprises 4 amino acids, e.g., a sequence FPRP (e.g., D-FPRP), and A² comprises 12 amino acids, e.g., NGDFEEIPEEYL. Any of the amino acid sequence can be linked to L by either N-terminus or C-terminus.

In some embodiments, the compound (biologically active molecule) has formula:

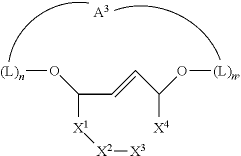

or a pharmaceutically acceptable salt thereof, where in each n is independently an integer from 0 to 100, each L is independently any of the L, L¹, L², and L³ groups described herein, and A³ is an amino acid sequence comprising form 2 to 15 amino acids, e.g., 3, 4, 5, 6, or 7 amino acids. In some embodiments, A³ comprises Mpr, Har, Gly, Asp, Trp, Pro, and/or Cys, or any combination thereof.

In some embodiments, the biologically active molecule is a protein. In some embodiments, the biologically active molecule is an antibody. In some embodiments, the biologically active molecule is a small-molecule drug. In some embodiments, the biologically active molecule, prior to incorporation of the moiety of formula (ii) in its chemical structure, is any one of the proteins, peptides, and small-molecules described for A or B in the conjugate of Formula (VI).

In some embodiments, the biologically active molecule, prior to incorporation of the moiety of formula (ii) in its chemical structure, is selected from bivalirudin, a madanin, a haemathrin, a variegin, an integrilin, and a cytokine (e.g., IL-2).

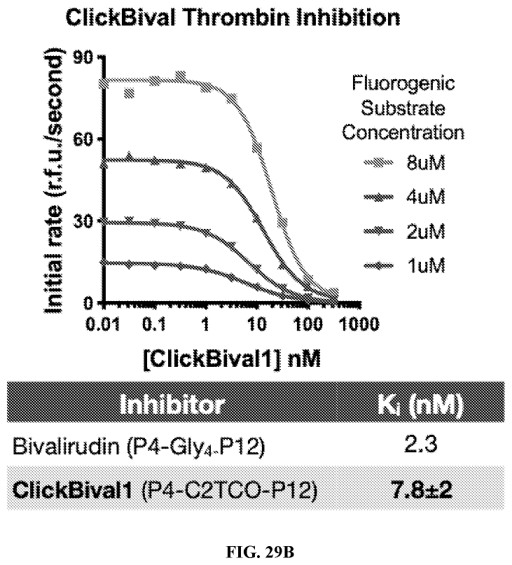

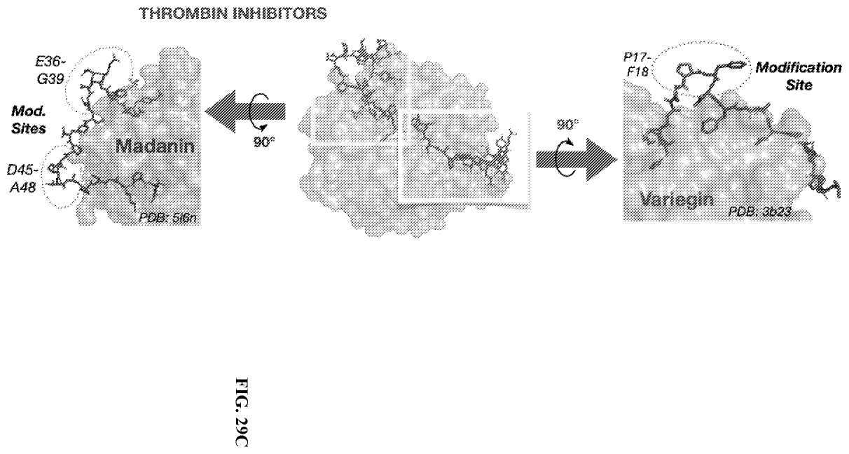

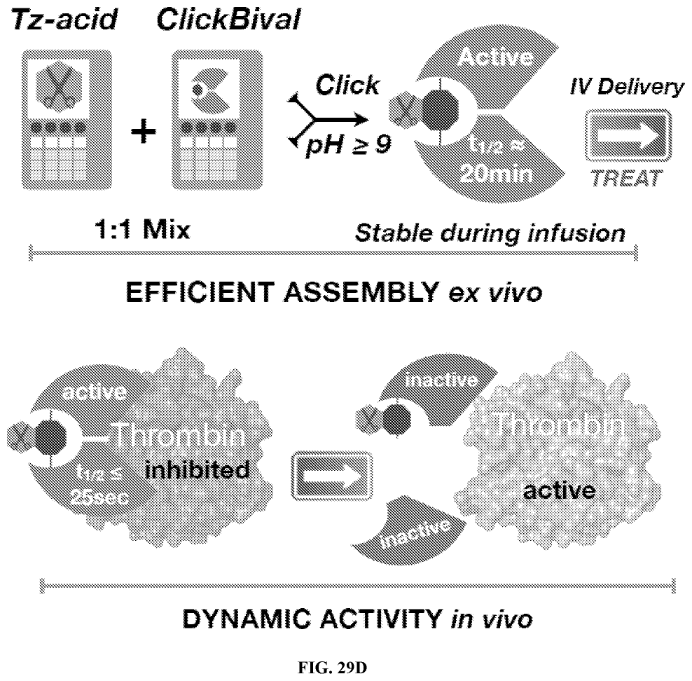

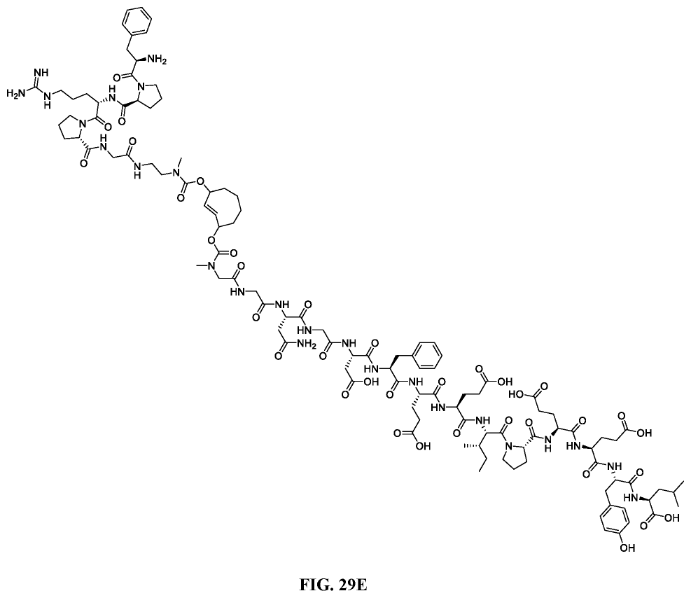

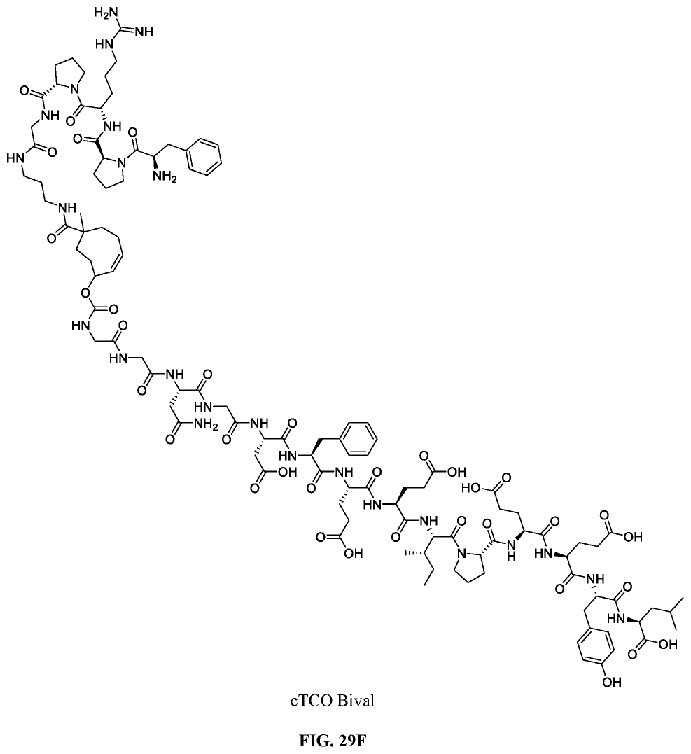

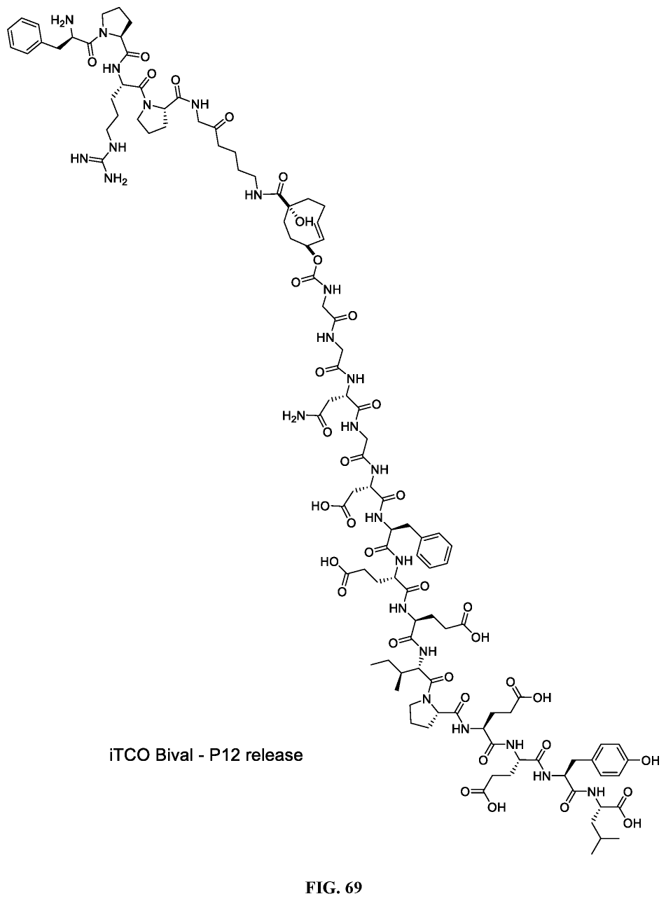

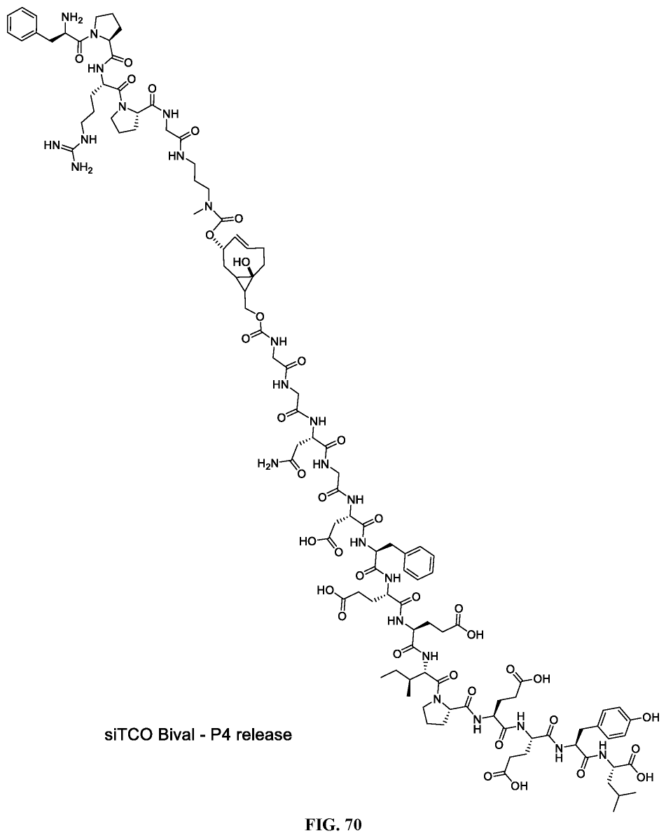

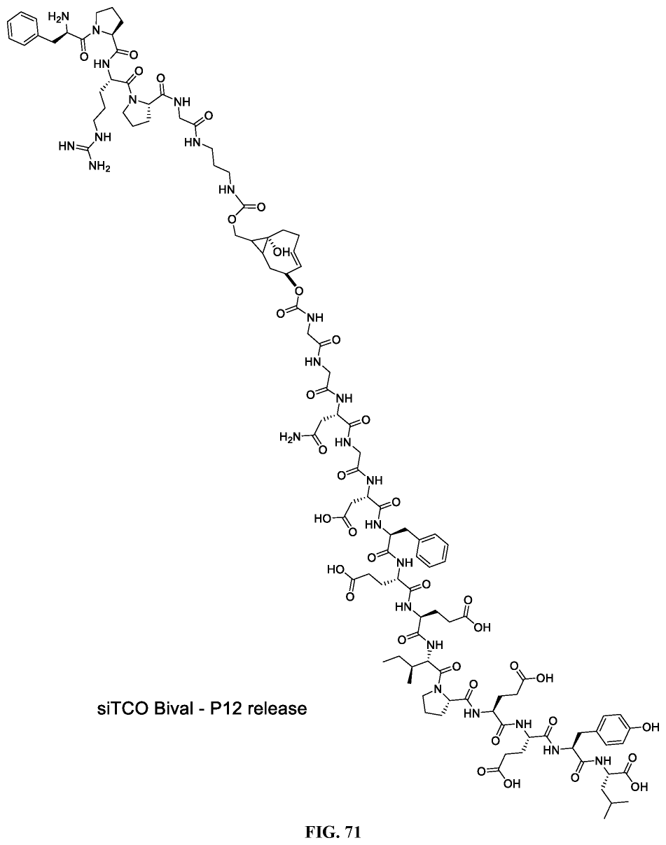

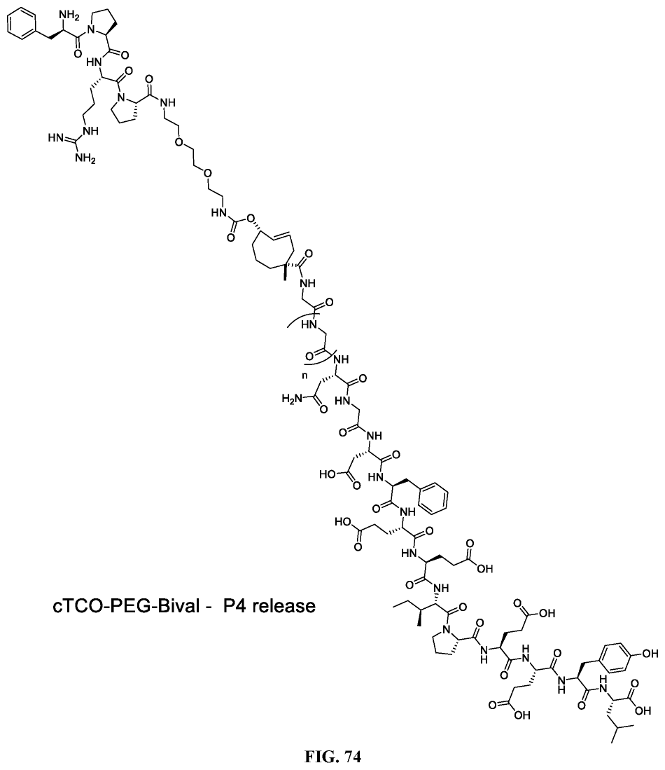

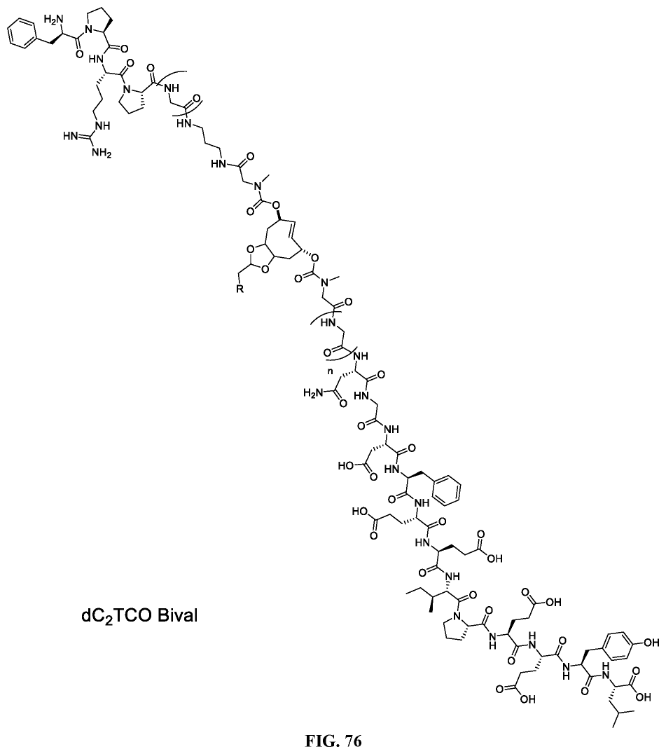

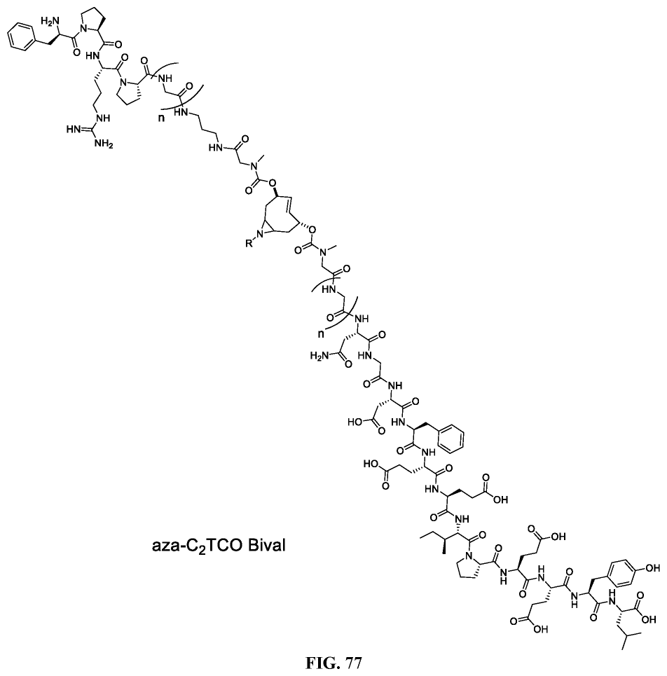

In some embodiments, the biologically active molecule comprising a moiety of formula (i) or a moiety of formula (ii) is selected from any one of the biologically active molecules depicted in FIGS. 29E, 29F, 34, 62, 64, 66, 68, 69, 70, 71, 73, 74, 76, and 77, or a pharmaceutically acceptable salt thereof. In some embodiments, the biologically active molecule comprising a moiety of formula (i) or a moiety of formula (ii) is selected from any one of the biologically active molecules depicted in FIGS. 29E, 29F, and 34, or a pharmaceutically acceptable salt thereof.

Tetrazine (“Tz”)-Containing Reagents

In some embodiments, the present disclosure provides a compound of Formula (II):

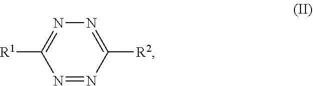

or a pharmaceutically acceptable salt thereof, wherein:
R¹ and R² are each independently selected from H, C₁₋₆ alkyl, C₆₋₁₀ aryl, 5-6-membered heteroaryl, L₁-W, and L₂-OH, wherein said C₁₋₆ alkyl, C₆₋₁₀ aryl, and 5-6-membered heteroaryl are each optionally substituted with 1 or 2 substituents independently selected from halo, C₁₋₆ alkoxy, C₁₋₆ haloalkoxy, a reactive chemical group, and L₃-X;
L₁ is C₁₋₃ alkylene, optionally substituted with 1 or 2 substituents independently selected from halo, C₁₋₆ alkoxy, C₁₋₆ haloalkoxy, a reactive chemical group, and L₃-X;
W is selected from OH, NH₂, and C(O)OH;
L₂ is C₆₋₁₀ arylene, C₃₋₁₀ cycloalkylene, 4-7-membered heterocycloalkylene, and 5-6-membered heteroarylene, each of which is optionally substituted with 1 or 2 substituents independently selected from halo, C₁₋₆ alkoxy, C₁₋₆ haloalkoxy, a reactive chemical group, and L₃-X;
L₃ is a linker; and
X is selected from a fluorophore, a fluorescence quencher, a targeting group, a reactive chemical group, and a biologically active molecule, or a fragment thereof.

In some embodiments, R¹ and R² are each independently selected from H, C₁₋₆ alkyl, C₆₋₁₀ aryl, and 5-6-membered heteroaryl, wherein said C₁₋₆ alkyl, C₆₋₁₀ aryl, and 5-6-membered heteroaryl are each optionally substituted with 1 or 2 substituents independently selected from halo, C₁₋₆ alkoxy, C₁₋₆ haloalkoxy, a reactive chemical group, and L₃-X.

In some embodiments, R¹ and R² are each C₁₋₆ alkyl.

In some embodiments, R¹ and R² are each 5-6-membered heteroaryl.

In some embodiments, R¹ is C₁₋₆ alkyl and R² is 5-6-membered heteroaryl.

In some embodiments, at least one of R¹ and R² is selected from L₁-W and L₂-OH. In some embodiments, at least one of R¹ and R² is L₁-W. In some embodiments, at least one of R¹ and R² is L₂-OH.

In some embodiments, R¹ and R² are each independently selected from L₁-W and L₂-OH. In some embodiments, R¹ is L₁-W and R² is L₁-W. In some embodiments, R¹ is L₂-OH and R² is L₂-OH.

In some embodiments, L₁ is C₁₋₃ alkylene.

In some embodiments, L₁ is C₁₋₃ alkylene substituted with a reactive chemical group (e.g., any one of the reactive chemical groups described herein for Formula (I)).

In some embodiments, L₁ is C₁₋₃ alkylene substituted with L₃-X.

In some embodiments, W is OH. In some embodiments, W is NH₂. In some embodiments, W is C(O)OH.

In some embodiments, L₂ is C₆₋₁₀ arylene, optionally substituted with a reactive chemical group (e.g., any one of the reactive chemical groups described herein for Formula (I)).

In some embodiments, L₂ is C₆₋₁₀ arylene, optionally substituted with L₃-X.

In some embodiments, L₂ is C₃₋₁₀ cycloalkylene, optionally substituted with a reactive chemical group (e.g., any one of the reactive chemical groups described herein for Formula (I)).

In some embodiments, L₂ is C₃₋₁₀ cycloalkylene, optionally substituted with L₃-X.

In some embodiments, L₂ is 4-7-membered heterocycloalkylene, optionally substituted with a reactive chemical group (e.g., any one of the reactive chemical groups described herein for Formula (I)).

In some embodiments, L₂ is 4-7-membered heterocycloalkylene, optionally substituted with L₃-X.

In some embodiments, L₂ is 5-6-membered heteroarylene, optionally substituted with a reactive chemical group (e.g., any one of the reactive chemical groups described herein for Formula (I)).

In some embodiments, L₂ is 5-6-membered heteroarylene, optionally substituted with L₃-X.

In some embodiments, OH group is attached to the carbon atom of L₂ that is in ortho position with respect to the carbon atom of L₂ that is attached to tetrazine.

In some embodiments, L₃ is a linker as described for (L₁)ₙ and (L₂)ₘ for the compounds of Formula (I) and Formula (A) (including PEG fragments, amino acids, self-immolative groups, and moieties formed by a click reaction, in addition to the other linker groups).

In some embodiments, X is any of the fluorophores, fluorescence quenchers, targeting groups, reactive chemical groups, and biologically active molecules as described for any of the formulae disclosed herein.

In some embodiments, the compound of Formula (II) has formula:

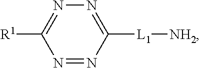

or a pharmaceutically acceptable salt thereof. In some embodiments, R¹ is selected from C₆₋₁₀ aryl, L₁-OH, L₁-C(O)OH, and L₂-OH. In some embodiments, R¹ is selected from L₁-OH, L₁-C(O)OH, and L₂-OH.

In some embodiments, the compound of Formula (II) has formula:

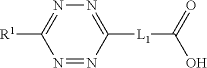

or a pharmaceutically acceptable salt thereof. In some embodiments, R¹ is selected from H, C₆₋₁₀ aryl, 5-6-membered heteroaryl, L₁-NH₂, L₁-OH, and L₂-OH. In some embodiments, R¹ is selected from L₁-NH₂, L₁-OH, and L₂-OH.

In some embodiments, the compound of Formula (II) has formula:

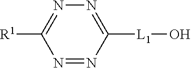

or a pharmaceutically acceptable salt thereof. In some embodiments, R¹ is selected from H, C₁₋₆ alkyl, C₆₋₁₀ aryl, L₁-OH, L₁-C(O)OH, L₁-NH₂, and L₂-OH. In some embodiments, R¹ is selected from L₁-OH, L₁-C(O)OH, L₁-NH₂, and L₂-OH.

In some embodiments, the compound of Formula (II) has formula:

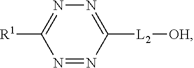

or a pharmaceutically acceptable salt thereof.

In some embodiments, the compound of Formula (II) has formula:

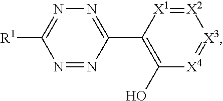

or a pharmaceutically acceptable salt thereof. In some embodiments, X¹, X², X³, and X⁴ are each independently selected from CH, N, and CR³; and each R³ is selected from halo, C₁₋₆ alkoxy, C₁₋₆ haloalkoxy, a reactive chemical group, and L₃-X. In some embodiments, X¹, X², X³, and X⁴ are each independently selected from CH and N. In some embodiments, only one of X¹, X², X³, and X⁴ is N. In some embodiments, no more than two of X¹, X², X³, and X⁴ are N.

In some embodiments, the compound of Formula (II) has formula:

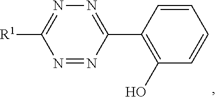

or a pharmaceutically acceptable salt thereof.

In some embodiments, the compound of Formula (II) has formula:

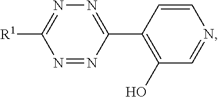

or a pharmaceutically acceptable salt thereof.

In some embodiments, the compound of Formula (II) has formula:

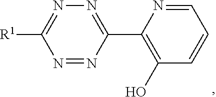

or a pharmaceutically acceptable salt thereof.

In some embodiments, R¹ is selected from C₁₋₆ alkyl, C₆₋₁₀ aryl, and 5-6-membered heteroaryl.

In some embodiments, the compound of Formula (II) has formula:

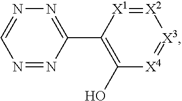

or a pharmaceutically acceptable salt thereof.

In some embodiments, the compound of Formula (II) has formula:

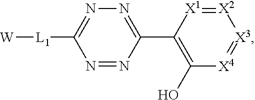

or a pharmaceutically acceptable salt thereof.

In some embodiments, the compound of Formula (II) has formula:

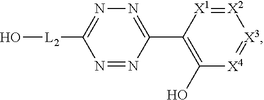

or a pharmaceutically acceptable salt thereof.

In some embodiments, the compound of Formula (II) has formula:

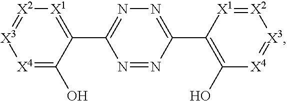

or a pharmaceutically acceptable salt thereof.

In some embodiments of the compound of Formula (II):
R¹ and R² are each independently selected from L₁-W and L₂-OH;
L₁ is C₁₋₃ alkylene, optionally substituted with 1 or 2 substituents independently selected from halo, C₁₋₆ alkoxy, C₁₋₆ haloalkoxy, a reactive chemical group, and L₃-X;
W is selected from OH, NH₂, and C(O)OH;
L₂ is C₆₋₁₀ arylene, C₃₋₁₀ cycloalkylene, 4-7-membered heterocycloalkylene, and 5-6-membered heteroarylene, each of which is optionally substituted with 1 or 2 substituents independently selected from halo, C₁₋₆ alkoxy, C₁₋₆ haloalkoxy, a reactive chemical group, and L₃-X;
L₃ is a linker; and
X is selected from a fluorophore, a fluorescence quencher, a targeting group, a reactive chemical group, and a biologically active molecule, or a fragment thereof.

In some embodiments, the compound of Formula (II) is selected from any one of the following compounds:

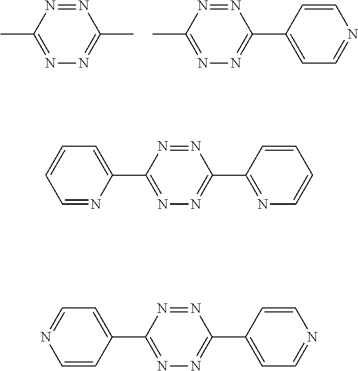

or a pharmaceutically acceptable salt thereof.

In some embodiments, the compound of Formula (II) is selected from any one of the compounds of Table 1, or a pharmaceutically acceptable salt thereof.

In some embodiments, the compound of Formula (II) is selected from any one of the following compounds:

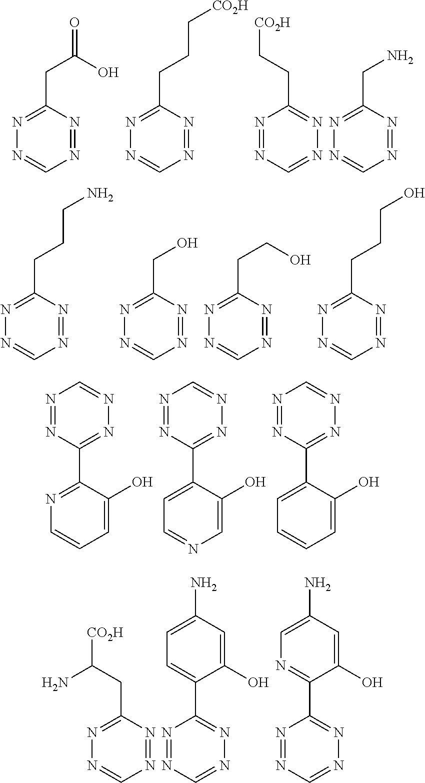

or a pharmaceutically acceptable salt thereof.

In some embodiments, the compound of Formula (II) is selected from any one of the compounds of Table 2, or a pharmaceutically acceptable salt thereof.

In some embodiments, the compound of Formula (II) is selected from any one of the following compounds:

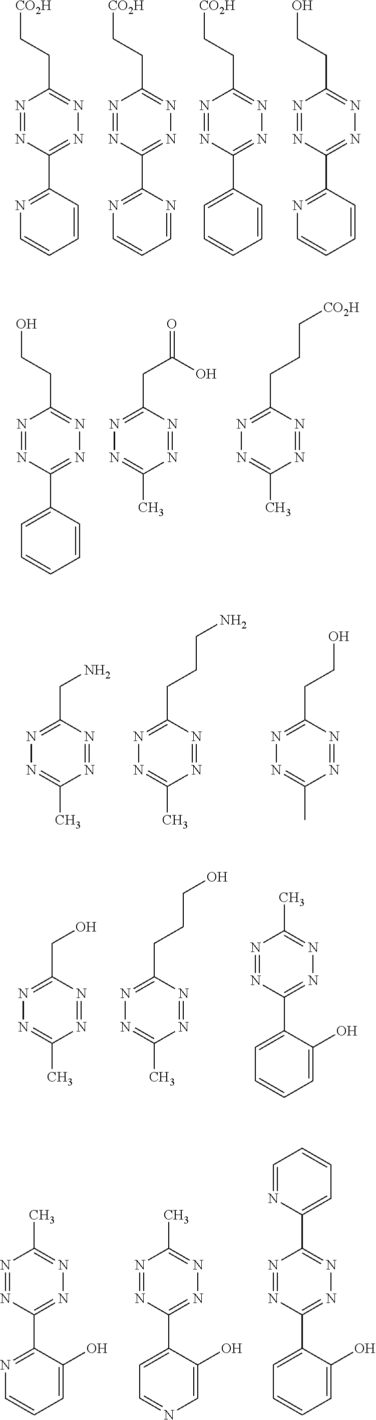

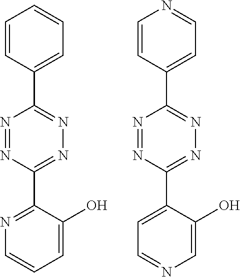 [SMILES: Oc1cccnc1-c1nnc(-c2ccccc2)nn1 ; Oc1cnccc1-c1nnc(-c2ccncc2)nn1]

or a pharmaceutically acceptable salt thereof.

In some embodiments, the compound of Formula (II) is selected from any one of the compounds of Table 3, or a pharmaceutically acceptable salt thereof.

In some embodiments, the compound of Formula (II) is selected from any one of the following compounds:

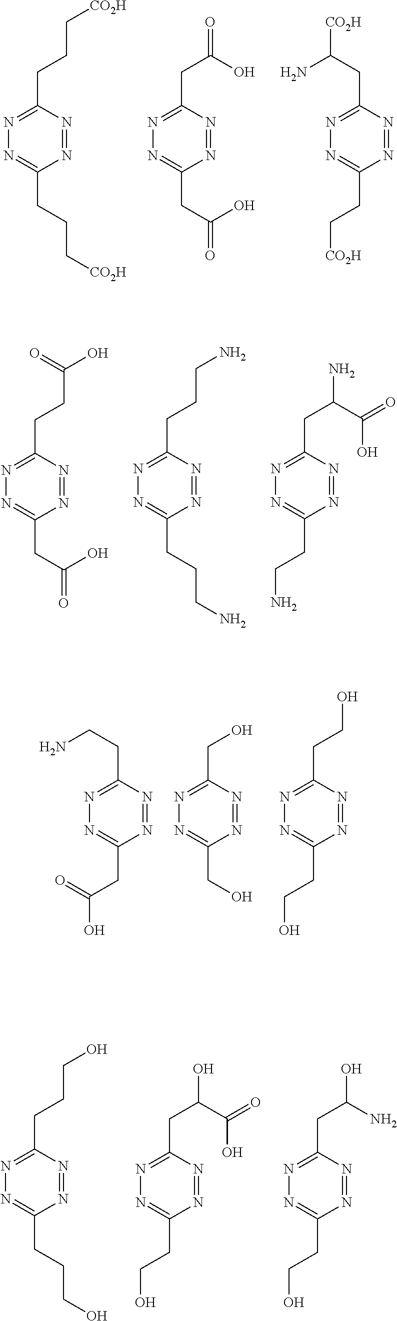

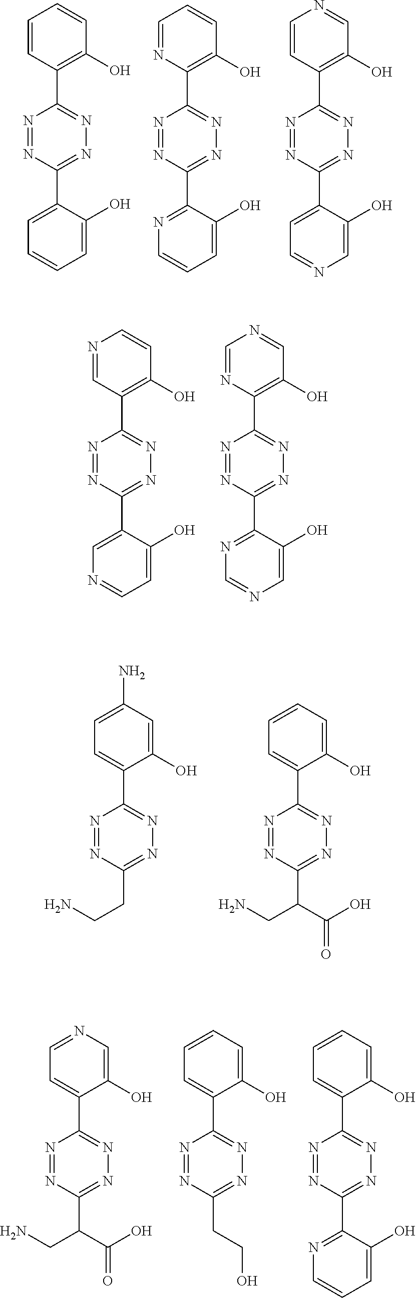

or a pharmaceutically acceptable salt thereof.

In some embodiments, the compound of Formula (II) is any one of the compounds depicted in FIG. 36B, or a pharmaceutically acceptable salt thereof.

In some embodiments, the compound of Formula (II) is not any one of the following compounds:

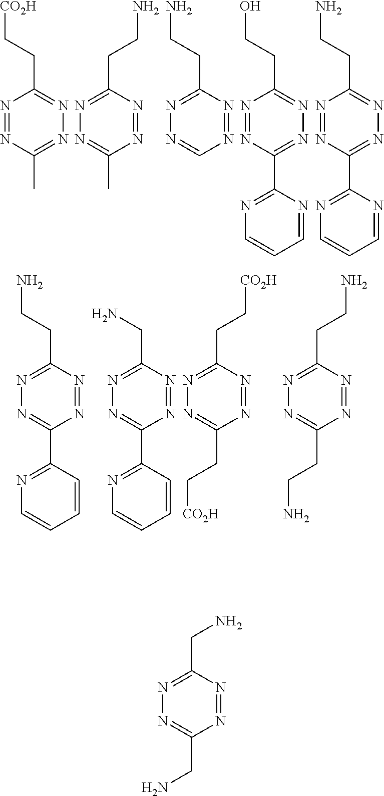

In some embodiments, the compound of Formula (II) is not any one of the following compounds:

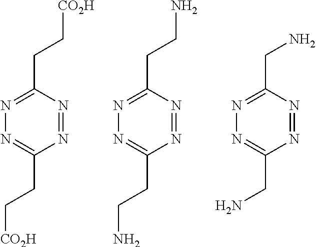 [SMILES: O=C(O)CCc1nnc(CCC(=O)O)nn1 ; NCCc1nnc(CCN)nn1 ; NCc1nnc(CN)nn1]

Compositions and Pharmaceutically Acceptable Compositions

In some embodiments, the present disclosure provides a composition comprising a compound, a conjugate, or a biologically active molecule as described herein, or a salt thereof (e.g., a pharmaceutically acceptable salt thereof), and an inert carrier (e.g., a pharmaceutically acceptable carrier). In some embodiments, the composition is an aqueous solution (i.e., the inert carrier is water). The aqueous solution may be a buffer, such as any buffer containing inert carrier such as water, phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, polyethylene glycol, or any combination thereof. Some examples of buffers include Dulbecco's phosphate-buffered saline (DPBS), phosphate buffered saline, and Krebs-Henseleit Buffer. The pH of the buffer may be from about 5 to about 9, for example pH may be 6-8. In one example, the compound, or a salt thereof, containing a reactive group, may be admixed with the protein (e.g., antibody) or a small-molecule drug in any of the aqueous solutions described here to obtain a conjugate or a biologically active molecule containing a TCO fragment as described herein.

A composition (e.g., an aqueous solution) comprising the compound or conjugate of the present disclosure, may be used to treat a cell (e.g., a cell containing a biomarker) to image the cell using a fluorophore in the compound or conjugate.

The present application also provides pharmaceutical compositions comprising an effective amount of a compound, a conjugate, or a biologically active molecule of the present disclosure, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compound, conjugate, biologically active molecule, or therapeutic agent disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein. In compositions can also be administered on a tip of a catheter.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

Dosages and Regimens

In the pharmaceutical compositions of the present application, the compound, conjugate, biologically active molecule is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound, conjugate, or biologically active molecule can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

Kits

The present invention also includes pharmaceutical kits useful, for example, in the treatment of disorders, diseases and conditions referred to herein, which include one or more containers containing a pharmaceutical composition comprising a therapeutically effective amount of the compound, conjugate, or biologically active molecule of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein. In some embodiments, the kit includes a container with a compound, a conjugate, or a biologically active molecule containing TCO fragment, and another container with a compound containing a Tz moiety.

Combinations

The compounds of the present disclosure can be used on combination with at least one medication or therapy useful, e.g., in treating or alleviating symptoms of any one of the disease and conditions described herein. Suitable examples of such medications include any one of the antibodies, therapeutic peptides and proteins, and small-molecule drugs described for various groups A and B of conjugates of the present disclosure, or a pharmaceutically acceptable salt thereof. The compound, conjugate, or biologically active molecule of the present disclosure may be administered to the patient simultaneously with the additional therapeutic agent (in the same pharmaceutical composition or dosage form or in different compositions or dosage forms) or consecutively (the additional therapeutic agent may be administered in a separate pharmaceutical composition or dosage form before or after administration of the compound, conjugate, or biologically active molecule of the present disclosure).

Methods of Using the Compounds and Conjugates of the Present Disclosure

In some embodiments, the conjugates of the present disclosure can be used for examining a cell or a tissue. In these embodiments, at least one A or B group is a fluorophore (with the other A or B group being, e.g., a targeting moiety, a small-molecule drug, or an antibody specific for a biomarker of a disease or condition).

Accordingly, in a general aspect the present disclosure provides a method of examining a cell, a component of a cell (e.g., nucleus of a cell), or a tissue, the method comprising:
(i) contacting the cell, the component of the cell, or the tissue with a conjugate of Formula (III) or Formula (VI) comprising a fluorophore, or a salt thereof (e.g., a pharmaceutically acceptable salt thereof), or a composition (e.g., a pharmaceutical composition) comprising same;
(ii) imaging the cell, the component of the cell, or the tissue with an imaging technique; and
(iii) after (ii), contacting the cell, the component of the cell, or the tissue with a compound of Formula (II), or a salt thereof (e.g., a pharmaceutically acceptable salt thereof), or a composition (e.g., a pharmaceutical composition) comprising same.

In some embodiments, contacting the cell, the component of the cell, or the tissue with a compound of Formula (II) results in a reaction (e.g., “click” reaction) of the compound of Formula (II) with a TCO moiety within the Formula (III) or Formula (VI), leading to cleavage and molecular disassembly of the Formula (III) or Formula (VI).

Accordingly, in some embodiments, the step (iii) includes a method of making a compound selected from:

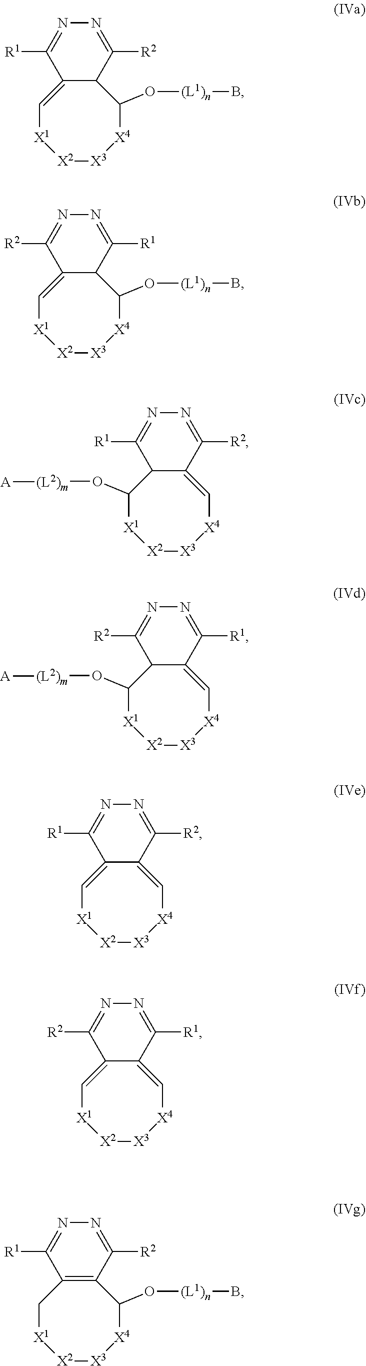

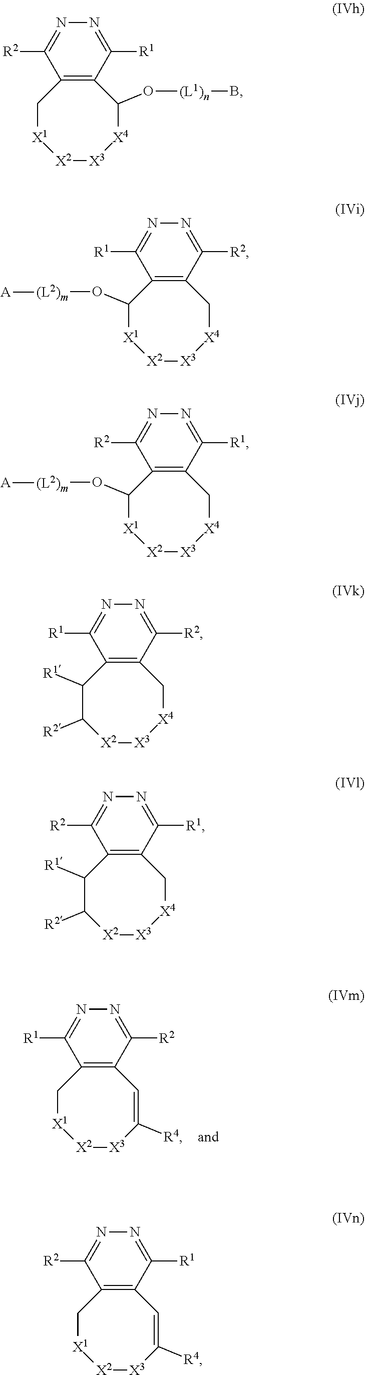

wherein R^{1′} and R^{2′} are as described for the conjugates disclosed herein, or a salt thereof (e.g., a pharmaceutically acceptable salt thereof), or any combination thereof, the method comprising reacting a conjugate of Formula (III), or a salt thereof (e.g., a pharmaceutically acceptable salt thereof), with a compound of Formula (II).

In some embodiments, the step (iii) includes reacting a conjugate of Formula (III), or a salt thereof (e.g., a pharmaceutically acceptable salt thereof), with a compound of Formula (II), or a salt thereof (e.g., a pharmaceutically acceptable salt thereof), to form a compound selected from:

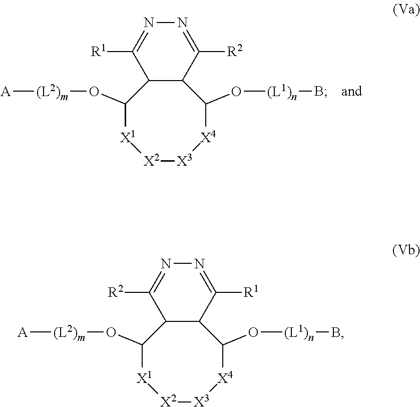

or a salt thereof (e.g., pharmaceutically acceptable salt), or a combination thereof. In some embodiment, the compound of Formula (Va) or Formula (Vb) undergoes an intramolecular cleavage cascade to form a compound selected from Formulae (IVa)-(IVn), or a salt thereof (e.g., a pharmaceutically acceptable salt thereof), or any combination thereof. In these embodiments, the fluorophore group A or B is chemically disconnected from the conjugate, and can be washed off from the cell or the tissue, such that substantially no fluorescence may be detected from the cell or the tissue. In some embodiments, the compound of Formula (Va) or Formula (Vb), or a combination thereof, may be formed in vitro. In some embodiments, the compound of Formula (Va) or Formula (Vb), or a combination thereof, may be formed in vivo. In some embodiments, the compound of Formula (Va) or Formula (Vb), or a combination thereof, may be formed ex vivo.

Accordingly, in some embodiments, the step (iii) includes a method of making a compound selected from:

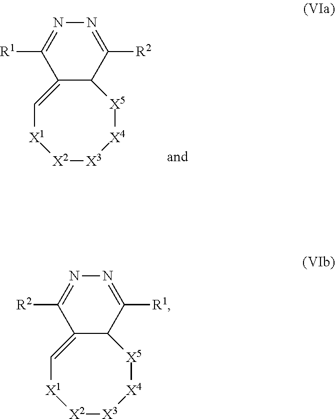

or a salt thereof (e.g., a pharmaceutically acceptable salt thereof), or any combination thereof, the method comprising reacting a conjugate of Formula (VI), or a salt thereof (e.g., a pharmaceutically acceptable salt thereof), with a compound of Formula (II).

In some embodiments, the step (iii) includes reacting a conjugate of Formula (VI), or a salt thereof (e.g., a pharmaceutically acceptable salt thereof), with a compound of Formula (II), or a salt thereof (e.g., a pharmaceutically acceptable salt thereof), to form a compound selected from:

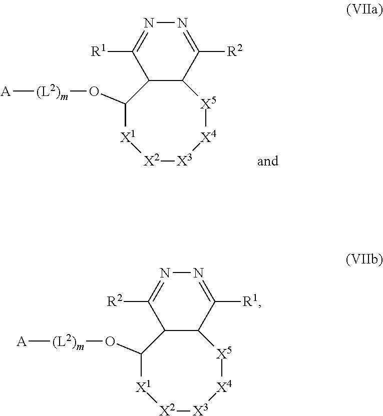

or a salt thereof (e.g., pharmaceutically acceptable salt), or a combination thereof. In some embodiment, the compound of Formula (VIIa) or Formula (VIIb) undergoes molecular cleavage cascade to form a compound selected from Formulae (VIa)-(VIb), or a salt thereof (e.g., a pharmaceutically acceptable salt thereof), or any combination thereof. In these embodiments, the fluorophore group A or B is chemically disconnected from the conjugate, and can be washed off from the cell or the tissue, such that substantially no fluorescence may be detected from the cell or the tissue. In some embodiments, the compound of Formula (VIIa) or Formula (VIIb), or a combination thereof, may be formed in vitro. In some embodiments, the compound of Formula (VIIa) or Formula (VIIb), or a combination thereof, may be formed in vivo. In some embodiments, the compound of Formula (VIIa) or Formula (VIIb), or a combination thereof, may be formed ex vivo.

In a general aspect the present disclosure also provides a method of examining a cell, a component of a cell (e.g., nucleus of a cell), or a tissue, the method comprising:
(i) reacting a conjugate of Formula (III) or Formula (VI) comprising a fluorophore, or a salt thereof (e.g., a pharmaceutically acceptable salt thereof) and a compound of Formula (II) to form a compound of Formula (Va) or (Vb) (from Formula (III)) or a compound of Formula (VIIa) or (VIIb) (from Formula (VI)), or a salt thereof (e.g., a pharmaceutically acceptable salt thereof), or a combination thereof;
(ii) contacting the cell, the component of the cell, or the tissue with the compound of Formula (Va) or (Vb), or the compound of Formula (VIIa) or (VIIb), or a salt thereof (e.g., a pharmaceutically acceptable salt thereof), or a composition (e.g., a pharmaceutical composition) comprising same; and
(iii) imaging the cell, the component of the cell, or the tissue with an imaging technique.

In some embodiments, after the imaging step (iii), the compound of Formula (Va) or (Vb) undergoes in intramolecular cleavage cascade to form a compound selected from Formulae (IVa)-(IVn), or a salt thereof (e.g., a pharmaceutically acceptable salt thereof), or any combination thereof. In some embodiments, an amount of time for the compound of Formula (Va) or (Vb) to undergo cleavage is from about 20 sec to about 2 h, from about 10 min to about 2 h, from about 20 min to about 1 h, or from about 30 min to about 1 h.

In some embodiments, after the imaging step (iii), the compound of Formula (VIIa) or (VIIb) undergoes in intramolecular cleavage cascade to form a compound selected from Formulae (VIa) or (VIb), or a salt thereof (e.g., a pharmaceutically acceptable salt thereof), or any combination thereof. In some embodiments, an amount of time for the compound of Formula (VIIa) or (VIIb) to undergo cleavage is from about 20 sec to about 2 h, from about 10 min to about 2 h, from about 20 min to about 1 h, or from about 30 min to about 1 h.

In some embodiments, the imaging technique of any of the imaging steps of the methods of the present disclosure is a fluorescence imaging, such as microscopy, imaging probes, and spectroscopy. The fluorescence imaging devices include an excitation source, the emitted light collection source, optionally optical filters, and a means for visualization (e.g., a digital camera for taking fluorescence imaging photographs). Suitable examples of fluorescence imaging include internal reflection fluorescence microscopy, light sheet fluorescence microscopy, and fluorescence-lifetime imaging microscopy. Suitable imaging techniques are described, for example, in Rao, J. et al., Fluorescence imaging in vivo: recent advances, Current Opinion in Biotechnology 18, (1), 2007, 17-25, which is incorporated herein by reference in its entirety.

In some embodiments, the present disclosure provides a method of profiling a cell, the method comprising (i) obtaining the cell from a subject, and (ii) examining the cell according to the methods of cellular analysis described herein.

In some embodiments, the present disclosure provides a method of examining a cell using a cytometry technique, the method comprising (i) obtaining the cell from a subject, and (ii) examining the cell according to the method of cellular analysis described herein. Suitable examples of cytometry techniques include image cytometry, holographic cytometry, Fourier ptychography cytometry, and fluorescence cytometry.

In some embodiments, the present disclosure provides a method of diagnosing a disease or condition of a subject by examining pathology of a cell obtained from the subject, the method comprising (i) obtaining the cell from a subject, and (ii) examining the cell according to the method of cellular analysis described herein.

In some embodiments, the present disclosure provides a method of monitoring progression of disease or condition (or monitoring efficacy of treatment of disease or condition) of a subject by examining pathology of a cell obtained from the subject, the method comprising (i) obtaining the cell from the subject, and (ii) examining the cell according to the method of cellular analysis described herein.

In some embodiments, the present disclosure provides a method of detecting a disease biomarker in a cell, the method comprising (i) obtaining the cell from a subject, and (ii) examining the cell according to the method of cellular analysis described herein.

In some embodiments, the cell is obtained from the subject using image-guided biopsy, fine needle aspiration (FNA), surgical tissue harvesting, punch biopsy, liquid biopsy, brushing, swab, touch-prep, fluid aspiration or blood analysis. In some embodiments, the cell is obtained from the subject using fine needle aspiration (FNA). In some embodiments, the cell is selected from a cancer cell, an immune system cell, and a host cell (the methods of the present disclosure are useful for hepatocyte profiling in liver disease etc.). In some embodiments, the cell is a cancer cell.

In some embodiments, the disease or condition (which can be diagnosed, monitored, or biomarker of which can be detected using the present methods) is cancer. Suitable examples of cancer include lymphoma, breast cancer, skin cancer, lymphoma nodes, head and neck cancer, and oral cancer. Other examples of cancers include colorectal cancer, gastric (gastrointestinal) cancer, leukemia, melanoma, and pancreatic cancer, hepatocellular carcinoma, ovarian cancer, endometrial cancer, fallopian tube cancer, lung cancer, medullary thyroid carcinoma, mesothelioma, sex cord-gonadal stromal tumor, adrenocortical carcinoma, synovial sarcoma, bladder cancer, smooth muscle sarcoma, skeletal muscle sarcoma, endometrial stromal sarcoma, glioma (astrocytoma, ependymoma), rhabdomyosarcoma, small, round, blue cell tumor, neuroendocrine tumor, small-cell carcinoma of the lung, thyroid cancer, esophageal cancer, and stomach cancer. The technology is useful for any cancer detectable and biospiable by image guidance.

Methods of Treatment

Any of the biologically active molecules described herein, such as those containing a TCO moiety of formula (i) or formula (ii), can be used as a “double-edged” therapeutic. That is, the biologically active molecule can be used in the methods of treating a disease or condition (such as undesired blood clotting and coagulation) that is advantageously treatable by the biologically active molecule. However, when the biological activity of the molecule in the patient's body is no longer desired, for example, due to toxicity or other adverse events, the biologically active molecule can be deactivated on demand by reaction with the compound of Formula (II). The compound of Formula (II) can be administered to the patient in a separate composition or dosage form to react with the biologically active molecule in the patient's body, resulting in decomposition of the molecule, and concomitant decrease or loss of its biological activity.

Accordingly, in some embodiments, the present disclosure includes a method of treating a disease or condition, the method comprising administering to a subject in need thereof a therapeutically effective amount of a biologically active molecule comprising a moiety of formula (i) or formula (ii), or a pharmaceutically acceptable salt thereof, or a pharmaceutical composition comprising same. In some embodiments, the method further includes administering to the subject a compound of Formula (II), or a pharmaceutically acceptable salt thereof, or a pharmaceutical composition comprising same. In some embodiments, the method includes reacting the biologically active molecule with a compound of Formula (II) (e.g., after both the biologically active molecule and the compound of Formula (II) are administered to the subject). In some embodiments, reacting the compound of Formula (II) with a biologically active molecule comprising moiety (ii) results in forming a compound selected from:

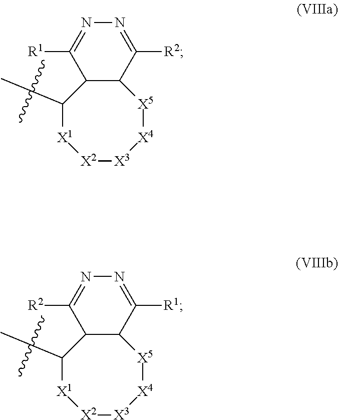

or a combination thereof.

In some embodiments, reacting the compound of Formula (II) with a biologically active molecule comprising moiety (i) results in forming a compound selected from:

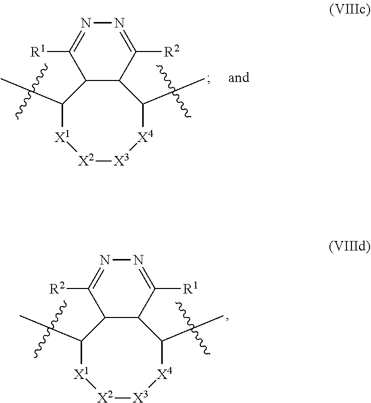

or a combination thereof.

Without being bound by a theory, it is believed that the biologically active molecule of any one of Formulae (VIIIa)-(VIIId) undergoes an intramolecular cleavage cascade leading to molecular disassembly of the biologically active molecule and a decrease or loss of its function.

In some embodiments, the present disclosure includes a method of treating a disease or condition, the method comprising administering to a subject in need thereof a therapeutically effective amount of a biologically active molecule comprising a moiety of any one of Formulae (VIIIa)-(VIIId), or a pharmaceutically acceptable salt thereof, or a pharmaceutical composition comprising same. In some embodiments, prior to administration of the Formulae (VIIIa)-(VIIId), or a pharmaceutically acceptable salt thereof, to the subject, the compound is formed by reacting a biologically active molecule comprising moiety (i) or (ii) with a compound of Formula (II). In these embodiments, the biologically active molecule of any one of Formulae (VIIIa)-(VIIId) is a “timed therapeutic,” losing its biological activity or function within a period of time determined by kinetics of intramolecular cleavage reaction. In some embodiments, the biologically active molecule remains active for a period of time from about 20 sec to about 2 h, from about 10 min to about 2 h, from about 20 min to about 1 h, or from about 30 min to about 1 h.

Suitable examples of diseases or conditions treatable by biologically active molecules of the present disclosure include an endocrine disorder (e.g., diabetes), a growth hormone deficiency, cancer (e.g., bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, or testicular cancer), an inflammatory disease or condition (e.g., arthritis, multiple sclerosis, psoriasis, or gout), a disease or condition is associated with immunodeficiencies, a disease or condition is associated with immunoregulation (e.g., chronic hepatitis C infection, hairy cell leukaemia, chronic myelogenous, leukaemia, Kaposi's sarcoma, hepatitis B, melanoma, Kaposi's sarcoma, follicular lymphoma, hairy-cell leukaemia, condylomata acuminata, hepatitis C, condylomata acuminata, multiple sclerosis, chronic granulomatous disease, severe osteopetrosis, metastatic renal cell cancer, or melanoma), disease or condition is associated with enzymatic degradation of macromolecules (e.g., dystonia, debridement of chronic dermal ulcers and severely burned areas, cystic fibrosis, respiratory tract infections, respiratory tract infections in selected patients with FVC greater than 40% of predicted, debridement of necrotic tissue, or debridement of necrotic tissue or liquefication of slough in acute and chronic lesions, pressure ulcers, varicose and diabetic ulcers, burns, postoperative wounds, pilonidal cyst wounds, carbuncles, and other wounds).

In some embodiments, the disease or condition is associated with haematopoiesis. In some embodiments, the disease or condition is associated with haematopoiesis is anaemia, myleodysplasia, anaemia due to renal failure or chemotherapy, preoperative preparation, anaemia in patients with chronic renal insufficiency and chronic renal failure (+/−dialysis), neutropaenia, neutropaenia in AIDS or post-chemotherapy or bone marrow transplantation, severe chronic neutropaenia, leukopaenia, myeloid reconstitution post-bone-marrow transplantation, or thrombocytopaenia (especially after myelosuppressive chemotherapy).

In some embodiments, the disease or condition is associated with haemostasis and thrombosis. In some embodiments, the disease or condition associated with haemostasis and thrombosis is selected from haemophilia A, haemophilia B, hereditary AT-III deficiency in connection with surgical or obstetrical procedures or for thromboembolism, venous thrombosis and purpura fulminans in patients with severe hereditary protein C deficiency, pulmonary embolism, myocardial infarction, acute ischaemic stroke, occlusion of central venous access devices, acute myocardial infarction, haemorrhage in patients with haemophilia A or B and inhibitors to factor VIII or factor IX, severe sepsis with a high risk of death, heparin-induced thrombocytopaenia, blood-clotting risk in coronary angioplasty, acute evolving transmural myocardial infarction, deep vein thrombosis, arterial thrombosis, occlusion of arteriovenous cannula, and thrombolysis in patients with unstable angina.

Anticoagulation

Anticoagulation (“AC”)—the management and prevention of blood clotting—is a foundational element of modern medical care, a cardinal issue in neurology, cardiology, and surgery, and relevant to every hospitalized patient. In spite of ongoing development of new therapeutic agents/antidotes, two major perennial challenges remain: i) medications that modulate coagulation invariably increase the risk of bleeding, creating a narrow therapeutic window; ii) patients often suffer paradoxically from simultaneous clotting and bleeding, creating irreconcilable dilemmas in which enhanced anticoagulation is needed in one physiologic territory/circuit and enhanced hemostasis in another.

In this context there are multiple high-acuity scenarios in which no safe approach exists, e.g., i) clinically significant pulmonary embolism juxtaposed with GI or CNS bleeding; ii) surgery in patients with a compelling need for ongoing anticoagulation or acute venous thrombosis after an operation; iii) obligatory anticoagulation for mechanical life support devices in patients with concurrent bleeding. While catheter-directed approaches enable semi-selective local delivery of fibrinolytics to treat life-threatening clots in a subset of these scenarios, they nonetheless confer a significant systemic bleeding risk. There is no corresponding approach for locoregional therapeutic AC; systemic doses can be escalated carefully, but binary decision-making is ultimately required.

The biologically active compounds and methods of the present disclosure advantageously allow to spatially restrict the scope of anticoagulation to affected regions within the circulation. Examples of such use include selective (i) cardiopulmonary AC for pulmonary embolism, atrial fibrillation, or mechanical heart valve prophylaxis, sparing hemostasis at peripheral loci of active bleeding/risk; (ii) in the surgical context, a local antidote to modulate AC selectively at the operative site; and (iii) selective extra-corporeal AC within a life-support circuit (e.g., ECMO, VAD, cardiopulmonary bypass) that requires AC even when the patient does not, minimizing hemostatic impact on the patient.

Given continuous recirculation in the blood stream, the half-lives of conventional anticoagulants are far too long to achieve selective local or locoregional delivery downstream from the site of infusion. Therapeutic half-life of such anticoagulants remains dependent on the complex and molecularly extrinsic interplay of biodistribution, metabolism, and excretion.

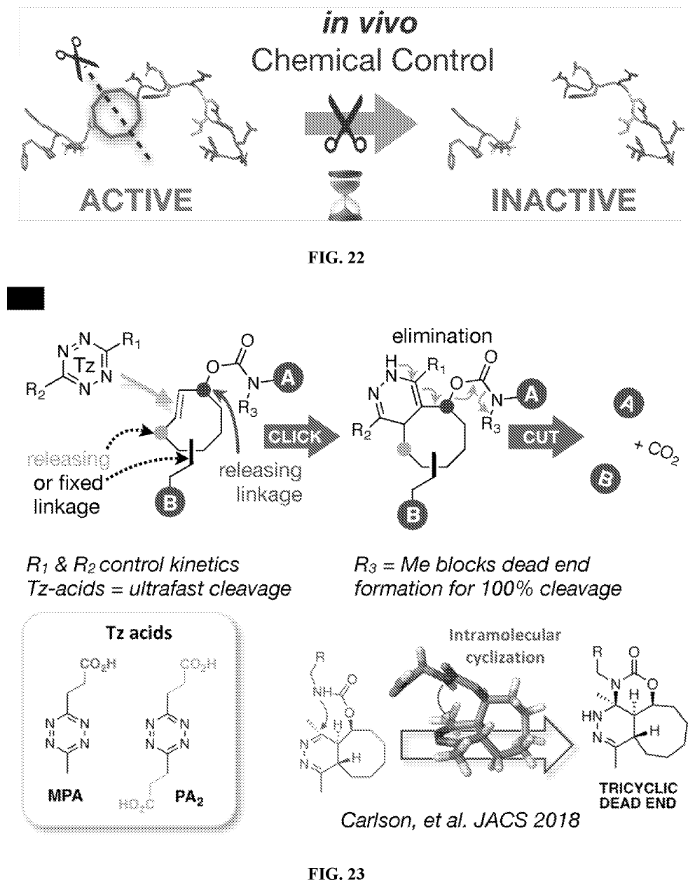

The approach within the present claims for modular tuning of an accelerated, molecularly intrinsic half-life of a biologically active compound is illustrated in FIG. 22. The approach shows broad utility in selective delivery of therapies to specific vascular beds, tissues, or tumors, and allows for management of bleeding and thrombosis and therefore effective regulation of anticoagulation.

Including bioorthogonal moiety within the chemical structure of a biologically active compound allows for spatiotemporal control to localize activity or mitigate systemic toxicity. This is accomplished by inactivating the biologically active compound using a bioorthogonal reagent systemically at a desired time or locally at a tissue within the patient's body where the biological activity of the compound is no longer desired, as described herein.

Kinetic Objectives for Self-Destructing Drugs

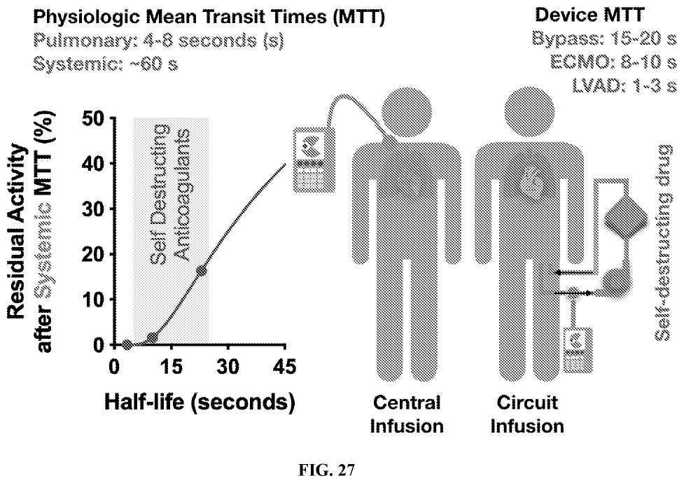

Mean transit times through the pulmonary, systemic, and life-support device circulation are key to estimating the inactivation rates needed to achieve local therapy and avoid systemic AC. The target half-lives are between 5-25 seconds (FIG. 27), depending on the application, >100-fold shorter than existing short-acting anticoagulants, such as heparin. The reagents and methods of the present disclosure provide a means to fine-tune the half-life in granular increments (by tuning the kinetics of the Tz/TCO chemistry). Advantageously, the various combinations of bioorthogonal linkers and cleaving reagents of the present disclosure allow to fine tune cleavage kinetics (including the rates of the click step and the release step), enabling both the ultra-rapid on-demand clearance as well as slower self-destructing approaches (t_{1/2} engineering). For on-demand neutralization, click reaction rates need to be as fast as possible (e.g., >10⁴ M⁻¹ s⁻¹), so that a safe and pharmacologically achievable dose of an antidote (a Tz reagent) drives the reaction to completion in a clinically relevant time frame. In contrast, click rates for self-destructing agents (slowly decomposing the (bio)molecule after administration) can be fast, but do not have to be ultrafast (e.g., >200 M⁻¹ s⁻¹). This enables flexible and precision control of conditions (time, concentration, solution pH, mixing) for reproducibility, thus facilitating development.

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “carboxy” refers to a —C(O)OH group.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl. The term “arylene” refers to a divalent aryl group, such as a phenylene.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl. The term “cycloalkylene” refers to a divalent cycloalkyl group, such as cyclopropylene.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

### EXAMPLES

The following Examples describe certain embodiments of chemical tools and probes that facilitate fast and complete cleavage for bioorthogonal “on” and “off” control.

### Example 1—Synthesis and Testing of C₂TCO

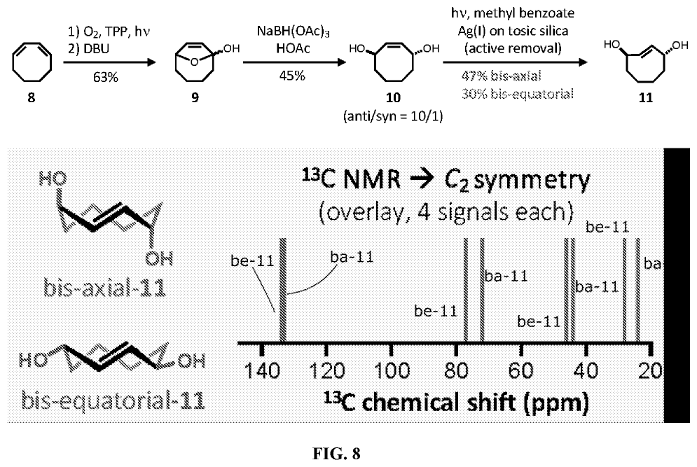

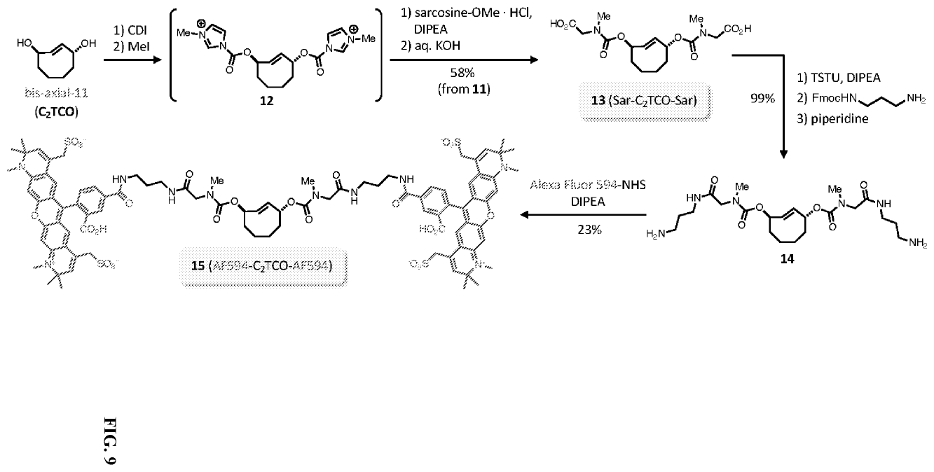

This example provides a C2-symmetric trans-cyclooctene (C₂TCO) with leaving groups at each of the two allylic positions of TCO. As shown in FIG. 8, starting from 1,3-cyclooctadiene (8), the desired C₂TCO-diol (bis-axial 11) was prepared in three steps. Tetraphenylporphyrin (TPP)-sensitized photooxygenation of 8 followed by DBU-catalyzed in-situ Kornblum-DeLaMare rearrangement afforded lactol 9 (63%), which was then reduced by reaction with sodium triacetoxyborohydride to obtain cyclooct-2-ene-1,4-diol (10) with an anti/syn-ratio of (45%). Flow-photoisomerization of 10 and continuous active removal of trans-cyclooctene products by using Ag(I) immobilized on tosic silica gel (TAg silica), finally gave bis-axial-11 (47%) and bis-equatorial-11 (30%). ¹³C NMR analysis of the separated products showed only four signals for each compound, strongly indicating the C2-symmetry of both obtained TCOs (FIG. 8, overlay of ¹³C NMR spectra), which results from the rigid ‘crown’ (or twist) conformation of the trans-cyclooctene core structure.

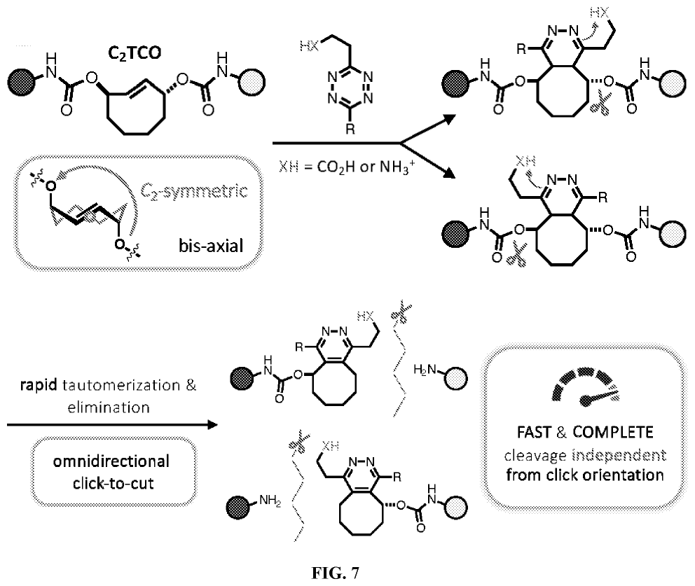

Upon click reaction with a Tz reagent, the directing group (CO₂H, NH₃⁺) on the Tz inevitably aligns (head-to-head) with one of the two release positions, irrespective of the click orientation, leading to fast and complete omnidirectional click-to-cut (FIG. 7).

Examples of C₂TCO containing linkers include:

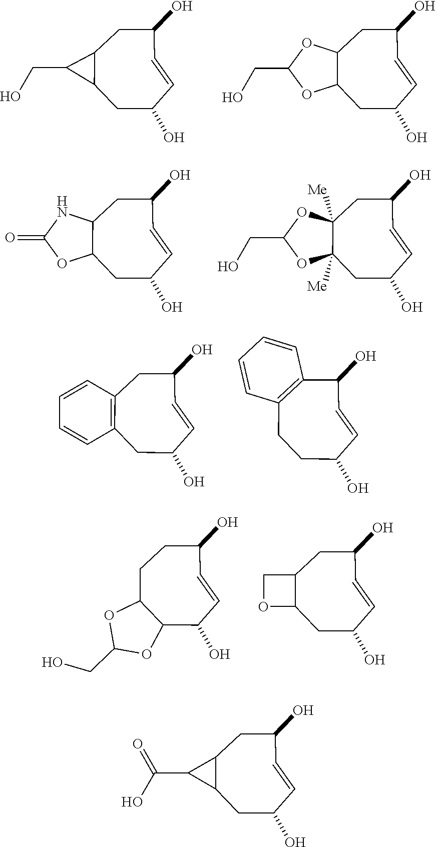

### Example 1a—Synthesis of Exemplified C₂TCO Reagents and Linkers (See FIGS. 8 and 9)

4-Hydroxycyclooct-2-ene-1-one (9)

In a PESCHL UV-Photoreactor (Borosilicate glass; THQ 150 Z1 immersion lamp, 150 W) 1,3-cyclooctadiene 8 (4.3 g, 5 mL, 40.2 mmol) was dissolved in dry DCM (400 mL). Meso-tetraphenylporphyrin (50 mg, 0.08 mmol) was added and the mixture was cooled to −5° C. Molecular oxygen was bubbled through the solution and the UV-lamp was switched on. The solution was irradiated under constant oxygen flow, while the temperature was maintained between −10° C. and 0° C. The reaction was monitored via ¹H NMR, drawing samples every hour. After 5 h further meso-tetraphenylporphyrin (50 mg, 0.08 mmol) was added to the brownish solution, as the photosensitizer had been degraded. After irradiating for additional 9 h the oxygen-bubbling was stopped and the solution was transferred to a 500 mL round bottom flask. DBU (630 μL, 4.2 mmol) was added and the mixture was stirred at room temperature for 13 h. The solvent was evaporated and the residual solid was purified by column chromatography (33-50% EtOAc in hexanes, gradient elution) to give 9 (2.01 g, 63%) as a white solid. Analytical data matched that reported in the literature; ¹H NMR (200 MHz, CDCl₃) δ 6.0 (d, J=5.3 Hz, 1H), 5.81 (d, J=5.3 Hz, 1H), 4.98 (d, J=6.1 Hz, 1H), 2.82 (bs, 1H), 2.02-1.42 (m, 8H)

anti-cyclooct-2-ene-1,4-diol (Anti-10)

Under an atmosphere of argon, a solution of sodium triacetoxyborohydride (1.51 g, 7.13 mmol) in dry DCM (360 mL) was cooled to 0° C. 4-Hydroxycyclooct-2-en-1-one 9 (500 mg, 5.13 mmol) dissolved in dry DCM (20 mL) was added followed by glacial acetic acid (500 μL). The slightly turbid solution was allowed to warm to room temperature and stirred for 8 days. Additional sodium triacetoxyborohydride (1.51 g, 7.13 mmol) and glacial acetic acid (500 μL) were added on days 3, 5, and 7. After complete consumption of the starting material, the reaction was neutralized by addition of Na₂CO₃, basified with 1N aqueous NaOH solution and extracted with DCM (3×500 mL). The combined organic layer was dried over Na₂SO₄ and concentrated. Column chromatography (50-100% EtOAc in hexanes, gradient elution) gave 10 (452.3 mg, 45%) as a white solid (mixture of diastereomers; anti-10/syn-10=syn-10: ¹H NMR (400 MHz, CD₃OD) δ 5.47 (dd, J=4.4 Hz, J=1.1 Hz, 4.47 (m, 0.2H), 1.82-1.75 (m, 0.2H), 1.72-1.61 (m, 0.4H), 1.59-1.51 (m, 0.2H); ¹³C NMR (150 MHz, CD₃OD) δ 133.94, 69.74, 39.6, 24.7; anti-10: ¹H NMR (400 MHz, CD₃OD) δ 5.55 (dd, J=3.2 Hz, J=1.1 Hz, 2H), 4.72 (m, 2H), 1.82-1.75 (m, 2H), 1.72-1.61 (m, 4H), 1.59-1.51 (m, 2H); ¹³C NMR (150 MHz, CD₃OD) δ 133.9, 69.7, 36.9, 24.1; GC-MS [M+H]⁺ m/z calcd. 143.11 for C₈H₁₅O₂⁺. found 143.11.

anti-cyclooct-2-ene-1,4-diol; C2TCO (bis-axial-11)

A mixture of anti- and syn-10 (1.06 g, 7.45 mmol) and methyl benzoate (1.9 mL, 14.9 mmol) were dissolved in Et₂O/i-PrOH=10:1 (500 mL), and the mixture was purged with argon while sonicated for 30 min. The solution was irradiated in continuous flow in a UV-photoreactor⁴ equipped with a Biotage SNAP cartridge containing 17.5 g of tosic silica⁵ for 21 h. After complete consumption of the starting material, the column was dried with air and then flushed with a mixture of Et₂O/i-PrOH=5:1 (500 mL). The crude product was eluted with 7 N methanolic ammonia solution (200 mL). After evaporation of the solvent, column chromatography (10-80% MTBE in n-hexane, gradient elution) yielded bis-axial-11 (499 mg, 47%) and bis-equatorial-11 (304 mg, 30%) as white solids; bis-equatorial-11: ¹H NMR (400 MHz, CD₃OD) δ 5.63 (dd, J=5.8 Hz, J=2.8 Hz, 2H), 4.17 (m, 2H), 2.17-2.09 (m, 2H), 1.80-1.73 (m, 2H), 1.43 (dt, J=12.2 Hz, J=10.3 Hz, 2H), 0.91-0.82 (m, 2H); ¹³C NMR (151 MHz, CD₃OD) δ 134.2, 77.2, 45.5, 28.1; bis-axial-11: ¹H NMR (400 MHz, CD₃OD) δ 6.00 (s, 2H), 4.61 (s, 2H), 2.00-1.92 (m, 2H), 1.76-1.67 (m, 2H), 1.60-1.53 (m, 2H), 1.22-1.12 (m, 2H); ¹³C NMR (151 MHz, CD₃OD) δ 133.1, 71.9, 44.5, 24.2; GC-MS [M+H]⁺ m/z calcd. 143.11 for C8H1502+. found 143.08.

Sar-C2TCO-Sar (13)

Bis-axial-11 (100 mg, 0.70 mmol) and CDI (342.1 mg, 2.10 mmol) were dissolved in dry THF (1.4 mL) and the solution was stirred for 3 h under an atmosphere of argon. The reaction mixture was diluted with Et₂O (50 mL) and washed with H₂O (3×50 mL) and brine (1×20 mL), dried over Na₂SO₄ and concentrated. The residual white solid was dissolved in dry MeCN (2.8 mL), methyl iodide (435 μL, 7.00 mmol) was added dropwise, and the mixture was stirred at room temperature for 19 h. The solvent and volatiles were evaporated to obtain the methylated imidazolium carbamate 12 as a yellow solid (343 mg). 12 was immediately dissolved in dry DMF (2.8 mL) and DIPEA (980 μL, 5.6 mmol) followed by sarcosine methylester hydrochloride (390.8 mg, 2.8 mmol) were added. After stirring for 1 h, 4 M aqueous KOH solution (2.8 mL, 11.2 mmol) was added. Complete saponification was observed after 30 min (as confirmed by LC-MS). The mixture was cooled to 0° C. and acidified with formic acid (528 μL, 14 mmol). The volatiles were evaporated and reversed phase chromatography (C18, H₂O/MeCN gradient elution, 0.1% formic acid) gave 13 (152 mg, 58%) as a white solid after lyophilization; ¹H NMR (400 MHz, CD₃OD) δ 6.00-5.88 (m, 2H), 5.37-5.31 (m, 2H), 3.91-3.78 (m, 4H), 3.03-2.94 (m, 6H), 2.18-2.03 (m, 2H), 1.93-1.60 (m, 4H), 1.28-1.11 (m, 2H); ¹³C NMR (151 MHz, CD₃OD) δ 176.1 (2C), 157.8, 157.6, 131.2, 131.0, 76.1, 76.0, 53.4 (2C), 42.1, 42.0, 41.9, 41.9, 36.3, 35.6, 25.1, 25.0; ESI-MS [M+H]⁺ m/z calcd. 373.16 for C₁₆H₂₅N₂O₈⁺. found 373.15.

Synthesis of PEG2-C2TCO-PEG2 (26)

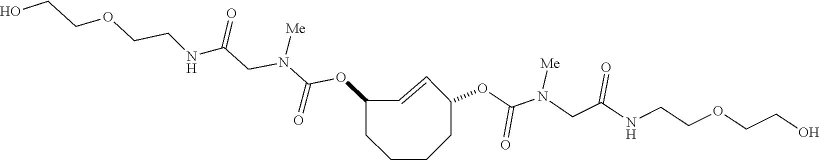

Sar-C₂TCO-Sar (25 mg, 67 μmol) was dissolved in dry DMSO (375 μL) and TSTU (62 mg, 0.21 mmol) and DIPEA (84 μl, 0.45 mmol) were added. The mixture was stirred at room temperature for 6 h until LCMS showed full conversion (after quenching a sample by addition of an excess of 2-(2-aminoethoxy)ethanol). The reaction mixture was quenched by the addition of 2-(2-aminoethoxy)ethanol (134 μL, 1.34 mmol) and stirred for 30 min until it was diluted with 0.1% formic acid in water (1.2 mL). Purification by preparative reversed phase column chromatography (H₂O/MeCN gradient elution, 0.1% formic acid) gave the desired product as a colorless oil (32 mg, 88%); ¹H NMR (600 MHz, DMSO-d₆, mixture of rotamers) δ 8.20 (bs, 1H), 8.02 (t, J=4.6 Hz, 1H), 7.93 (t, J=5.3 Hz, 1H), 5.85-5.72 (m, 2H), (m, 2H), 3.89-3.76 (m, 4H), 3.49 (t, J=5.2 Hz, 4H), 3.41 (t, J=5.0 Hz, 8H), 3.25-3.21 (m, 4H), 2.93 (d, J=4.5 Hz, 2.4H), 2.86 (s, 3.6H), 1.96 (dd, J=44.7 Hz, 14.3 Hz, 2H), 1.80-1.51 (m, 4H), 1.15-0.93 (m, 2H); ¹³C NMR (151 MHz, DMSO-d₆, mixture of rotamers) δ 168.7, 168.3, 155.1, 154.7, 129.6, 129.4, 73.64, 73.59, 72.1, 69.02, 68.96, 60.2, 51.2, 51.1, 40.5, 40.4, 40.1, 38.6, 36.1, 35.1, 23.44, 23.40, 23.31, 23.25, 21.1; ESI-MS [M+H]⁺ m/z calcd. 547.30 for C₂₄H₄₃N₄O₁₀⁺. found 547.30.

### Example 1b—Preparation of Exemplified C₂TCO Fluorophore Conjugates

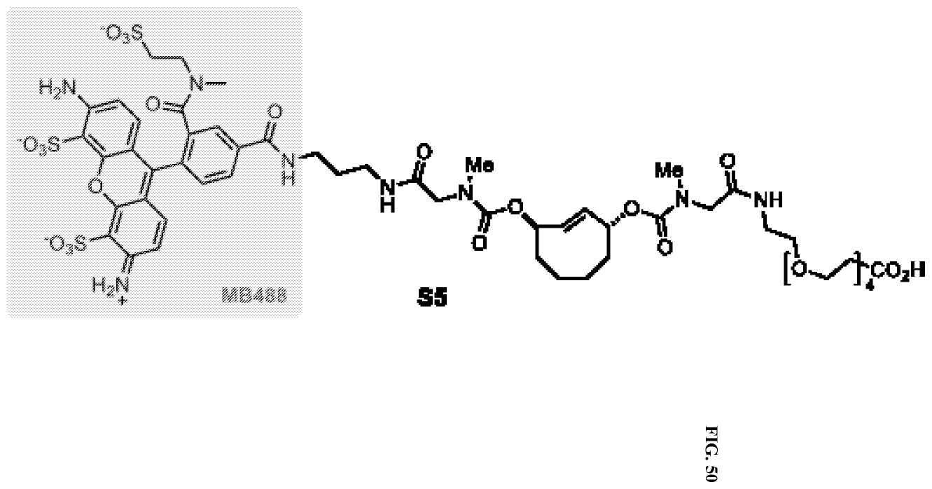

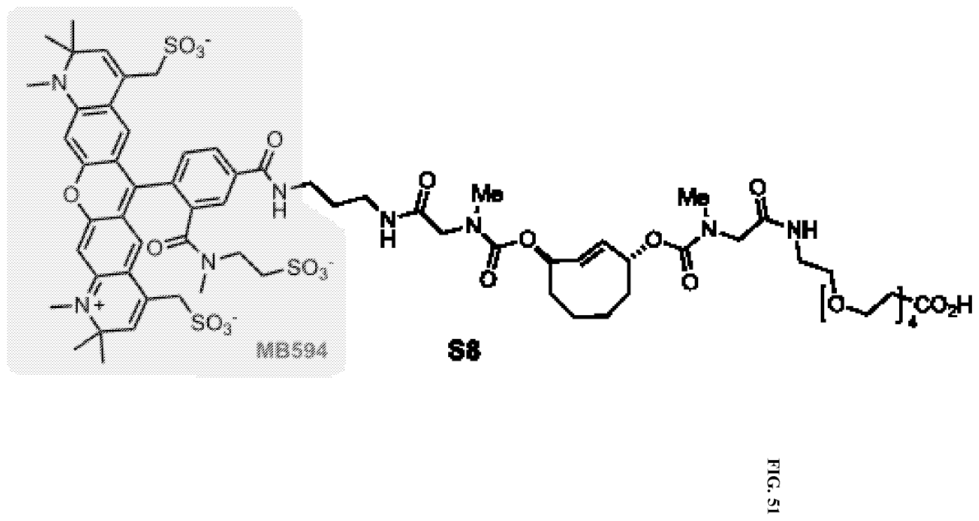

Synthesis of fluorophore-C2TCO-PEG₄-CO₂H reagents is shown schematically in FIG. 48.

DAP-C2TCO-PEG4-CO2H (S3)

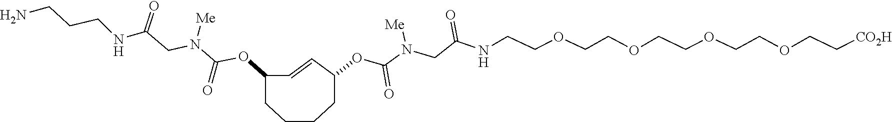

Sar-C₂TCO-Sar (S1) (147 mg, 0.39 mmol) was dissolved in dry DMSO (1 mL) and a solution of TSTU (261 mg, 0.87 mmol) in dry DMSO (1 mL) and DIPEA (444 μl, 2.57 mmol) were added. The mixture was stirred at room temperature for 90 min until LCMS analysis (after quenching a sample by addition of an excess of 2-(2-aminoethoxy)ethanol) confirmed full conversion to the bis-NHS ester S2. A solution of NH₂-PEG₄-COOH (34.6 mg, 0.13 mmol) in dry DMSO (300 μL) was added and the mixture was stirred at room temperature for 150 min until LCMS showed full conversion (after quenching a sample with an excess of 1,3-diaminopropane (DAP)). To the reaction mixture was added formic acid (315 μL) while cooling in an ice bath and water (2 mL). The solution was directly loaded onto a C18 column, followed by preparative reversed phase column chromatography (H₂O/MeCN gradient elution, formic acid) to obtain S3 as a pale-yellow oil (40.5 mg, 47%); ¹H NMR (600 MHz, DMSO-d₆, mixture of rotamers) δ 8.43-7.99 (m, 3H), 5.79 (t, J=37 Hz, 2H), 5.25 (t, J=31 Hz, 2H), 3.89-3.77 (6H), 3.57-3.42 (14H), 3.22-3.07 (5H), 2.97-2.71 (9H), 2.38-2.25 (2H), 1.95 (dd, J=47 Hz, 11.7 Hz, 2H), 1.78-1.46 (m, 7H), 1.15-0.84 (m, 2H); B C NMR (151 MHz, DMSO-d₆, mixture of rotamers) δ 173.7, 170.5, 169.0, 168.8, 168.7, 168.3, 165.7, 155.1, 154.8, 154.7, 129.9, 129.7, 129.6, 129.5, 129.4, 73.7, 69.80, 69.77, 69.74, 69.62, 69.53, 69.1, 69.0, 67.3, 67.2, 66.8, 51.3, 40.4, 38.6, 36.7, 36.6, 36.5, 36.22, 36.18, 35.7, 35.2, 35.1, 29.3, 27.8, 23.4, 23.3, 23.2, 21.1; ESI-MS [M+H]⁺ m/z calcd. 676.38 for C₂₄H₄₂N₅O₉⁺. found 676.31.

General Procedure for Conjugation of the Fluorophore to S3

DAP-C₂TCO-PEG₄-CO₂H (S3) (1 eq.) was dissolved in dry DMSO (50 μL/μmol TCO) and a solution of fluorophore-NHS ester (1.2 eq.) in dry DMSO (500 μL) and DIPEA (4 eq.) were added. The reaction mixture was stirred at room temperature until LCMS showed full conversion. The mixture was diluted with aqueous ammonium formate buffer (1 mL, 2.5 mM, pH 9.2) and directly loaded onto a C18 column. Preparative reversed phase column chromatography (ammonium formate buffer (2.5 mM, pH 9.2)/MeCN gradient elution) gave the desired product. To remove excess ammonium formate, the residue was dissolved in water (1.5 mL), loaded onto a 12 g Biotage SNAP C18 column, which was then flushed with water for 20 min. The product was obtained by elution with 95% MeCN in water and evaporation of the solvent.

AF488-C2TCO-PEG4-CO2H (S4) (Chemical Structure Shown in FIG. 37A)

Synthesis was performed following the general procedure using DAP-C₂TCO-PEG₄-COOH (S3) (5.75 mg, 8.5 μmol), AF488-NHS (6.45 mg, 10.2 μmol) and DIPEA (5.9 μL, 34 μmol). S4 was obtained after desalting as an orange oil (7.7 mg, 76%); ¹H NMR (600 MHz, D₂O, mixture of rotamers) δ 8.32 (s, 1H), 8.01 (d, J=8.0 Hz, 1H), 7.45 (d, J=8.2 Hz, 1H), 7.22-7.18 (m, 2H), 6.96 (d, J=9.3 Hz, 2H), 5.99-5.56 (m, 2H), 5.45-5.22 (m, 2H), 4.15-3.85 (m, 4H), 3.81-3.34 (m, 24H), 3.21-2.88 (m, 7H), 2.72 (s, 1H), 2.55 (t, J=6.3 Hz, 2H), 2.15-1.56 (m, 9H), 1.42-1.08 (m, 3H), (bs, 1H); ESI-MS [M−H]⁻ m/z calcd. 1190.36 for C₅₁H₆₄N₇O₂₂S₂⁻. found 1190.40.

MB488-C2TCO-PEG4-CO2H (S5) (Chemical Structure is Shown in FIG. 50)

Synthesis was performed following the general procedure using DAP-C₂TCO-PEG₄-COOH (S3) (5 mg, 7.4 μmol), MB488-NHS (6.7 mg, 8.9 μmol) and DIPEA (5.2 μL, 29.6 μmol). S5 was obtained after desalting as a red oil (4.8 mg, 51%); ¹H NMR (600 MHz, D₂O, mixture of rotamers) δ 8.13-8.01 (m, 2H), 7.88-7.79 (m, 1H), 7.65 (t, J=8.9 Hz, 1H), 7.24-7.18 (m, 3H), 7.00 (d, J=9 Hz, 2H), 5.98-5.66 (m, 2H), (m, 2H), 4.10-3.86 (m, 4H), 3.75-3.31 (m, 27H), 3.18 (q, J=7.5 Hz, 1H), 3.08-2.82 (m, 10H), 2.72 (s, 1H), 2.67 (d, J=7.4 Hz, 1H), 2.52 (t, J=6.3 Hz, 2H), 2.15-1.50 (m, 8H), 1.38-1.26 (m, 4H), 1.10 (d, J=6.8 Hz, 1H), 0.92-0.82 (m, 1H); ESI-MS [M−H]⁻ m/z calcd. 1311.37 for C₅₄H₇₁N₈O₂₄S₃⁻. found 1311.31.

AF555-C2TCO-PEG4-CO2H (S6) (Chemical Structure is Shown in FIG. 37B)

Synthesis was performed following the general procedure using DAP-C₂TCO-PEG₄-COOH (S3) (5 mg, 7.4 μmol), AF555-NHS (8.4 mg, 8.9 μmol) and DIPEA (5.2 μL, 29.6 μmol). S6 was obtained after desalting as a purple oil (7.5 mg, 68%); ¹H NMR (600 MHz, D₂O, mixture of rotamers) δ 8.57 (t, J=13.7 Hz, 1H), 8.44 (s, 1H), 7.95 (s, 1H), 7.88 (t, J=9.6 Hz, 3H), 7.45 (t, J=7.7 Hz, 2H), 6.51 (t, J=14.1 Hz, 2H), 5.93-5.57 (m, 2H), 5.40-5.22 (m, 2H), 4.31 (t, J=7.2 Hz, 2H), 4.20 (t, J=7.2 Hz, 2H), 4.02-3.84 (m, 4H), 3.75 (t, J=6.4 Hz, 2H), 3.67-3.60 (m, 15H), 3.41 (t, J=4.8 Hz, 2H), 3.16-3.06 (m, 6H), 3.00-2.92 (m, 6H), 2.69 (t, J=8.3 Hz, 2H), 2.52 (t, J=8.2 Hz, 2H), 2.47-2.23 (m, 7H), 2.06-1.87 (m, 4H), 1.80-1.77 (m, 10H), 1.68-1.57 (m, 11H), 1.39-1.30 (m, 3H), 1.08-1.04 (m, 2H), 0.89-0.78 (m, 2H); ESI-MS [(M+H)/2]⁺ m/z calcd. 752.27 for [C₆₅H₉₇N₇O₂₅S₄]⁺/2. found 752.1.

AF594-C2TCO-PEG4-CO2H (S7) (Chemical Structure is Shown in FIG. 37C)

Synthesis was performed following the general procedure using DAP-C₂TCO-PEG₄-COOH (S3) (5.75 mg, 8.5 μmol), AF594-NHS (8.4 mg, 10.2 μmol) and DIPEA (5.9 μL, 34 μmol). S7 was obtained after desalting as a dark purple oil (8.5 mg, 72%); ¹H NMR (600 MHz, D₂O, mixture of rotamers) δ 8.34 (s, 1H), 8.05-8.01 (m, 1H), 7.42-7.7.40 (m, 1H), 7.12-7.10 (m, 2H), 6.69-6.66 (m, 2H), 5.98-5.74 (m, 4H), 5.35-5.21 (m, 2H), 4.06-3.79 (m, 7H), 3.74 (t, J=6.2 Hz, 2H), 3.69-3.48 (m, 20H), 3.42-3.32 (m, 4H), 3.08-2.92 (m, 13H), 2.72 (s, 0.5H), 2.54 (t, J=6.3 Hz, 2H), 2.20-1.60 (m, 9H), 1.50 (s, 13H), 1.39-1.26 (m, 0.5H), 1.21-1.10 (m, 0.5H), 0.99-0.81 (m, 1.5); ESI-MS [M+H]⁺ m/z calcd. 1380.53 for C₆₅H₈₆N₆N₇O₂₂S₂⁺. found 1380.55.

MB594-C2TCO-PEG4-CO2H (S8) (Chemical Structure is Shown in FIG. 51)

Synthesis was performed following the general procedure using DAP-C₂TCO-PEG4-COOH (S3) (5 mg, 7.4 μmol), MB594-NHS (8.4 mg, 8.9 μmol) and DIPEA (5.2 μL, 29.6 μmol). S8 was obtained after desalting as a dark blue oil (7.3 mg, 65%); ¹H NMR (600 MHz, D₂O, mixture of rotamers) δ 8.13-8.00 (m, 2H), 7.63-7.55 (m, 1H), 7.15-7.11 (m, 2H), 6.80-6.70 (m, 2H), 6.05-5.64 (m, 4H), 5.33-5.25 (m, 2H), 4.06-3.91 (m, 6H), 3.81-3.58 (m, 19.5H), 3.54-3.36 (m, 8H), 3.12-2.91 (m, 13H), 2.72-2.66 (m, 3.5H), 2.55 (t, J=6.3 Hz, 2H), 2.25 (t, J=8.1 Hz, 2H), 2.13-1.51 (m, 1.42-1.26 (m, 1H), 1.19-1.11 (bs, 0.5H), 0.99-0.86 (m, 1.5H); ESI-MS [M+H]⁺ m/z calcd. 1501.55 for C₆₇H₉₃N₈O₂₄S₃⁺. found 1501.55.

AF647-C2TCO-PEG4-COOH (S9) (Chemical Structure is Shown in FIG. 37D)

Synthesis was performed following the general procedure using DAP-C₂TCO-PEG₄-COOH (S3) (4 mg, 5.9 μmol), AF647-NHS (6.9 mg, 7.1 μmol) and DIPEA (4.2 μL, 23.7 μmol). S9 was obtained after desalting as a blue oil (6.2 mg, 68%); ¹H NMR (600 MHz, D₂O, mixture of rotamers) δ 8.13 (q, J=13.6 Hz, 2H), 7.90-7.84 (m, 4H), 7.37 (dd, J=24.2 Hz, J=8.9 Hz, 2H), 6.67 (t, J=12.2 Hz, 1H), 6.42 (d, J=13.2 Hz, 2H), 5.92-5.70 (m, 2H), 5.26-5.21 (m, 2H), 4.26 (bs, 2H), 4.16 (bs, 2H), 4.04-3.84 (m, 4H), 3.75 (t, J=6.3 Hz, 2H), 3.68-3.58 (m, 15H), 3.39 (bs, 2H), 3.16-2.92 (m, 12.5H), 2.72-2.68 (m, 3.5H), 2.38-2.21 (m, 6H), 2.02-1.84 (m, 4H), 1.72-1.50 (m, 20H), 1.35 (bs, 2H), 0.99 (bs, 1H), 0.87 (m, 1H), 0.71 (bs, 1H); ESI-MS [M+H]⁺ m/z calcd. 1530.56 for C₆₇H₁₀₀N₇O₂₅S₄⁺. found 1530.50.

Synthesis of AF594-C2TCO-AF594

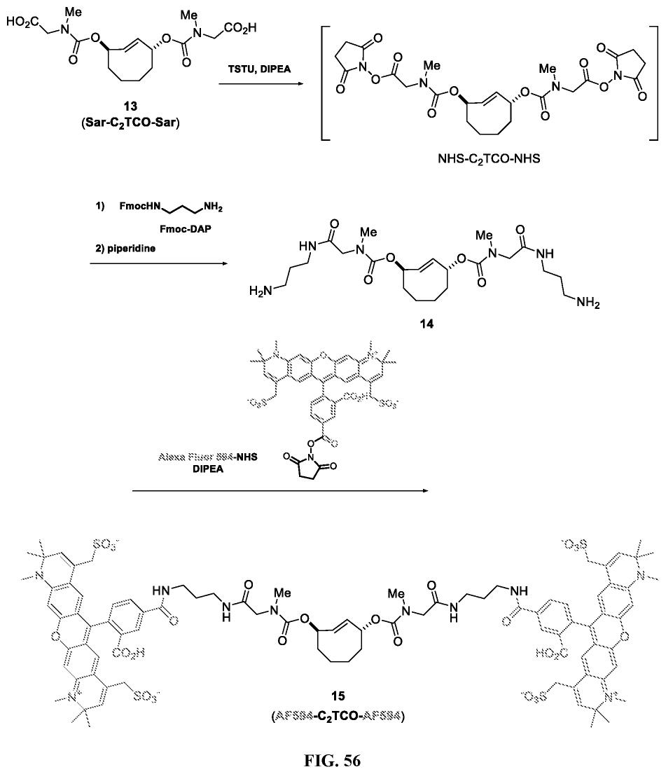

The synthesis is schematically shown in FIG. 56.

DAP-C2TCO-DAP (14)

Sar-C₂TCO-Sar (13) (13.8 mg, 0.04 mmol) was dissolved in dry DMSO (375 μL) and TSTU (23.5 mg, 0.08 mmol) and DIPEA (27.2 μl, 0.16 mmol) were added. The mixture was stirred at room temperature for 10 min until LCMS showed full conversion (after quenching a sample by addition of an excess of 1,3-diaminopropane (DAP)). A solution of Fmoc-DAP hydrochloride (25.9 mg, mmol) dissolved in dry DMSO (300 μL) was added and the mixture was stirred at room temperature for 15 min until LCMS showed full conversion (ESI-MS of the intermediate [Fmoc-DAP-C₂TCO-DAP-Fmoc+H]⁺ m/z calcd. 929.4 for C₅₂H₆₁N₆O₁₀⁺. found 929.5). Piperidine (220 μL) was added and the mixture was stirred for 20 min, quenched with formic acid (100 μL) and then centrifuged for 3 min at 6000 rpm. The supernatant was directly loaded onto a C18 column and reversed phase column chromatography (H₂O/MeCN gradient elution, 0.1% formic acid) gave the desired product as a pale-yellow oil (18 mg, 99%); ¹H NMR (600 MHz, D₂O, mixture of rotamers) δ 8.43 (bs, 2H), 5.96-5.75 (m, 2H), 5.27-5.26 (m, 2H), 4.07-3.90 (m, 4H), 3.45 (q, J=5.8 Hz, 2H), 3.36-3.25 (m, 4H), 3.04-3.03 (m, 2H), 2.99-2.95 (m, 6H), 2.70-2.68 (m, 4H), 1.89-1.82 (m, 4H), 1.76-1.53 (m, 6H), 1.32-1.29 (m, 2H); ¹³C NMR (151 MHz, D₂O, mixture of rotamers) δ 172.24, 172.21, 171.8, 171.7, 170.6, 157.6, 157.6, 157.0, 156.9, 129.4, 129.3, 75.9, 75.8, 75.8, 75.6, 54.3, 52.11, 52.1, 52.0, 47.0, 45.0, 44.5, 43.4, 42.5, 40.5, 40.4, 40.3, 40.2, 38.6, 37.9, 37.0, 36.9, 36.1, 35.9, 35.4, 27.7, 26.7, 26.6, 25.8, 25.2, 25.1, 23.7, 23.3, 23.2, 23.1, 22.2, 21.4, 17.6, 16.2, 12.1; HRMS [M+Na]⁺ m/z calcd. 507.2902 for C₂₂H₄₀N₆NaO₆⁺. found 507.2928.

AF594-C2TCO-AF594 (15)

To AF594-NHS (20.8 mg, 25.4 μmol) dissolved in dry DMSO (300 μL) were added DIPEA (14.1 μL, 80.5 μmol) and a solution of DAP-C₂TCO-DAP (14) (6 mg, 12.4 μmol) in dry DMSO (60 μL). The dark-blue mixture was stirred at room temperature for 80 min and then quenched by addition of formic acid (3.4 Purification by reversed phase column chromatography (H₂O/MeCN gradient elution, formic acid) afforded 15 as a dark-blue crystalline solid (5.3 mg, 23%); ¹H NMR (600 MHz, D₂O, mixture of rotamers) δ 8.57-8.46 (m, 2H), 8.15-8.02 (m, 2H), 7.31-7.15 (m, 2H), 6.98-6.90 (m, 4H), 6.48-6.38 (m, 4H), 5.83 (d, J=7.0 Hz, 4H), (s, 2H), 5.37-5.21 (m, 1H), 4.06-3.93 (m, 3H), 3.77-3.61 (m, 4H), 3.47-3.25 (m, 12H), 3.09-2.87 (m, 20H), 2.21-1.95 (m, 2H), 1.91-1.72 (m, 6H), 1.68-1.13 (m, 30H), 1.10-0.86 (m, 2H); ¹³C NMR (151 MHz, D₂O, mixture of rotamers) δ 171.8, 168.2, 166.4, 157.3, 157.2, 157.0, 154.9, 153.2, 153.1, 138.3, 138.2, 136.1, 135.6, 131.8, 131.2, 129.4, 123.2, 122.9, 121.5, 121.4, 120.7, 120.6, 112.9, 112.8, 95.1, 75.6, 60.0, 59.9, 59.9, 59.8, 52.6, 52.5, 52.3, 40.5, 37.5, 37.0, 36.4, 32.7, 32.6, 32.5, 28.3, 27.9, 27.8, 27.8, 27.8, 27.7, 23.5; ESI-MS [(M+H)/2]⁺ m/z calcd. 947.6 for [C₉₂H₁₀₅N₁₀O₂₆S₄]⁺/2. found 947.8.

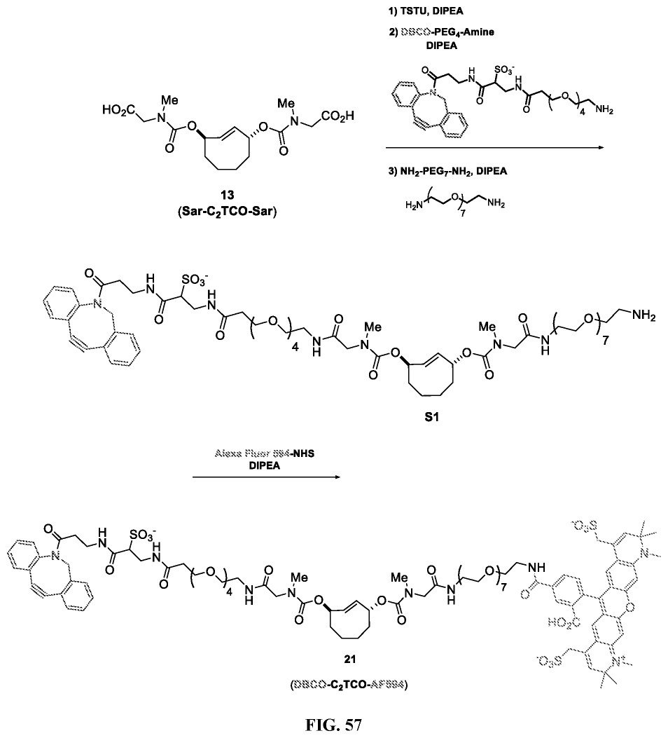

Synthesis of DBCO-C2TCO-AF594 (See FIGS. 16 and 57)

DBCO-C2TCO-PEG7 (S1)

To a solution of Sar-C₂TCO-Sar (13) (20 mg, 0.05 mmol) in dry DMSO (560 μL) were added TSTU (34 mg, 0.11 mmol) and DIPEA (39.0 μl, 0.23 mmol). The mixture was stirred at room temperature for 40 min until LCMS analysis showed full conversion (after quenching a sample with an excess of 2-(2-aminoethoxy)ethanol). DBCO-PEG4-amine (12.1 mg, 17.7 μmol) dissolved in dry DMSO (120 μL) was added and the mixture was stirred at room temperature for 2.5 h. A solution of NH₂-PEG₇-NH₂ (99 mg, 0.27 mmol) in dry DMSO (40 μL) was added and stirring was continued for 35 min. The solution was quenched with formic acid (9.1 μL) and directly loaded onto a C18 column. Purification by reversed phase column chromatography (H₂O/MeCN gradient elution, 0.1% formic acid) gave the desired product as an oil (4.8 mg, 20%); ¹H NMR (600 MHz, D₂O) δ 7.65 (d, J=7.9 Hz, 1H), 7.52-7.34 (m, 7H), 5.94-5.68 (m, 2H), 5.30-5.20 (m, 2H), 5.08 (dd, J=14.1 Hz, J=3.9 Hz, 1H), 4.54-4.47 (m, 1H), 4.00-3.85 (m, 4H), 3.80-3.47 (m, 48H), 3.41-3.35 (m, 4H), 3.21-3.15 (m, 4H), 3.12-3.04 (m, 2H), 2.99-2.90 (m, 6H), 2.58-2.38 (m, 3H), 2.17-1.92 (m, 3H), 1.79-1.56 (m, 4H), 1.16-0.83 (m, 2H); ESI-MS [M]⁻ m/z calcd. 1377.6 for C₆₄H₉₇N₈O₂₃S⁻. found 1377.6.

DBCO-C2TCO-AF594 (21)

To a solution of AF594-NHS (1.7 mg, 2.05 μmol) in dry DMSO (100 μL) were added DBCO—C₂TCO-PEG₇ (S1) (2.7 mg, 1.96 μmol) dissolved in dry DMSO (100 μL) and DIPEA (1.79 μL, 9.79 μmol). The mixture was stirred at room temperature and additional portions of AF594-NHS (0.2 mg each, 0.25 μmol) were added after 2, 5 and 22 h after which LCMS analysis showed full consumption of S1. The solution was quenched with formic acid (0.8 μL) and directly loaded onto a C18 column. Purification by reversed phase column chromatography (H₂O/MeCN gradient elution, 0.1% formic acid) afforded the desired product as a dark-blue solid (3.8 mg, 93%); ¹H NMR (600 MHz, DMSO-d₆) δ 8.99 (t, J=5.1 Hz, 1H), 8.45 (bs, 1H), 8.19 (d, J=7.6 Hz, 1H), 8.14 (s, 4H), 8.10-8.05 (m, 1H), 8.00-7.89 (m, 2H), 7.64-7.58 (m, 2H), 7.55-7.43 (m, 3H), 7.41-7.22 (m, 4H), 5.86-5.70 (m, 2H), 5.59 (bs, 1H), (m, 2H), 5.04 (dd, J=14.1, 3.9 Hz, 1H), 4.22-4.17 (m, 1H), 3.89-3.74 (m, 4H), 3.63-3.44 (m, 48H), 3.24-3.18 (m, 8H), 3.12-3.04 (m, 6H), 2.95-2.79 (m, 12H), 2.66-2.58 (m, 2H), 2.38 (s, 1H), 2.35-2.26 (m, 2H), 2.02-1.95 (m, 1H), 1.93-1.87 (m, 1H), 1.80-1.43 (m, 6H), 1.36-1.20 (m, 11H), 1.15-0.89 (m, 2H); ESI-MS [Fragment]^{·-} -m/z calcd. 1380.5 for C₆₆H₈₈N₆O₂₂S₂·⁻. found 1380.6.

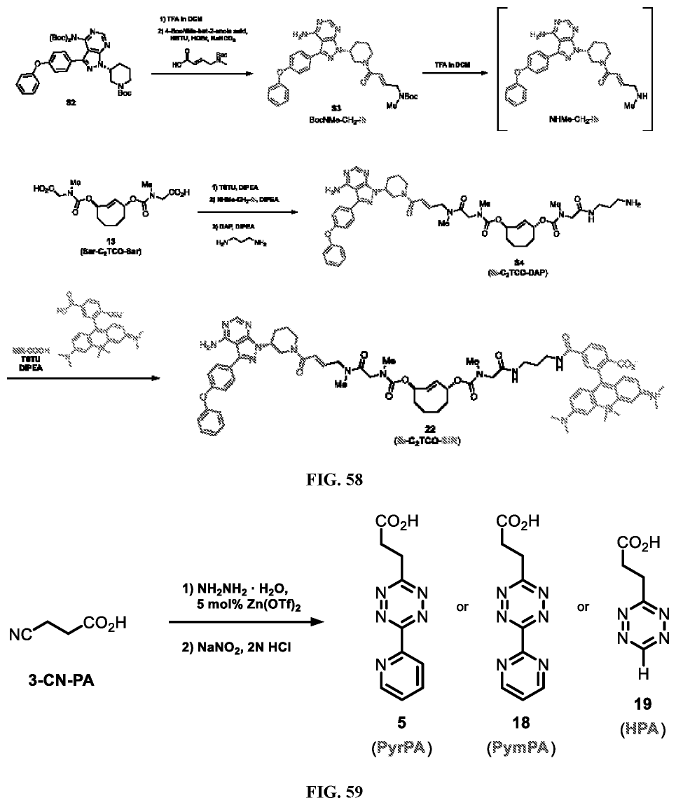

Synthesis of Ib-C2TCO—SiR (See FIG. 58)

BocNMe-CH₂-Ib (S3)

S2 (44 mg, 64.1 μmol) was dissolved in dry DCM (800 μL) and TFA (200 μL) was added dropwise. The solution was stirred at room temperature for 90 min until LCMS analysis indicated complete deprotection. The reaction mixture was diluted with MeCN/DCM (1:1) and concentrated. (E)-4-((tert-butoxycarbonyl)(methyl)amino)but-2-enoic acid (4-BocNMe-but-2-enoic acid)⁷ (15.2 mg, 70.5 μmol) was dissolved in dry DMF (1 mL) and HBTU (26.8 mg, 70.5 μmol), HOBt (10 mg, 70.5 μmol) and NaHCO₃ (20 mg, 224.2 μmol) were added. The mixture was stirred at room temperature for 15 min. A solution of deprotected S2 in dry DMF (500 μL) and NaHCO₃ (20 mg, 224.2 μmol) were added. The reaction mixture was stirred at room temperature for 43 h, acidified with formic acid (50 μL) and diluted with 0.1% formic acid in water (1 mL). Reversed phase column chromatography (H₂O/MeCN gradient elution, 0.1% formic acid) gave the product as a bright yellow solid (27 mg, 73%); ¹H NMR (600 MHz, CDCl₃, mixture of rotamers) δ 8.34 (d, J=15.2 Hz, 1H), 7.62 (d, J=8.3 Hz, 2H), 7.39 (t, J=7.6 Hz, 2H), 7.19-7.14 (m, 3H), 7.09 (d, J=7.8 Hz, 2H), 6.81-6.69 (m, 1H), 6.37-6.23 (m, 1H), 6.20-5.73 (m, 1H), 4.91-4.81 (m, 1.5H), 4.66-4.53 (m, 0.5H), 4.15 (d, J=11.9 Hz, 0.5H), 4.05-3.87 (m, 2.5H), 3.74 (t, J=11.1 Hz, 0.5H), 3.36 (t, J=11.1 Hz, 0.5H), 3.23-3.11 (m, 1H), 2.93-2.76 (m, 4H), 2.43-2.29 (m, 1H), 2.28-2.20 (m, 1H), 1.99 (d, J=13.7 Hz, 1H), 1.78-1.67 (m, 1H), 1.50-1.42 (m, 5H), 1.39-1.30 (m, 4H); ¹³C NMR (151 MHz, CDCl₃, mixture of rotamers) δ 165.4, 159.0, 157.3, 156.8, 156.3, 155.7, 154.7, 154.1, 153.7, 153.5, 144.7, 144.5, 141.4, 140.8, 130.1, 130.0, 127.3, 124.3, 121.6, 120.3, 119.8, 119.3, 98.5, 98.3, 79.9, 53.7, 53.6, 52.9, 50.4, 50.2, 49.7, 46.2, 46.0, 42.3, 34.5, 34.4, 30.5, 30.2, 28.5, 28.4, 25.4, 24.0; ESI-MS [M+H]⁺ m/z calcd. 584.3 for C₃₂H₃₈N₇O₄⁺. found 584.0.

Ib-C2TCO-DAP (S4)

Me-NBoc-CH₂-Ib (S3) (13 mg, 0.02 mmol) was dissolved in dry DCM (200 μL) and TFA (50 μL) was added dropwise. The solution was stirred at room temperature for 90 min until LCMS indicated complete deprotection. The reaction mixture was diluted with MeCN/DCM (1:1) and concentrated under reduced pressure three times. Sar-C₂TCO-Sar (13) (24.47 mg, 0.07 mmol) was dissolved in dry DMSO (490 μL) and TSTU (41.5 mg, 0.14 mmol) and DIPEA (71.0 μl, 0.41 mmol) were added. The mixture was stirred at room temperature for 80 min. Deprotected S3 (10.6 mg, 0.02 mmol) dissolved in dry DMSO (100 μL) and DIPEA (11.5 μL, 0.07 mmol) were then added and stirring was continued for 1 h. DAP (123 mg, 1.65 mmol) was added and the mixture was stirred for 25 min. The solution was quenched with 10% formic acid in water (920 μL) while cooling in an ice bath. Purification by reversed phase column chromatography (H₂O/MeCN gradient elution, 0.1% formic acid) afforded the desired product as a pale-yellow oil (7.2 mg, 37%); ¹H NMR (400 MHz, DMSO-d₆) δ 8.30-8.09 (m, 2H), 7.65 (d, J=8.1 Hz, 2H), 7.44 (t, J=8.6 Hz, 2H), 7.21-7.11 (m, 5H), 6.72-6.41 (m, 2H), 5.87-5.57 (m, 2H), 5.32-5.14 (m, 2H), 4.76-4.61 (m, 2H), 4.08-3.72 (m, 13H), 3.27-3.08 (m, 3H), 3.02-2.71 (m, 9H), 2.35-2.10 (m, 2H) 2.03-1.83 (m, 3H), 1.74-1.45 (m, 8H), 1.14-0.75 (m, 2H); ESI-MS [M+H]⁺ m/z calcd. 894.5 for C₄₆H₆₀N₁₁O₈⁺. found 894.5.

Ib-C2TCO—SiR (22)

To a solution of SiR—COOH⁸ (4.95 mg, 10.5 μmol) in dry DMSO (100 μL) were added TSTU (3.4 mg, 11.3 μmol) and DIPEA (6.3 μL, 36.2 μmol) The mixture was stirred at room temperature for 3h. Ib-C₂-DAP (S4) (7.2 mg, 8.05 μmol) dissolved in dry DMSO (700 μL) was then added and the stirring was continued for 3.5 h. The solution was cooled to 0° C. and quenched by addition of 0.1% formic acid in water (1.2 mL). Purification by reversed phase column chromatography (H₂O/MeCN gradient elution, 0.1% formic acid) gave the desired product as a cyan-blue solid (3.1 mg, 29%); ¹H NMR (400 MHz, DMSO-d₆) δ 8.72 (bs, 1H), 8.29-7.87 (m, 5H), 7.74-7.62 (m, 3H), 7.43 (t, J=7.8 Hz, 2H), 7.22-7.10 (m, 5H), 7.00 (s, 2H), 6.72-6.39 (m, 6H), 5.87-5.56 (m, 2H), 5.42-5.04 (m, 2H), 4.67-4.47 (m, 1H), 4.28-3.55 (m, 9H), 3.12-3.05 (m, 3H), 3.00-2.64 (m, 23H), 2.40-2.05 (m, 2H), 2.00-1.74 (m, 3H), 1.67-1.36 (m, 6H), 1.29-0.73 (m, 4H), 0.63 (s, 3H), 0.50 (s, 3H); ESI-MS [M+H]⁺ m/z calcd. 1348.6 for C₇₃H₈₆N₁₃O₁₁Si⁺. found 1348.7.

### Example 2—Click-to-Release Reactivity of C₂TCO

To investigate the click and release reactions of C₂TCO, both allylic OH— groups were modified to obtain symmetrical compounds, which simplified post-click analysis. Due to the unbiased click with Tz, it cannot be controlled which one of the two groups is released. While this randomness is immaterial for bioorthogonal “turn-off” applications (whichever side is cleaved, the TCO conjugate is “turned-off,” or inactivated), analytical experiments reacting an asymmetric C₂TCO derivative with an asymmetric Tz yield complex mixtures that are difficult to analyze.

Referring to FIG. 9, bis-imidazolium-carbamate 12 was obtained by reacting bis-axial-11 with 1,1′-carbonyldiimidazole (CDI) followed by N-methylation using methyl iodide. In situ conversion of 12 with sarcosine methyl ester followed by saponification gave bis(sarcosinyl)-C₂TCO (13). TSTU-mediated coupling of 13 with mono-Fmoc-1,3-diaminopropane and subsequent deprotection afforded bis-amine 14. This compound was reacted with Alexa Fluor 594 (AF594)-NHS to obtain AF594-C₂TCO-AF594 (15) as a fluorescently labeled C₂TCO probe.

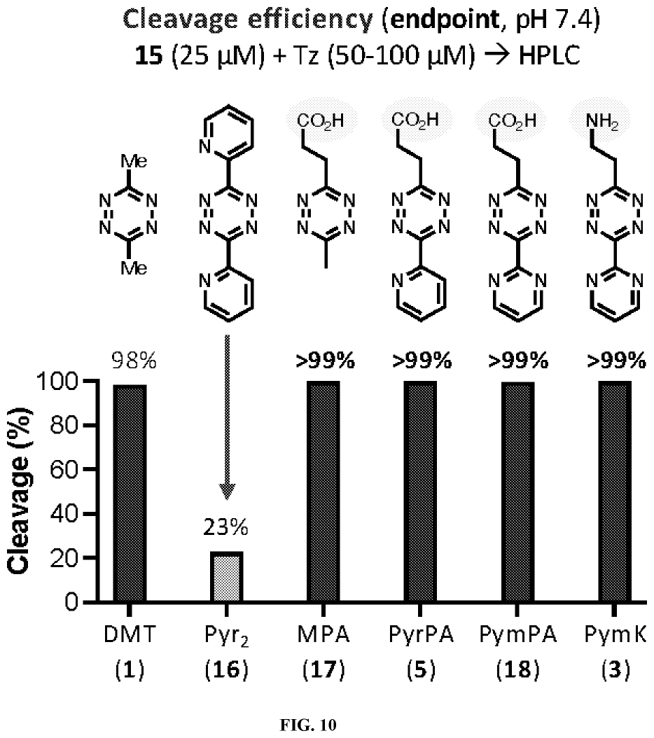

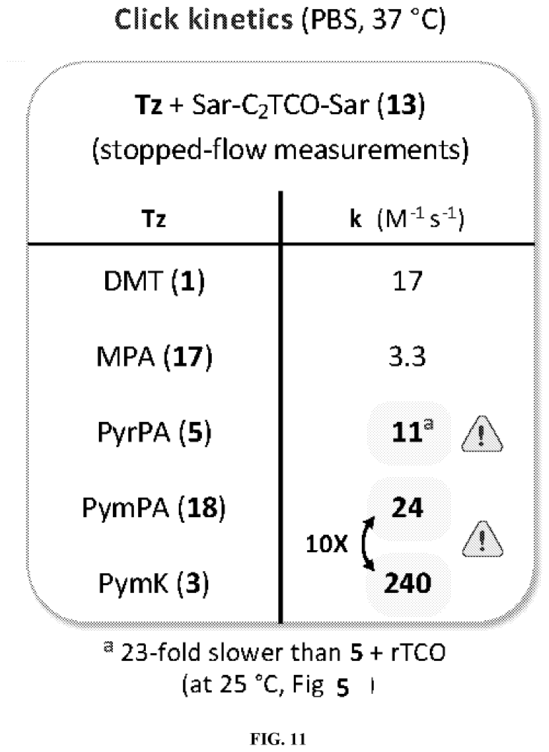

As an initial assessment of cleavage efficiency, 15 was reacted with an excess of selected tetrazines in buffered solution at pH 7.4 and analyzed the reaction mixtures at an endpoint of 24 h by HPLC with fluorescence detection. Nearly complete cleavage was observed with DMT (1) and quantitative cleavage (>99%) with the Tz-acids MPA (17), PyrPA (5) and PymPA (18) as well as with PymK (3). In contrast, reaction with 3,6-bis(2-pyridyl)tetrazine (Pyr2Tz 16) resulted in only 23% cleavage (FIG. 10), in line with previous observations for rTCO. One hypothesis is that this is not only due to slow tautomerization, but also limited elimination of the respective B1/B2 intermediate. The inability to achieve complete release with Pyr2Tz highlights the advantages of a cleavage directing group.

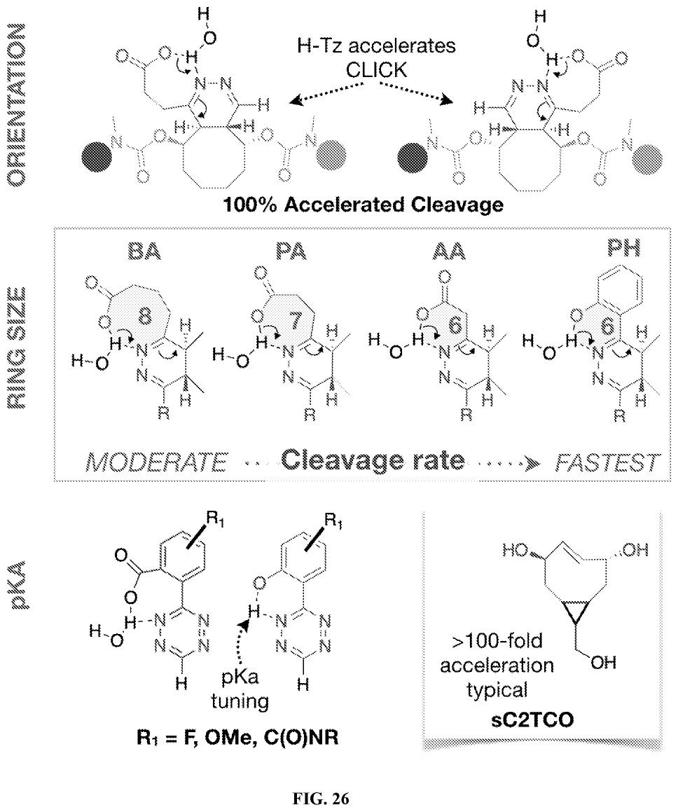

A particular advantage of C₂TCO as the cleavable linker is that it achieves ultrafast and complete cleavage with asymmetric Tz having one directing group, such as a carboxylic acid group, irrespective of the click orientation and the second Tz substituent. Another advantage is that this also allows tuning of the cleavage rate by varying proximity and pKa of the directing group. FIG. 26 illustrates this. In case of bivalirudin (see example below), the fast click kinetics of hydroxyaryl-H-Tzs enable complete click reactions in ˜3s at the infusion concentration of clinical bivalirudin solutions (5 mg/mL, ˜2.3 mM).

### Example 3—“Click” Kinetics of the C₂TCO Conjugates

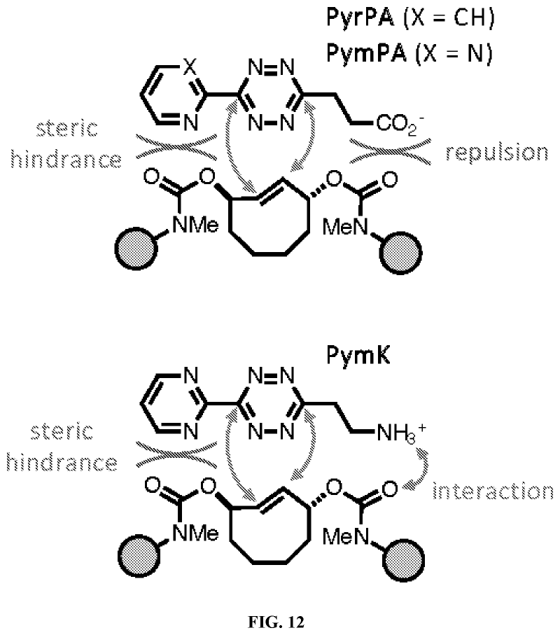

Release kinetics of 13 were determined by serial HPLC measurement, which requires rapid click conversion to kinetically segregate click and release and enable monitoring of the release process exclusively. The second order rate constants were first determined for the click reaction of C₂TCO with a range of tetrazines. Stopped-flow spectrophotometry in PBS revealed rate constants ranging from 3.3 M⁻¹ s⁻¹, for the reaction of MPA with 13, to 240 M⁻¹s⁻¹ for PymK+13 (FIG. 11). One hypothesis is that the PA-carboxylate anion (pKa of PA-Tz: ˜4.5) leads to repulsion with the carbamate functionality of the leaving group, in contrast to the previously observed click enhancement caused by interaction of ammonium substituents (Sarris et al.). These effects (FIG. 12) might thus account for the 5-fold lower reactivity of MPA (carboxylate-repulsion) compared to DMT, and the 10-fold higher rate constant for PymK (NH₃⁺-interaction) than for PymPA (FIG. 11). More importantly, comparing the click reactions of PyrPA with bis(sarcosinyl)-C₂TCO (13) and the rTCO derivative 6 (FIG. 5) revealed a 23-fold lower reactivity of C₂TCO in contrast to rTCO. This significant drop in reactivity can be attributed to the increased steric hindrance of an additional allylic modification (FIG. 12), which is consistent with previous reports showing approximately 20-fold lower reactivity upon modification of one allylic position of the TCO.

### Example 4—Tetrazine Reagents for Fast and Complete Cleavage of C₂TCO Conjugates

To reduce the adverse effect of steric hindrance in reactions with C₂TCO (See Example 3), H-tetrazines (H-Tz) modified with CO₂H and NH₃⁺ directing groups were developed.

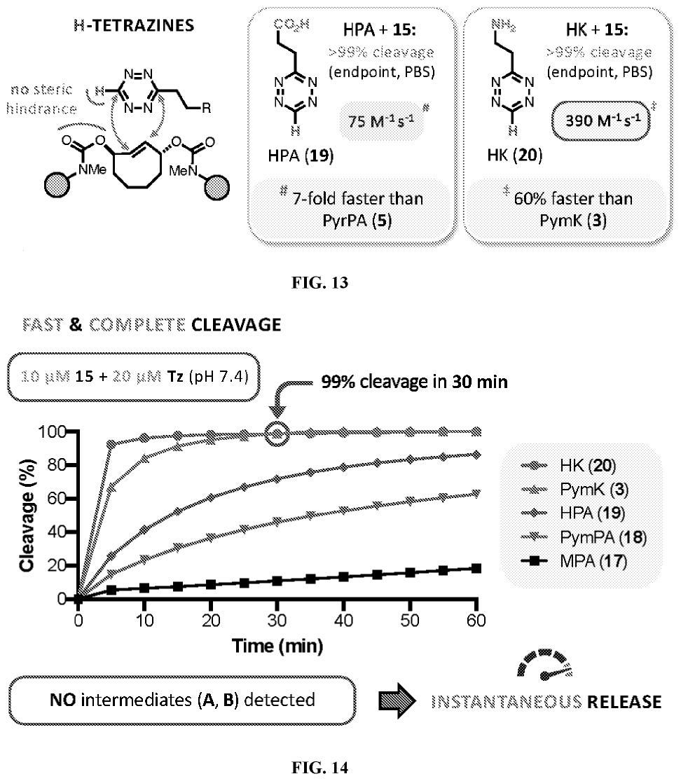

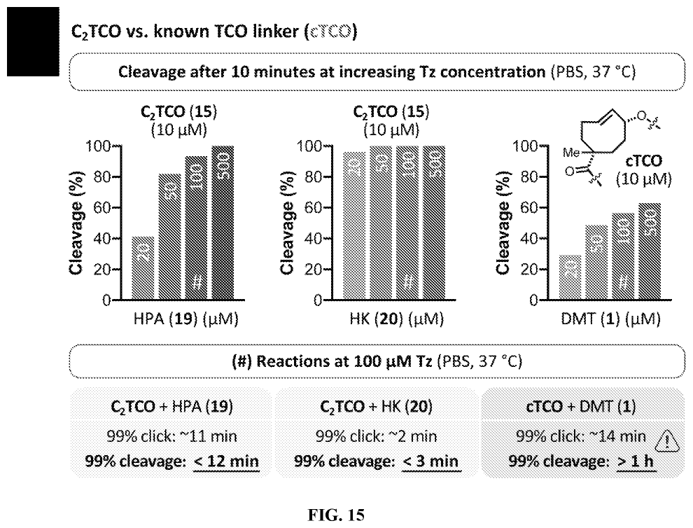

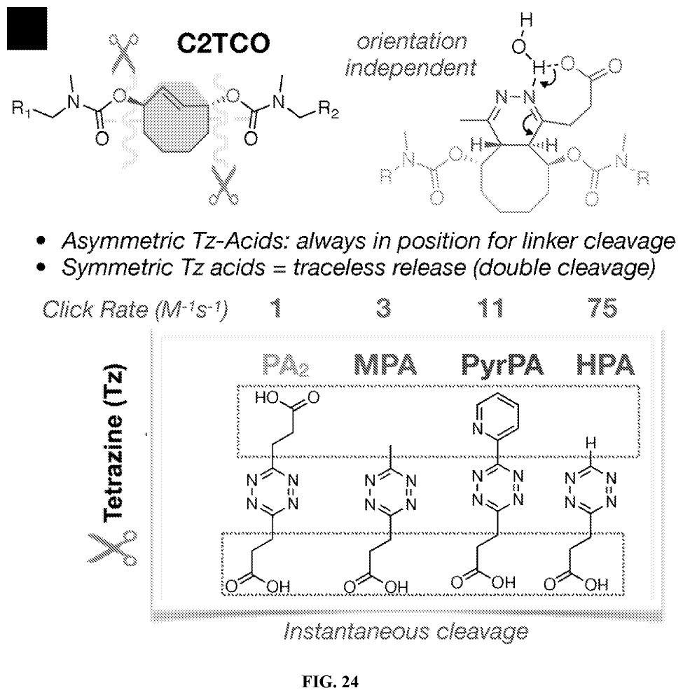

Unexpectedly and surprisingly, in case of C₂TCO, the significantly reduced steric demand of H-Tz lead to even higher click reactivities (FIG. 13) while still being able to achieve fast and complete release. H-aryl Tz have been shown to lead to only poor or even negligible release with rTCO; the mechanism of this effect is as yet unresolved, but sufficiently robust that its pH dependence can be exploited to enforce non-release of rTCO species (See Ko et al., ACIE, 2020, 59, 6839-6846). In this case, however, given that reaction with C₂TCO leads to a directing group being in head-to-head position irrespective of the click orientation (analogous to aryl-Tz, see, e.g., FIG. 7), the rapid release-directing effect supersedes whatever reason impedes H-Tz-mediated release for rTCO. Thus, HPA (19) and HK (20) were prepared and their global click/release performance was assessed. As for their pyridyl and pyrimidyl-substituted counterparts, HPA and HK achieved quantitative cleavage (>99%) and, moreover, significantly increased reaction rates (FIG. 13). At higher concentrations (up to 200 μM) of the fastest tetrazines (HK and PymK), complete cleavage was observed when analyzing the mixture after a reaction time of just 2 minutes, making it challenging to disentangle the release rate from the initial click reaction. Reacting AF594-C₂TCO-AF594 (15) with selected tetrazines at lower concentrations (10 C₂TCO+20 μM Tz) confirmed that release/cleavage is no longer the rate determining step, as observed release rates track tightly with click kinetics. Even with less reactive tetrazines, such as MPA or PymPA, no post-click intermediates were detected (e.g. A, B1, B2), only the starting material and the cleaved product. On the timescale of seconds to minutes, directing groups trigger near-instantaneous release, such that cleavage of C₂TCO is controlled only by click kinetics and Tz concentration. For the fastest tetrazines tested, this enables complete click+cleavage (≥99%) with HK (20) and PymK (3) within 30 min (FIG. 14) at just 20 μM concentration. More generally, C₂TCO-Tz pairs within the present disclosure enable direct control of the time required to achieve complete release, as varying the Tz concentration can tune cleavage rates to the priorities of a particular application (FIG. 15). A brief summary of this approach is shown in FIG. 24.

Examples of H-Tz reagents include those described in Table 1:

TABLE 1

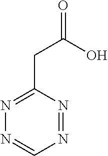

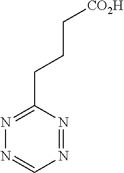 [SMILES: O=C(O)CCCc1nncnn1]

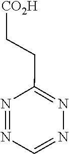 [SMILES: O=C(O)CCc1nncnn1]

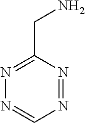

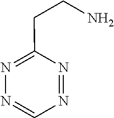

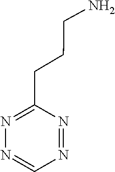

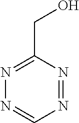

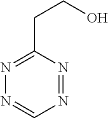

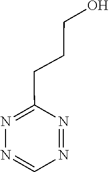

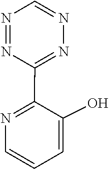 [SMILES: Oc1cccnc1-c1nncnn1]

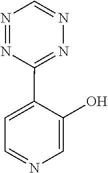 [SMILES: Oc1cnccc1-c1nncnn1]

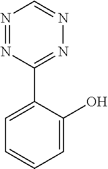 [SMILES: Oc1ccccc1-c1nncnn1]

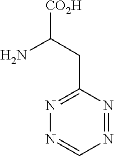 [SMILES: NC(Cc1nncnn1)C(=O)O]

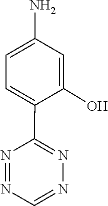 [SMILES: Nc1ccc(-c2nncnn2)c(O)c1]

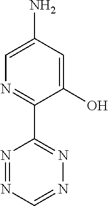 [SMILES: Nc1cnc(-c2nncnn2)c(O)c1]

Other examples of Tz reagents having one directing group include those described in Table 2:

TABLE 2

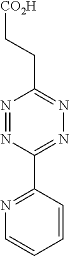 [SMILES: O=C(O)CCc1nnc(-c2ccccn2)nn1]

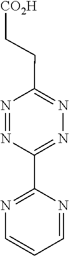 [SMILES: O=C(O)CCc1nnc(-c2ncccn2)nn1]

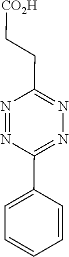 [SMILES: O=C(O)CCc1nnc(-c2ccccc2)nn1]

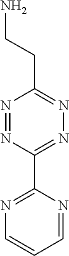 [SMILES: NCCc1nnc(-c2ncccn2)nn1]

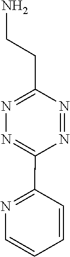 [SMILES: NCCc1nnc(-c2ccccn2)nn1]

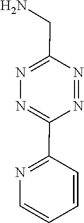 [SMILES: NCc1nnc(-c2ccccn2)nn1]

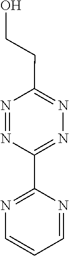 [SMILES: OCCc1nnc(-c2ncccn2)nn1]

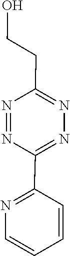 [SMILES: OCCc1nnc(-c2ccccn2)nn1]

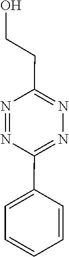 [SMILES: OCCc1nnc(-c2ccccc2)nn1]

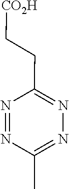

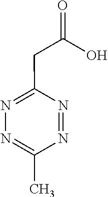

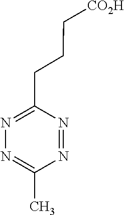

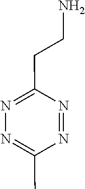

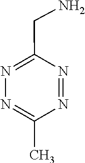

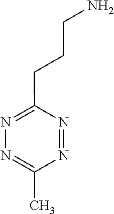

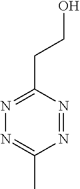

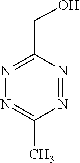

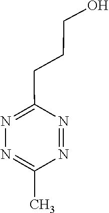

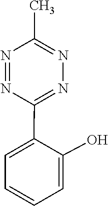

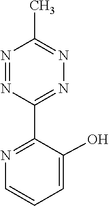

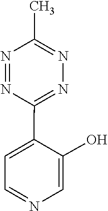

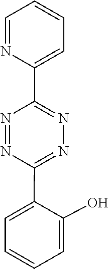 [SMILES: Oc1ccccc1-c1nnc(-c2ccccn2)nn1]

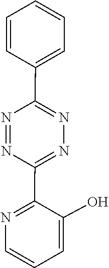 [SMILES: Oc1cccnc1-c1nnc(-c2ccccc2)nn1]

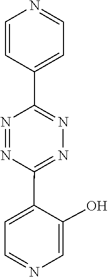 [SMILES: Oc1cnccc1-c1nnc(-c2ccncc2)nn1]

### Example 5—Extracellular Cleavage of Antibody-C₂TCO Conjugates

The reagents such as those described in Examples 1-4 are applicable in bioorthogonal turn-off strategies, such as the rapid disassembly of fluorescently labeled antibodies. Bis(sarcosinyl)-C₂TCO (13) was modified to obtain DBCO—C₂TCO-AF594 (21) as a stable multifunctional click- and cleavable fluorescent tag (FIG. 16). The anti-EGFR-antibody cetuximab was labeled with 6-azidohexanoic acid sulfo-NHS ester and the resulting cetuximab N3 was then click-conjugated to 21 via strain-promoted azide-alkyne cycloaddition (FIG. 16).

Azido-antibodies were tested at different degrees of labeling (DOL) and the C₂TCO cleavage of the clicked conjugates was analyzed after reaction with Tz. No cross-reactivity of 21 with azides was observed (at concentrations of up to 100 mM) in various solvents (incl. PBS), but when clicked to the antibody, a consistent trend toward lower C₂TCO reactivity was noted as the number of azides per antibody increased. Conversely, cleavage yields improved as a function of the concentration of 21 in the antibody labeling reaction (higher concentrations=faster intermolecular N₃-DBCO click), strongly suggestive of an intramolecular reaction of antibody-bound N₃-groups with C₂TCO.

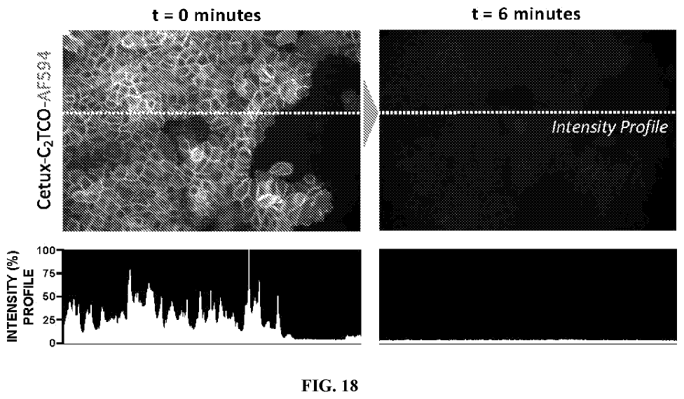

At an average DOL of 0.9, Cetux-C₂TCO-AF594 prepared with a high concentration of 21 (˜250 μM) achieved >97% cleavage and the respective probe was therefore carried forward for further investigation. EGFR-positive A431 cells were stained with Cetux-C₂TCO-AF594, washed and imaged to establish target-specific staining and baseline brightness (ON-state). Cells were then treated with highly water-soluble HPA (19) at a concentration of 500 μM in PBS to ensure complete click in less than 3 min (FIG. 17). Serial imaging of the same field of view demonstrated rapid cleavage upon addition of HPA, with a dramatic reduction in brightness by the first measurement at two minutes and near-complete elimination of the signal within six minutes of Tz addition (OFF-state). A quantitative line-intensity profile demonstrates the excellent cleavage efficiency, with a residual background signal of less than 3% of the peak staining intensity and no discernible difference between the regions with and without cells (FIG. 18). In contrast to image cycling based on click-driven quenching, bioorthogonal disassembly of antibody-C₂TCO probes enables multiplexed imaging without any potential rebound in brightness (e.g., caused by degradation of quenched conjugates) as the dye is cleaved and thus removed upon click-to-cut reaction with Tz.

### Example 6—Stability of C₂TCO-Probes Under Physiological Conditions

C₂TCO can be incorporated into probes that bind to intracellular targets. C₂TCO is stable under physiological conditions: incubation of bis(sarcosinyl)-C₂TCO (13) in full cell growth media (10% FBS) at 37° C. followed by titration with DMT revealed superb stability (>97%) of C₂TCO for up to 48 h.

### Example 7—Intracellular Cleavage of Fluorescent C₂TCO-Probes

Covalent BTK (Bruton's tyrosine kinase) inhibitor ibrutinib (Ib) can be modified with several fluorescent dyes (e.g. silicon rhodamine, SiR) while maintaining selective binding to BTK (Kim et al., Bioconjugate Chemistry, 2015, 26, 1513-1518).

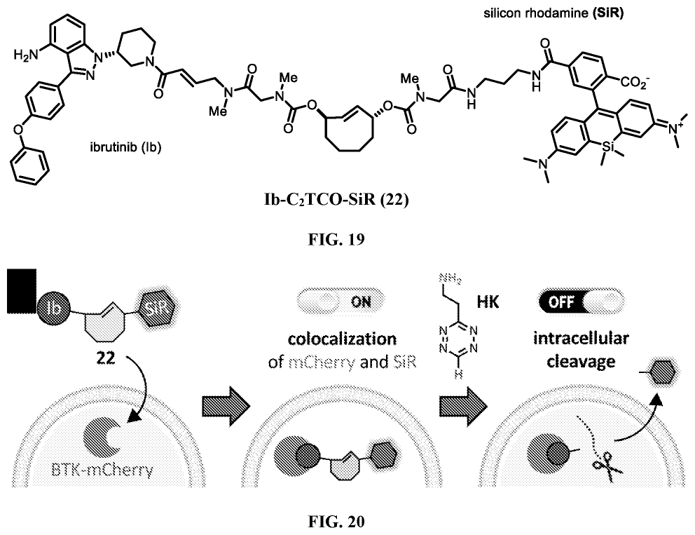

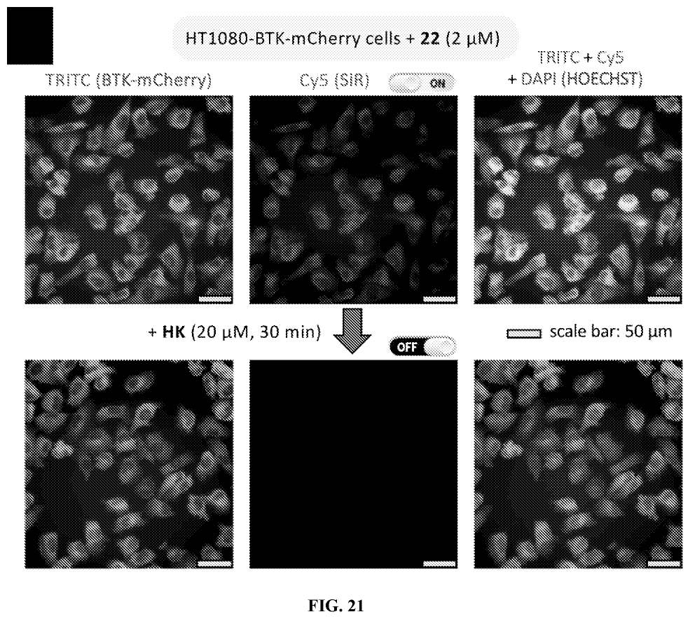

Ib-C₂TCO—SiR (22) (See FIG. 19) was prepared and tested for its cell uptake and binding to BTK in live HT1080 cells stably transfected with BTK-mCherry, followed by intracellular cleavage of C₂TCO with HK (FIG. 20). Live cells were incubated with 22 (2 μM) for 5 h, then washed, fixed and permeabilized (to facilitate efficient removal of released SiR-compounds, which would otherwise interfere with monitoring intracellular cleavage kinetics). Cell imaging by fluorescence microscopy confirmed uptake and selective binding of 22 to BTK, as shown by excellent colocalization of mCherry and SiR with a Pearson coefficient of 0.89 (FIG. 21, on-state, top row). The cells were then treated with HK (20 μM) for 30 min, rinsed with PBS and imaged, showing complete bioorthogonal turn-off with a cleavage efficiency of >98% (FIG. 21, off-state, bottom row). In control experiments with native (BTK-negative) HT1080 cells, only minor nonspecific binding of 22 was observed. Even with the relatively long incubation time of 5 h to achieve sufficient cell uptake of 22, it was shown that (i) C₂TCO can be incorporated into molecular probes binding to intracellular targets, and (ii) these compounds can be disassembled on-target via fast and complete bioorthogonal cleavage.

### Comparative Example 8

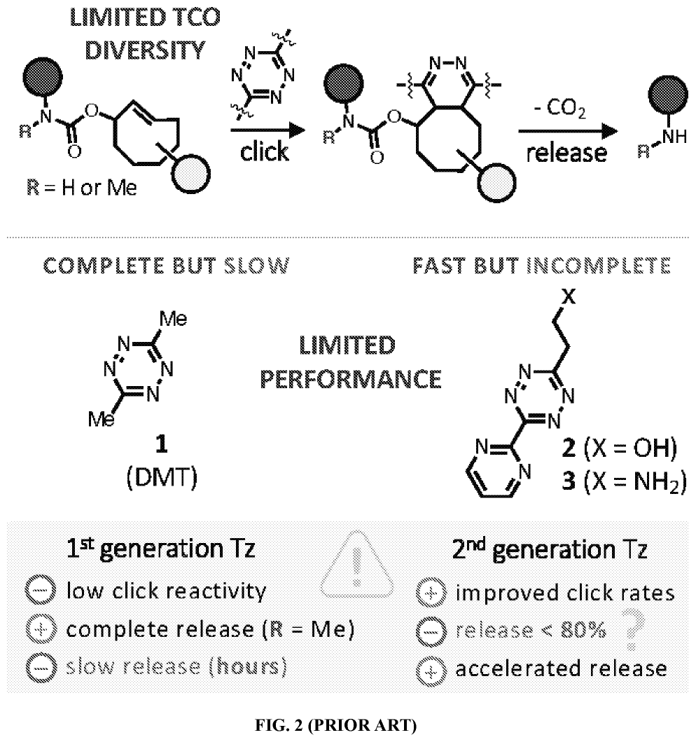

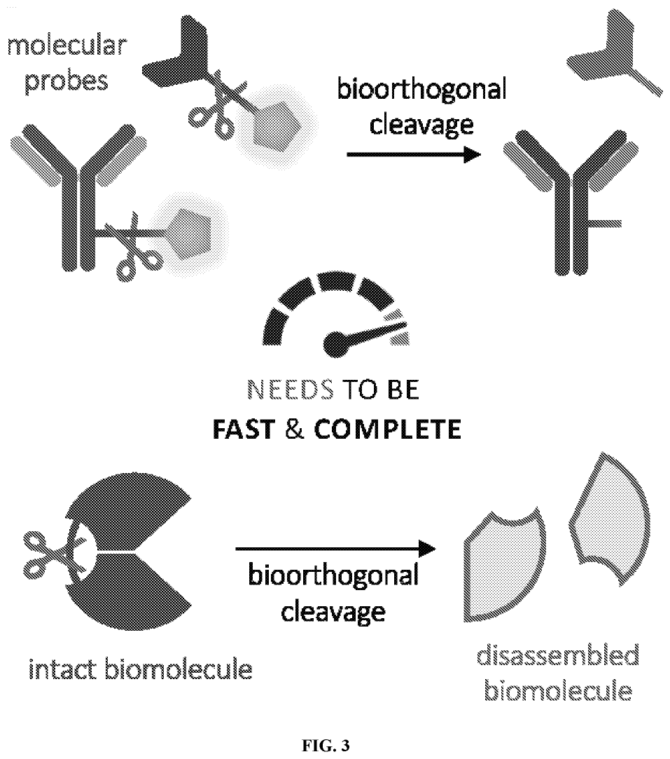

Relatively low click reactivity of bis-alkyl-tetrazines (when compared to other Tz) and the slow release of the resulting dihydropyridazine intermediates limit further applications (See FIG. 2). Second-generation 2-pyrimidyl-substituted tetrazines 2 and 3 (FIG. 2) achieve higher click rates and accelerated release, especially when using the aminoethyl-substituted Tz 3 (PymK) (See Fan et al., ACIE, 2016, 55, 14046-14050; Sarris et al., Chemistry—A European Journal, 2018, 24, 18075-18081). These improvements, however, come with the penalty of incomplete release (<80%)—consistent with previous findings showing limited rTCO cleavage with aryl-tetrazines—even though no intramolecular cyclization was observed (Sarris et al.; for discussion of intramolecular cyclization and its effect on incomplete release, see Carlson et al., Journal of the American Chemical Society, 2018, 140, 3603-3612).

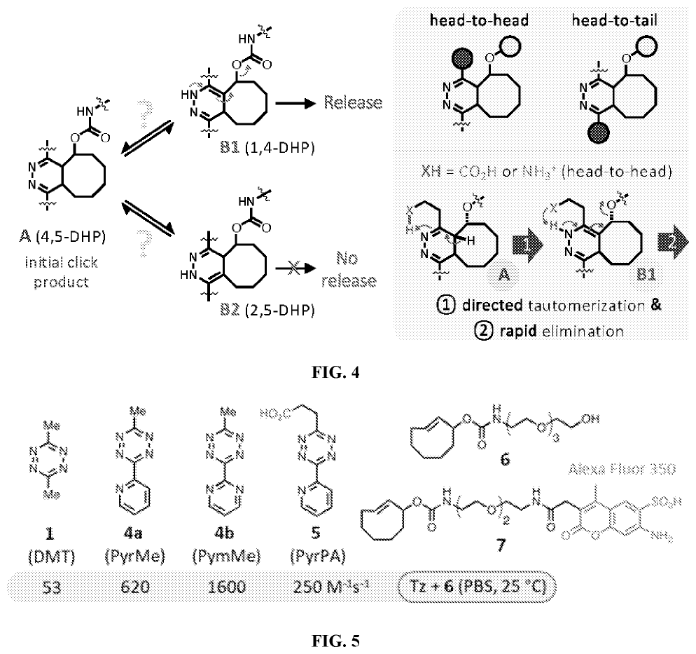

Upon IEDDA reaction of Tz and rTCO-conjugates, it is known that the initial click product A, a 4,5-dihydropyridazine (4,5-DHP), can tautomerize to either the 1,4-dihydropyridazine B1 or its 2,5-isomer B2; release proceeds via 1,4-elimination from the B1 tautomer, while formation of B2 leads to a non-releasing and thus undesired intermediate (FIG. 4). In theory, aryl substituents could alter these equilibria to interfere with release via either preferential formation of the non-releasing B2 tautomer or inefficient elimination from B1. In prior work, it was discovered that modified Tz capable of intramolecular proton donation (Tz-acids) can significantly accelerate tautomerization and subsequent elimination, a capability shared by pH-independent ammonium-functionalized Tz. (Sarris et al.) Directed tautomerization to B1, however, is possible if the directing substituent is in ‘head-to-head’ position to the leaving group on the TCO scaffold (FIG. 4, at right), while the reciprocal ‘head-to-tail’ click product leads to preferred formation of B2. It was previously shown that this results in (i) biphasic release when using Tz-monoacids (fast via B1, and slow via B2⇄A⇄B1) and (ii) slow release (hours) when using a Tz-diacid at physiologic pH. (Carlson et al.)

In both cases, the slow release step takes up to several hours, which not only increases the risk of oxidation of the DHP-intermediates, thus preventing further release, but moreover makes the Tz-acid/rTCO combination unsuitable for “turn-off” applications that require complete and rapid cleavage (i.e., minutes, not hours).

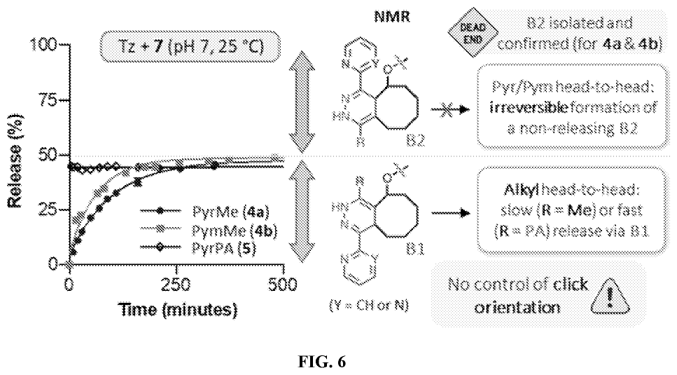

Alkyl-aryl-Tz are known to be significantly more reactive in the initial click step than bis-alkyl-Tz, (See Karver et al., Bioconjugate Chemistry, 2011, 22, 2263-2270) which was verified by measuring the second order rate constants for the reactions of DMT (1), PyrMe (4) and PyrPA (5, PA=propionic acid) with the PEGylated rTCO 6 (See FIG. 6), observing a rate enhancement of up to 12-fold (PyrMe vs. DMT, FIG. 5). Similar to the previous study (Carlson et al.), an rTCO-AlexaFluor 350 conjugate was used (7, FIG. 5) to further investigate the release step. Reactions were carried out in buffered aqueous solution at pH 7 and 25° C., selecting concentrations of PyrMe or PyrPA (200 μM) and 7 (125 μM) such that the click reaction was done in less than 4 minutes, allowing temporal monitoring of post-click reactions by LCMS. With PyrMe, slow monophasic release was observed that reached a yield of approximately 50% within ˜8 hours. For PyrPA, an initial fast phase of release (too rapid to resolve by HPLC) reached a very similar release yield (FIG. 6), whereupon no further release was observed (>24h). These different kinetic profiles result from the methyl group (slow release) and the propionic acid (PA) moiety (fast release) being in head-to-head position, as was demonstrated for PA-functionalized alkyl Tz (Carlson et al.), implying in turn that the non-releasing fraction is due to formation of the respective head-to-tail intermediates, with 2-pyridyl (Pyr) in head-to-head position. To verify these hypotheses, PyrMe was reacted with rTCO-glycine, the release reaction was allowed to proceed to completion, and the non-releasing isomer was then isolated. NMR analysis confirmed the Pyr substituent in the head-to-head position, as hypothesized, and revealed this undesired product to be the respective B2 tautomer (FIG. 6, top). This result showed that tautomerization is no longer an equilibrium (FIG. 4) and that incomplete release for pyridyl Tz is due to the preferential and irreversible formation of a stable B2 tautomer for one of the two possible click orientations (Pyr in head-to-head). This key finding is in line with the observations of Sarris et al., who achieved approx. 80% release with PymK (3) and invoked the preferred formation of the releasing adduct with the ethylammonium moiety in head-to-head position. (due to pre-click interaction of NH₃⁺ with the carbamate functionality of the leaving group) to explain this increased conversion.

### Summary of Examples 1-8

In summary, C₂TCO is a new chemical tool to achieve fast and complete bioorthogonal cleavage. The C₂ symmetry is important to achieve omni-directional click-to-cut with various tetrazines, including acid- and ammonium-functionalized Tz, irrespective of the orientation of the initial IEDDA click reaction. Increased steric hindrance due to the additional leaving group in the allylic position of the TCO scaffold can be circumvented by using less sterically demanding H-tetrazines modified with directing groups (e.g., CO₂H, NH₃⁺) that accelerate post-click tautomerization and enhance 1,4-elimination. Second order rate constants for the initial click reaction of up to ˜400 M⁻¹ s⁻¹, and instantaneous release that is no longer the rate-determining step. Complete cleavage (>99%) of C₂TCO conjugates can be achieved within 30 min even at low concentrations of both reactants (e.g., 10-20 μM) and accelerated accordingly at higher Tz concentrations.

Incorporating C₂TCO into an antibody-dye conjugate presents a strategy to achieve fast extracellular bioorthogonal cleavage within minutes. In a similar approach, using the covalent inhibitor ibrutinib as a targeting moiety, fast and complete intracellular disassembly of an ibrutinib-C₂TCO-dye conjugate bound to its target (BTK) was achieved. Hence, C₂TCO can be used as a cleavable linker in (bio)molecular probes to achieve bioorthogonal cleavage, and thus rapid turn-off, upon reaction with, for example, tetrazines.

Fast and complete disassembly is a prerequisite for bioorthogonal turn-off and efficient molecular inactivation. The development of C₂TCO probes thus enables not only new methods for advanced multiplexed imaging, but even Tz-triggered cleavage in vivo, for instance to remove fluorescent tags or radionuclides from long-circulating compounds and thus to reduce background signal or prolonged radiation of healthy tissue. C₂TCO-fused biomolecules allow temporal control of molecular function in complex biological environments (e.g., inside cells), significantly expanding the scope of bioorthogonal on/off control.

### Example 9—Tetrazine Reagents for Fast and Complete Cleavage of Symmetric and Asymmetric TCO Linkers

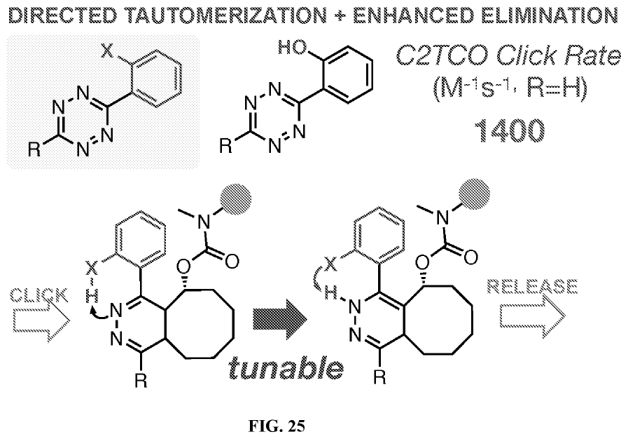

FIG. 23 shows using tetrazine reagents for cleavage of probes and bio(molecules) containing in their structures both symmetric and asymmetric TCO cleavable linkers. FIG. 26 shows effect of various directing groups (such as phenolic groups or carboxylic acid groups) on release rates. FIG. 25 shows tetrazine (Tz) reagents containing various phenolic directing functional groups and their effect on C₂TCO release rates. Examples of Tz reagents having two directing groups and therefore useful for cleaving asymmetric TCO linkers such as rTCO and cTCO linkers, include those described in Table 3:

TABLE 3

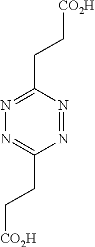 [SMILES: O=C(O)CCc1nnc(CCC(=O)O)nn1]

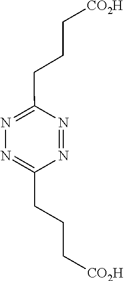 [SMILES: O=C(O)CCCc1nnc(CCCC(=O)O)nn1]

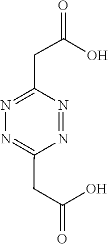 [SMILES: O=C(O)Cc1nnc(CC(=O)O)nn1]

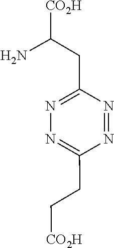 [SMILES: NC(Cc1nnc(CCC(=O)O)nn1)C(=O)O]

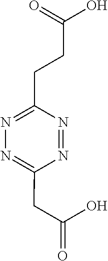 [SMILES: O=C(O)CCc1nnc(CC(=O)O)nn1]

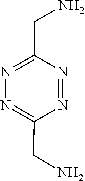

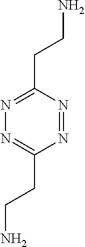 [SMILES: NCCc1nnc(CCN)nn1]

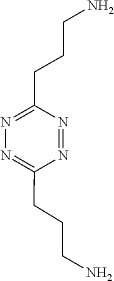 [SMILES: NCCCc1nnc(CCCN)nn1]

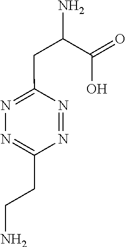 [SMILES: NCCc1nnc(CC(N)C(=O)O)nn1]

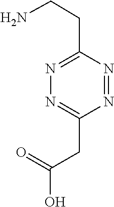 [SMILES: NCCc1nnc(CC(=O)O)nn1]

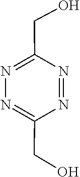

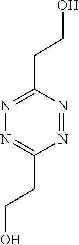 [SMILES: OCCc1nnc(CCO)nn1]

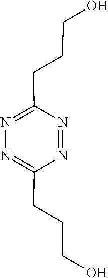 [SMILES: OCCCc1nnc(CCCO)nn1]

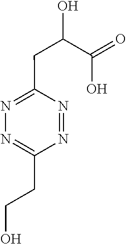 [SMILES: O=C(O)C(O)Cc1nnc(CCO)nn1]

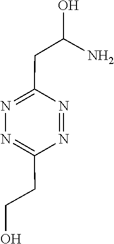 [SMILES: NC(O)Cc1nnc(CCO)nn1]

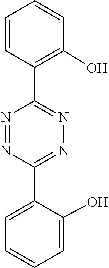 [SMILES: Oc1ccccc1-c1nnc(-c2ccccc2O)nn1]

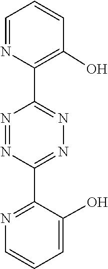 [SMILES: Oc1cccnc1-c1nnc(-c2ncccc2O)nn1]

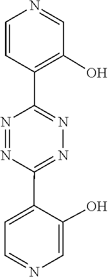 [SMILES: Oc1cnccc1-c1nnc(-c2ccncc2O)nn1]

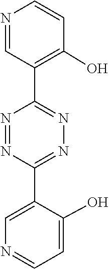 [SMILES: Oc1ccncc1-c1nnc(-c2cnccc2O)nn1]

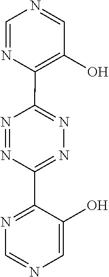 [SMILES: Oc1cncnc1-c1nnc(-c2ncncc2O)nn1]

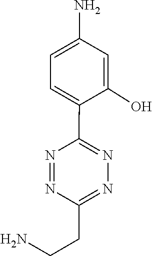 [SMILES: NCCc1nnc(-c2ccc(N)cc2O)nn1]

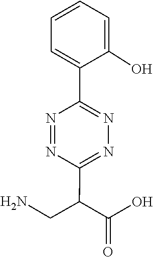 [SMILES: NCC(C(=O)O)c1nnc(-c2ccccc2O)nn1]

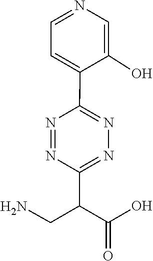 [SMILES: NCC(C(=O)O)c1nnc(-c2ccncc2O)nn1]

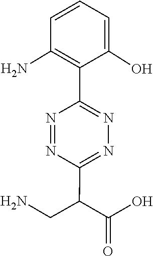 [SMILES: NCC(C(=O)O)c1nnc(-c2c(N)cccc2O)nn1]

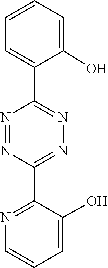 [SMILES: Oc1ccccc1-c1nnc(-c2ncccc2O)nn1]

Synthesis of Exemplified Tetrazines

Synthesis of tetrazines: DMT (1), PymK (3), PyrMe (4a), PymMe (4b), MPA (17) and HK (20) were prepared according to known procedures.

General Procedure for Tz-Acids (See FIG. 59)

A well-blended mixture of nitrile #1 (1 eq.) and nitrile #2 or formamidine acetate salt (5 eq.) and Zn(OTf)₂ (5 mol %) was treated dropwise with hydrazine monohydrate (25 eq.) while cooling in an ice bath. The mixture was allowed to warm up to room temperature and stirred for 10 min, after which it was stirred at 60° C. for the specified time. The crude reaction mixture was poured onto ice-water (50 mL). After addition of NaNO₂ (4 eq.) the solution was acidified with aqueous 2N HCl solution. The mixture was extracted with EtOAc, dried over Na₂SO₄, filtered and concentrated. The crude product was purified by column chromatography.

PyrPA (5): Synthesis was performed according to the general procedure using 3-cyanopropanoic acid (200 mg, 1.65 mmol), 2-pyridinecarbonitrile (860 mg, 8.26 mmol), Zn(OTf)₂ (180 mg, 0.5 mmol) and hydrazine monohydrate (2 mL, 41.3 mmol). After stirring at 60° C. for 25 min the reaction mixture was oxidized using NaNO₂ (456 mg, 6.6 mmol) and 2N HCl, and extracted with EtOAc (4×120 mL). Purification was performed by reversed phase column chromatography (H₂O/MeCN gradient elution, 0.1% formic acid) to give the desired product as a dark-red solid (40 mg, 11%), ¹H NMR (400 MHz, CD₃OD) δ 8.83 (d, J=4.3 Hz, 1H), 8.67 (d, J=8.2 Hz, 1H), 8.14 (td, J=7.8, 1.6 Hz, 1H), 7.69 (dd, J=4.7, 7.8 Hz, 1H), 3.69 (t, J=7.0 Hz, 2H), 3.11 (t, J=7.0 Hz, 2H); ¹³C NMR (100 MHz, CD₃OD) δ 176.0, 171.3, 164.7, 151.5, 151.4, 139.5, 128.0, 125.2, 31.5, 31.0; HRMS [M+H]⁺ m/z calcd. 232.0829 for C₁H₁₀N₅O₂⁺. found 232.0824.

PymPA (18): Synthesis was performed according to the general procedure using 3-cyanopropanoic acid (472 mg, 4.76 mmol), 2-pyrimidinecarbonitrile (100 mg, 0.95 mmol), Zn(OTf)₂ (104 mg, 0.3 mmol) and hydrazine monohydrate (1.1 mL, 23.8 mmol). After stirring at 60° C. for 2.5 h the reaction mixture was oxidized using NaNO₂ (263 mg, 3.8 mmol) and 2N HCl, and extracted with EtOAc (7×80 mL). Purification was performed by reversed phase column chromatography (H₂O/MeCN gradient elution, 0.1% formic acid) to obtain the desired product as a red solid (67 mg, 31%); ¹H NMR (400 MHz, CD₂Cl₂) δ 9.08 (d, J=5.1 Hz, 2H), 7.59 (t, J=4.9 Hz, 1H), 3.78 (t, J=7.0 Hz, 2H), 3.21 (t, J=7.0 Hz, 2H); ¹³C NMR (100 MHz, CD₂Cl₂) δ 175.7, 171.8, 164.2, 159.7 (2C), 159.4, 124.4, 31.3, 31.0; HRMS [M+Na]⁺ m/z calcd. 255.1922 for C₉H₈N₆NaO₂⁺. found 255.1998.

HPA (19): Synthesis was performed according to the general procedure using 3-cyanopropanoic acid (1 g, 8.26 mmol), formamidine acetate salt (4.3 g, 41.3 mmol), Zn(OTf)₂ (0.9 g, 2.5 mmol) and hydrazine monohydrate (10 mL, 206 mmol). After stirring at 60° C. for 60 min the reaction mixture was oxidized using NaNO₂ (2.3 g, 33 mmol) and 2N HCl, and extracted with EtOAc (6×150 mL). Purification was performed by reversed phase column chromatography (H₂O/MeCN gradient elution, 0.1% formic acid) to afford the desired product as red crystals (45 mg, 4%); 1H NMR (600 MHz, CD₃OD) δ 10.30 (s, 1H), 3.61 (t, J=6.9 Hz, 2H), 3.06 (t, J=7.0 Hz, 2H); ¹³C NMR (151 MHz, CD₃OD) δ 175.8, 173.1, 159.4, 31.3, 31.2; HRMS [M+Na]⁺ m/z calcd. 177.1182 for C₅H₆N₄NaO₂⁺. found 177.1136.

### Example 9a—Synthesis of Exemplified Tetrazine Conjugates

Chemical synthesis of tetrazine conjugates is schematically shown in FIG. 48.

N-Boc-β-(1,2,4,5-tetrazinyl)-L-alanine (1)

To a well-blended mixture of N-Boc-β-cyano-1-alanine (51) (600 mg, 2.8 mmol), formamidine acetate salt (1.46 g, 14 mmol) and Zn(OTf)₂ (255 mg, 0.7 mmol) was added dry dioxane (8.4 mL, 98 mmol) and hydrazine monohydrate (6.8 mL, 140 mmol) and the vial was sealed immediately. The reaction mixture was stirred at 30° C. for 72 h after which it was poured onto ice-water (50 mL). After addition of NaNO₂ (3.9 g, 56 mmol) the solution was acidified with aqueous 2N HCl solution (60 mL). The mixture was extracted with EtOAc (5×70 mL), the combined organic layer was dried over MgSO₄, filtered and concentrated. Purification was performed by preparative reversed phase column chromatography (C18, H₂O/MeCN, gradient elution, 0.1% formic acid) to afford tetrazine 1 (35.4 mg, 5%) as a pink oil; 41-NMR (600 MHz, CD₃OD) δ 10.33 (s, 1H), 4.81 (dd, J=7.5 Hz, 5.9 Hz, 1H), 3.90 (dd, J=14.8 Hz, 5.6 Hz, 1H), 3.67 (dd, J=15.2 Hz, 8.8 Hz, 1H), 1.37 (s, 9H); ¹³C NMR (151 MHz, CD₃OD) δ 173.9, 171.4, 159.4, 157.6, 80.7, 53.6, 38.9, 28.6; ESI-MS [M+H]⁺ calcd. 270.12 for C₆H₉N₄O₂⁺. found 270.10.

H-Tz-BocAla-PEG4-CO2H (S11)

TSTU (5.4 mg, 17.8 μmol) and DIPEA (5.2 μL, 29.7 μmol) were added to a solution of tetrazine 1 (4 mg, 14.9 μmol) in dry DMSO (250 μL). The mixture was stirred at room temperature for 40 min. NH₂-PEG₄-CO₂H (7.9 mg, 29.7 μmol) suspended in dry DMSO (300 μL) was added and stirring was continued for 1.5 h. The reaction mixture was quenched by addition of 1% formic acid in water (900 μL) while cooling in an ice bath. Purification by reversed phase column chromatography (H₂O/MeCN gradient elution, 0.1% formic acid) afforded S11 (4.9 mg, 65%); NMR (400 MHz, CD₃OD) δ 10.34 (s, 1H), 4.75 (dd, J=8.3 Hz, 5.8 Hz, 1H), 3.83 (dd, J=14.4 Hz, 5.2 Hz, 1H), 3.73 (t, J=6.2 Hz, 2H), 3.65-3.53 (m, 18H), 3.38 (q, J=4.6 Hz, 2H), 2.53 (t, J=6.0 Hz, 2H), 1.37 (s, 9H); ¹³C NMR (101 MHz, CD₃OD) δ 176.4, 172.8, 171.5, 157.4, 80.9, 71.57, 71.54, 71.46, 71.4, 71.2, 70.4, 68.1, 54.7, 39.2, 36.3, 28.6; ESI-MS [M+H]⁺ m/z calcd. 517.26 for C₂₁H₃₇N₆O₉⁺. found 517.46.

BHQ3-NBoc-Tz (S12)

S11 (4.9 mg, 9.49 μmol) was dissolved in dry DMSO (260 μL) and TSTU (3.4 mg, 11.4 μmol) and DIPEA (3.3 μL, 18.9 μmol) were added. The mixture was stirred at room temperature for 30 min. BHQ3-amine (5 mg, 9.6 μmol) dissolved in dry DMSO (300 μL) was added and stirring was continued for 2 h. The reaction mixture was quenched by addition of formic acid (15 μL) and 0.1% formic acid in water (900 μL) while cooling in an ice bath. Purification by reversed phase column chromatography (10 mM ammonium formate buffer pH 9/MeCN gradient elution) afforded S12 as a dark blue solid (5.95 mg, 62%); ¹H NMR (400 MHz, CD₃OD) δ (s, 1H), 8.40-8.12 (m, 3H), 7.97-7.91 (m, 4H), 7.80 (bs, 2H), 7.69 (d, J=7.2 Hz, 2H), 7.37 (bs, 1H), 6.86 (d, J=9.2 Hz, 2H), 5.90 (bs, 1H), 4.69 (dd, J=8.4 Hz, Hz, 1H), 3.81-3.73 (m, 3H), 3.62-3.55 (m, 16H), 3.48 (t, J=5.6 Hz, 2H), 3.35-3.33 (m, 1H), 3.27 (t, J=6.5 Hz, 2H), 2.45 (t, J=5.9 Hz, 2H), 2.19 (q, J=7.1 Hz, 3H), 1.86 (quin, J=7.0 Hz, 2H), 1.60 (bs, 3H), 1.38-1.29 (m, 15H), 1.22-1.00 (m, 4H), 0.90 (t, J=6.5 Hz, 1H); ESI-MS [M+H]⁺ m/z calcd. 1014.53 for C₅₃H₆₈N₁₃O₈⁻. found 1014.56.

BHQ3-N-Tz (2) (Chemical Structure is Shown in FIG. 36B)

3 mg, 2.95 μmol) was dissolved in dry DCM (1 mL) and TFA (200 μL) was added dropwise. The solution was stirred at room temperature for 40 min until LCMS analysis indicated complete deprotection. The reaction mixture was diluted with MeCN/DCM (1:1, 2 mL), concentrated under reduced pressure and the residue was co-evaporated three times with MeCN (3×1.5 mL) to obtain BHQ3-N-Tz (2) as a dark blue oil (2.7 mg, >99%); ¹H NMR (400 MHz, CD₃OD) δ 8.38 (d, J=9.3 Hz, 1H), 8.25 (d, J=9.2 Hz, 1H), 8.16 (d, J=9.9 Hz, 1H), 7.97-7.91 (m, 4H), 7.81 (d, J=9.3 Hz, 2H), 7.68 (d, J=7.5 Hz, 2H), 7.39 (s, 1H), 6.85 (d, J=9.1 Hz, 2H), 5.90 (d, J=2.2 Hz, 1H), 5.34 (t, J=4.8 Hz, 0.5H), 4.57 (dd, J=7.7 Hz, 5.4 Hz, 0.5H), 3.93-3.82 (m, 3H), 3.74 (t, J=5.8 Hz, 3H), 3.64-3.57 (m, 15H), 3.26 (t, J=6.6 Hz, 2H), 3.14 (s, 3H), 2.46 (t, J=5.9 Hz, 2H), 2.28 (t, J=7.0 Hz, 2H), 2.19 (t, J=7.4 Hz, 2H), 1.86 (t, J=6.2 Hz, 2H), 1.60 (bs, 3H), 1.16 (d, J=8.3 Hz, 3H), 0.92-0.87 (m, 6H); ESI-MS [M+H]⁺ m/z calcd. 917.50 for C₄₈H₆₃N₁₃O₆²⁺. found 917.82.

### Example 9b—Tetrazine-Triggered Double-Release of C₂TCO

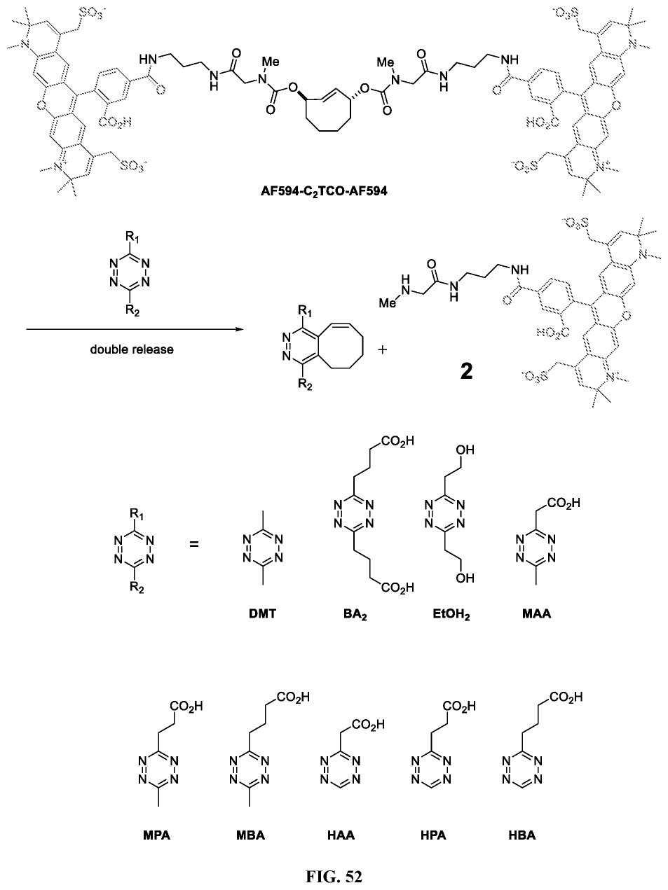

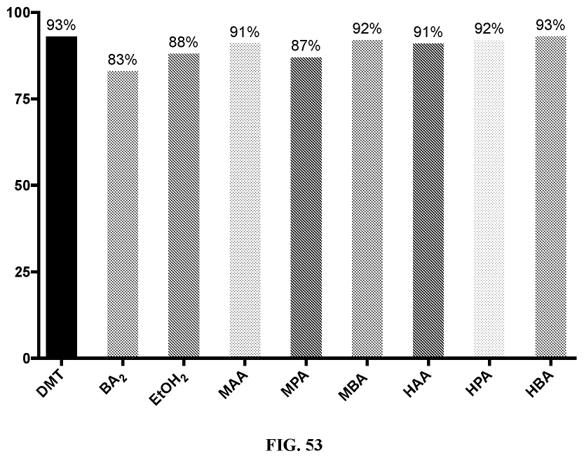

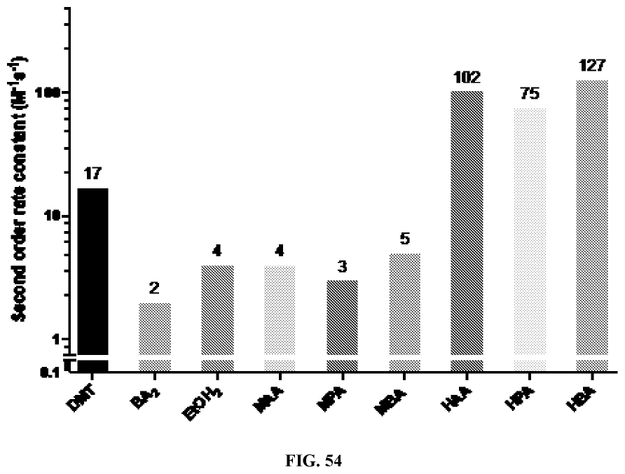

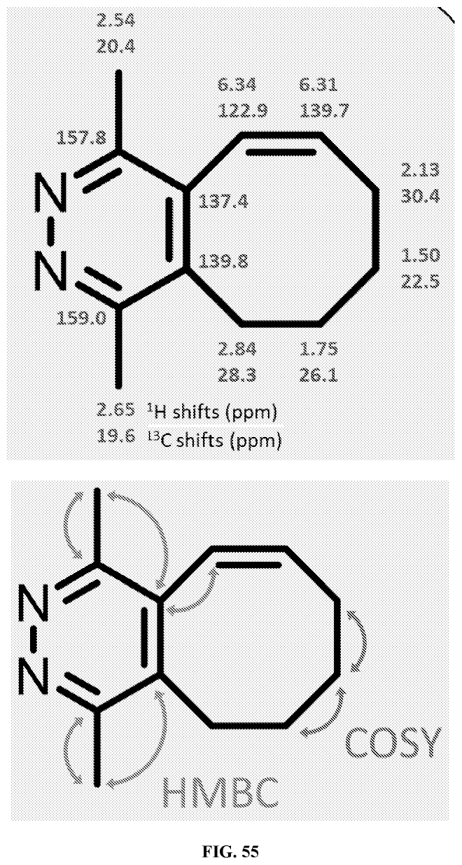

Chemical structures of the C₂TCO reagent and the tetrazine quenchers are shown in FIG. 52. FIG. 53 shows corresponding yields of the double-release reactions with different tetrazines. FIG. 54 shows Click kinetics (second order rate constants) for the reactions of FIG. 52. Synthesis of PEG2-C2TCO-PEG2 (26) is provided above in Example 1a.

General methods: unless otherwise noted, reactions were carried out under an atmosphere of argon in air-dried glassware with magnetic stirring. Air- and/or moisture-sensitive liquids were transferred via syringe. All reagents were purchased from commercial sources without further purification. Dichloromethane, methanol, THF and diethyl ether were dried using PURESOLV-columns (Inert Corporation, USA). Solvents used for flash column chromatography were purchased from Donau Chemie AG (Austria). Dry acetonitrile and dry DMF were obtained from Sigma-Aldrich (Germany) and ACROS Organics (Belgium), respectively, and stored under argon. Thin layer chromatography was performed using TLC plates on aluminum support (Merck, silica gel 60, fluorescent indicator 254). Column chromatography was performed using a BUCHI Sepacore Flash System (2×BUCHI Pump Module C-605, BUCHI Pump Manager C-615, BUCHI UV Photometer C-635, and BUCHI Fraction Collector C-660) and a Reveleris® X2 Flash Chromatography/Prep Purification Systems (BUCHI). Silica gel 60 (40-63 μm) was obtained from Merck. A Kinetex® 5 μm C18 100 Å, AXIA LC column (100×30.0 mm, Phenomenex) was used for preparative HPLC. HPLC grade solvents were purchased from VWR (USA).

¹H and ¹³C NMR spectra were recorded on a Bruker AC 200 MHz, Bruker Avance UltraShield 400 MHz or Bruker Ascend 600 MHz spectrometer at 20° C. Chemical shifts are reported in ppm (δ) relative to tetramethylsilane and calibrated using solvent residual peaks. Data is shown as follows: Chemical shift, multiplicity (s=singlet, d=doublet, t=triplet, q=quartet, quint=quintet, m=multiplet, b=broad signal), coupling constants (J, Hz) and integration.

GC/MS experiments were done with a Thermo Finnigan GC 8000 Top gaschromatograph on a BGB5 column (l=30 m, di=0.32 mm, 1 μm coating thickness) coupled to a Voyager Quadrupol mass spectrometer (electron ionization, 70 eV).

HPLC analysis was performed on a Nexera X2® UHPLC system (Shimadzu®) comprised of LC-30AD pumps, SIL-30AC autosampler, CTO-20AC column oven and DGU-20A_{5/3} degasser module. Detection was done using an SPD-M20A photo diode array, an RF-20Axs fluorescence detector, and ELS-2041 evaporative light scattering detector (JASCO®) and an LCMS-2020 mass spectrometer (ESI/APCI). If not stated otherwise, all separations were performed using a Waters® XSelect® CSH™ C18 2.5 μm (3.0×50 mm) column XP at 40° C., and a flowrate of 1.7 mL/min with water/acetonitrile+0.1% formic acid gradient elution.

HRMS analysis was carried out using methanol solutions (concentration: 10 ppm) on an Agilent 6230 LC TOFMS mass spectrometer equipped with an Agilent Dual AJS ESI-Source. The mass spectrometer was connected to a liquid chromatography system of the 1100/1200 series from Agilent Technologies (Palo Alto, CA, USA). The system consisted of a 1200SL binary gradient pump, a degasser, column thermostat, and an HTC PAL autosampler (CTC Analytics AG, Zwingen, Switzerland).

Synthesis of MAA

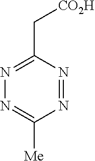 [SMILES: Cc1nnc(CC(=O)O)nn1]

In a pressure tube vial cyanoacetic acid (250 mg, 2.9 mmol), dry acetonitrile (0.8 mL, 14.7 mmol) and Zn(OTf)₂ (320 mg, 0.88 mmol) were mixed and the vial was sealed. The mixture was cooled in an ice-batch and hydrazine monohydrate (1.45 mL, 29.4 mmol) was added dropwise. The reaction mixture was stirred at 60° C. for 10 min after which it was diluted with water (5 mL) and NaNO₂ (406 mg, 5.9 mmol) was added. The solution was acidified with aqueous 2N HCl solution (15 mL) and the mixture was extracted with EtOAc (5×20 mL), the combined organic layer was dried over MgSO₄, filtered and concentrated. Purification was performed by preparative reversed phase column chromatography (C18, H₂O/MeCN, gradient elution, 0.1% formic acid) to afford the desired product as a pink oil (29 mg, 7%); ¹H-NMR (400 MHz, CD₂Cl₂) δ 5.75 (bs, 1H), 4.42 (s, 2H), 3.06 (s, 3H); ¹³C NMR (101 MHz, CD₂Cl₂) δ 172.4, 168.6, 164.6, 40.7, 21.4; ESI-MS [M+H]⁺ calcd. 155.06 for C₅H₇N₄O₂⁺. found 155.05.

Synthesis of MBA and BA₂

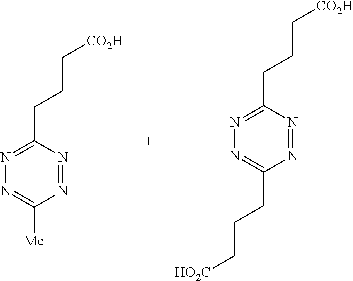 [SMILES: Cc1nnc(CCCC(=O)O)nn1 ; O=C(O)CCCc1nnc(CCCC(=O)O)nn1]

In a pressure tube 4-cyanobutanoic acid (170 mg, 1.5 mmol), dry acetonitrile (0.4 mL, 7.5 mmol) and Zn(OTf)₂ (164 mg, 0.45 mmol) were mixed and the vial was sealed. The mixture was cooled in an ice-batch and hydrazine monohydrate (0.75 mL, 15 mmol) was added dropwise. The reaction mixture was stirred at 60° C. for 35 min after which it was diluted with water (5 mL) and NaNO₂ (207 mg, 3 mmol) was added. The solution was acidified with aqueous 2N HCl solution (15 mL) and the mixture was extracted with EtOAc (4×20 mL), the combined organic layer was dried over MgSO₄, filtered and concentrated. Purification was performed by preparative reversed phase column chromatography (C18, H₂O/MeCN, gradient elution, 0.1% formic acid) to afford MBA (20 mg, 9%) and BA₂ (3.5 mg, 6%) as pink oils; MBA: ¹H-NMR (600 MHz, CD₂Cl₂) δ 3.36 (t, J=7.7 Hz, 2H), 3.01 (s, 3H), 2.54 (t, J 7.3 Hz, 2H), 2.27 (quin, J=7.4 Hz, 2H); ¹³C NMR (151 MHz, CD₂Cl₂) δ 177.1, 169.6, 168.1, 34.1, 32.9, 23.1, 21.3; ESI-MS [M+H]⁺ calcd. 183.09 for C₇H₁₁N₄O₂⁺. found 183.10. BA₂: ¹H NMR (600 MHz, CD₃OD) δ 3.34 (t, J=7.5 Hz, 4H), 2.47 (t, J=7.2 Hz, 4H), 2.23 (quin, J=7.3 Hz, 4H); ¹³C NMR (151 MHz, CD₃OD) δ 176.7, 170.9, 34.9, 33.9, 24.1; ESI-MS [M+H]⁺ calcd. 255.11 for C₁₀H₁₅N₄O₄⁺. found 255.10.

Synthesis of HAA

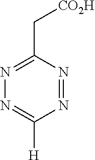

To a well-blended mixture of cyanoacetic acid (400 mg, 4.7 mmol), formamidine acetate salt (2.45 g, 23.5 mmol) and Zn(OTf)₂ (512 mg, 1.41 mmol) was added hydrazine monohydrate (5.7 mL, 118 mmol) while cooling in an ice-bath. The reaction mixture was stirred at 60° C. for 60 min after which it was poured onto ice-water (50 mL). After addition of NaNO₂ (1.3 g, 19 mmol) the solution was acidified with aqueous 2N HCl solution (100 mL). The mixture was extracted with EtOAc (4×100 mL), the combined organic layer was dried over MgSO₄, filtered and concentrated. Purification was performed by preparative reversed phase column chromatography (C18, H₂O/MeCN, gradient elution, 0.1% formic acid) to afford the desired product as a dark red oil (12.5 mg, 2%); ¹H NMR (400 MHz, CD₂Cl₂) δ 10.30 (s, 1H), 4.48 (s, 2H); ¹³C NMR (101 MHz, CD₂Cl₂) δ 172.9, 167.5, 158.8, 41.3; ESI-MS [M+H]⁺ calcd. 141.04 for C₄H₅N₄O₂⁺. found 141.05.

Synthesis of HBA

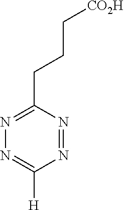 [SMILES: O=C(O)CCCc1nncnn1]

To a well-blended mixture of 4-cyanobutanoic acid (200 mg, 1.8 mmol), formamidine acetate salt (920 mg, 8.8 mmol) and Zn(OTf)₂ (193 mg, 0.53 mmol) was added hydrazine monohydrate (2.15 mL, 44.2 mmol) while cooling in an ice bath. The reaction mixture was stirred at 60° C. for 60 min after which it was poured onto ice-water (25 mL). After addition of NaNO₂ (490 mg, 7.1 mmol) the solution was acidified with aqueous 2N HCl solution (40 mL). The mixture was extracted with EtOAc (4×60 mL), the combined organic layer was dried over MgSO₄, filtered and concentrated. Purification was performed by preparative reversed phase column chromatography (C18, H₂O/MeCN, gradient elution, 0.1% formic acid) to afford the desired product as a dark red oil (13.7 mg, 5%); ¹H NMR (400 MHz, CD₂Cl₂) δ 10.21 (s, 1H), 3.43 (t, J=7.8 Hz, 2H), 2.57 (t, J=7.4 Hz, 2H), 2.30 (quin, J=7.4 Hz, 2H); ¹³C NMR (101 MHz, CD₂Cl₂) δ 177.9, 172.8, 158.6, 34.7, 33.1, 23.0; ESI-MS [M+H]⁺ calcd. 169.07 for C₆H₉N₄O₂⁺. found 169.08.

Isolation and Structure Elucidation of Double Released Product

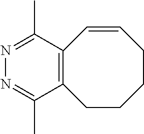

PEG2-C₂TCO-PEG₂ was dissolved in DMSO to prepare a 50 mM stock solution. 820.8 μL (41 μmol, 22.43 mg) of that solution were diluted into 101.8 mL of CitPhos buffer (10 mM, pH 7), for a final concentration of 400 μM C₂TCO. The solution was then sparged with argon for 15 minutes to reduce dissolved oxygen and minimize potential aromatization, whereupon 756.5 μL of an 81.4 mM stock solution of DMT (1.5 eq., 61 μmol, 6.8 mg) were then added. At these reagent concentrations the click reaction is expected to be done in <20 minutes. The release reaction was allowed to proceed under argon overnight, a timeline after which no further release was observed by serial HPLC analyses (24.5 h). The buffer solution was evaporated to near-dryness and then redissolved in 1.5 mL of DMSO. The DMSO solution was directly loaded onto a 30 g Biotage Snap C18 Ultra column and the double-released product isolated with an ammonium formate (2.5 mM, pH 8.4)/acetonitrile gradient as a white crystalline solid (6.4 mg, 83%); ¹H NMR (600 MHz, CD₃OD) δ 6.34 (d, J=12.1 Hz, 1H), 6.31-6.27 (m, 1H), 2.84 (bs, 2H), 2.65 (s, 3H), 2.54 (s, 3H), 2.13 (bs, 2H), 1.75 (bs, 2H), 1.50 (bs, 2H); ¹³C NMR (151 MHz, CD₃OD) δ 159.0, 157.8, 139.8, 139.7, 137.4, 122.9, 30.4, 28.3, 26.1, 22.5, 20.4, 19.6; ESI-MS [M+H]⁺ m/z calcd. 189.14 for C₁₂H₁₇N₂⁺. found 189.12. Elucidated chemical structure following 1D/2D NMR analysis (¹H, ¹³C, COSY, HSQC, HMBC) is shown in FIG. 54.

Click Kinetics

Sample preparation: Sar-C₂TCO-Sar was dissolved in PBS (pH 7.4, 10 mM) to reach an approximate concentration above 2 mM. The exact concentration was determined by absorbance titration with DMT (extinction coefficient 510 M⁻¹cm⁻¹ at 520 nm), quantifying the decrease in tetrazine absorbance upon reaction with TCO. The initial PBS stock was diluted to prepare solutions for stopped-flow analysis at a final Sar-C₂TCO-Sar concentration of 2 mM.

Tetrazine stock solutions of 10 mM in DMSO were prepared. Serial dilution into PBS (pH 7.4, 10 mM) was used to prepare solutions for stopped-flow analysis at a tetrazine concentration of 100 μM.

Stopped-flow spectrophotometry: Stopped-flow measurements were performed using an SX20-LED stopped-flow spectro-photometer (Applied Photophysics) equipped with a 535 nm LED (optical pathlength 10 mm, full width half-maximum 34 nm) to monitor the characteristic tetrazine visible light absorbance (520-540 nm). The reagent syringes were loaded with tetrazine and Sar-C₂TCO-Sar solutions and the instrument was primed. Subsequent data were collected in triplicate to sextuplicate for each tetrazine. Reactions were conducted at 37° C. and recorded automatically at the time of acquisition.

Data analysis: Data sets were analyzed by fitting an exponential decay using Prism 6 (Graphpad) to calculate the observed pseudo-first order rate constants that were converted into second order rate constants by dividing through the concentration of excess Sar-C₂TCO-Sar.

Release Experiments

Instrument & solvents: all release experiments were performed on a Nexera X2® UHPLC system (Shimadzu®) with a temperature-controlled autosampler at 37° C. For buffered LCMS conditions, the aqueous solvent was prepared by addition of 625 μL of 10 M ammonium formate (BioUltra, Sigma-Aldrich) to 2.5 L of HPLC-grade water followed by adjusting the pH to 8.5 by addition of 100 μL of 25% aqueous ammonia (for HPLC, LiChropur, Merck). The pH of this volatile buffer declines over time and was thus freshly prepared each day. HPLC-grade acetonitrile was used without any additives.

Release measurements with AF594-C₂TCO-AF594: Analytical stock solutions and buffers. 10 mM stock solutions of the release probe AF594-C₂TCO-AF594 was prepared and further diluted with citrate-phosphate buffer (as indicated below), so that the final concentration of DMSO in buffer was never higher than 2%. Tetrazine stock solutions were prepared in DMSO at concentrations ranging from 10-100 mM and diluted with citrate-phosphate buffer (as indicated below), so that the final concentration of DMSO in buffer was never higher than 2%. Citrate-phosphate buffer (10 mM) was prepared by dilution from standard stock solutions of 0.1 M citric acid and 0.2 M Na₂HPO₄ with water and the pH verified and adjusted as needed to within ±0.05 pH units by digital pH metering.

Endpoint measurements with AF594-C₂TCO-AF594. Analytical samples for endpoint-release reactions were prepared as follows: AF594-C₂TCO-AF594 stock solution was diluted with citrate-phosphate buffer to a final concentration of 50 μM directly in an LCMS sample vial. Tetrazine stock solutions were diluted with aqueous buffer to give 100 or 200 μM concentrations, added to the TCO sample and mixed (1:1) to initiate the cleavage/release reaction. Samples were kept at 37° C. and HPLC analyses were done after 24 h. The released AF594-amine was detected using an RF-20Axs fluorescence detector and confirmed by a LCMS-2020 mass spectrometer (ESI) implemented in the Nexera X2® UHPLC system.

### Example 10—Bivalirudin Containing a Cleavable TCO Linker

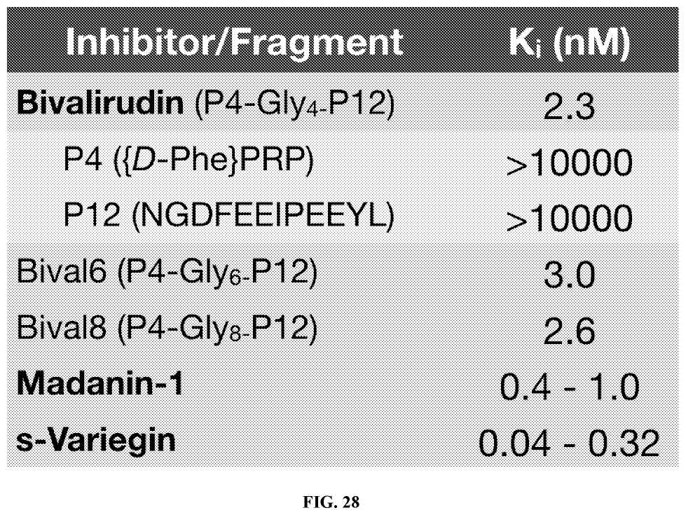

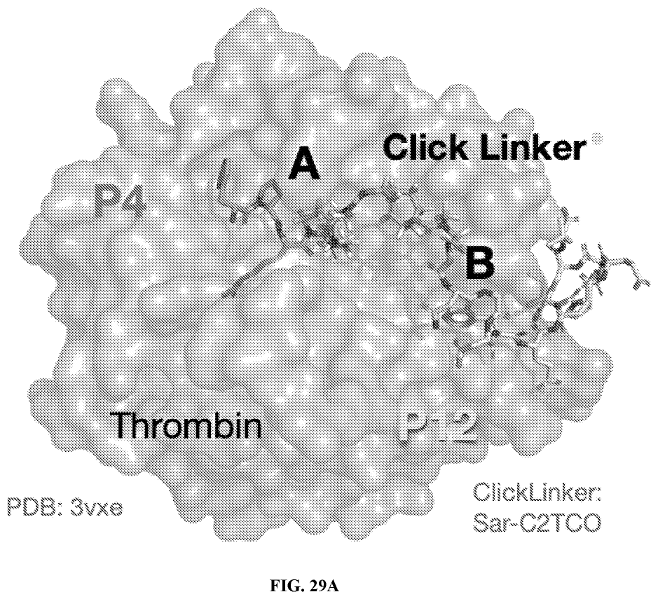

Bivalirudin is a clinical anticoagulant drug. Bivalirudin is a 20-amino acid peptide that binds to two distinct sites on the critical coagulation factor thrombin; the two binding elements (P4 and P12 hereafter) are linked by a flexible glycine tetrapeptide to form a highly potent inhibitor, but individually generate no anticoagulant effect even at high μM levels. Strikingly, the potency of the drug is also unchanged when the linker is extended from 4 to 6 to 8 glycines (FIG. 28).

The crystal structure of bivalirudin bound to human thrombin reveals no electron density for the Gly4 linker, indicating that it is disordered in the complex and does not occupy a defined binding site, validating the enzymologic data. A 3D model (FIG. 29A) informed present synthesis of a C₂TCO-linked bivalirudin analog (ClickBival1). The compound has excellent in vitro potency, comparable to the parent drug, bivalirudin itself (FIG. 29B).

Additional examples of ClickBival derivatives are prepared from commercially sourced peptide building blocks (Genscript, NJ, USA) and C₂TCO linker precursors of the present disclosure. The library is based on a P4-A-C₂TCO—B—P12 scaffold, where the AB flanking elements are varied to provide a range of short linkers (e.g., peptide or ethylene glycol based).

Tetrazine-TCO conjugates have been broadly nontoxic in animal pilot experiments for other applications. In the event of any unanticipated downstream pharmacologic or immunologic consequences from the P4/P12 fragments with ClickLinker remnants, the data indicates that bifunctional Tz acids (e.g., PA₂-Tz, bis-hydroxyaryl-Tz) produce >99% traceless cleavage, fully excising the bioorthogonal linker from both peptides, which is advantageous.

Programmed self-destruction of ClickBival is schematically shown in FIG. 29D. The Bival click adduct with the Tz reagent may be formed ex vivo, and then administered to a patient, e.g., by an injection or an infusion, with the engineered t_{1/2} of several minutes (e.g., 20 min). Conversely, the ClickBival can be infused to the patient, followed by administration of the inactivating Tz reagent. In this case, the click adduct is formed in vivo, with the engineered t_{1/2} of less than 25 seconds. The t_{1/2} can be tuned depending on the application and mode of administration, for example, by varying pH or the directing group of the Tz reagent. The fast click kinetics of, e.g., hydroxyaryl-H-Tzs enable complete click reactions in about 3s at the infusion concentration of clinical bivalirudin solutions (5 mg/mL, 2.3 mM). FIGS. 29E and 29F contains chemical structures of ClickBival compounds containing various TCO cleavable linkers.

C2TCO Bival

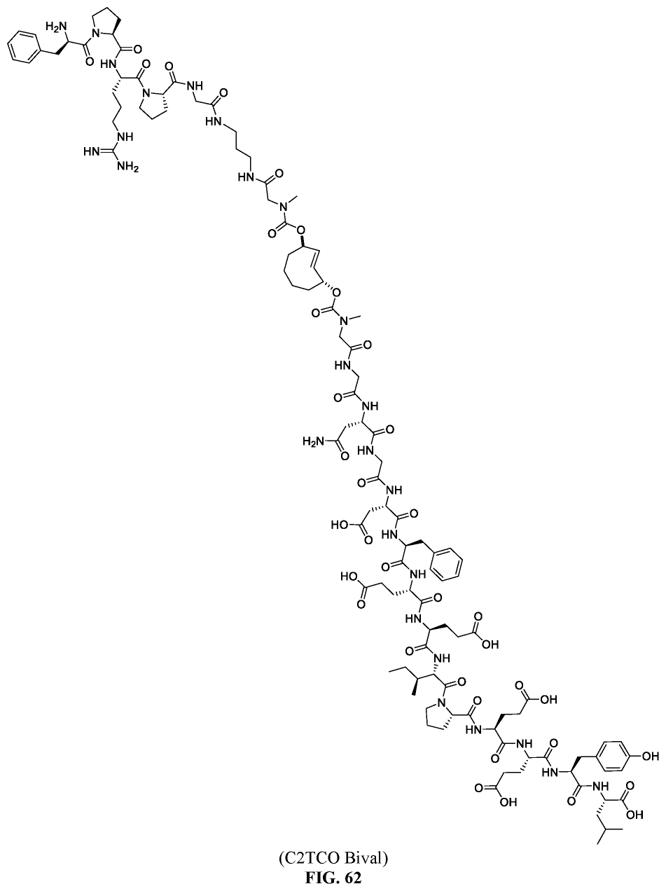

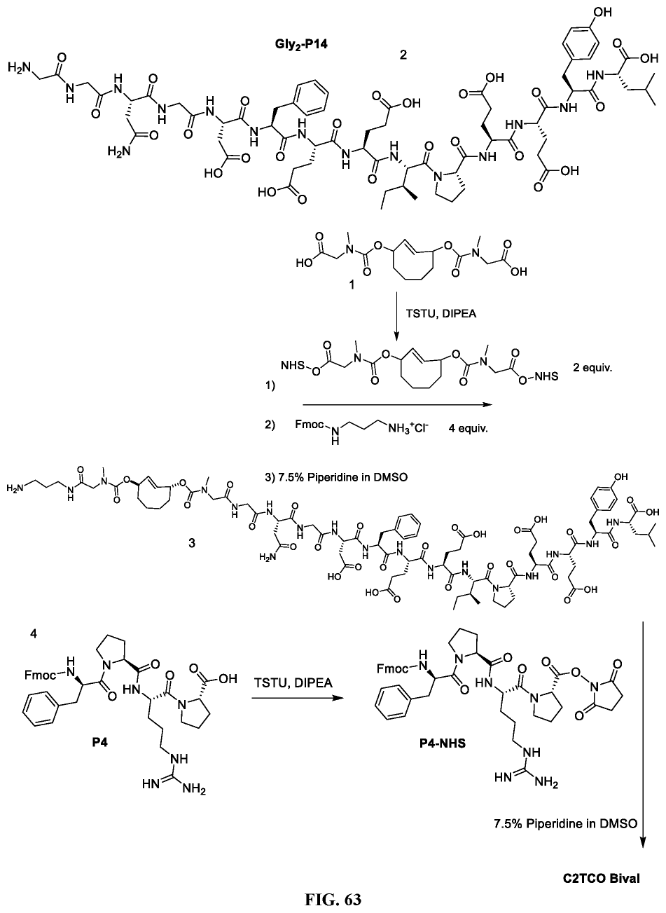

Chemical structure of C₂TCO Bival is shown in FIG. 62. Synthesis of C₂TCO Bival is shown in FIG. 63.

Referring to FIG. 63, Compound 1 was prepared as specified in Wilkovitsch et al. Compounds 2 and 4 were purchased as a custom-synthesized peptide (Purity >98%) from Genscript (New Jersey, USA). Compound 3 was prepared as follows: to a solution of Sar-C₂TCO-Sar (1) in 1 mL of dry DMSO were added triethanolamine (13.7 μL, 1M solution in DMSO), and TSTU (15.7 mg, 6.2 equivalents). The reaction was allowed to proceed for 2 hours at room temperature, whereupon a solution of 2 (Gly₂-P12, 12.7 mg, 1 equivalent) in 300 μL of dry DMSO was added. The reaction proceeded for an additional 1 hour at room temperature, followed by the addition of 1,3-diaminopropane (21.4 μL, 25 equivalents). After a further 30 minutes of reaction time, the reaction was neutralized by the addition of formic acid (20 μL) and then purified by reversed phase column chromatography (H₂O/MeCN gradient elution), yielding 8.3 mg of 3. ESI-MS [M−H]⁻ m/z calcd. 1919.86 for C₈₆H₁₂₃N₁₈O₃₂S₂⁻. found 1919.6.

C2TCO Bival: In a dry vial at room temperature were combined P4-CO₂H 4 (2.4 mg, 1.4 equivalents), TSTU (0.84 mg, 1.3 equivalents), and triethanolamine (21.6 μL, 1M solution in DMSO). The reaction was allowed to proceed for 30 minutes at room temperature, at which point LCMS verified conversion to the activated P4-NHS. To this reaction mixture were then added 4.15 mg of compound 3 (1 equivalent) in 5004, of DMSO. The coupling reaction was allowed to proceed for 3 hours at room temperature, at which point no further product formation was observed. Piperidine (118 μL) was then added, and the mixture was allowed to react for 15 minutes at room temperature before the addition of formic acid (50 μL) to neutralize the reaction. The reaction mixture was directly injected onto a reversed phase column and purified by gradient elution (H₂O/MeCN, 0.1% formic acid), yielding 2.4 mg of C₂TCO Bival as a white crystalline film. ESI-MS [M−2H]⁻ m/z calcd. 1236.57 for C₁₁₃H₁₆₀N₂₆O₃₇²⁻. found 1236.24.

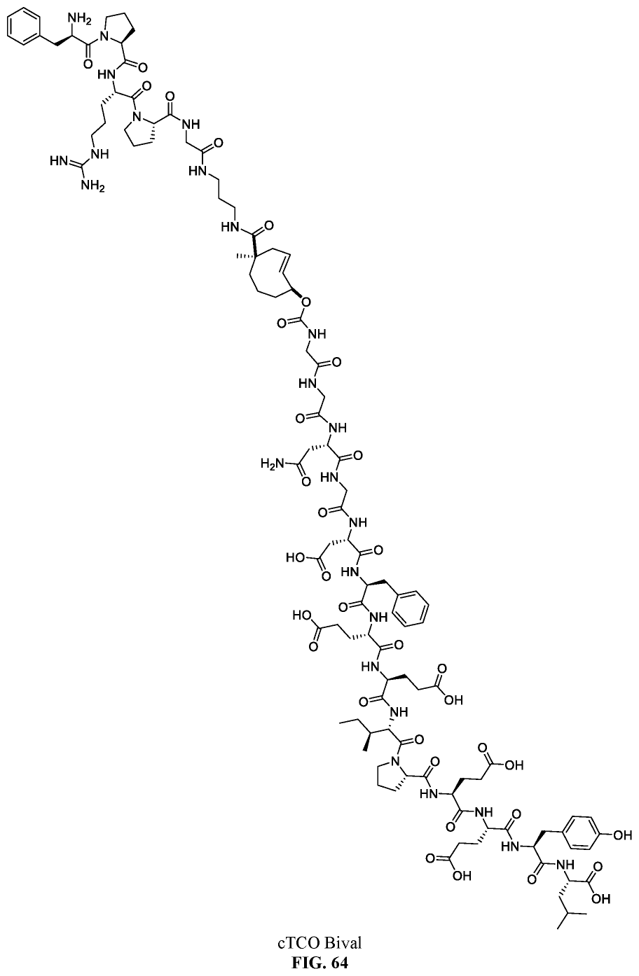

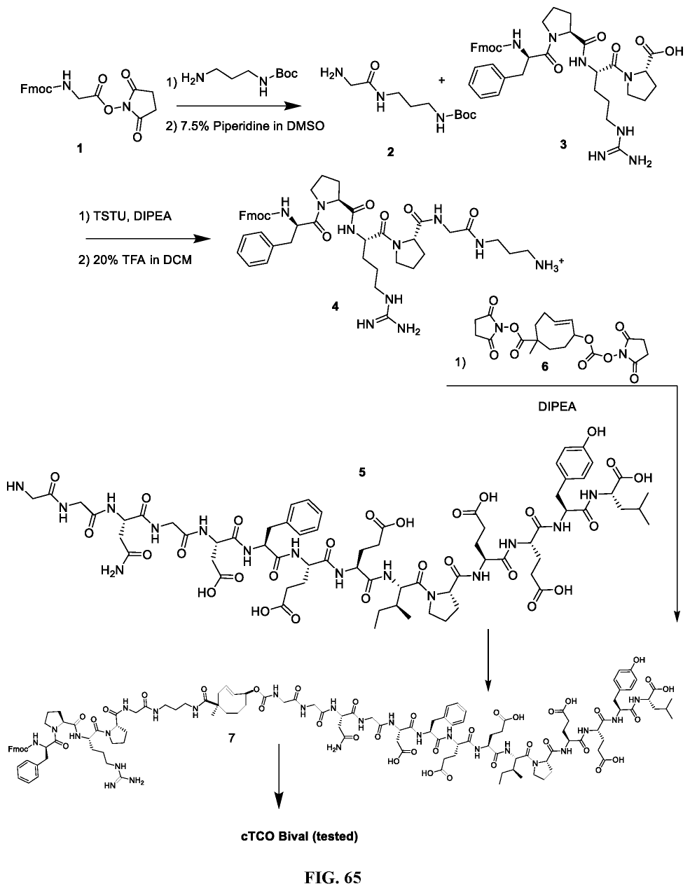

cTCO Bival Chemical structure of cTCO Bival is shown in FIG. 64. Chemical synthesis of the compound is shown in FIG. 65.

Referring to FIG. 65, Compound 2: to a solution of Fmoc-Gly-NHS (60 mg, 0.152 mmol, ChemImpex #02420) in 1 mL of anhydrous DMSO were added 28 mg of N-boc-diaminopropane (1.05 equivalents) and 23.3 μL of DIPEA (16.9 mg, 1.1 equivalents). The reaction mixture was vortexed assertively in an Eppendorf tube. After 5 minutes, complete conversion was observed by LCMS and 45 uL of piperidine were added for Fmoc deprotection. After a further five minutes, the reaction mixture was injected directly onto a 25 g SNAP Bio C18 column (Biotage) and purified with an ammonium formate (pH 8.5):acetonitrile gradient. Compounds 3, 4 and 5 were purchased as custom-synthesized peptides from Genscript (New Jersey, USA). Compound 6 was prepared as described: Rossin et al (10.1021/acs.bioconjchem.6b00231). Compound 8: to a solution of 5 (12.7 mg, MW 1681.6 as TFA salt, 0.0075 mmol) in 300 uL anhydrous DMSO were added cTCO bis-NHS 6 (3.52 mg, 1.1 equivalents) and triethylamine (1.13 μL, 1 equivalent). After 8 minutes the reaction was checked by LCMS and found to be incomplete. An additional 2 equivalents (2.26 uL) of triethylamine were added and the mixture was allowed to react for 5 minutes. Repeat LCMS indicated complete conversion, whereupon 8.1 mg of 4 were added in one portion, followed by an additional 1.13 μL of TEA. The reaction was vortexed intermittently and allowed to react for 2 hours. Incomplete conversion was observed by LCMS, with a mixture of Fmoc-deptrotected and intact product, as well as the hydrolyzed NHS intermediate. The reaction mixture was loaded directly onto a 10 g SNAP Bio C18 column (Biotage) and purified with a gradient elution of acetonitrile and ammonium formate (2.5 mM, pH 8.5). Fractions containing pure product (7) were collected and rotovapped to dryness. The mixture of peptide and ammonium formate salts was resuspended in 300 μL of DMSO followed by addition of 22.5 μL of piperidine. After 3 minutes this reaction mixture was again loaded onto a 10 g SNAP Bio C18 column and purified by gradient elution. The purified product was rotovapped to dryness and then quantified by UV Vis (extinction coefficient 1390 M-1 cm-1 at 276 nm) for a final yield of 7.6 mg (39%) of cTCO Bival (8) over two steps.

cTCO-PEG-Bival

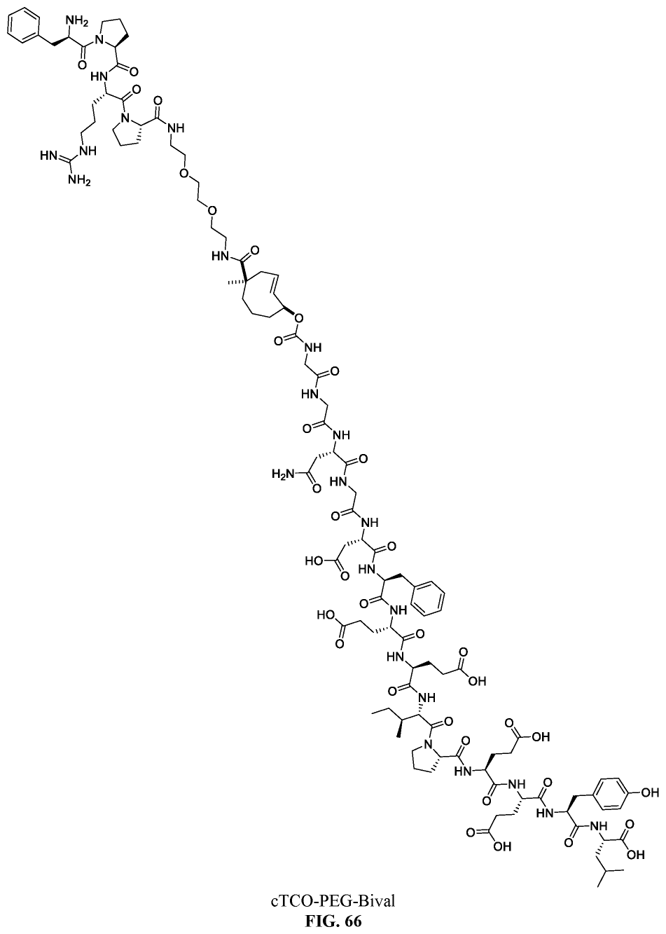

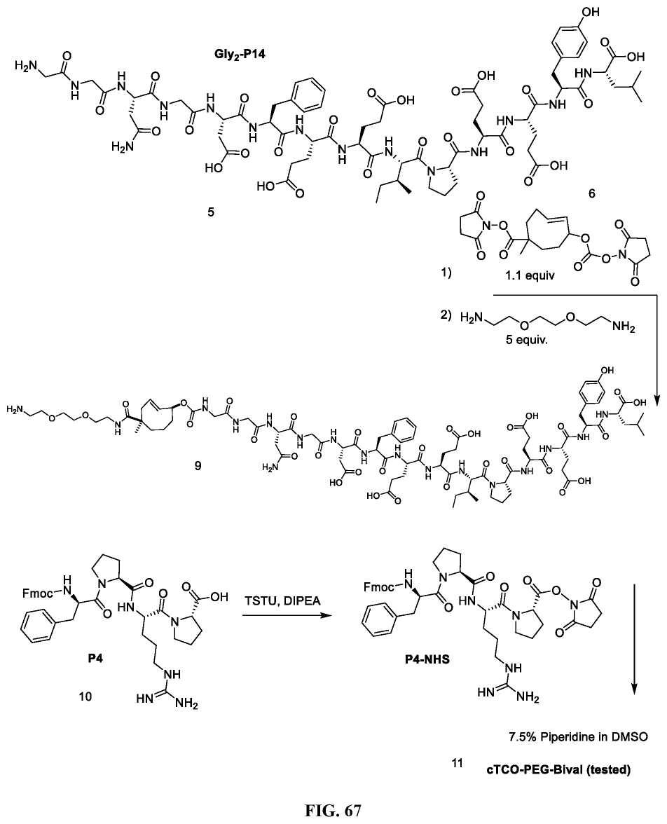

Chemical structure of cTCO Bival is shown in FIG. 66. Chemical synthesis of the compound is shown in FIG. 67.

Referring to FIG. 67, compound 9: To a solution of 15 mg of 5 (8.9 μmol; Genscript, NJ, USA) in 320 μL of DMSO were added 4.44 mg (1.1 equivalents) of cTCO—NHS₂ (6) and 2 equivalents of triethanolamine (2.54 μL, 2.86 mg). After ten minutes, 5 equivalents of 2,2′-(ethylenedioxy)bis(ethylamine) were added (7 μL, Aldrich #385506). After two hours the reaction was checked by LCMS and complete consumption of the NHS ester was observed, as a mixture of the desired product and the hydrolyzed intermediate. The reaction mixture was injected directly onto a 10 g SNAP Bio C18 column and purified by gradient elution in pH 8.5 ammonium formate:acetonitrile. Fractions containing the pure product were collected and rotovapped to dryness (15 mg, 82%). Compound 10: Purchased as a custom synthesized peptide (purity >98%, Genscript, NJ, USA). Compound 11: Compound 10 (12.8 mg) was dissolved in 300 μL of anhydrous DMSO, followed by the addition of 4.54 mg of TSTU (1 equivalent) and 2 equivalents of triethanolamine (2.1 μL, 2.34 mg). After 2 minutes, LCMS indicated complete activation of the terminal carboxylate to the N-hydroxysuccinimide ester, whereupon the reaction mixture was added to 15 mg of compound 9 from the prior step (0.5 equivalents). After 15 minutes, LCMS indicated complete conversion/consumption of the P4-NHS. Piperidine was then added to the reaction mixture (7.5% final concentration) to remove the N-terminal Fmoc group. After 5 minutes, the reaction mixture was then loaded directly onto a 10 g SNAP Bio C18 column and purified by gradient elution in pH 8.5 ammonium formate:acetonitrile, yielding 11.8 mg of pure 11 (57%) as well as an additional 8.6 mg of incompletely purified (˜85%) material that was reserved for subsequent isolation.

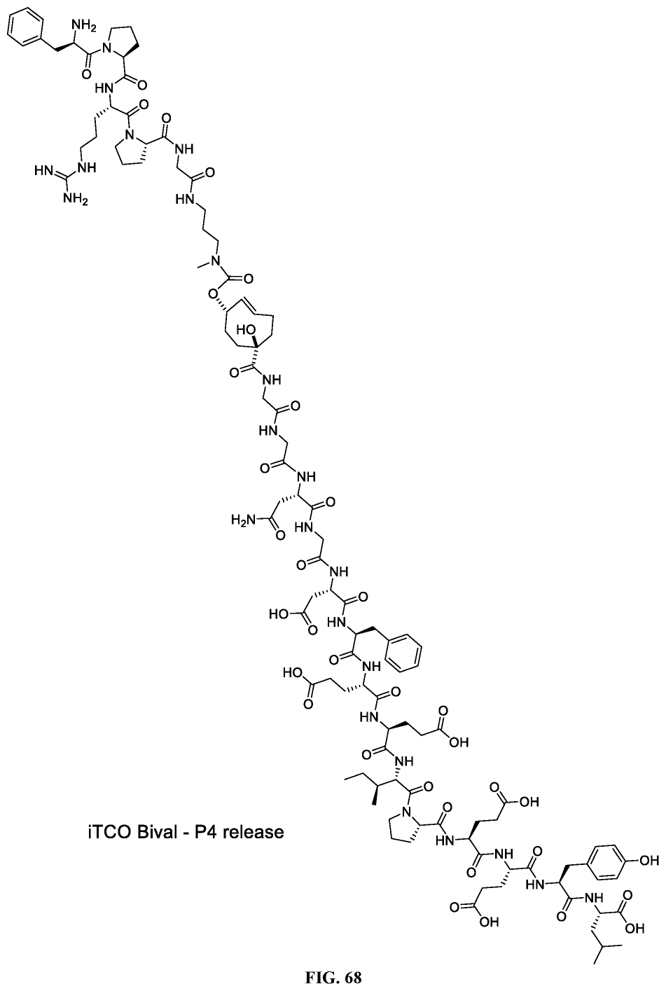

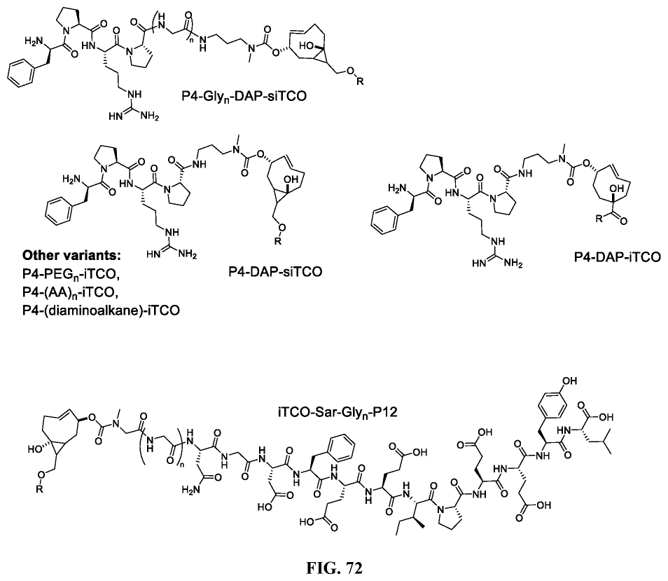

Chemical structures of Bival iTCO variants are shown in FIGS. 68-72.

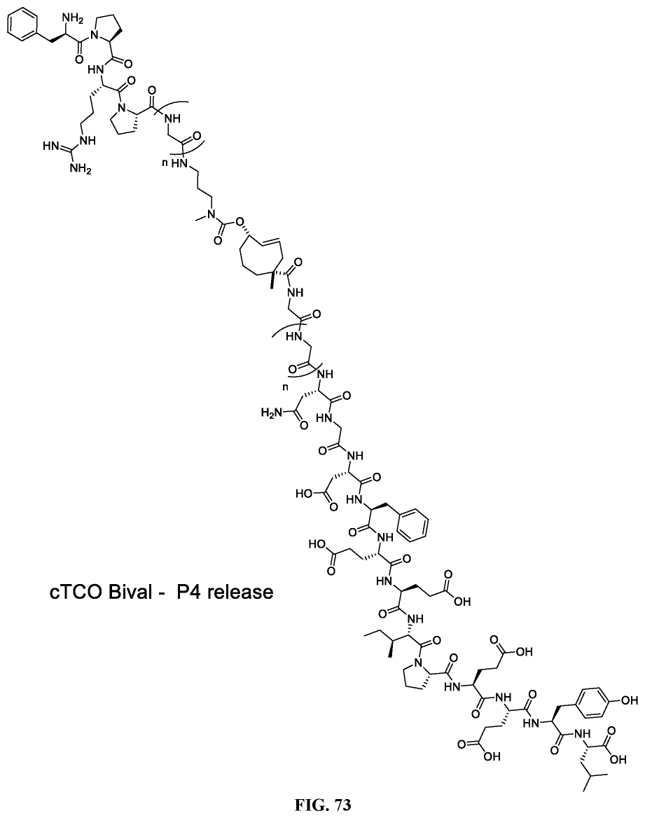

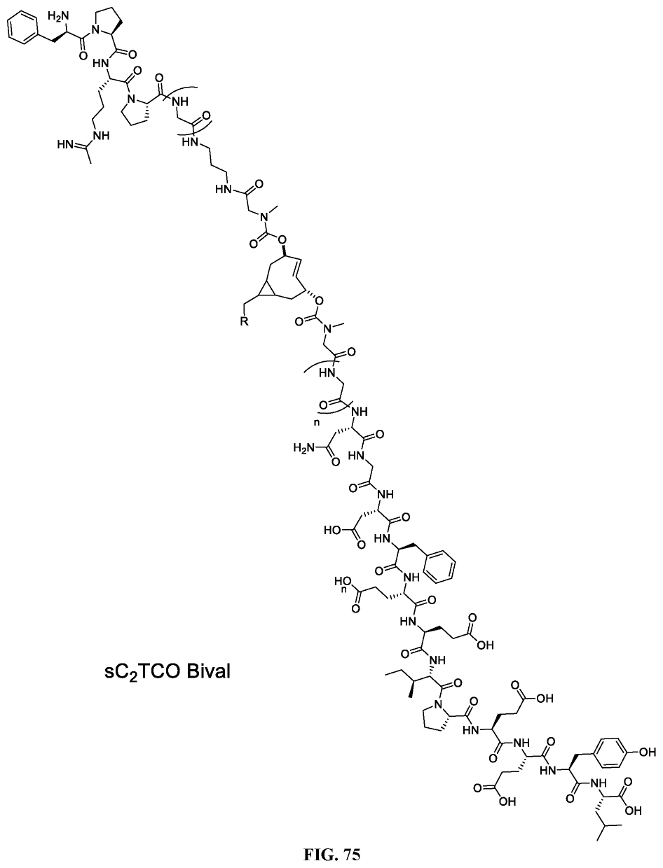

Chemical structures of additional Bival analogs are shown in FIGS. 73-77.

### Example 10a—C₂TCO Prodrug Approach

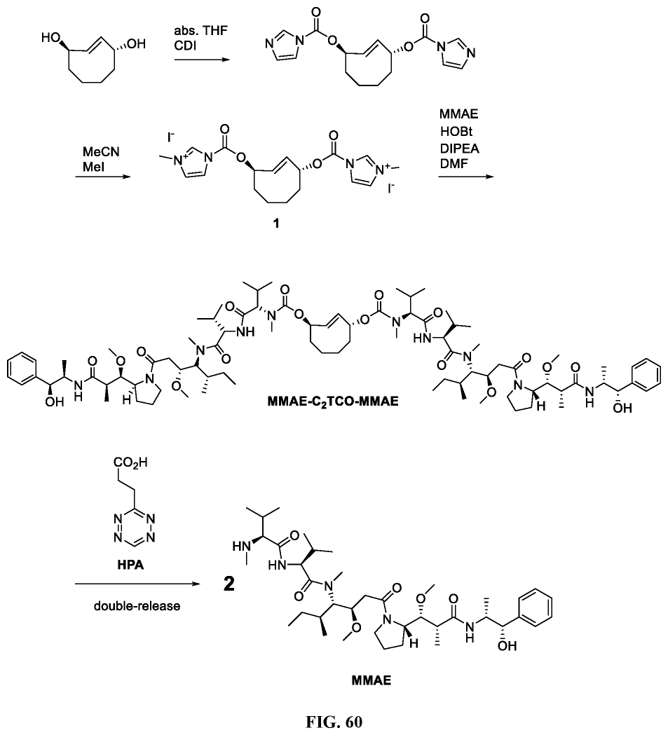

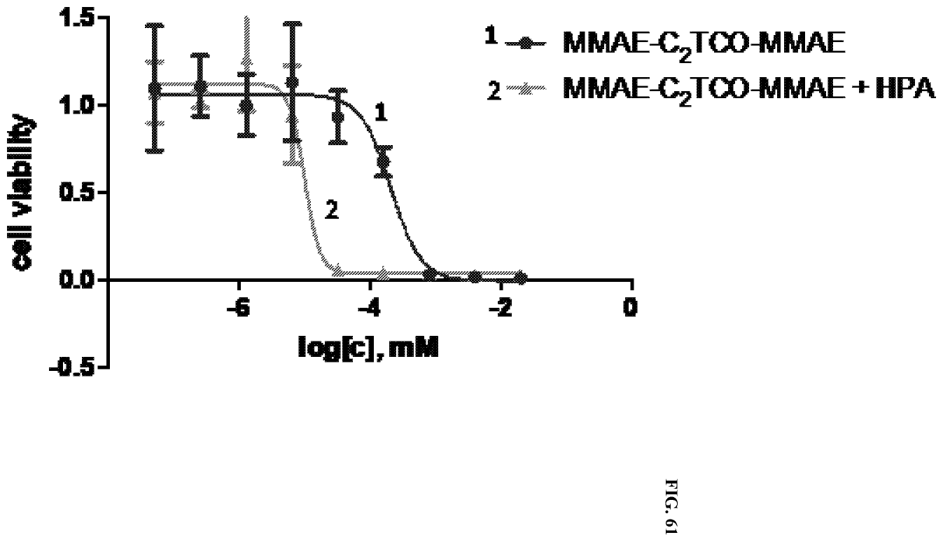

Chemical synthesis of the monomethyl auristatin E (MMAE) prodrug is shown in FIG. 60.

To a solution of MMAE (46.8 mg, 0.065 mmol) in dry DMF (1 mL) was added reagent 1 (20 mg, 0.032 mmol), DIPEA (25.2 mg, 0.195 mmol) and HOBt (8.8 mg, 0.065 mmol). The reaction mixture was stirred at room temperature for 5 hours. Purification by repeated reversed phase column chromatography (C18, 0.1% formic acid/MeCN gradient elution) gave MMAE-C₂TCO-MMAE (6.1 mg); ESI-MS: m/z 1630.1 (calc. for C₈₈H₁₄₅N₁₀O₁₈⁺). found: 1630.3.

Activation in cell culture (HT1080 cells; cell viability experiments; n=4) is shown in FIG. 61. IC₅₀ (MMAE-C₂TCO-M MAE): 206.4 nM. IC₅₀ (MMAE-C₂TCO-MMAE+HPA): 9.9 nM.

### Example 11—Thrombin Inhibitors Containing a Cleavable TCO Linker

Arthropod-derived thrombin inhibitors bind bivalently and potently to thrombin, including madanins and variegins, providing alternatives to bivalirudin (FIGS. 28 and 29A-C).

### Example 12—Integrilin Containing a Cleavable TCO Linker

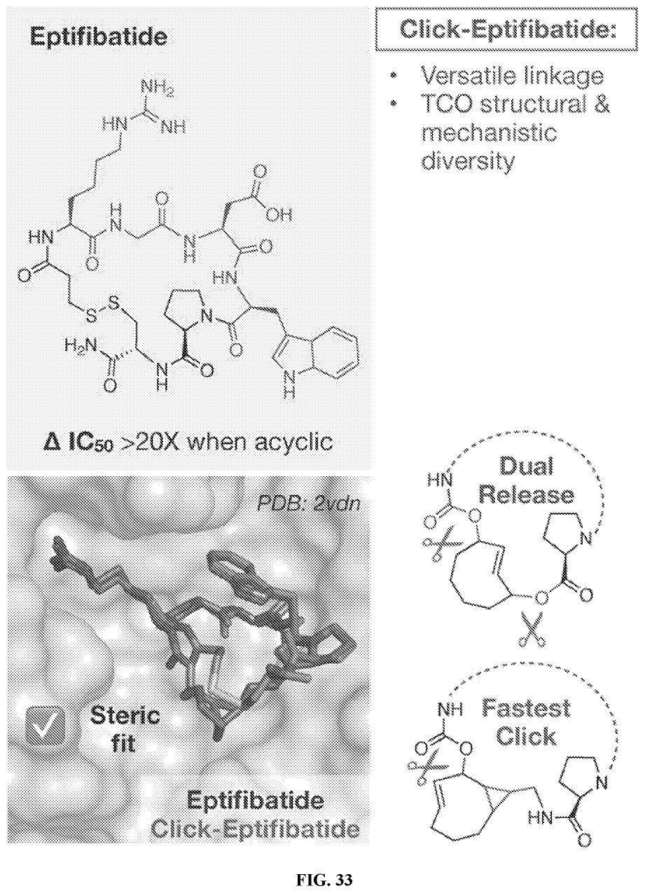

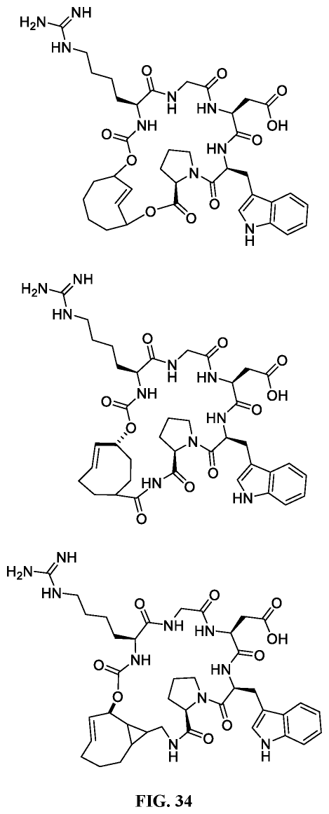

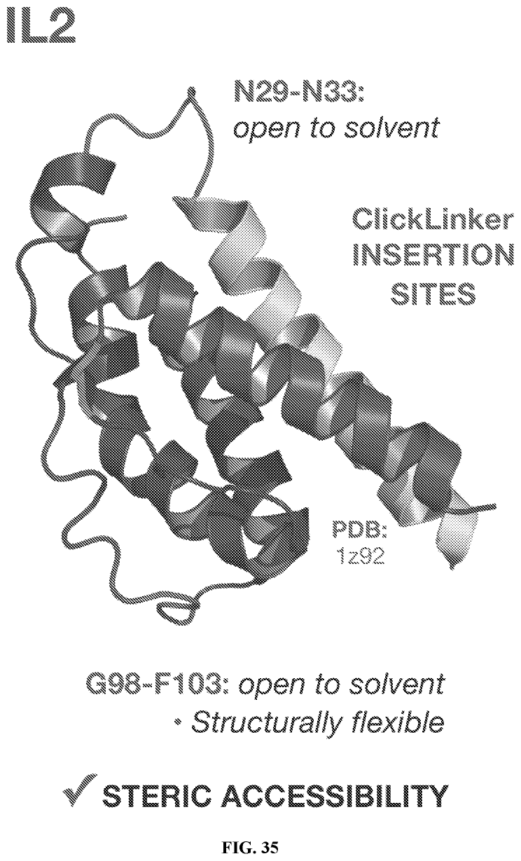

Eptifibatide is an infusional inhibitor of platelet aggregation and RGD mimetic that binds to the platelet surface receptor GPIIbIIIa. Its half-life of 3-4 hours is long enough that reversal in the event of problematic bleeding is clinically relevant. Its use in the context of interventional procedures in patients with unstable angina also creates the therapeutic equipoise where treatment discontinuation is an option—in contrast to applications such as clopidogrel (Plavix) and aspirin for stenting, where interruption of therapy is strongly contraindicated. The binding affinity of eptifibatide is markedly reduced by modifications that block cyclization. The crystal structure shows space in the binding pocket for TCO derivatives and an accessible conformation (FIG. 33). FIGS. 33 and 34 show chemical structures of integrilins containing TCO linkers within their structures.

Cleavable integrilins of this example allow for peri-infusional antidote concepts: i.e., delivery of a cleavable antiplatelet agent to the catheter tip during coronary artery interventions with systemic delivery of a rapid acting Tz antidote. With appropriate kinetic tuning and validation that cleavage of the drug disrupts receptor inhibition even after the target is already engaged, this approach allows locally intensive antiplatelet therapy with reduced systemic bleeding risk. The same method applies to catheter directed delivery of clickBival or click-tPA.

### Example 13—Chemoenzymatic Protease Cleavage, Click-tPA

The range of biomolecules for which spatiotemporal control is clinically relevant extends beyond the MW/size limit for which chemical synthesis is currently feasible, making direct ClickLinker regulation intractable for the critical hematologic proteins (e.g., thrombin, plasmin, tPA, fibrinogen, ranging from 40-340 kD) in the clotting cascade. This example shows non-direct inactivation using non-canonical amino acid (ncAA) incorporation.

A biologically active protein, such as tPA, has a domain within its structure that binds to a protease, and the protease then enzymatically hydrolyses the biologically active protein, rendering it inactive. An amino acid within the protease binding domain of the biologically active protein can be modified with a cleavable moiety such that the protease would no longer recognize and/or bind to the domain. Hence, the biologically active protein having an ncAA modified with the cleavable group would be protected from proteolytic degradation. Upon cleaving the cleavable group, however, the protease binding domain within the protein would be available for engaging the enzyme, leading to proteolytic degradation of the protein. Hence, introducing a ncAA into the protease binding domain of a biologically active protein allows for time-controlled, delayed, and indirect inactivation of the biologically active protein.

Efforts to optimize ncAA incorporation for protein labeling and fluorescence imaging have focused intensively on development of Lysine-TCO derivatives. One example of such compound is Lys-rTCO, which exhibits strongly Tz-dependent Lys release efficiency, such that the most commonly applied tetrazine labeling probes (e.g. bipyridyl-Tzs) yield the efficient tagging needed for imaging studies, while click with optimized Tz-acids generates quantitative release.

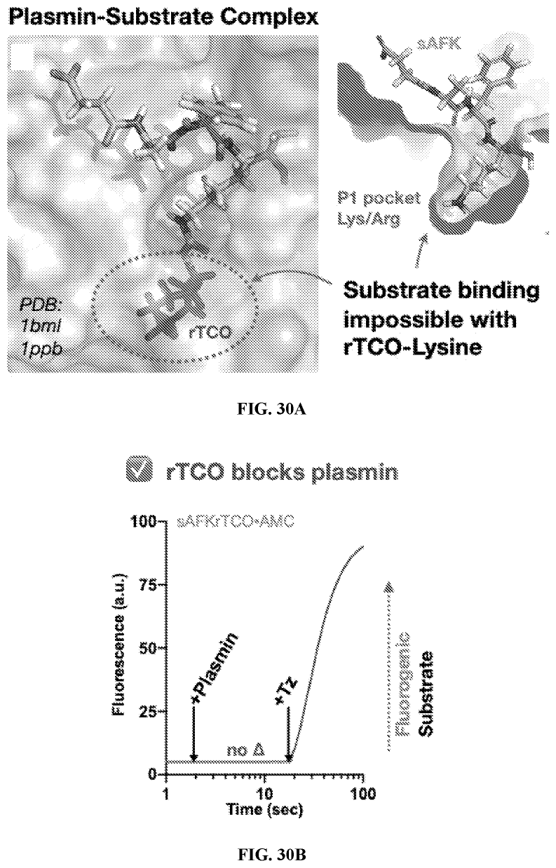

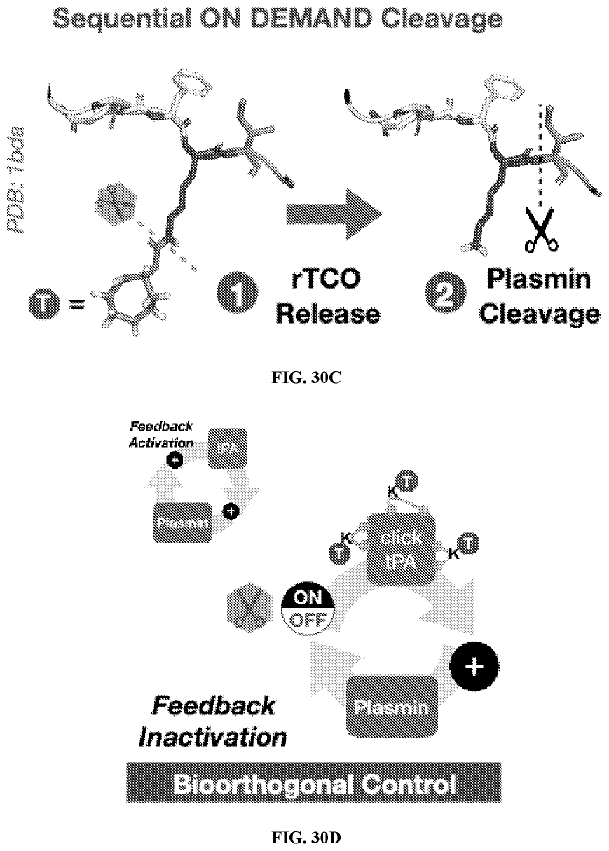

While experiments with Lys-rTCO have demonstrated the Tz-dependent uncapping of a catalytic Lys residue in luciferases/kinases, it has not been previously recognized that a protease substrate could be reversibly caged by this means. Trypsin- and thrombin-like serine proteases both recognize Lys/Arg residues in the P1 position. Therefore, Lys-rTCO is used here to link chemical and enzymatic cleavage. Crystallographic data indicates that the compact S1 binding pocket of the key fibrinolytic protease plasmin cannot fit an rTCO (FIG. 30A). This was experimentally verified with a fluorogenic rTCO-modified plasmin substrate (FIG. 30B). While any recombinant loop, linker, or fusion protein with a Lys-selective protease cleavage site can be masked until rTCO release (FIG. 30C), this approach provides a unique means to control the therapeutic thrombolytic enzyme tissue plasminogen activator (tPA) via feedback inactivation (FIG. 30D), in which Tz cleavage initiates the inactivation of tPA by plasmin.

tPA is used to treat acute stroke, myocardial infarction, and venous/arterial thrombosis; it specifically cleaves and activates plasminogen to plasmin, which then degrades fibrin clots. Native physiologic feedback from plasmin further activates tPA by cleaving it from a single chain to a dual chain form. The clearance of widely-used alteplase is rapid (t_{1/2} of about 5 min) and not proteolytic, but rather primarily hepatically mediated; recombinant tPA therapeutics have been developed with extended half-lives (15-25 minutes). All of these agents create a significant systemic bleeding risk that has led to carefully stratified approaches to determine which patients to treat. In spite of these precautions, there is a nontrivial risk of systemic fibrinogen depletion (and resulting coagulopathy) even with catheter-directed approaches, especially when long term infusions are used for severe clot burdens.

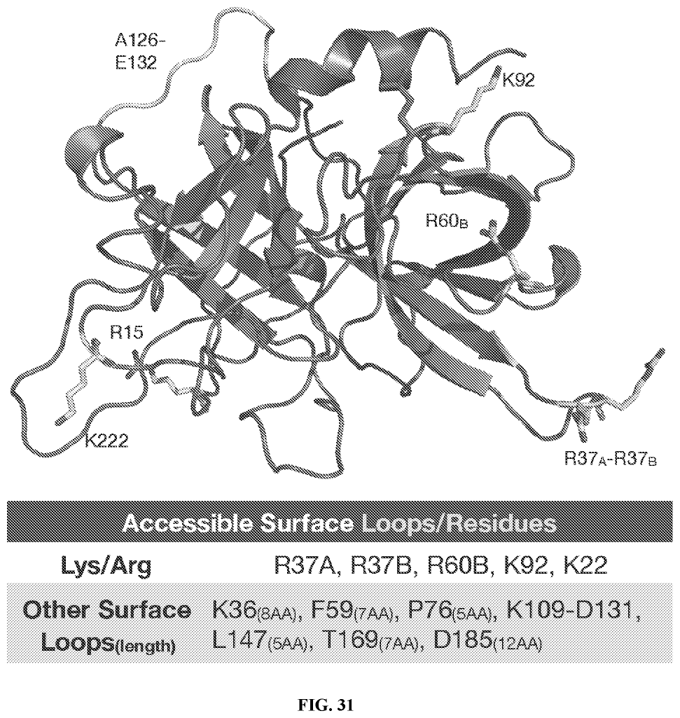

To generate Click-tPA, a site that can accommodate one or more rTCO-Lys residues within a compatible plasmin cleavage sequence is identified/introduced. FIG. 31 highlights Lys/Arg residues that enable modifications. Pyrrolysine tRNA/aminoacyl tRNA synthetase (AARS) system was used to incorporate Lys-rTCO at the relevant position and the ncAA-modified protein was purified for studies.

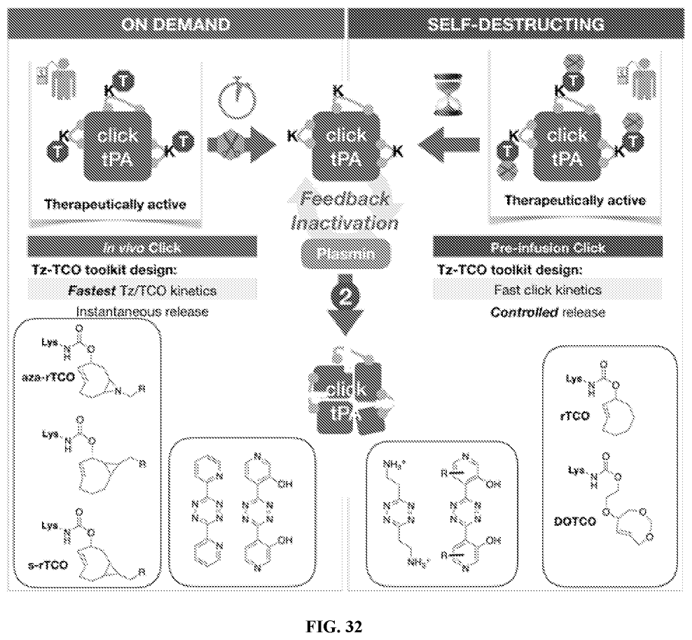

The clinical application of click-tPA (FIG. 32) can be used to tune a short half-life for truly local (rather than systemic) thrombolysis with catheter directed therapy. In addition, click-tPA can be applied to longer-acting applications to create an on-demand antidote compatible with potential in vivo reversal applications.

### Example 14—Dynamic Cytokine Control

Prior to the rise of checkpoint therapies, systemic IL2 treatment had a longstanding role in the treatment of immune-sensitive malignancies (e.g., melanoma, renal cell carcinoma) that was notable for: i) a low but nontrivial rate of curative responses; ii) profound systemic toxicity from the intense immunostimulation. The search for adjuvant approaches to enhance checkpoint blockade has included a wide range of efforts to deliver local immunomodulators into the tumor microenvironment without creating toxic systemic exposure.

This example shows a clickable assembly by splitting the N terminal or C terminal helix of IL2. The IL2 structure reveals that residues N29-N33 and G98-F103 are flexible and solvent accessible in the IL2-IL2R complex, providing space for ClickLinker elements without biological/functional interference.

### Example 15—Multiplexed Analysis of Living Cells with SAFE Bioorthogonal Cycling

Materials and Methods

Chemistry. Synthesis and characterization. Compounds were made according to well-accepted synthetic methods, including collecting and analyzing the NMR spectra and ESI-MS data. Fluorescence measurements were conducted with a PTI QuantaMaster 400 fluorimeter (Photon Technologies Incorporated, NJ, USA) equipped with a Pelletier apparatus for temperature control, multi-sample changer with integrated magnetic stirring, and a sample addition port for continuous kinetic measurements. UV-VIS absorption spectra were measured on a Horiba DualFL spectrophotometer (Horiba Instruments, NJ, USA) or Nandrop Spectrophotometer (ThermoFisher).

SAFE Probe activation. To a 500 μL eppendorf tube containing 10-30 μL of 4(a-d) in anhydrous DMSO were added 2 equivalents of DIPEA followed by 4 equivalents of N,N,N′,N′-tetramethyl-O—(N-succinimidyl)uronium tetrafluoroborate (TSTU) and the solution was quickly vortexed after capping the tube. After one minute, 5 equivalents of 4-(ethylamino)-butanoic acid hydrochloride (ENBA) were added followed by an additional 5 equivalents of DIPEA. Note: The reaction time prior to addition of ENBA is concentration dependent and may be complete in as little as 10 seconds at higher dye/TSTU concentrations. For a dye solution between 1-2 mM, the sixty second reaction time provides optimal discrimination between the PEG4 carboxylic acid and the rhodamine 2′-carboxylic acid, with minimal conversion to the double-NHS species. After addition of ENBA, the solution was again vortex-mixed and ready to use for protein labeling or LCMS analysis after thirty seconds. The chemistry is moderately moisture sensitive due to the risk of NHS hydrolysis and appropriate storage/handling conditions were applied.

Bioorthogonal Click and Scission Kinetics. A stock solution of BHQ3-N-Tz was prepared in DMSO at a concentration of 5 mM, verified by absorbance measurements (extinction coefficient 42,700M-lcm-1 in PBS pH 7.3) and subsequently diluted as needed in small aliquots to prepare a working solution at 250 μM. In parallel, cetuximab (Erbitux) was labeled with TSTU-activated AF647-C₂TCO to prepare a SAFE647 conjugate with a DOL of 2.6 (for detailed antibody labeling methods, vide infra). To a disposable polystryrene cuvette containing 2 mL of PBS and a magnetic stirring bar were then added 40 μL of unlabeled cetuximab (2 mg/mL) to block nonspecific adsorption (40 μg/mL, 275 nM), followed by 1 μL of the SAFE647-conjugated cetuximab (2040-fold dilution, 6 nM final concentration). Fluorescence intensity was measured (ex 645 nm, em 670 nm, monochromator slits 5 nm) to establish a stable background, the baseline intensity of the SAFE labeled antibody, and the time course of click/scission in a continuous trace. After measuring the initial signal, a micropipette was used to inject 8, 10, or 12 μL of BHQ3-N-Tz (250 mM) through the QM400 sample addition port into the stirred cuvette. Nonlinear fitting to a double exponential model in GraphPad Prism 9 (GraphPad software, LLC) was used to calculate a pseudo first order rate constant for the click reaction (falling signal) and a first order rate for the subsequent scission/cleavage (rising signal phase).

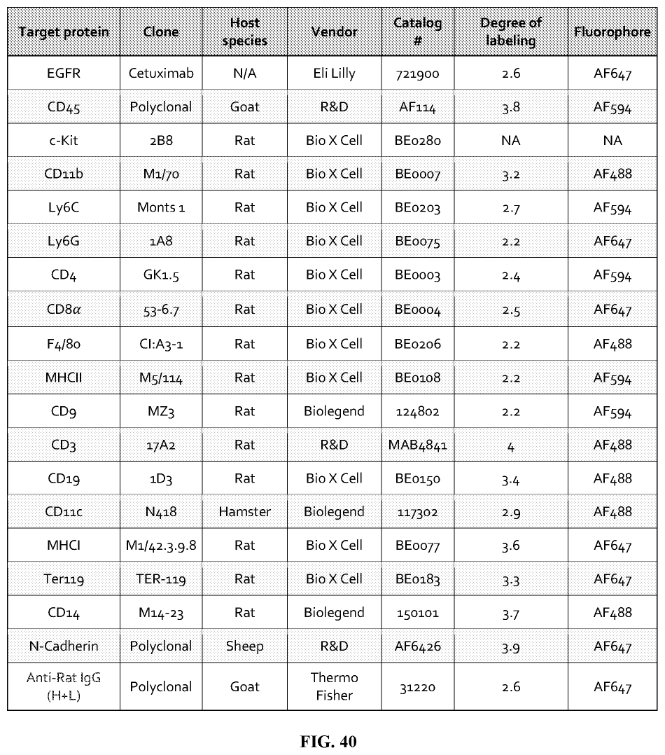

Bioconjugation and routine imaging methods. Antibodies. BSA free antibodies were purchased from established vendors and stored according to manufacturer recommendations. Cetuximab (anti-EGFR antibody, Erbitux) was used to optimize staining and quenching/scission methods. Antibodies used for all SAFE imaging throughout the study are tabulated in FIG. 40. All antibodies were validated with conventional immunofluorescence imaging (secondary antibody staining) on positive cell lines or mouse splenocytes before usage in SAFE imaging.

Antibody modification with SAFE probes. For functionalization with fluorophore-C2TCO conjugates, antibodies were first exchanged from their storage solution into PBS-bicarbonate buffer (PBS supplemented with 100 mM sodium bicarbonate, pH 8.4) using a 40 k Zeba Spin column (Thermo Fisher, 87765). After buffer exchange, antibodies were incubated with a 4- to 8-fold molar excess of the activated Dye-C2TCO—NHS linker for 25 mins at room temperature. The completed conjugation reaction was loaded onto another 40 k Zeba column (equilibrated with PBS) for desalting and removal of unreacted dye molecules. The absorbance spectrum of the conjugated antibody was measured using a Nanodrop 1000 (Thermo Scientific) to determine the degree of labeling (DOL), applying the known extinction coefficients of the dye, IgG antibody, and standard correction factor (CF280) for the dye absorbance at 280 nm. The SAFE-conjugated antibodies were stored in the dark at 4° C. in PBS until usage.

Immunostaining and fluorophore scission. Cells were stained with 5 μg/mL SAFE probes and incubated for 10 mins at 4° C. Hoxb8 cells were first blocked with BSA-PBS for 10 mins at 4° C. before staining. After imaging, a 600 nM solution of BHQ3-N-Tz (2) diluted in PBS was used to wash cells for quenching and scission; while signal removal is characteristically complete in 30-60 seconds, a routine interval of up to 3-5 minutes was used to insure complete removal of residual fluorescence. After incubation, remaining BHQ3-N-Tz was washed three times with PBS in immediate succession. After quenching/scission, the staining, imaging, and scission cycle was repeated for multiplexed protein profiling of the same cells. Imaging was performed in Live Cell Imaging Solution (Thermo Fisher, A14291DJ). Calcein AM (Thermo Fisher, C1430) and SYTOX Red (Thermo Fisher, S34859) were used for live and dead cell staining and Hoechst 33342 (Thermo Fisher, H3570) was used for nuclear staining in a subset of experiments. Hoechst staining was associated with an increased rate of phototoxic stress in live cell microscopy, especially in longitudinal imaging, so its use was reserved for short-term experiments and for those in which nuclear morphology was relevant.

Fluorescent imaging and analysis. An Olympus BX-63 upright automated epifluorescence microscope and a DeltaVision inverted fluorescence microscope were used to acquire fluorescent images. DAPI, FITC, TxRed/Cy3, and Cy5 filter cubes were used to excite Calcein AM live cell stains, AF488, AF594, and AF647 fluorophores respectively. ImageJ was used to measure fluorescent intensities in cells.

Cell culture methods. Cell Line culture. The A549 (CCL-185) and A431 (CRL-1555) cell lines were purchased from the American Tissue Culture Collection (ATCC) and used in method development experiments. A549 cells were passaged in F-12K (ATCC, 30-2004) with 10% FBS and 1% penicillin/streptomycin according to the specifications from ATCC. Cells were first grown in a 150 mm cell culture dish and then seeded on μ-Slide 8 Well Glass Bottom slides (Ibidi, 80826) for imaging. After 24-48 hours, confluency was assessed and cells were stained and imaged.

Microwell fabrication. The microwells used to culture and/or image nonadherent cells were fabricated at the Soft Materials Cleanroom (SMCR), Harvard Center for Nanoscale Systems (CNS). Each microwell array consists of 1681 total wells. The microwells (W: 60 μm, H: 80 μm) were made using soft lithography with SU-8 3050. Different widths were tested (60, 80, 100 μm) and 60 μm was chosen, a dimension that demonstrated no detectable cell loss throughout multiple experimental steps (e.g. media change, incubation, washing, culture, etc). Polydimethylsiloxane (PDMS) was spin coated on a patterned wafer, cured in 65° C. for an hour, peeled off from the wafer, and then transferred to μ-Slide 8 Well Glass Bottom slides (Ibidi) for cell culture and imaging.

Longitudinal imaging of A549 Cells. SAFE labeling. A549 cells were cultured in μ-Slide 8 Well Glass Bottom slides (Ibidi) for 24-48 hours before imaging. After 24-48 hours, cells were stained with 5 μg/mL of SAFE conjugated (AF647) anti-EGFR antibody for 10 mins at RT, and then rinsed with three successive washes of PBS, culminating with addition of live cell imaging solution (Thermo Fisher, A14291DJ). After image acquisition, quenching was performed by incubating the stained live cells with BHQ3-N-Tz (2) at 600 nM. Fluorescent images were acquired after staining, 1 min after quenching/scission, and 24 hours after scission. After 24 hours, cells were stained with calcein AM (Thermo Fisher, C1430) and SYTOX Red (Thermo Fisher, S34859) to check cell viability. A DeltaVision inverted fluorescence microscope was used to acquire all the fluorescent images in this study.

No scission control experiments. A549 cells were cultured and prepared as above and then stained with a 5 μg/mL of AF647-rTCO-conjugated (FAST647, Ko et al, ACIE 2020) anti-EGFR antibody for 10 mins at RT. This antibody is able to click with a BHQ3-Tz quencher but the linker/dye remain permanently attached to the antibody: i.e., no scission is possible, only quenching. After image acquisition, the stained live cells were incubated with the non-scissile BHQ3-Tz (600 nM, 1 min), as previously described. Fluorescent images were acquired after staining, 5 min after quenching, and 24 hours after quenching to quantify rebound. After imaging at 24 hours, cells were stained with Calcein AM (Thermo Fisher, C1430) and SYTOX Red (Thermo Fisher, S34859) to check cell viability.

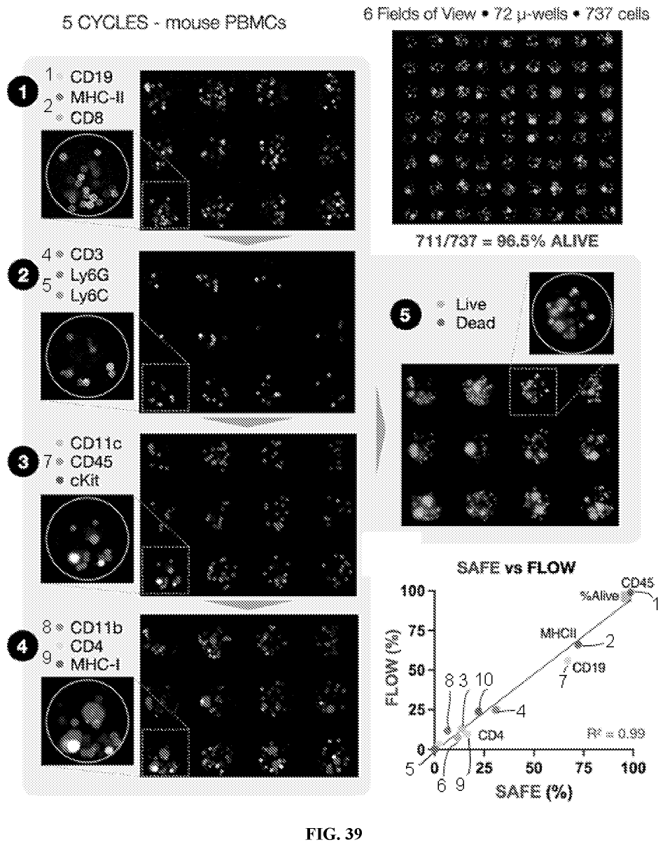

SAFE Imaging of mouse PBMCs (FIG. 39). Mouse PBMC isolation. Whole blood was collected from C57BL6/J mice (Jackson Laboratory) by cardiac puncture. Peripheral blood mononuclear cells (PBMC) were isolated by density gradient centrifugation. Lymphoprep (Stem Cell Technologies, 07801) was used as density gradient medium. Whole blood was diluted with an equal volume of PBS with 2% FBS, layered with Lymphoprep on top, and centrifuged at 800×g for 20 minutes at room temperature with break off. The upper plasma layer were discarded and cloudy mononuclear cell layer was gently collected in a separate tube. Collected cells were washed with PBS and cultured for imaging.

SAFE imaging in microwells. Isolated mouse PBMC were diluted in PBS to provide an average of 10 cells per well and loaded to microwell arrays for cycling and imaging. For each cycle, cells were stained with a cocktail of three SAFE antibodies (5 μg/mL) conjugated to three different fluorophores (AF488, AF594, AF647) for 10 mins at RT. Standard scission conditions used 2 (600 nM) for one to three minutes. For the 5th cycle, cells were stained with Calcein AM (Thermo Fisher, C1430) and SYTOX Red (Thermo Fisher, S34859) per the vendor protocol to check cell viability after the cumulative profiling experiment. Staining and quenching solutions were diluted in PBS and imaging was performed with cells in live cell imaging solution (Thermo Fisher, A14291DJ). Images were acquired using a DeltaVision inverted fluorescence microscope to capture 6 fields of view (72 microwells).

Flow Cytometry Control Experiments. Mouse PBMCs were incubated with True Stain FcX antibody (clone 93, BioLegend) in PBS with 2% FBS before staining with antibodies directly conjugated to fluorophores. Cells were divided into two tubes to stain for myeloid markers (CD11b, CD11c, Ly6C, Ly6G, MHC I, MHC II) or lymphoid markers (CD3, CD4, CD8, CD19, c-kit). Antibodies are summarized in FIG. 39B. DAPI was used to exclude dead cells from analysis. Cells were washed and strained through a 40 μm filter (BD Falcon, 352340) after staining and analyzed on a BD LSRII flow cytometer. AbC Total Antibody Compensation Bead Kit (Thermofisher Scientific, A10513) was used for single color compensation. Flow cytometry data were then analyzed using FlowJo software (Tree Star Inc.) to quantify the frequency of cells positive for each marker.

Mouse Bone Marrow Imaging. Mouse bone marrow isolation and culture.

Microcentrifuge tubes (0.5 mL) were prepared by removing the lids (scissors) and piercing a hole through the bottom of the tube using a 16G sterile needle. Wild type mice (C57Bl/6) were euthanized by carbon dioxide asphyxiation and the femurs removed in sterile fashion. The distal end of the femur was opened using scissors and the femur placed with the open end facing down into the bottom of the 0.5 mL microcentrifuge tube. This tube was placed inside a 1.5 mL microcentrifuge tube and the tube-within-a-tube centrifuged at 10,000 g for 15 seconds to “spin-flush” the bone marrow. The 0.5 mL microcentrifuge tube and “empty” femurs were discarded and the bone marrow pellet (˜25 μL) resuspended in PBS containing 2% FBS and 1 mM EDTA. Mononuclear cells were enumerated by staining a small aliquot with acridine orange (ThermoFisher, A3568) and counting using a fluorescence-based cell counter (Cellometer, Nexcelom).

Primary murine bone marrow cells were cultured in μ-Slide 8 Well Glass Bottom slides (Ibidi) for 24 hours in RPMI1640 media with glutamine, supplemented with 10% FBS, SCF (50 ng/mL), IL3 (10 ng/mL), and IL6 (10 ng/mL). To improve cell adhesion to the slides, various coating methods (Fibronectin, Poly-D-Lysine, Lipopolysaccharide, ibiTreat tissue culture-treated slides from Ibidi) were tested and ibiTreat slides were selected, as they showed the best cell adhesion. After 24 hours of cell culture, cells were stained with a cocktail of three SAFE antibodies (5 μg/mL, 10 mins at RT) and quenched with BHQ3-N-Tz (1 μM, 5 mins at RT) for each cycle.

Imaging. Cells were imaged in live cell imaging solution (Thermo Fisher, A14291DJ) at room temperature. Images were collected using a custom made Olympus FV1000 confocal multi-photon system using a 60×LUMFL N water immersion objective (NA=1.1, Olympus America). Fluorescent channels were imaged sequentially with 405 nm, 473 nm, 559 nm and/or 635 nm lasers using a 405/473/559/635 dichroic to separate excitation light and dichroic beam splitters SDM473, SDM560, and SDM640 to separate emission light. Fluorophore signals were further separated using emission filters BA430-455, BA490-540, BA575-620 and BA655-755. Each fluorescence channel was imaged sequentially using distinct excitation and emission filter sets to ensure minimal bleed-through between channels. All lasers, beam splitters, and emission filters were purchased from Olympus.

ER-Hoxb8 Differentiation Model. ER Hoxb8 Cell culture. ER-Hoxb8 cells were derived from the bone marrow of male ER-KO C57BL/6 CD45.1STEM mice (Mercier, 2016, Stem Cell Reports). Detailed protocols for construction of the retroviral vector encoding ER-Hoxb8, virus production, and transduction of bone marrow myeloid progenitor cells were described previously (Nature methods 2006). In brief, bone marrow mononuclear cells were isolated and expanded in medium containing SCF, IL-6, and IL-3 (10 ng/mL) for 48 hrs. Cells were placed in 12-well plates pre-coated with human fibronectin (Sigma F-0895) and spin-infected with murine stem cell virus encoding ER-Hoxb8, as previously described. Infected cells were cultured in RPMI medium containing SCF (20 ng/mL) and f3-estradiol (500 nM) for two days prior to addition of G418 for selection. The cells were passaged to new plates with fresh medium every 3-4 days and non-adherent immortalized cells grew out in about 3 weeks. These cells were maintained in culture no more than 4 weeks (6-well culture plate) until needed for microwell imaging experiments.

ER Hoxb8 longitudinal microwell imaging. ER-Hoxb8 cells were imaged longitudinally on Day 0, 2, 4, and 6. On Day 0, cells were stained with a cocktail of two SAFE conjugated antibodies (anti-CD45, anti-cKit) for 10 mins at 4° C. The antibodies were diluted to 5 μg/mL in cell culture media with β-estradiol as stated above. The stained cells were washed three times with the cell culture media with β-estradiol. After washing, live cell imaging solution (Thermo Fisher, A14291DJ) containing 0.1 mM Trolox (Vector Labs, CB1000-2) was added to the cells for imaging. Images were acquired with a DeltaVision inverted fluorescence microscope, with the exposure time set to 100 ms to minimize phototoxicity to the living cells across multiple imaging sessions. Quenching was performed with BHQ3-N-Tz (2) diluted to 500 nM in cell culture media with β-estradiol and incubated with cells for mins at RT. After quenching, cells were washed with cell culture media without β-estradiol for three times and placed back in the incubator for further culture. On Day 2, 4, and 6, the same procedure was conducted with two cycles of staining and quenching. The first cycle included staining with a cocktail of three SAFE conjugated antibodies (anti-CD45, anti-cKit, anti-F4/80) and the second cycle included staining with a cocktail of the remaining three SAFE conjugated antibodies (anti-Ly6C, anti-Ly6G, anti-CD11b). Each image captured a set of 12 microwells, and four imaging sites on the chip were captured in each profiling session (48 wells, total).

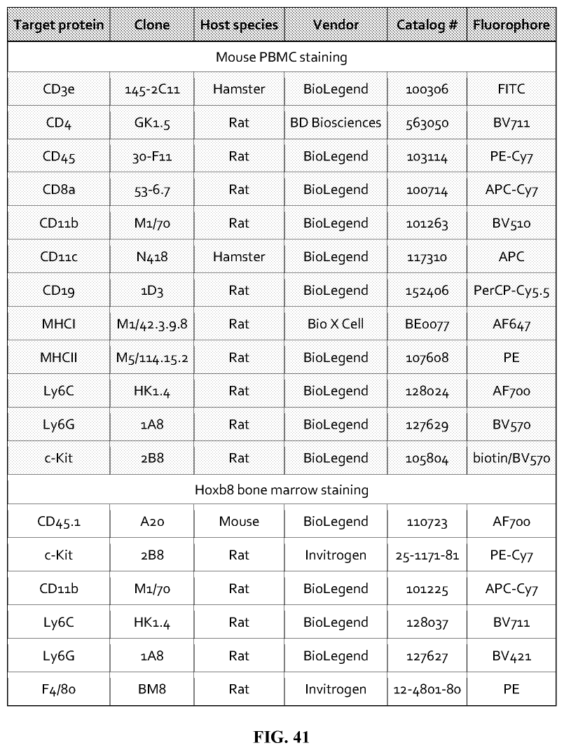

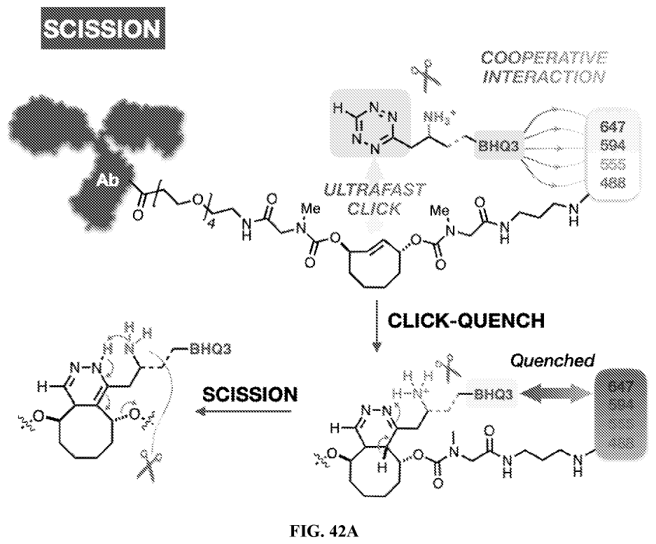

Neutrophil differentiation—flow cytometry control experiments. ER-Hoxb8 immortalized progenitors and SCF-dependent neutrophil progenitors were kept on ice for all steps if not stated otherwise. Cells were washed and resuspended in PBS, and stained with Zombie Aqua fixable viability dye (Biolegend 423101) to exclude dead cells. Cells were washed with PBS and incubated with FcBlock (clone 93, Biolegend) for 15 min at 4° C., followed by staining with fluorescently conjugated antibodies for min at 4° C. in staining buffer (0.5% BSA in PBS). The cells were washed with staining buffer and analyzed on a LSRII flow cytometer (BD). Following cell populations were identified based on cell marker expression: ER-Hoxb8 immortalized progenitors (CD45+CD11b− cKit/CD117+), neutrophils (CD45+CD11b+ cKit/CD117− Ly-6G+). Antibodies used here are shown in the FIG. 41.

Brief summary of the experimental findings: Cells in complex organisms undergo frequent functional changes as they differentiate and respond to their environment, but methods to achieve longitudinal readouts combined with deep profiling are limited. The present example describes a bioorthogonal toolkit for multiplexed temporospatial profiling of living cells, applying scission-accelerated fluorophore exchange (SAFE) to first quench and then completely and durably remove immunofluorescent signals from labeled cells. The results show that the chemistry is fast, achieving cycling in seconds at nanomolar concentrations, and accordingly nontoxic, enabling interrogation of cellular markers with high fidelity and minimal perturbation to underlying biological processes. The highly efficient scission machinery enables multiparameter (n≥14), nondisruptive characterization of murine peripheral blood mononuclear cells and bone marrow, allowing ongoing observation of characterized cells and serial profiling of cellular differentiation. The results demonstrate longitudinal multiplexed immunofluorescence imaging of bone marrow progenitor cells as they develop into neutrophils over 6 days, providing a proof of concept for an approach that finds broad utility for investigating physiologic dynamics in living systems.

Introduction: Temporal processes are often studied in living cells and organisms by introducing fluorescent proteins coupled to a promoter, protein, or biological compartment of interest, making otherwise invisible physiologic events observable by fluorescence microscopy. This strategy has been intensely successful in cell biology. However, the approach is not without shortcomings, including: i) the time required for creation and validation of cell lines; ii) the limited number of concurrent targets that can be visualized; and iii) the practical impossibility of using fluoroprotein labeling to analyze intact ensembles of primary cells, especially in complex contexts like development, differentiation, or immune function. Optimized monoclonal antibodies make immunofluorescence techniques a feasible alternative to study genetically-unmodified cells both in vitro and in vivo, but only for a handful (i.e., 1-4) of simultaneous targets. In aggregate, these liabilities sharply restrict the dimensionality of our view into living systems.

An array of analytical technologies, such as cellularly-resolved sequencing, multiplexed immunostaining, mass/imaging cytometry, and synergistic combinations thereof, can now achieve detailed snapshots of biomarker abundance, producing fine-grained but static profiles. These approaches have also been exceptionally fruitful. Nevertheless, the techniques are thus far incompatible with making measurements in live cells, due to inherently lethal physicochemical processing during the analytical workflow or dissociative single-cell detection, as in flow cytometry. Of the above tools, only the methods that rely on iterative immunostaining are potentially non-destructive, as the key step of antibody-antigen recognition is intrinsically biocompatible. The competing priorities, however, of fast/complete cycling (for efficient multiplexing with adequate signal to noise) and gentleness/non-toxicity (leaving cells both alive and unperturbed) require chemical conditions that have remained many orders of magnitude apart. As a result, highly multiplexed temporospatial profiling of living cells has been virtually impossible.

Lacking such methods, certain biological processes have remained challenging to investigate, for example: i) the role of intercellular communication during differentiation or immunotherapy, ii) functional exploration of novel cellular phenotypes, particularly in the space of immunology; iii) tracing of cellular lineages, e.g. in hematopoietic development. In each case, experiments can provide inferential evidence: patterns of ligand-receptor gene expression and spatial colocalization in fixed tissue samples allow deduction of likely cellular interactions; barcoding and functional readouts of knock-in/out experiments can identify cellular fate and physiologic roles. Methods that can directly observe these events offer the possibility of interrogating biological interactions, developmental transformations, and the downstream responses. Further development of such dynamic model systems has thus far been hindered by our inability to efficiently visualize both biomolecular complexity and (multi)cellular dynamics in real time.

The bioorthogonal chemistries of this disclosure achieve the capabilities needed for longitudinal multiplexed profiling in live cells (FIG. 38). This disclosure provides making and application of the scission-accelerated fluorophore exchange (SAFE) method, which leverages cooperative bioorthogonal mechanisms to first quench and then completely remove fluorescent signal from antibody-labeled cells. With a scaffold built around a cleavable C2-symmetric trans-cyclooctene (C2TCO) (e.g., any one the trans-cyclooctene molecules described herein), SAFE exploits noncovalent quencher-fluorophore interactions to accelerate already rapid tetrazine (Tz)-TCO click reactions, reaching >99% scission in seconds. Importantly, this makes bioorthogonal cycling feasible at nontoxic nanomolar concentrations which was not possible with prior methods, all while achieving the high signal to background needed for serial profiling.

As an initial matter, the results provide quantification of the accelerated chemical kinetics in analytical fluorescence assays and then verification the scission performance in the biological context by time-resolved imaging. Next, the accuracy and nontoxicity of 14 color imaging of living mouse peripheral blood mononuclear cells (PBMCs) were imaged, myeloid subsets in multicolor staining of living murine bone marrow were visualized. Finally, the results show the ability of serial bioorthogonal cycling to track the differentiation of immortalized hematopoietic progenitor cells into neutrophils in longitudinal profiling of the same cells over 6 days.

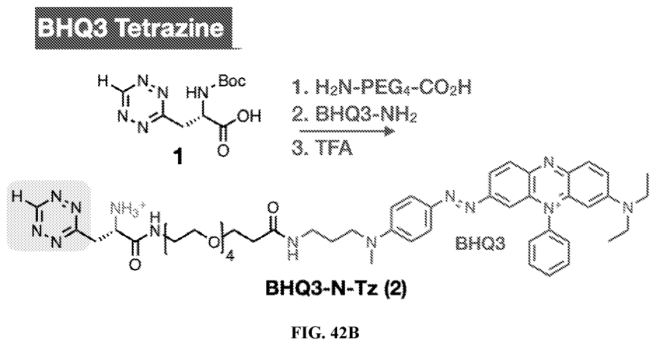

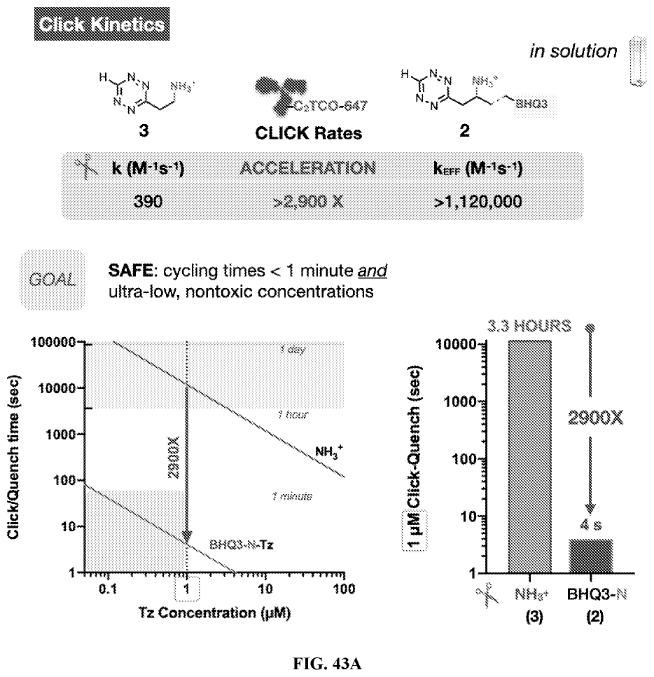

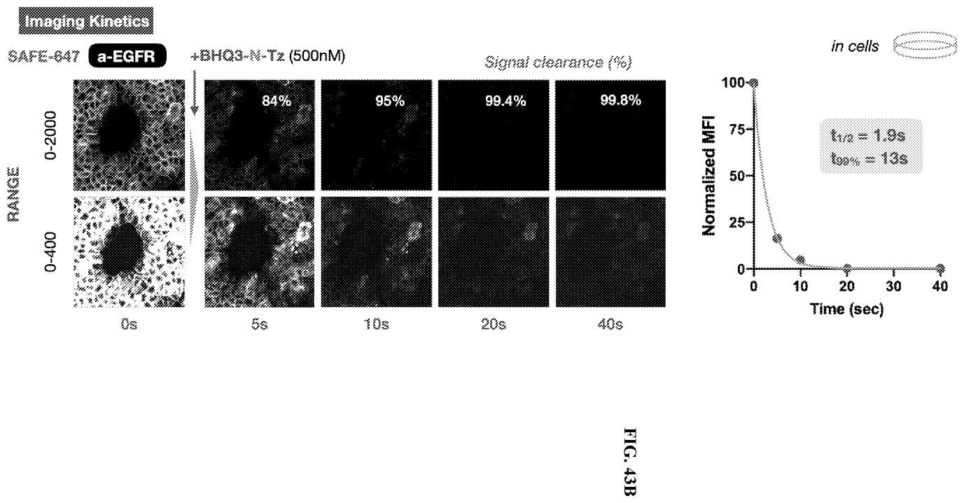

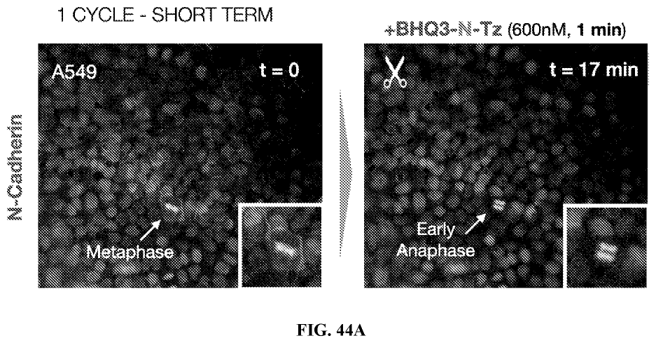

Experimental results: bioorthogonal cooperativity accelerates scission. For all but the most reactive molecules, chemical processes that reach completion in the timeframe of seconds to minutes require concentrations in the high micromolar to millimolar range. While this is no obstacle for in vitro organic synthesis, it poses challenges for bioorthogonal methods: i) these same concentration ranges correspond to the level at which many organic compounds become nonspecifically cytotoxic, barring use in living cells; ii) reagents with this highest degree of reactivity are typically metastable and/or vulnerable to rapid degradation, limiting their performance in the biological environment. To circumvent these issues, the design of SAFE exploits a cooperative interaction between the fluorescence quencher BHQ3 and Alexa Fluor dyes for rapid on/off scission of fluorophore-C₂TCO-labeled antibodies (FIG. 42A) at low, nontoxic reagent concentrations. Importantly, the BHQ3-dye interaction is expected to accelerate the Tz-TCO click reaction; in this initial stage, ligation yields immediate quenching (>95%) of the dye fluorescence. In turn, quantitative scission is driven by a functionalized tetrazine equipped with an intramolecular proton donor (NH₃⁺ at physiologic pH) that serves to accelerate tautomerization, functioning as a ‘Tz-scissors’. This click-to-cut scission reaction then releases the quenched fluorophores from antibodies, removing residual signal: i) optimizing signal to background, and ii) eliminating the downstream risk of rebounding signal from the quenched dye.

N-Boc-protected tetrazine (1) was synthesized and extended with a PEG4 amino acid linker, followed by coupling with BHQ3-amine and deprotection (FIG. 42B). The reaction of BHQ3-N-Tz (2) and an AF647-C₂TCO (SAFE647) labeled antibody was followed by monitoring fluorescence intensity vs time. At an optimized concentration of 2, the reaction process could be kinetically segregated into its two component phases, resolving the quenching of the click ligation from the subsequent scission reaction. Nonlinear fitting of the biphasic fluorescence time course yielded a rate constant of 0.133±0.005 s⁻¹ (t_{1/2}=5.2 sec) for the scission of the C₂TCO linker. For the click ligation itself, an effective second order rate constant ≥1.12×10⁶ M⁻¹s⁻¹ was measured, more than 2900-fold faster than the reaction of the parent Tz (3) with C₂TCO (FIG. 43A). C₂TCO itself is stable (>97% intact at t=48h in full cell growth media) and not very reactive until accelerated by the ‘cooperative’ SAFE effect resulting in stable click reagents, but also very fast kinetics. The cooperative acceleration shifts the kinetic regime from hours to seconds: a benchmark concentration of 1 μM is predicted to reach complete (>99%) reaction in 4 seconds, while nanomolar concentrations are sufficient for complete click-quench reactions in less than a minute, matching the design objectives (FIG. 43A). To validate the solution kinetics in a biological context, fixed A431 cells were stained with a SAFE647-labeled anti-EGFR antibody and collected serial images before and after the addition of BHQ3-N-Tz (500 nM, FIG. 43B). The quenching-cleavage reaction on the surface of the cells was both rapid (t_{1/2}=1.9 s) and complete, with a 99.8% reduction in signal intensity after 40 seconds, (˜8 scission half-lives). These kinetics integrate both steps of signal removal at the extracellular surface, confirming the magnitude of the acceleration and the high dynamic range of signal elimination, critical to preventing background accumulation across multicycle staining.

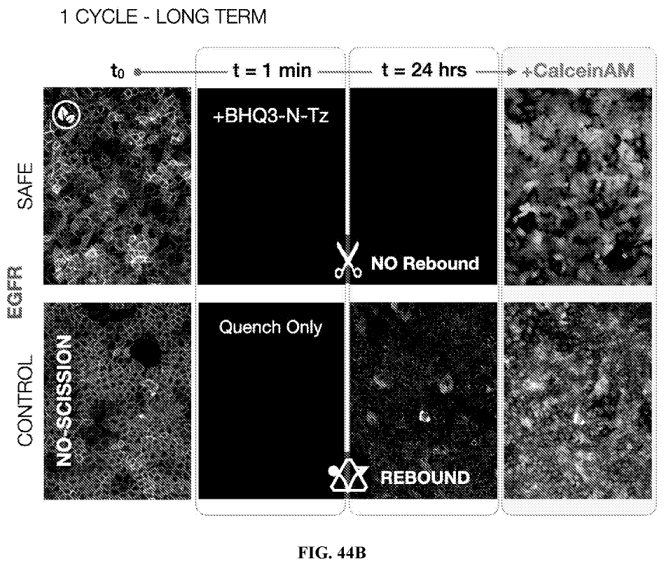

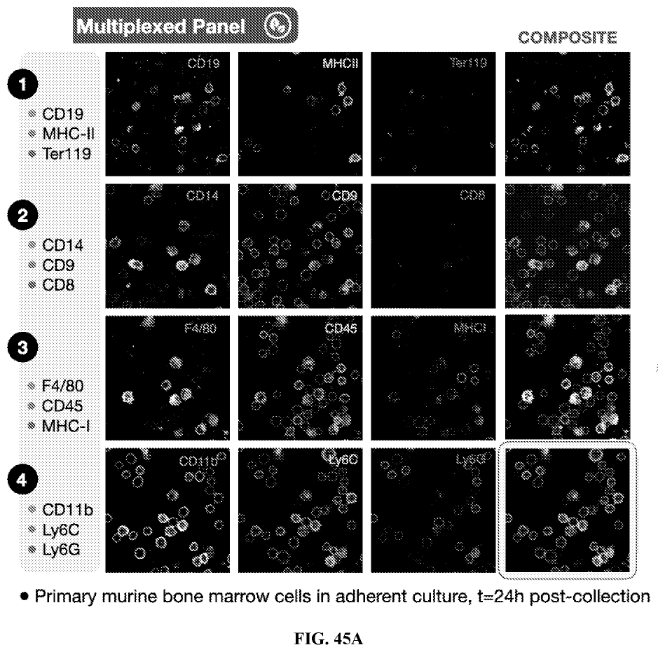

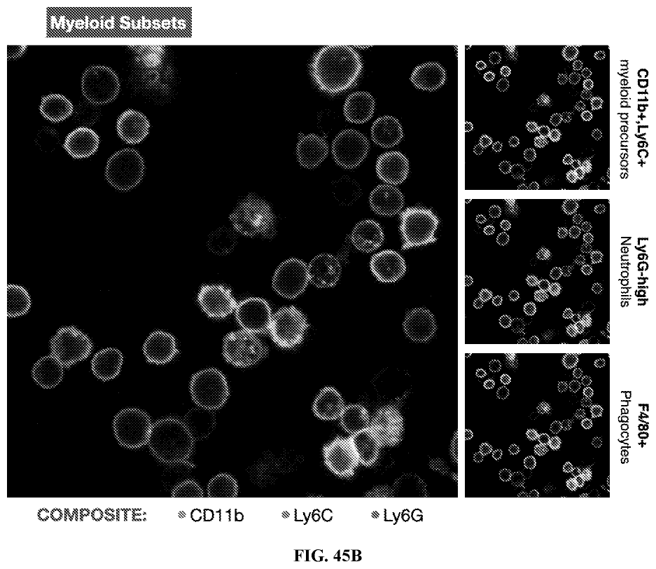

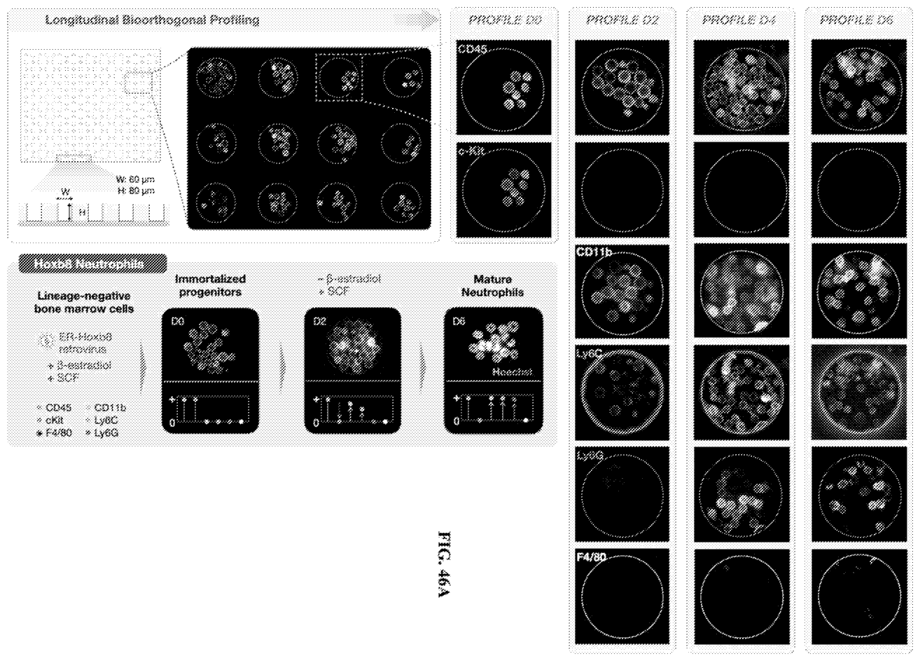

SAFE allows cycling in live cells. An initial short-term cycling experiment was performed on live A549 cells stained with Hoechst and a SAFE647-conjugated anti-N-Cadherin antibody. The N-Cadherin signal was efficiently eliminated with BHQ3-N-Tz (1 μM, 1 minute, FIG. 44A). A fraction of cells visually confirmed their live status by undergoing mitosis during the imaging process: in the example shown (FIG. 44A, inset) a cell is captured in metaphase before quenching and in early anaphase after quenching, 17 minutes of elapsed time later. For a longer-term assessment, A549 experiments were expanded into serial imaging to test scission performance and screen for cytoxicity. Cells were stained with a SAFE647-conjugated anti-EGFR antibody, quenched/cleaved with BHQ3-N-Tz, and then returned to routine culture conditions at 37° C., with images of the same cellular area collected at each stage. After 24 hours, the cells were re-imaged with live and dead cell staining dyes (live: calcein AM, dead: sytox red) to determine their viability (FIG. 44B, SAFE).

Consistent with complete scission of the quenched fluorophores, cells remained non-fluorescent in the 647 channel on day 2, with no signal rebound observed. The cells were calcein AM-bright and had increased in density, consistent with unhindered interim proliferation. As a control, a conventional TCO-Tz pair was used that can click for bioorthogonal quenching but cannot cleave fluorophores from the antibody. Although the cells were again calcein AM-positive and proliferating, a spontaneous increase in fluorescence signal was observed in the absence of scission (FIG. 44B, CONTROL). Black hole quenchers are known to be vulnerable to azo bond cleavage, particularly in vivo; the observed rebound is consistent with slow intracellular and/or extracellular degradation of the BHQ3, leading to loss of quenching and the reappearing signal. While both bioorthogonal methods achieve efficient short-term cycling in living cells, scission is important for serial profiling in longer timeframes where antibody internalization is expected and fluorescence rebound is a hazard.

SAFE live cell cycling was then extended to multiple cycles of staining beyond cancer cell lines, to validate nontoxicity in mixed populations of primary immune cells. To image living mouse peripheral blood mononuclear cells (PBMCs) in suspension, PDMS microwells were fabricated (80 μm diameter, 80 μm depth) using soft lithography. Both short-term imaging experiments and subsequent cell culture experiments (vide infra) were performed with these microwells, which showed excellent retention of the suspended cells, necessary for marker quantitation across cycles. Freshly harvested mouse PBMCs were isolated, pooled, and then loaded into microwells for imaging, reserving aliquots for parallel analysis by flow cytometry. 12 targets were visualized across 4 cycles of imaging and quenching in live cells over the course of <2 hours of elapsed time. At the end, the cell viability was determined (711 alive/737 total cells in 72 microwells, 96.5% alive) in a fifth round of imaging with calcein AM/sytox red (FIG. 39). The cells expressing each marker were counted and compared to the aliquot analyzed synchronously by flow cytometry for the same target panel. There was an excellent correlation (R²=0.985) between populations measured by SAFE (N=737) and flow cytometry (N>10⁵) in both their marker abundance and in the measured cell viability (96.2% vs 96.5%, FIG. 39), confirming i) the staining accuracy across multiple cycles of imaging and scission-mediated fluorophore exchange, ii) the ability to accurately quantify cellular abundance in small/sparse samples, and iii) the lack of toxicity to living PBMCs.

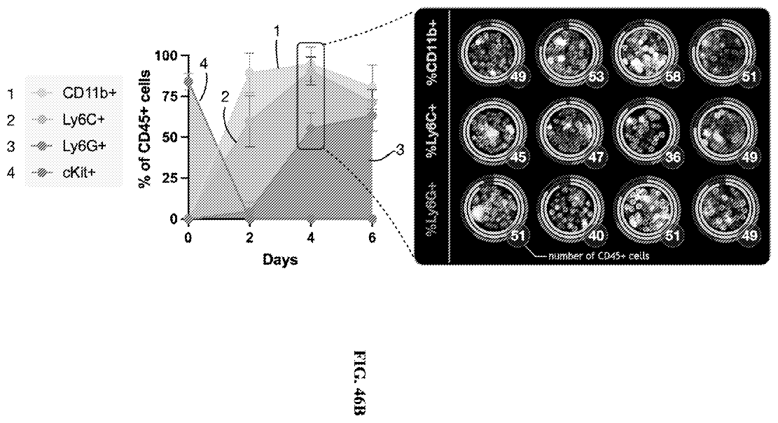

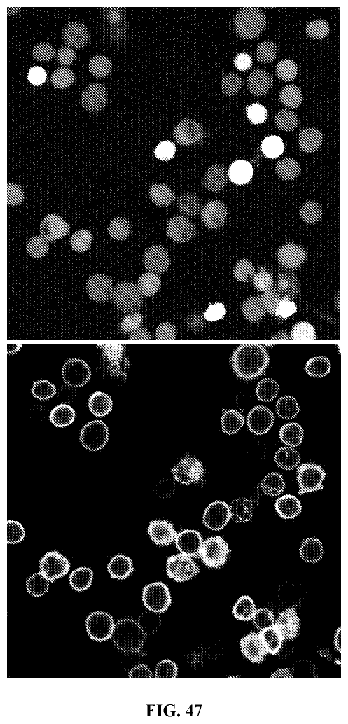

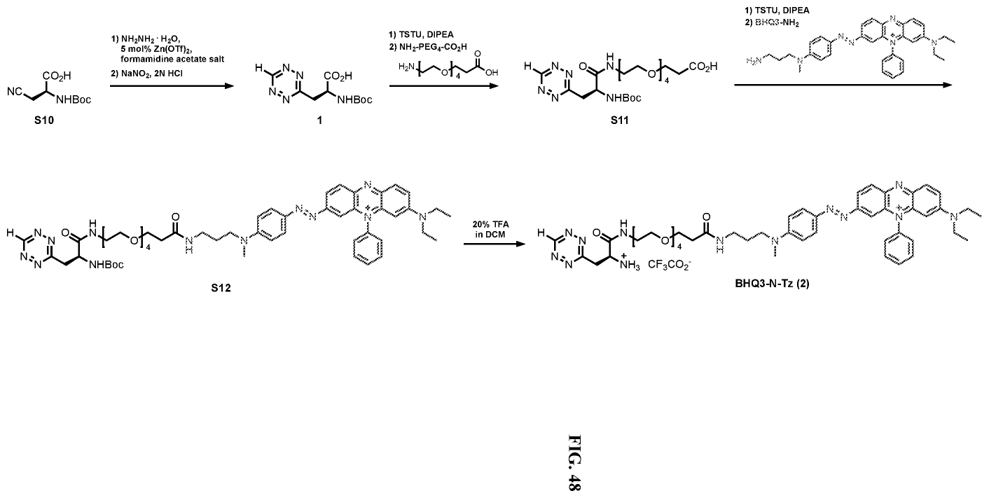

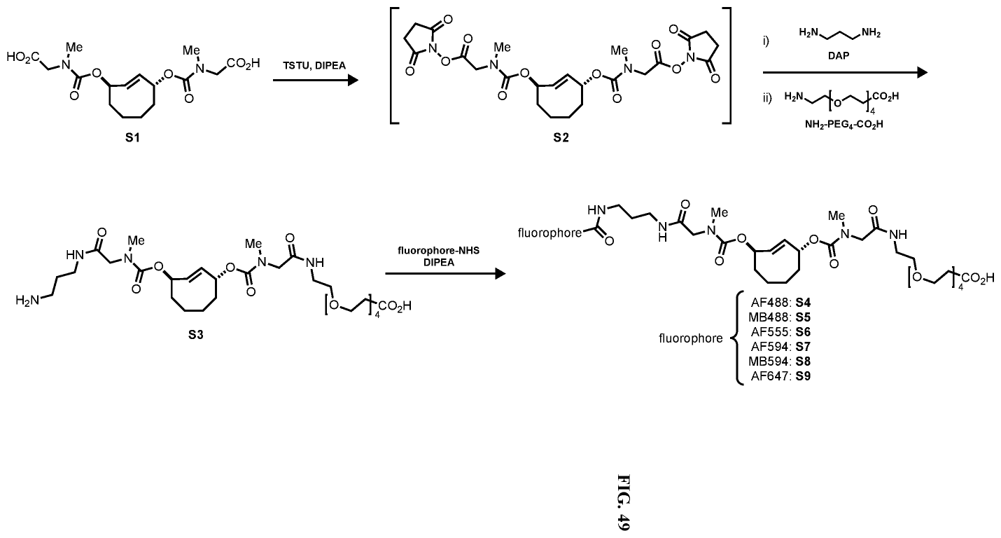

Profiling the differentiation of living bone marrow at high spatial resolution. Hematopoietic development generates phenotypic diversity as multipotent stem/progenitor cells differentiate into their repertoire of mature progeny. High-dimensional multiplexed immunohistochemical and flow cytometric profiling has mapped developmental hierarchies; likewise, single-cell gene expression analysis and barcoding technologies have traced hematopoietic lineages. Directly visualizing the bone marrow cells and their ensemble of surface markers amid these changes, however, has not been possible. As an initial test of the feasibility of SAFE profiling of living bone marrow, freshly harvested murine bone marrow cells were counted and put into culture overnight. 12 immune markers were profiled (3 markers/cycle, 4 cycles total), with a focus on myeloid precursors (FIG. 45A), using confocal imaging to distinguish membrane/cytoplasmic staining. Viability was confirmed by Calcein-AM staining at the end of the profiling sequence.

CD8 positive cells were rare, indicating that non-adherent T-cells were not efficiently retained in this staining format. Images demonstrated the presence of erythroid (Ter119+), lymphoid (CD19+), and myeloid (CD11b+) subsets within the population, including cells across a spectrum of functional differentiation. A continuum of CD11b, Ly6C, and Ly6G expression was evident in the high resolution composite image (FIG. 45B), with myeloid precursors (CD11b+, Ly6C+) that render as blue-cyan, and mature Ly6G+ neutrophils that appear magenta/red. A subset of phagocytically-proficient cells (CD14+ and F4/80+ in cycles 2 and 3, respectively) exhibited a pattern of multi-color intracellular staining consistent with antibody internalization prior to scission, in addition to the ON/OFF cycling of specific cell-surface markers. Apart from this subpopulation, negligible cytoplasmic signal was observed after four complete cycles, indicating the feasibility of accurate, iterative staining and scission in this cellular context.

To implement longitudinal bioorthogonal profiling on longer time scales, i.e. across not just multiple cycles but multiple days, a model system was selected with well-defined cellular dynamics. Estrogen-regulated Hoxb8 (ER-Hoxb8) hematopoietic progenitor cells are conditionally immortalized by retroviral infection of myeloid bone marrow progenitors. Removal of ß-estradiol from culture media triggers the ER-Hoxb8 differentiation program and cells become mature neutrophils by day 4-6, a developmental sequence that has been well-characterized by flow cytometry and gene expression. Loss of cKit expression and sequential upregulation of CD11b, Ly6C, and Ly6G occur in tandem with population expansion.

A sparse density of ER-Hoxb8 cells were loaded into PDMS microwells (W: 60 μm, H: 80 μm, FIG. 46A), fabricated as in the PBMC studies (vide supra). The same cells were imaged longitudinally during differentiation (DO, D2, D4, D6) in two profiling cycles at each time point, staining for CD45 and F4/80 as positive/negative controls, respectively, in addition to the quartet of differentiation markers. At DO, all cells were CD45+/cKit+ and negative for the remaining panel, as expected. Rapid population growth by D2 tracked with broad upregulation of CD11b and a subset of Ly6C positive cells. The density of viable cells peaked on D4, concomitantly with the appearance of bright Ly6G staining. Given the short lifespan of differentiated granulocytes, population density had decreased slightly by D6, consistent with the fraction of dead cells normally observed by flow cytometry. F4/80 staining remained negative throughout, as expected for differentiation under these neutrophil-selective culture conditions. Cell growth during temporospatial profiling was robust, allowing quantitative analysis of both proliferation and differentiation across the individual microwells (FIG. 46B).

Discussion: The bioorthogonal framework of SAFE creates a new method for multiplexed longitudinal profiling of live cells, applied here to demonstrate 14 color imaging in living primary hematopoietic and immune populations, across a timescale spanning minutes to days. Methods that incorporate multiple cycles of fluorescent staining and quenching were originally developed for paraffin-embedded tissue sections that can withstand harsh destaining conditions. A gentler DNA-barcoded antibody technologies were developed for cellular profiling, but these too require long destaining times and solvent/buffer conditions incompatible with live cell analysis. The recent method for immunostaining with Tz/TCO-driven bioorthogonal quenching achieves its speed by circumventing the need to remove fluorophores. While capable of rapid temporal cycling at nontoxic reagent concentrations, the quenching is reversed by biochemical degradation over longer timeframes in living systems, leading to signal rebound (FIG. 44B). In contradistinction, the SAFE method bypasses the above shortcomings and allows extremely fast cycling in the seconds time frame (>99% signal clearance in <30 sec, FIG. 43B), while using nanomolar concentrations of reagents for gentle and irreversible fluorophore scission. To the best of our knowledge, SAFE is currently the only technique that allows such temporospatial profiling in living cells.

Bioorthogonal reactions have long been described in live cells as an alternative route to biomolecular tagging, with incorporation of functionalized noncanonical amino acids into proteins, modified nucleosides into DNA/RNA, metabolic building blocks into glycans, or clickable tags into chemical probes, followed by subsequent click-mediated labeling. The impetus for developing these strategies has primarily been visualization of the different classes of biomolecules in living systems, exploiting the small footprint of compact chemical tags to follow biological trafficking or create a readout for replication/metabolism. Such tags have also featured in strategies for superresolution microscopy. In this disclosure, chemical tools are applied not to deliver labels but rather to remove them via bioorthogonal bond cleavage, pursuing highly multiplexed temporal analysis of cell-surface protein expression. A new generation of cleavable linkers was utilized based on a new scaffold (C₂TCO) and optimized acid-functionalized tetrazine-scissors—the first bioorthogonal reagents capable of achieving both complete (>99.5%) and rapid (seconds) scission. Simultaneously, the superb stability of C₂TCO (>97% at 48 hrs) is critical to maintaining ultra-efficient cleavage over time. By accelerating the scission sequence with the cooperative BHQ3-fluorophore-driven click kinetics (FIG. 43A), the SAFE reagents achieve a unique combination of intrinsic biochemical stability and high reactivity that arises as an emergent property of the system.

The proof-of principle experiments shown here could be further expanded to increase the number of cycles with each profile and/or to broaden the number of proteins that are simultaneously detectable. One of the key advantages of the SAFE method is that the underlying bioorthogonal scission reaction is compatible with any conjugatable dye, including polymeric fluorochromes (e.g., BV dye series) that are otherwise difficult to quench. Since a range of bright polymer dyes can be excited in the violet/UV, their use together with organic rhodamine/cyanine dyes could enable broader multiplexing to ˜8 channels per cycle with minimal spectral overlap. Over 10 cycles (feasible with observed scission efficiencies >99.5%), this would allow analysis of up to 80 protein biomarkers in live cells. Finally, while the focus of the present disclosure was on temporospatial analysis of living cells in culture, the above approaches are entirely compatible with fixed cell and tissue analyses, expanding the analytical capabilities of clinical sample testing. This offers the potential for deeper immune cell profiling, single cell pathway analysis, and drug response testing in biopsy samples, and opens the door to multiplexed functional profiling of living cells and tissues in vivo or following explanation.

### REFERENCES

1. R. M. Versteegen, R. Rossin, W. Hoeve, H. M. Janssen, M. S. Robillard, Angewandte Chemie International Edition 2013, 52, 14112-14116.
2. J. Ko, J. Oh, M. S. Ahmed, J. C. T. Carlson, R. Weissleder, Angewandte Chemie International Edition 2020, 59, 6839-6846.
3. J. C. Carlson, H. Mikula, R. Weissleder, Journal of the American Chemical Society 2018, 140, 3603-3612.
4. X. Fan, Y. Ge, F. Lin, Y. Yang, G. Zhang, W. S. C. Ngai, Z. Lin, S. Zheng, J. Wang, J. Zhao, J. Li, P. R. Chen, Angewandte Chemie International Edition 2016, 14046-14050.
5. A. J. C. Sarris, T. Hansen, M. A. R. de Geus, E. Maurits, W. Doelman, H. S. Overkleeft, J. D. C. Codee, D. V Filippov, S. I. van Kasteren, Chemistry 2018, 24, 18075-18081.
6. M. R. Karver, R. Weissleder, S. A. Hilderbrand, Bioconjugate Chemistry 2011, 22, 2263-2270.
7. A. Darko, S. J. Boyd, J. M. Fox, Synthesis 2018, 50, 4875-4882.
8. M. T. Taylor, M. L. Blackman, O. Dmitrenko, J. M. Fox, Journal of the American Chemical Society 2011, 133, 9646-9649.
9. M. K. Leong, V. S. Mastryukov, J. E. Boggs, Journal of Molecular Structure 1998, 445, 149-160.
10. N. L. Allinger, J. T. Sprague, Journal of the American Chemical Society 1972, 94, 5734-5747.
11. R. Rossin, S. M. J. van Duijnhoven, W. ten Hoeve, H. M. Janssen, L. H. J. Kleijn, F. J. M. Hoeben, R. M. Versteegen, M. S. Robillard, Bioconjugate Chemistry 2016, 27, 1697-1706.
12. J. Li, S. Jia, P. R. Chen, Nat Chem Biol 2014, 10, 1003-1005.
13. E. Kim, K. S. Yang, R. H. Kohler, J. M. Dubach, H. Mikula, R. Weissleder, Bioconjugate Chemistry 2015, 26, 1513-1518.
14. A. Turetsky, E. Kim, R. H. Kohler, M. A. Miller, R. Weissleder, Scientific Reports 2014, 4, 4782.
15. Fredenburgh J C, Gross P L, Weitz J I. Emerging anticoagulant strategies. Blood. 2017; 129(2) 147-154.
16. Vedantham S, Goldhaber S Z, Julian J A et al. Pharmacomechanical Catheter-Directed Thrombolysis for Deep-Vein Thrombosis. N Engl J Med. 2017; 377(23) 2240-2252.
17. Li J, Chen P R. Development and application of bond cleavage reactions in bioorthogonal chemistry. Nat Chem Biol. 2016; 12(3) 129-137.
18. Zavorsky G S, Walley K R, Russell J A. Red cell pulmonary transit times through the healthy human lung. Exp Physiol. 2003; 88(2) 191-200.
19. Magder S. Volume and its relationship to cardiac output and venous return. Crit Care. 2016; 20 271.
20. Maraganore J M, Bourdon P, Jablonski J, Ramachandran K L, Fenton J W. Design and characterization of hirulogs: a novel class of bivalent peptide inhibitors of thrombin. Biochemistry. 1990; 29(30) 7095-7101.
21. Scarborough R M, Naughton M A, Teng W et al. Design of potent and specific integrin antagonists. Peptide antagonists with high specificity for glycoprotein IIb-IIIa. J Biol Chem. 1993; 268(2) 1066-1073.
22. Thompson R E, Liu X, Ripoll-Rozada J et al. Tyrosine sulfation modulates activity of tick-derived thrombin inhibitors. Nature chemistry. 2017; 9 909.
23. Koh C Y, Kumar S, Kazimirova M et al. Crystal structure of thrombin in complex with S-variegin: insights of a novel mechanism of inhibition and design of tunable thrombin inhibitors. PloS one. 2011; 6(10) e26367.
24. Shammas N W. Bivalirudin: pharmacology and clinical applications. Cardiovasc Drug Rev. 2005; 23(4) 345-360.
25. Carlson J C, Meimetis L G, Hilderbrand S A, Weissleder R. BODIPY-tetrazine derivatives as superbright bioorthogonal turn-on probes. Angew Chem Int Ed Engl. 2013; 52(27) 6917-6920. doi:10.1002/anie.201301100. PubMed PMID: 23712730. PMCID: PMC3875324.
26. Meimetis L G, Carlson J C, Giedt R J, Kohler R H, Weissleder R. Ultrafluorogenic coumarin-tetrazine probes for real-time biological imaging. Angew Chem Int Ed Engl. 2014; 53(29) 7531-7534. doi:10.1002/anie.201403890. PubMed PMID: 24915832. PMCID: PMC4122131.
27. Carlson J C, Jena S S, Flenniken M, Chou T F, Siegel R A, Wagner C R. Chemically controlled self-assembly of protein nanorings. J Am Chem Soc. 2006; 128(23) 7630-7638. doi:10.1021/ja060631e. PubMed PMID: 16756320.
28. Meimetis L G, Boros E, Carlson J C, Ran C, Caravan P, Weissleder R. Bioorthogonal Fluorophore Linked DFO-Technology Enabling Facile Chelator Quantification and Multimodal Imaging of Antibodies. Bioconjug Chem. 2016; 27(1) 257-263. doi:10.1021/acs.bioconjchem.5b00630. PubMed PMID: 26684717. PMCID: PMC4858350.
29. Love J E, Ferrell C, Chandler W L. Monitoring direct thrombin inhibitors with a plasma diluted thrombin time. Thromb Haemost. 2007; 98(1) 234-242.
30. André P, Larocca T, Delaney S M et al. Anticoagulants (thrombin inhibitors) and aspirin synergize with P2Y12 receptor antagonism in thrombosis. Circulation. 2003; 108(21) 2697-2703.
31. Iversen N K, Malte H, Baatrup E, Wang T. The normal acid-base status of mice. Respir Physiol Neurobiol. 2012; 180(2-3) 252-257.
32. Nikić I, Girona G E, Kang J H et al. Debugging Eukaryotic Genetic Code Expansion for Site-Specific Click-PAINT Super-Resolution Microscopy. Angewandte Chemie International Edition. 2016; 55(52) 16172-16176.
33. Hervio L S, Coombs G S, Bergstrom R C, Trivedi K, Corey D R, Madison E L. Negative selectivity and the evolution of protease cascades: the specificity of plasmin for peptide and protein substrates. Chem Biol. 2000; 7(6) 443-453.
34. Serfling R, Lorenz C, Etzel M et al. Designer tRNAs for efficient incorporation of non-canonical amino acids by the pyrrolysine system in mammalian cells. Nucleic acids research. 2017; 46(1) 1-10.
35. Kozma E, Nikić I, Varga B R et al. Hydrophilic trans-Cyclooctenylated Noncanonical Amino Acids for Fast Intracellular Protein Labeling. ChemBioChem. 2016; 17(16) 1518-1524.
36. O'shea J C, Tcheng J E. Eptifibatide: a potent inhibitor of the platelet receptor integrin glycoprotein IIb/IIIa. Expert Opin Pharmacother. 2002; 3(8) 1199-1210.
37. Agasti et al., 2012, J Am Chem Soc, 134, 18499-502
38. Alon et al., 2021, Science, 371, eaax2656
39. Angelo et al., 2014, Nat Med, 20, 436-42
40. Arlauckas et al., 2017, Sci Transl Med, 9; Zindel et al., 2021, Science, 371, eabe0595
41. Armingol et al., 2021, Nat Rev Genet, 22, 71-88
42. Bechtel et al., 2021, Nat Chem Biol, 17, 641-652
43. Beliu et al., 2019, Commun Biol, 2, 261
44. Bendall et al., 2011, Science, 332, 687-96
45. Calieque et al., 2018, Nature Reviews Chemistry, 2, 202-215
46. Carlson et al., 2018, J Am Chem Soc, 140, 3603-3612
47. Cho et al., 2021, Cell, 184, 3559-3572.e22
48. Costantini et al., 2015, Nat Commun, 6, 7670
49. de la Torre and Chin, 2021, Nat Rev Genet, 22, 169-184
50. Eng et al., 2019, Nature, 568, 235-239
51. Engblom et al., 2017, Science, 358, eaa15081
52. Gerdes et al., 2013, Proc Natl Acad Sci USA, 110, 11982-7
53. Giedt et al., 2018, Nat Commun, 9, 4550
54. Guo et al., 2019, Nat Commun, 10, 4377
55. Hartmann and Bendall, 2020, Nat Rev Rheumatol, 16, 87-99
56. Hidalgo et al., 2019, Trends Immunol, 40, 584-597
57. Jackson et al., 2020, Nature, 578, 615-620
58. Jenkins et al., 2018, Cancer Discov, 8, 196-215
59. Katzenelenbogen et al., 2020, Cell, 182, 872-885.e19
60. Ko et al., 2020, Angew Chem Int Ed Engl, 59, 6839-6846
61. Lang and Chin, 2014, Chem Rev, 114, 4764-806
62. Lin et al., 2015, Nat Commun, 6, 8390
63. Linder et al., 2011, Bioconjug Chem, 22, 1287-97
64. Loughran et al., 2020, Exp Hematol, 88, 1-6
65. Mandessian et al., 2021, Nature, 590, 649-654
66. McKinnon, 2018, Curr Protoc Immunol, 120, 5.1.1-5.1.11
67. Nguyen and Prescher, 2020, Nat Rev Chem, 4, 476-489
68. Nikić et al., 2014, Angew Chem Int Ed Engl, 53, 2245-9
69. Oh et al., 2021, Clin Cancer Res, clincanres.1252.2021
70. Palaniappan and Bertozzi, 2016, Chem Rev, 116, 14277-14306
71. Pascual-Reguant et al., 2021, Nat Commun, 12, 1737
72. Patriarchi et al., 2020, Nat Methods, 17, 1147-1155
73. Pfirschke et al., 2020, Cell Rep, 32, 108164
74. Polonsky et al., 2018, Science, 360, eaaj1853
75. Prescher and Bertozzi, 2005, Nat Chem Biol, 1, 13-21
76. Pucella et al., 2020, Annu Rev Cell Dev Biol, 36, 529-550
77. Qiu et al., 2011, Nat Biotechnol, 29, 886-91
78. Radtke et al., 2020, Proc Natl Acad Sci USA, 117, 33455-33465
79. Rodriguez et al., 2017, Trends Biochem Sci, 42, 111-129
80. Saka et al., 2019, Nat Biotechnol, 37, 1080-1090
81. Salic and Mitchison, 2008, Proc Natl Acad Sci USA, 105, 2415-20
82. Sarris et al., 2018, Chemistry, 24, 18075-18081
83. Schubert et al. Nat Biotechnol 24:1270
84. Stickels et al., 2021, Nat Biotechnol, 39, 313-319
85. Stoeckius et al., 2017, Nat Methods, 14, 865-868
86. Sullivan et al., 2021, Science, 371, eaba8310
87. Sykes et al., 2016, Cell, 167, 171-186.e15
88. Ullal et al., 2014, Sci Transl Med, 6, 219ra9
89. Versteegen et al., 2013, Angew Chem Int Ed Engl, 52, 14112-6
90. Voabil et al., 2021, Nature Medicine, 1-12
91. Wang et al., 2006, Nat Methods, 3, 287-93
92. Wang et al., 2021, ACS Cent Sci, 7, 929-943
93. Werther et al., 2020, Angew Chem Int Ed Engl, 59, 804-810
94. Wilkovitsch et al., 2020, J Am Chem Soc, 142, 19132-19141
95. Yang et al., 2013, Angew Chem Int Ed Engl, 52, 10593-7
96. Zaretsky et al., 2012, Lab on a chip, 12, 5007-5015
97. Mercier et al., 2016, Stem Cell Reports 6, 985-992
98. Zindel, J. et al., 2021, Science 371, eabe0595

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A compound of Formula (I): 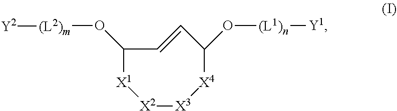
or a pharmaceutically acceptable salt thereof, wherein:
X¹ is selected from O and CHR¹;
X² is selected from O and CHR²;
X³ is selected from O and CHR³;
X⁴ is selected from O and CHR⁴;
R¹, R², R³, and R⁴ are each independently selected from H, halo, C₁₋₆ alkyl, C₁₋₆ haloalkyl, C₁₋₃ alkoxy, and C₁₋₃ haloalkoxy;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000;
each R^{N} is independently selected from H, C₁₋₃ alkyl, and C₁₋₃ haloalkyl; and
each Y¹ and Y² are independently selected from H, NH₂, OH, C(O)OH, a protected amino group, a protected carboxyl group, a protected hydroxyl group, a reactive chemical group, a fluorophore, and a fluorescence quencher.

2. The compound of claim 1, having a formula: 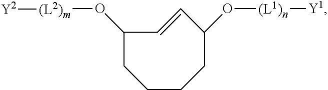
or a pharmaceutically acceptable salt thereof.

3. The compound of claim 2, having a formula: 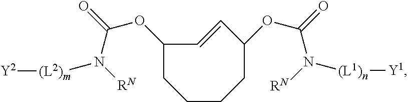
or a pharmaceutically acceptable salt thereof.

4. The compound of claim 1, having any one of the following formulae: 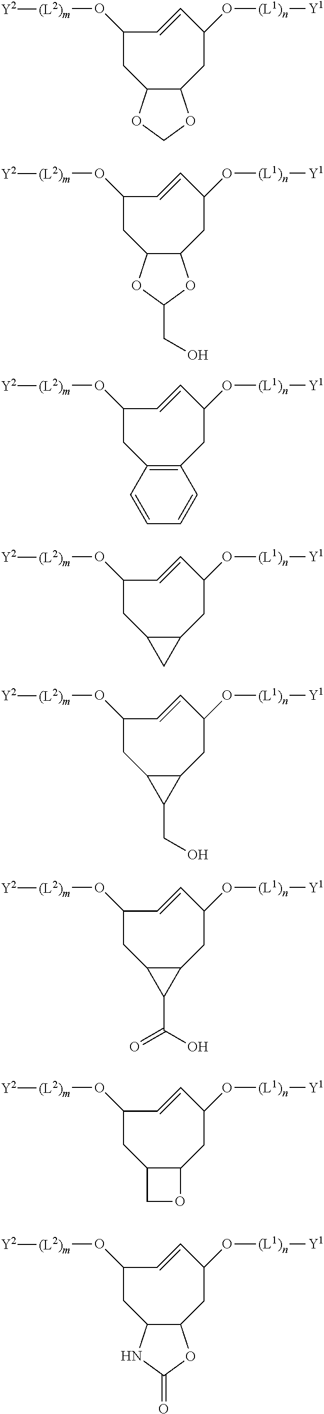
or a pharmaceutically acceptable salt thereof.

5. The compound of any one of claims 1-4, wherein:
n is an integer from 1 to 10;
m is an integer from 1 to 10;
Y¹ is selected from NH₂, OH, C(O)OH, a protected amino group, a protected carboxyl group, and a protected hydroxyl group; and
Y² is selected from NH₂, OH, C(O)OH, a protected amino group, a protected carboxyl group, and a protected hydroxyl group.

6. The compound of any one of claims 1-4, wherein:
n is an integer from 1 to 10;
m is an integer from 1 to 10;
Y¹ is selected from NH₂, OH, C(O)OH, a protected amino group, a protected carboxyl group, and a protected hydroxyl group; and
Y² is a reactive chemical group.

7. The compound of any one of claims 1-4, wherein:
n is an integer from 1 to 10;
m is an integer from 1 to 10;
Y¹ is a fluorophore; and
Y² is a reactive chemical group.

8. The compound of claim 1, wherein the compound is selected from any one of the following compounds: 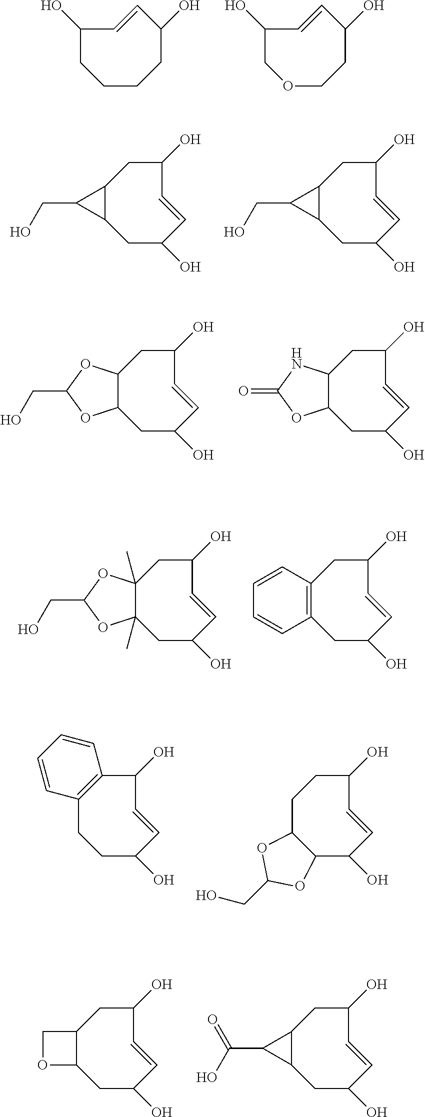
or a pharmaceutically acceptable salt thereof.

9. The compound of claim 1, selected from any one of the following compounds: 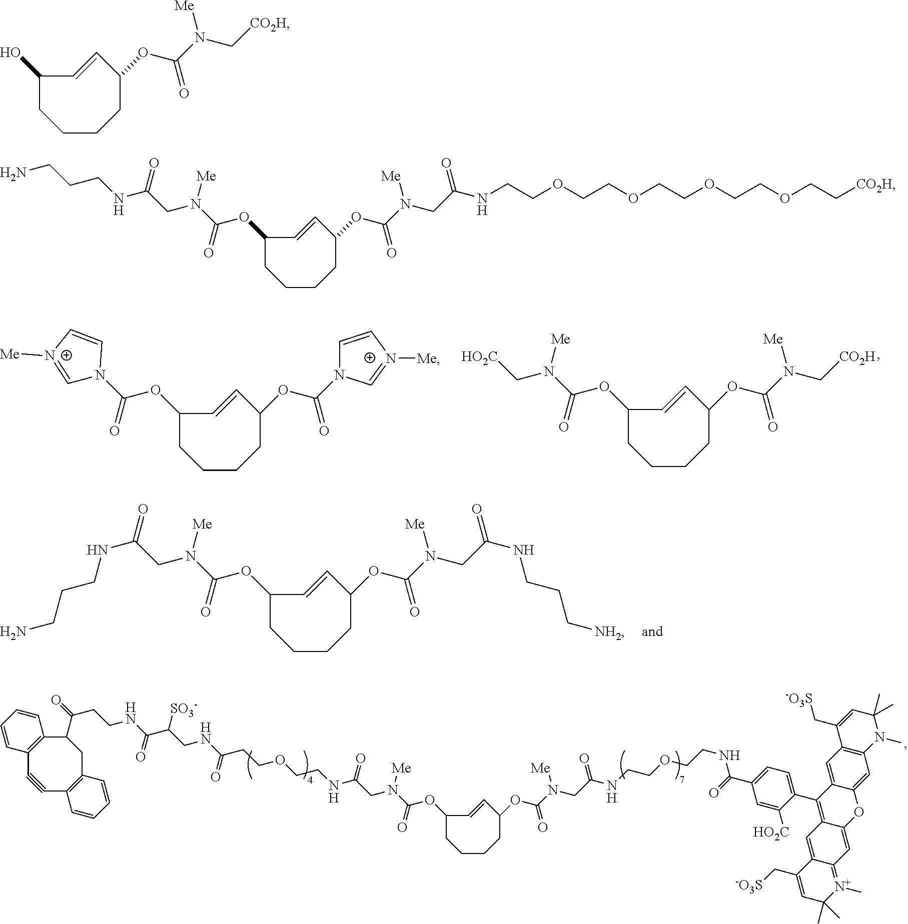
or a pharmaceutically acceptable salt thereof.

10. The compound of claim 1, wherein the compound is selected from any one of the compounds depicted in FIGS. 37A-37D, or a pharmaceutically acceptable salt thereof.

11. A compound of Formula (II): 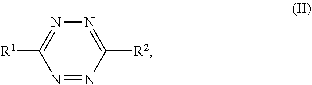
or a pharmaceutically acceptable salt thereof, wherein:
R¹ and R² are each independently selected from H, C₁₋₆ alkyl, C₆₋₁₀ aryl, 5-6-membered heteroaryl, L₁-W, and L₂-OH, wherein said C₁₋₆ alkyl, C₆₋₁₀ aryl, and heteroaryl are each optionally substituted with 1 or 2 substituents independently selected from halo, C₁₋₆ alkoxy, C₁₋₆ haloalkoxy, a reactive chemical group, and L₃-X;
L₁ is C₁₋₃ alkylene, optionally substituted with 1 or 2 substituents independently selected from halo, C₁₋₆ alkoxy, C₁₋₆ haloalkoxy, a reactive chemical group, and L₃-X;
W is selected from OH, NH₂, and C(O)OH;
L₂ is C₆₋₁₀ arylene, C₃₋₁₀ cycloalkylene, 4-7-membered heterocycloalkylene, and 5-6-membered heteroarylene, each of which is optionally substituted with 1 or 2 substituents independently selected from halo, C₁₋₆ alkoxy, C₁₋₆ haloalkoxy, a reactive chemical group, and L₃-X;
L₃ is a linker;
X is selected from a fluorophore, a fluorescence quencher, a targeting group, a reactive chemical group, and a biologically active molecule, or a fragment thereof;
provided that
at least one of R¹ and R² is selected from L₁-W and L₂-OH, and the compound of Formula (II) is not any one of the following compounds:

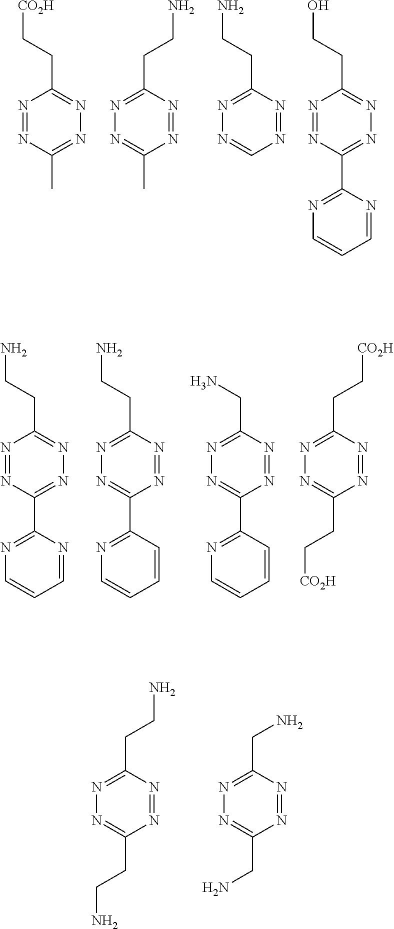

12. The compound of claim 11, having formula: 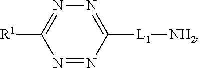
or a pharmaceutically acceptable salt thereof, wherein:
R¹ is selected from C₆₋₁₀ aryl, L₁-OH, L₁-C(O)OH, and L₂-OH.

13. The compound of claim 11, having formula: 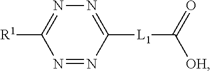
or a pharmaceutically acceptable salt thereof, wherein:
R¹ is selected from H, C₆₋₁₀ aryl, 5-6-membered heteroaryl, L₁-NH₂, L₁-OH, and L₂-OH.

14. The compound of claim 11, having formula: 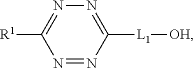
or a pharmaceutically acceptable salt thereof, wherein:
R¹ is selected from H, C₁₋₆ alkyl, C₆₋₁₀ aryl, L₁-OH, L₁-C(O)OH, L₁-NH₂, and L₂-OH.

15. The compound of claim 11, having formula: 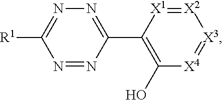
or a pharmaceutically acceptable salt thereof, wherein:
X¹, X², X³, and X⁴ are each independently selected from CH, N, and CR³; and
each R³ is selected from halo, C₁₋₆ alkoxy, C₁₋₆ haloalkoxy, a reactive chemical group, and L₃-X.

16. The compound of claim 15, having formula: 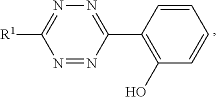
or a pharmaceutically acceptable salt thereof.

17. The compound of claim 15, having formula: 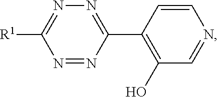
or a pharmaceutically acceptable salt thereof.

18. The compound of claim 15, having formula: 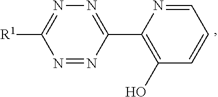
or a pharmaceutically acceptable salt thereof.

19. The compound of any one of claims 15-18, wherein R¹ is selected from C₁₋₆ alkyl, C₆₋₁₀ aryl, and 5-6-membered heteroaryl.

20. The compound of claim 15, having formula: 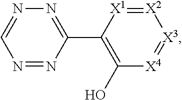
or a pharmaceutically acceptable salt thereof.

21. The compound of claim 15, having formula: 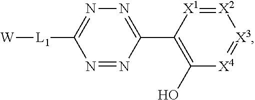
or a pharmaceutically acceptable salt thereof.

22. The compound of claim 15, having formula: 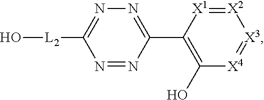
or a pharmaceutically acceptable salt thereof.

23. The compound of claim 11, selected from any one of the following compounds: 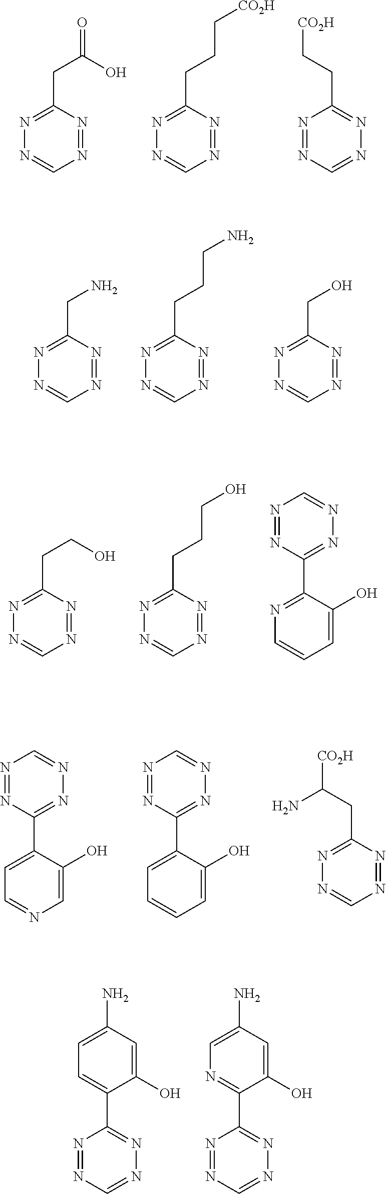
and any one of the compounds depicted in FIG. 36B, or a pharmaceutically acceptable salt thereof.

24. The compound of claim 11, selected from any one of the following compounds: 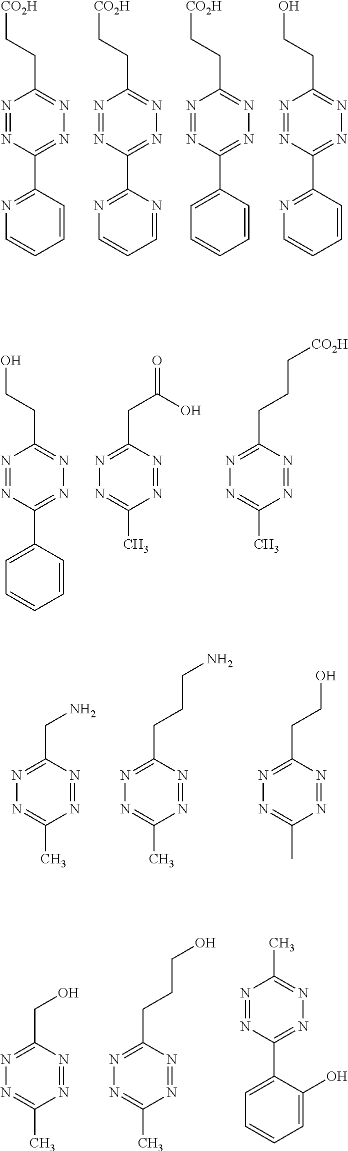 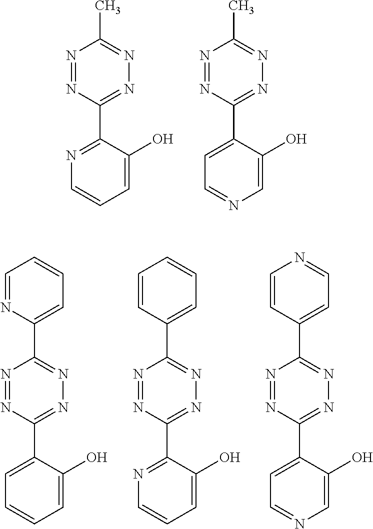 [SMILES: Cc1nnc(-c2ncccc2O)nn1 ; Cc1nnc(-c2ccncc2O)nn1 ; Oc1ccccc1-c1nnc(-c2ccccn2)nn1 ; Oc1cccnc1-c1nnc(-c2ccccc2)nn1 ; Oc1cnccc1-c1nnc(-c2ccncc2)nn1]
or a pharmaceutically acceptable salt thereof.

25. The compound of claim 11, selected from any one of the following compounds: 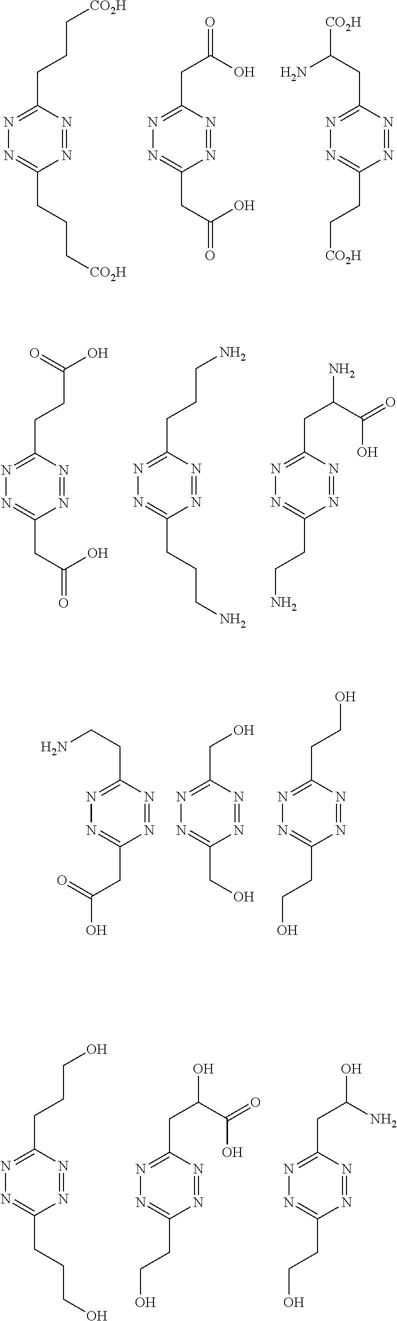 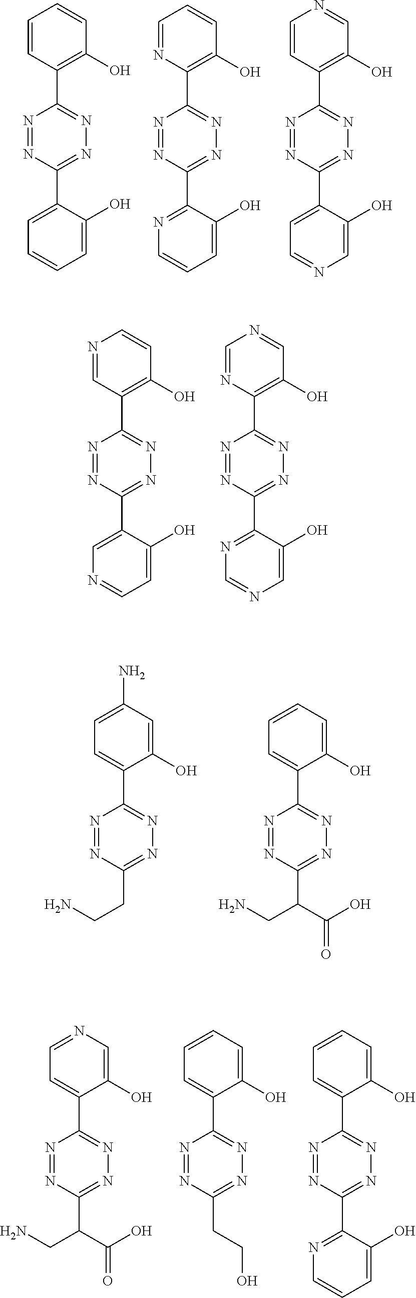
or a pharmaceutically acceptable salt thereof.

26. A conjugate of Formula (III): 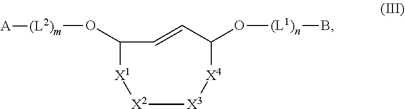
or a pharmaceutically acceptable salt thereof, wherein:
X¹ is selected from O and CHR¹;
X² is selected from O and CHR²;
X³ is selected from O and CHR³;
X⁴ is selected from O and CHR⁴;
R², R³, and R⁴ are each independently selected from H, halo, C₁₋₆ alkyl, C₁₋₆ haloalkyl, C₁₋₃ alkoxy, and C₁₋₃ haloalkoxy;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000;
each R^{N} is independently selected from H, C₁₋₃ alkyl, and C₁₋₃ haloalkyl; and
A and B are each independently selected from an antibody, an antibody fragment, an engineered antibody, a peptide, a biologically active protein, a small-molecule drug, a fluorophore, and a fluorescence quencher.

27. The conjugate of claim 26, having a formula: 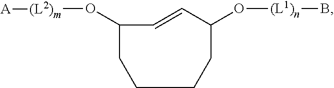
or a pharmaceutically acceptable salt thereof.

28. The conjugate of claim 26, having a formula: 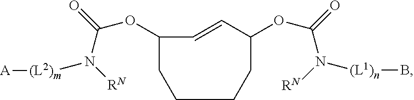
or a pharmaceutically acceptable salt thereof.

29. The conjugate of claim 26, having any one of the following formulae: 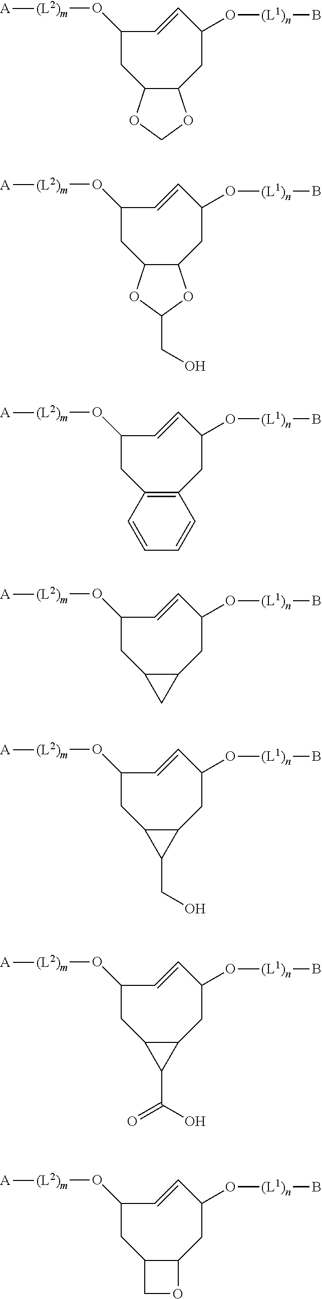 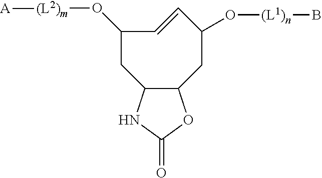
or a pharmaceutically acceptable salt thereof.

30. The conjugate of any one of claims 26-29, wherein:
n is an integer from 1 to 10;
m is an integer from 1 to 10;
A is selected from an antibody, an antibody fragment, an engineered antibody, a peptide, a targeting moiety, and a small-molecule drug; and
B is selected from a fluorophore and a fluorescence quencher.

31. The conjugate of any one of claims 26-29, wherein:
n is an integer from 1 to 10;
m is an integer from 1 to 10;
A is selected from an antibody, an antibody fragment, an engineered antibody, a peptide, and a targeting moiety; and
B is a small-molecule drug.

32. The conjugate of any one of claims 26-29, wherein:
A is an antibody that is specific to an antigen which is a biomarker of a disease or condition; and
B is a fluorophore.

33. The conjugate of any one of claims 26-29, wherein:
A is an antibody that is specific to an antigen which is a biomarker of a disease or condition; and
B is a small-molecule drug useful in treating the disease or condition.

34. The conjugate of claim 26, wherein the conjugate is selected from any one of the conjugates depicted in FIGS. 16, 17, 19, and 36A, or a pharmaceutically acceptable salt thereof.

35. A composition comprising a conjugate of any one of claims 26-34, or a pharmaceutically acceptable salt thereof, and an inert carrier.

36. A pharmaceutical composition comprising a conjugate of any one of claims 26-34, or a pharmaceutically acceptable salt thereof, and a pharmaceutically acceptable carrier.

37. A method of making a compound selected from: 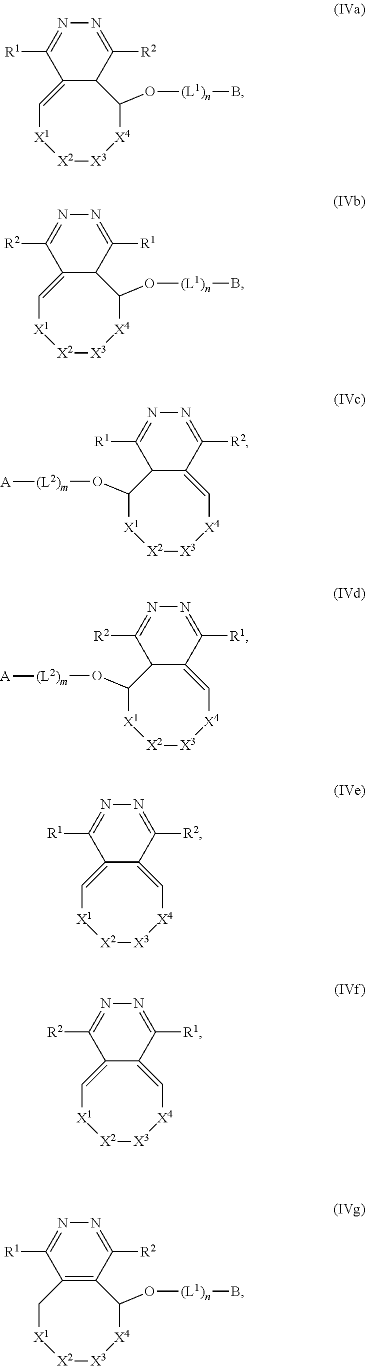 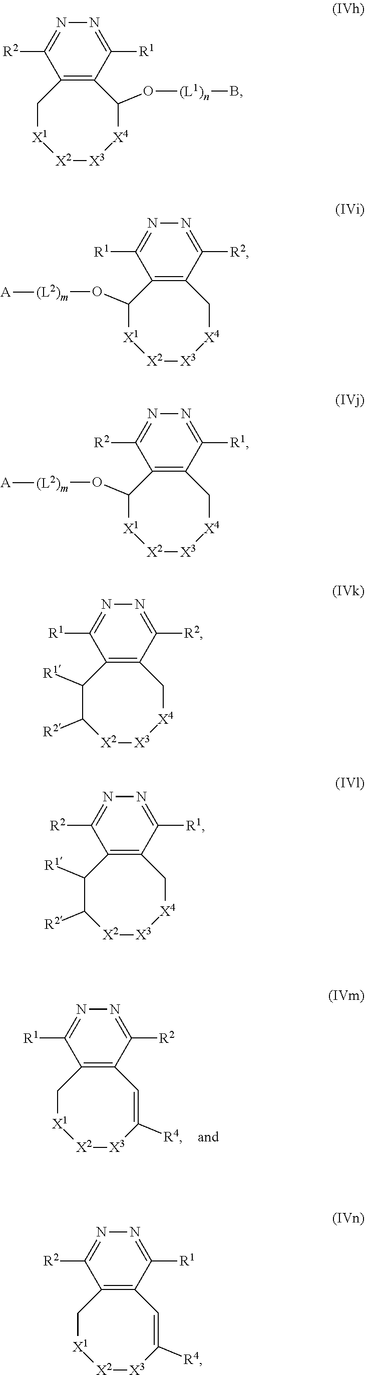
wherein R^{1′} and R^{2′} are as described herein for R¹ and R² of the conjugate of any one of claims 26-34, or a pharmaceutically acceptable salt thereof, or any combination thereof, the method comprising reacting a conjugate of any one of claims 26-34, or a pharmaceutically acceptable salt thereof, with a compound of Formula (II): 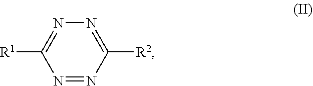
or a pharmaceutically acceptable salt thereof, wherein:
R¹ and R² are each independently selected from H, C₁₋₆ alkyl, C₆₋₁₀ aryl, 5-6-membered heteroaryl, L₁-W, and L₂-OH, wherein said C₁₋₆ alkyl, C₆₋₁₀ aryl, and 5-6-membered heteroaryl are each optionally substituted with 1 or 2 substituents independently selected from halo, C₁₋₆ alkoxy, C₁₋₆ haloalkoxy, a reactive chemical group, and L₃-X;
L₁ is C₁₋₃ alkylene, optionally substituted with 1 or 2 substituents independently selected from halo, C₁₋₆ alkoxy, C₁₋₆ haloalkoxy, a reactive chemical group, and L₃-X;
W is selected from OH, NH₂, and C(O)OH;
L₂ is C₆₋₁₀ arylene, C₃₋₁₀ cycloalkylene, 4-7-membered heterocycloalkylene, and 5-6-membered heteroarylene, each of which is optionally substituted with 1 or 2 substituents independently selected from halo, C₁₋₆ alkoxy, C₁₋₆ haloalkoxy, a reactive chemical group, and L₃-X;
L₃ is a linker;
X is selected from a fluorophore, a fluorescence quencher, a targeting group, a reactive chemical group, and a biologically active molecule, or a fragment thereof.

38. The method of claim 37, wherein reacting the conjugate of any one of claims 26-24, or a pharmaceutically acceptable salt thereof, with the compound of Formula (II), or a pharmaceutically acceptable salt thereof, comprises forming a compound selected from: 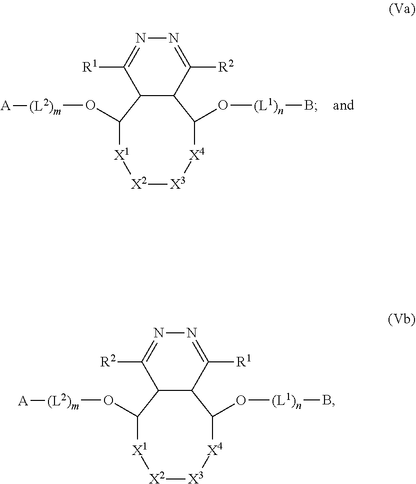
or a combination thereof.

39. The method of claim 38, comprising forming the compound of Formula (Va) or Formula (Vb), or a combination thereof, in vitro, in vivo, or ex vivo.

40. The method of any one of claims 37-39, wherein R¹ and R² are each independently selected from H, C₁₋₆ alkyl, C₆₋₁₀ aryl, 5-6-membered heteroaryl, wherein said C₁₋₆ alkyl, C₆₋₁₀ aryl, and 5-6-membered heteroaryl are each optionally substituted with 1 or 2 substituents independently selected from halo, C₁₋₆ alkoxy, C₁₋₆ haloalkoxy, a reactive chemical group, and L₃-X.

41. The method of claim 40, wherein R¹ and R² are each C₁₋₆ alkyl.

42. The method of claim 40, wherein R¹ and R² are each 5-6-membered heteroaryl.

43. The method of any one of claims 37-39, wherein at least one of R¹ and R² is selected from L₁-W and L₂-OH.

44. The method of any one of claims 37-39, wherein at least one of R¹ and R² is selected from L₁-W and L₂-OH.

45. The method of any one of claims 37-39, wherein both R¹ and R² are independently selected from L₁-W and L₂-OH.

46. A method of making a compound selected from: 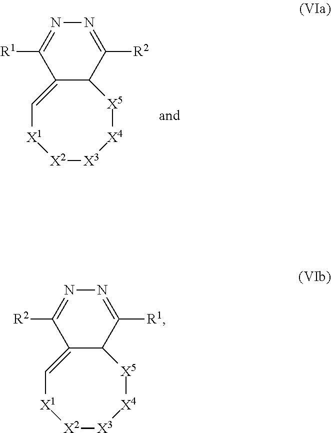
or a pharmaceutically acceptable salt thereof, or a combination thereof, the method comprising reacting a conjugate of Formula (VI): 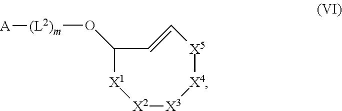
or a pharmaceutically acceptable salt thereof, wherein:
X¹ is selected from O and CHR¹;
X² is selected from O and CHR²;
X³ is selected from O and CHR³;
X⁴ is selected from O and CHR⁴;
X⁵ is selected from O and CHR⁵;
R¹, R², R³, R⁴, and R⁵ are each independently selected from H, halo, C₁₋₆ alkyl, C₁₋₆ haloalkyl, C₁₋₃ alkoxy, C₁₋₃ haloalkoxy, and (L¹)ₙ-B;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L¹)ₙ-B;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000;
each R^{N} is independently selected from H, C₁₋₃ alkyl, and C₁₋₃ haloalkyl; and
A and B are each independently selected from an antibody, an antibody fragment, an engineered antibody, a peptide, a biologically active protein, a small-molecule drug, a fluorophore, and a fluorescence quencher;
with a compound of Formula (II): 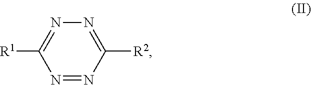
or a pharmaceutically acceptable salt thereof, wherein:
R¹ and R² are each independently selected from L₁-W and L₂-OH;
L₁ is C₁₋₃ alkylene, optionally substituted with 1 or 2 substituents independently selected from halo, C₁₋₆ alkoxy, C₁₋₆ haloalkoxy, a reactive chemical group, and L₃-X;
W is selected from OH, NH₂, and C(O)OH;
L₂ is C₆₋₁₀ arylene, C₃₋₁₀ cycloalkylene, 4-7-membered heterocycloalkylene, and 5-6-membered heteroarylene, each of which is optionally substituted with 1 or 2 substituents independently selected from halo, C₁₋₆ alkoxy, C₁₋₆ haloalkoxy, a reactive chemical group, and L₃-X;
L₃ is a linker;
X is selected from a fluorophore, a fluorescence quencher, a targeting group, a reactive chemical group, and a biologically active molecule, or a fragment thereof;
provided that the compound of Formula (II) is not any one of the following compounds:

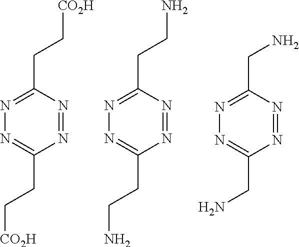

47. The method of claim 46, wherein reacting the conjugate of Formula (VI), or a pharmaceutically acceptable salt thereof, with the compound of Formula (II), or a pharmaceutically acceptable salt thereof, comprises forming a compound selected from: 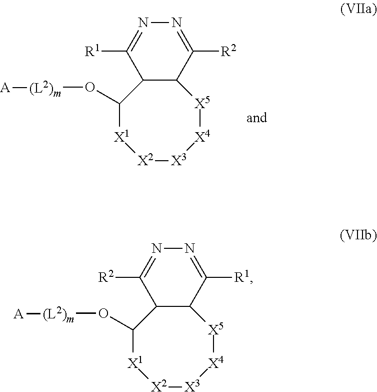
or a combination thereof.

48. The method of claim 47, comprising forming the compound of Formula (VIIa) or Formula (VIIb), or a combination thereof, in vitro, in vivo, or ex vivo.

49. The method of claim 46, wherein the conjugate of Formula (VI) has formula: 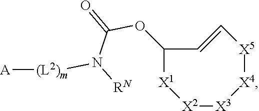
or a pharmaceutically acceptable salt thereof.

50. The method of claim 46, wherein:
A is selected from an antibody, an antibody fragment, an engineered antibody, a peptide, a targeting moiety, and a small-molecule drug; and
B is selected from a fluorophore and a fluorescence quencher.

51. The method of claim 46, wherein:
A is a small-molecule drug; and
B is selected from an antibody, an antibody fragment, an engineered antibody, a peptide, and a targeting moiety.

52. The method of claim 46, wherein:
A is an antibody, an antibody fragment, an engineered antibody, a peptide, and a small-molecule drug; and B is absent.

53. The method of claim 46, wherein the compound of Formula (II) has formula: 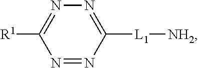
or a pharmaceutically acceptable salt thereof, wherein:
R¹ is selected from L₁-OH, L₁-C(O)OH, and L₂-OH.

54. The method of claim 46, wherein the compound of Formula (II) has formula: 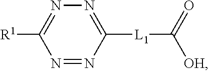
or a pharmaceutically acceptable salt thereof, wherein:
R¹ is selected from L₁-NH₂, L₁-OH, and L₂-OH.

55. The method of claim 46, wherein the compound of Formula (II) has formula: 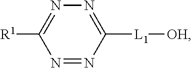
or a pharmaceutically acceptable salt thereof, wherein:
R¹ is selected from L₁-OH, L₁-C(O)OH, L₁-NH₂, and L₂-OH.

56. The method of claim 46, wherein the compound of Formula (II) has formula: 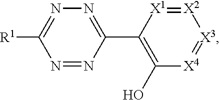
or a pharmaceutically acceptable salt thereof, wherein:
X², X³, and X⁴ are each independently selected from CH, N, and CR³; and
each R³ is selected from halo, C₁₋₆ alkoxy, C₁₋₆ haloalkoxy, a reactive chemical group, and L₃-X.

57. A biologically active molecule comprising moiety of formula (i): 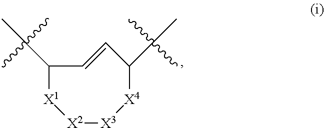
or a pharmaceutically acceptable salt thereof, wherein:
X¹ is selected from O and CHR¹;
X² is selected from O and CHR²;
X³ is selected from O and CHR³;
X⁴ is selected from O and CHR⁴;
R¹, R², R³, and R⁴ are each independently selected from H, halo, C₁₋₆ alkyl, C₁₋₆ haloalkyl, C₁₋₃ alkoxy, and C₁₋₃ haloalkoxy;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, and NH₂—C₁₋₃ alkylene.

58. A biologically active molecule comprising a moiety of formula: 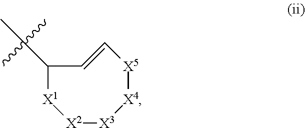
or a pharmaceutically acceptable salt thereof, wherein:
X¹ is selected from O and CHR¹;
X² is selected from O and CHR²;
X³ is selected from O and CHR³;
X⁴ is selected from O and CHR⁴;
X⁵ is selected from O and CHR⁵;
R¹, R², R³, R⁴, and R⁵ are each independently selected from H, halo, C₁₋₆ alkyl, C₁₋₆ haloalkyl, C₁₋₃ alkoxy, C₁₋₃ haloalkoxy, and a moiety of formula (iii): 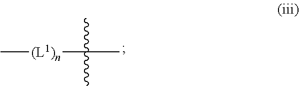
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (iii);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (iii);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (iii);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (iii);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (iii);
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each x is independently an integer from 1 to 2,000; and
each R^{N} is independently selected from H, C₁₋₃ alkyl, and C₁₋₃ haloalkyl, provided that the moiety of formula (ii) comprises at least one moiety of formula (iii).

59. The biologically active molecule of claim 57 or 58, which is a peptide or a protein.

60. The biologically active molecule of claim 59, wherein the peptide or the protein, prior to incorporation of the moiety of formula (i) or formula (ii) into its chemical structure, is selected from bivalirudin, a madanin, a haemathrin, a variegin, an integrilin, and a cytokine.

61. The biologically active molecule of claim 57 or 58, selected from any one of the biologically active molecules depicted in FIGS. 29E, 29F, and 34, or a pharmaceutically acceptable salt thereof.

62. A method of modulating activity of a biologically active molecule of formula (i) as recited in claim 57 or formula (ii) as recited in claim 58, the method comprising reacting the biologically active molecule with a compound of Formula (II): 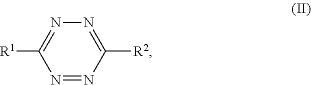
or a pharmaceutically acceptable salt thereof, wherein:
R¹ and R² are each independently selected from H, C₁₋₆ alkyl, C₆₋₁₀ aryl, 5-6-membered heteroaryl, L₁-W, and L₂-OH, wherein said C₁₋₆ alkyl, C₆₋₁₀ aryl, and heteroaryl are each optionally substituted with 1 or 2 substituents independently selected from halo, C₁₋₆ alkoxy, and C₁₋₆ haloalkoxy;
L₁ is C₁₋₃ alkylene, optionally substituted with 1 or 2 substituents independently selected from halo, C₁₋₆ alkoxy, and C₁₋₆ haloalkoxy;
W is selected from OH, NH₂, and C(O)OH; and
L₂ is C₆₋₁₀ arylene, C₃₋₁₀ cycloalkylene, 4-7-membered heterocycloalkylene, and 5-6-membered heteroarylene, each of which is optionally substituted with 1 or 2 substituents independently selected from halo, C₁₋₆ alkoxy, and C₁₋₆ haloalkoxy.

63. The method of claim 62, wherein reacting the biologically active compound, or a pharmaceutically acceptable salt thereof, with the compound of Formula (II), or a pharmaceutically acceptable salt thereof, comprises forming a compound selected from: 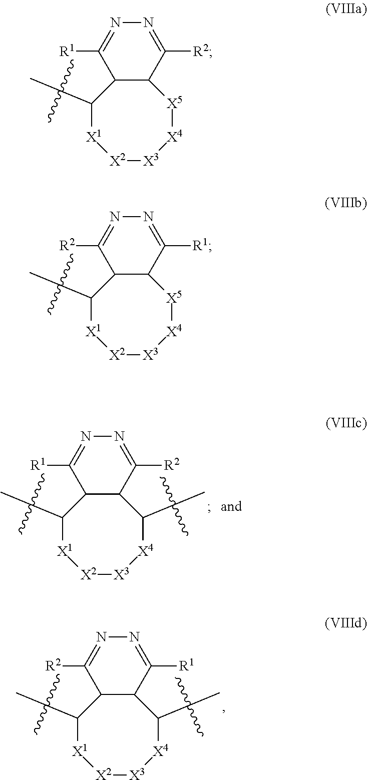
or a combination thereof.

64. The method of claim 63, wherein comprising forming the compound of any one of the Formulae (VIIIa)-(VIIId), or a combination thereof, in vitro, in vivo, or ex vivo.

65. The method of claim 62, wherein reacting the biologically active molecule with a compound of Formula (II) results in decrease or loss of biological function of the biologically active molecule.
